(19) United States
(12) Patent Application Publication
(10) Pub. No.: US 2024/0117002 A1
(43) Pub. Date: Apr. 11, 2024

(54) COMPOSITIONS AND METHODS FOR TCR REPROGRAMMING USING FUSION PROTEINS

(71) Applicant: TCR2 THERAPEUTICS Inc., Cambridge, MA (US)

(72) Inventors: Robert TIGHE, Cambridge, MA (US); Pooja ARORA, Cambridge, MA (US); Robert HOFMEISTER, Scituate, MA (US); Dario GUTIERREZ, Waltham, MA (US); Derrick MCCARTHY, Cambridge, MA (US); Michelle FLEURY, Arlington, MA (US)

(21) Appl. No.: 18/269,214

(22) PCT Filed: Dec. 23, 2021
(86) PCT No.: PCT/US21/65069

§ 371 Date: Jun. 22, 2023

Related U.S. Application Data
(60) Provisional application No. 63/129,829, filed on Dec. 23, 2020.
(60) Provisional application No. 63/225,821, filed on Jul. 26, 2021.
(60) Provisional application No. 63/250,329, filed on Sep. 30, 2021.
(60) Provisional application No. 63/257,345, filed on Oct. 19, 2021.

(51) Int. Cl. C07K 14/725; A61K 39/00; A61P 35/00; C07K 14/54; C07K 14/705; C07K 14/715; C07K 16/18; C07K 16/28

(52) U.S. Cl. CPC C07K 14/7051; A61K 39/4611; A61K 39/4632; A61K 39/4633; A61P 35/00; C07K 14/5443; C07K 14/70521; C07K 14/7155; C07K 16/18; C07K 16/2803; A61K 38/00; C07K 2319/02; C07K 2319/03; C07K 2319/50

(57) ABSTRACT

Provided herein are recombinant nucleic acids encoding a T cell receptor (TCR) fusion protein (TFP) and an interleukin-15 (IL-15) polypeptide and/or an interleukin-15 receptor alpha (IL-15Rα) polypeptide, modified T cells expressing the encoded molecules, and methods of use thereof for the treatment of diseases, including cancer.

### CROSS-REFERENCE

This application claims the benefit of U.S. Provisional Application 63/129,829, filed Dec. 23, 2020, U.S. Provisional Application 63/225,821, filed Jul. 26, 2021, U.S. Provisional Application 63/250,329, filed Sep. 30, 2021, and U.S. Provisional Application 63/257,345, filed Oct. 19, 2021, each of which is incorporated herein by reference in its entirety.

### BACKGROUND OF THE INVENTION

Most patients with late-stage solid tumors are incurable with standard therapy. In addition, traditional treatment options often have serious side effects. Numerous attempts have been made to engage a patient's immune system for rejecting cancerous cells, an approach collectively referred to as cancer immunotherapy. However, several obstacles make it rather difficult to achieve clinical effectiveness. Although hundreds of so-called tumor antigens have been identified, these are often derived from self and thus can direct the cancer immunotherapy against healthy tissue or are poorly immunogenic. Furthermore, cancer cells use multiple mechanisms to render themselves invisible or hostile to the initiation and propagation of an immune attack by cancer immunotherapies.

Human T cell therapies rely on enriched or modified human T cells to target and kill cancer cells in a patient. To increase the ability of T cells to target and kill a particular cancer cell, methods have been developed to engineer T cells to express constructs which direct T cells to a particular target cancer cell. Chimeric antigen receptors (CARs) and engineered T cell receptors (TCRs), which comprise binding domains capable of interacting with a particular tumor antigen, allow T cells to target and kill cancer cells that express the particular tumor antigen.

Besides the ability of genetically modified T cells expressing a CAR or an engineered TCR to recognize and destroy respective target cells in vitro/ex vivo, successful patient therapy with engineered T cells may require the T cells to be capable of strong activation, expansion, persistence overtime, effective tumor targeting, and, in case of relapsing disease, enabling a ‘memory’ response.

### SUMMARY OF THE INVENTION

There is a clear need to improve genetically engineered T cells to more broadly act against various human malignancies and to enhance longevity of genetically engineered T cells to generate durable responses in cancer patients.

Described herein are recombinant nucleic acids expressing fusion proteins of TCR subunits, including CD3 epsilon, CD3gamma, CD3 delta, TCR gamma, TCR delta, TCR alpha and TCR beta chains with binding domains specific for cell surface antigens that have the potential to overcome limitations of existing approaches. Said nucleic acid molecules can express a second protein that comprises IL-15, or a fragment thereof, or IL-15-Rα, or a fragment thereof. In some embodiments, said IL-15-Rα fusion protein comprises the extracellular domain of PD-1 fused to the intracellular domain of CD28, with the intracellular domain of IL-15-Rα fused to the C-terminus of the intracellular domain of CD28. Said recombinant nucleic acids can be expressed in a T cell. Advantageously, when these IL-15 and/or IL-15-Rα proteins are expressed in combination with the TCR fusion proteins described above, they can confer increased persistence, prolonged activity, and increased efficacy on the T cells for treating the malignancies described herein. These modified T cells may have the ability to kill target cells more efficiently than CARs but release comparable or lower levels of pro-inflammatory cytokines. These modified T cells and methods of their use may represent an advantage for these cells relative to CARs because elevated levels of these cytokines have been associated with dose-limiting toxicities for adoptive CAR T therapies.

Provided herein are modified T cells comprising T-cell receptor (TCR) fusion protein (TFP) and an IL-15 or IL-15Rα polypeptide, methods of producing the modified T cells, and methods of use thereof for the treatment of diseases.

In some aspects, provided herein is a recombinant nucleic acid molecule comprising a first nucleic acid sequence encoding a T cell receptor (TCR) fusion protein (TFP) wherein the TFP comprises a TCR subunit comprising at least a portion of a TCR extracellular domain, and a TCR transmembrane domain, and an antigen binding domain; and wherein the TCR subunit and the antigen binding domain are operatively linked, and a second nucleic acid sequence encoding an interleukin-15 (IL-15) polypeptide or a fragment thereof.

In some embodiments, the TFP further comprise a TCR intracellular signaling domain. In some embodiments, the first nucleic acid sequence and the second nucleic acid sequence are operatively linked by a first linker. In some embodiments, the first linker comprises a protease cleavage site. In some embodiments, the protease cleavage site is a 2A cleavage site.

In some embodiments, the IL-15 polypeptide is secreted when expressed in a T cell. In some embodiments, the IL-15 polypeptide comprises a sequence of SEQ ID NO: 375. In some embodiments, the second nucleic acid sequence further encodes an IL-15 receptor (IL-15R) subunit or a fragment thereof. In some embodiments, the IL-15R subunit is IL-15R alpha (IL-15Ra). In some embodiments, IL-15 and IL-15Rα are operatively linked by a second linker. In some embodiments, the second linker is not a cleavable linker. In some embodiments, the second linker comprises a sequence comprising (G₄S)ₙ, wherein G is glycine, S is seine, and n is an integer from 1 to 10. In some embodiments, n is an integer from 1 to 4. In some embodiments, n is 3. In some embodiments, the second linker comprises a sequence of SEQ ID NO: 378. In some embodiments, the second linker comprises a sequence of SEQ ID NO: 405.

In some embodiments, the second nucleic acid sequence encodes a fusion protein comprising the IL-15 polypeptide linked to the IL-15Rα subunit. In some embodiments, the IL-15 polypeptide is linked to N-terminus of the IL-15Rα subunit. In some embodiments, the fusion protein comprises amino acids 30-162 of IL-15. In some embodiments, the fusion protein comprises amino acids 31-267 of IL-15Ra. In some embodiments, the fusion protein further comprises a sushi domain. In some embodiments, the fusion protein comprises a sequence of SEQ ID NO: 389.

In some embodiments, the fusion protein is expressed on cell surface when expressed in a T cell. In some embodiments, the fusion protein is secreted when expressed in a T cell.

In some embodiments, the recombinant nucleic acid molecule further comprises a third nucleic acid sequence encoding a PD-1 polypeptide which is operably linked via its C-terminus to the N-terminus of an intracellular domain of a costimulatory polypeptide. In some embodiments, the recombinant nucleic acid molecule further comprises a third nucleic acid sequence encoding an anti-PD-1 antibody, or antigen binding fragment thereof, which is operably linked via its C-terminus to the N-terminus of an intracellular domain of a costimulatory polypeptide. In some embodiments, the PD-1 polypeptide or anti-PD-1 antibody is linked to the intracellular domain of the costimulatory polypeptide via the transmembrane domain of PD-1. In some embodiments, the costimulatory poly-peptide is selected from the group consisting of OX40, CD2, CD27, CDS, ICAM-1, ICOS (CD278), 4-1BB (CD137), GITR, CD28, CD30, CD40, BAFFR, HVEM, CD7, LIGHT, NKG2C, SLAMF7, NKp80, CD160, CD226, FcγRI, FcγRII, and FcγRIII.

In some embodiments, the recombinant nucleic acid molecule comprises a sequence of SEQ ID NO: 377, SEQ ID NO: 380, or SEQ ID NO: 381.

In some aspects, provided herein is a recombinant nucleic acid molecule comprising a first nucleic acid sequence encoding a T cell receptor (TCR) fusion protein (TFP) wherein the TFP comprises a TCR subunit comprising at least a portion of a TCR extracellular domain, and a TCR transmembrane domain, and an antigen binding domain and wherein the TCR subunit and the antigen binding domain are operatively linked, and a second nucleic acid sequence encoding an interleukin-15 receptor alpha (IL-15Ra) polypeptide or a fragment thereof.

In some embodiments, the first nucleic acid sequence and the second nucleic acid sequence are operatively linked by a first linker. In some embodiments, the first linker comprises a protease cleavage site. In some embodiments, the protease cleavage site is a 2A cleavage site.

In some embodiments, the second nucleic acid sequence further encodes PD-1 or a fragment thereof. In some embodiments, the second nucleic acid sequence encodes the extracellular domain of PD-1. In some embodiments, the second nucleic acid sequence encodes the extracellular and transmembrane domain of PD-1. In some embodiments, the second nucleic acid sequence further encodes CD28 or a fragment thereof. In some embodiments, the second nucleic acid sequence encodes the intracellular domain of CD28. In some embodiments, the second nucleic acid sequence encodes a fusion protein comprising the PD-1 extracellular domain and transmembrane domain linked to the CD28 intracellular domain linked to IL-15Ra. In some embodiments, the CD28 intracellular domain is linked to the intracellular domain of IL-15Ra. In some embodiments, the second nucleic acid sequence comprises a sequence of SEQ ID NO: 390.

In some embodiments, the recombinant nucleic acid molecule further comprises a third nucleic acid sequence encoding an interleukin-15 (IL-15) polypeptide or a fragment thereof. In some embodiments, the IL-15 polypeptide or a fragment thereof is secreted when expressed in a T cell.

In some embodiments, the recombinant nucleic acid molecule comprises a sequence of SEQ ID NO: 361, SEQ ID NO: 376, SEQ ID NO: 377, or SEQ ID NO: 381.

In some embodiments, the TFP functionally interacts with an endogenous TCR complex when expressed in a T cell. In some embodiments, the TCR intracellular do-main comprises a stimulatory domain from an intracellular signaling domain of CD3 gamma, CD3 delta, or CD3 epsilon. In some embodiments, the TCR intracellular domain comprises an intracellular domain from TCR alpha, TCR beta, TCR gamma, or TCR delta. In some embodiments, the antigen binding domain is connected to the TCR extracellular domain by a third linker sequence. In some embodiments, the third linker is 120 amino acids in length or less. In some embodiments, the third linker sequence comprises (G₄S)ₙ, wherein G is glycine, S is serine, and n is an integer from 1 to 10. In some embodiments, n is an integer from 1 to 4.

In some embodiments, at least two of the TCR extracellular domain, the TCR transmembrane domain, and the TCR intracellular domain are from the same TCR subunit. In some embodiments, at least two of the TCR extracellular domain, the TCR transmembrane domain, and the TCR intracellular domain are from TCR alpha. In some embodiments, at least two of the TCR extracellular domain, the TCR transmembrane domain, and the TCR intracellular domain are from TCR beta. In some embodiments, at least two of the TCR extracellular domain, the TCR transmembrane domain, and the TCR intracellular domain are from TCR gamma. In some embodiments, at least two of the TCR extracellular domain, the TCR transmembrane domain, and the TCR intracellular domain are from TCR delta. In some embodiments, at least two of the TCR extracellular domain, the TCR transmembrane domain, and the TCR intracellular domain are from CD3 epsilon. In some embodiments, at least two of the TCR extracellular domain, the TCR transmembrane domain, and the TCR intracellular domain are from CD3 delta. In some embodiments, at least two of the TCR extracellular domain, the TCR transmembrane domain, and the TCR intracellular domain are from CD3 gamma.

In some embodiments, all three of the TCR extracellular domain, the TCR transmembrane domain, and the TCR intracellular domain are from the same TCR subunit. In some embodiments, the TCR extracellular domain, the TCR transmembrane domain, and the TCR intracellular domain are from CD3 epsilon. In some embodiments, the TCR extracellular domain, the TCR transmembrane domain, and the TCR intracellular domain are from CD3 delta. In some embodiments, the TCR extracellular domain, the TCR transmembrane domain, and the TCR intracellular domain are from CD3 gamma. In some embodiments, the TCR extracellular domain, the TCR transmembrane domain, and the TCR intracellular domain comprise or consist of the constant domain of TCR alpha. In some embodiments, the constant domain of TCR alpha is murine. In some embodiments, the TCR extracellular domain, the TCR transmembrane domain, and the TCR intracellular domain comprise or consist of the constant domain of TCR beta. In some embodiments, the constant domain of TCR beta is murine. In some embodiments, the TCR extracellular domain, the TCR transmembrane domain, and the TCR intracellular domain comprise or consist of the constant domain of TCR gamma. In some embodiments, the TCR extracellular domain, the TCR transmembrane domain, and the TCR intracellular domain comprise or consist of the constant domain of TCR delta.

In some embodiments, the antigen binding domain is a camelid antibody or binding fragment thereof. In some embodiments, the antigen binding domain is a murine antibody or binding fragment thereof. In some embodiments, the antigen binding domain is a human or humanized antibody or binding fragment thereof. In some embodiments, the antigen binding domain is a single-chain variable fragment (scFv) or a single domain antibody (sdAb) domain. In some embodiments, the antigen binding domain is a single domain antibody (sdAb). In some embodiments, the sdAb is a V_{HH}. In some embodiments, the antigen binding do-main is selected from the group consisting of an anti-CD19 binding domain, an anti-B-cell maturation antigen (BCMA) binding domain, and an anti-mesothelin (MSLN) binding domain, an anti-CD20 binding domain, an anti-CD70 binding domain, anti-MUC16 binding domain, an anti-Nectin-4 binding domain, an anti-GPC3 binding domain, and an anti-TROP-2 binding domain.

In some embodiments, a T cell expressing the TFP inhibits tumor growth when expressed in a T cell. In some embodiments, recombinant nucleic acid molecule described herein further comprise a leader sequence. In some embodiments, the recombinant nucleic acid molecule is selected from the group consisting of a DNA and an RNA. In some embodiments, the recombinant nucleic acid molecule is an mRNA. In some embodiments, the recombinant nucleic acid molecule is a circRNA. In some embodiments, the recombinant nucleic acid molecule comprises a nucleotide analog. In some embodiments, the nucleotide analog is selected from the group consisting of 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA) modified, a locked recombinant nucleic acid molecule (LNA), an ethylene recombinant nucleic acid molecule (ENA), a peptide recombinant nucleic acid molecule (PNA), a 1′,5′-anhydrohexitol recombinant nucleic acid molecule (HNA), a morpholino, a methylphosphonate nucleotide, a thiolphosphonate nucleotide, and a 2′-fluoro N3-P5′-phosphoramidite. In some embodiments, recombinant nucleic acid molecule described herein further comprise a promoter. In some embodiments, the recombinant nucleic acid molecule is an in vitro transcribed nucleic acid. In some embodiments, the recombinant nucleic acid molecule further comprises a sequence encoding a poly(A) tail. In some embodiments, the recombinant nucleic acid molecule further comprises a 3′UTR sequence.

In some aspects, provided herein is a polypeptide encoded by the recombinant nucleic acid molecule disclosed herein. In some aspects, provided herein is a vector comprising the recombinant nucleic acid molecule disclosed herein. In some aspects, provided herein is a cell comprising the recombinant nucleic acid molecule disclosed herein, the polypeptide disclosed herein, or the vector disclosed herein. In some embodiments, the cell is a T cell. In some embodiments, the T cell is a human T cell. In some embodiments, the T cell is a CD8+ or CD4+ T cell. In some embodiments, the T cell is a human αβ T cell. In some embodiments, the T cell is a human γδ T cell. In some embodiments, the cell is a human NKT cell. In some embodiments, the cell is an allogeneic cell or an autologous cell.

In some aspects, provided herein is a cell comprising a first nucleic acid sequence encoding a T cell receptor (TCR) fusion protein (TFP) wherein the TFP comprises a TCR subunit comprising at least a portion of a TCR extracellular domain, and a TCR transmembrane domain, and an antigen binding domain; and wherein the TCR subunit and the antigen binding domain are operatively linked, and a second nucleic acid sequence encoding an interleukin-15 (IL-15) polypeptide or a fragment thereof.

In some embodiments, the first nucleic acid sequence and the second nucleic acid sequence are included in a single nucleic acid molecule. In some embodiments, the first nucleic acid sequence and the second nucleic acid sequence are included in two separate nucleic acid molecules. In some embodiments, the first nucleic acid sequence and the second nucleic acid sequence are operatively linked by a first linker. In some embodiments, the first linker comprises a protease cleavage site. In some embodiments, the protease cleavage site is a 2A cleavage site.

In some embodiments, the TFP further comprise a TCR intracellular signaling domain. In some embodiments, the IL-15 polypeptide is secreted when expressed in a T cell. In some embodiments, the IL-15 polypeptide comprises a sequence of SEQ ID NO: 375. In some embodiments, the second nucleic acid sequence further encodes an IL-15 receptor (IL-15R) subunit or a fragment thereof. In some embodiments, the IL-15R subunit is IL-15R alpha (IL-15Ra). In some embodiments, IL-15 and IL-15Rα are operatively linked by a second linker. In some embodiments, the second linker is not a cleavable linker. In some embodiments, the second linker comprises a sequence comprising (G₄S)ₙ, wherein G is glycine, S is serine, and n is an integer from 1 to 10. In some embodiments, n is an integer from 1 to 4. In some embodiments, n is 3. In some embodiments, the second linker comprises a sequence of SEQ ID NO: 378. In some embodiments, the second linker comprises a sequence of SEQ ID NO: 405.

In some embodiments, the second nucleic acid sequence encodes a fusion protein comprising the IL-15 polypeptide linked to the IL-15Rα subunit. In some embodiments, the IL-15 polypeptide linked to N-terminus of the IL-15Rα subunit. In some embodiments, the fusion protein comprises amino acids 30-162 of IL-15. In some embodiments, the fusion protein comprises amino acids 31-267 of IL-15Ra. In some embodiments, the fusion protein further comprises a sushi domain. In some embodiments, the fusion protein comprises a sequence of SEQ ID NO: 389.

In some embodiments, the fusion protein is expressed on cell surface when expressed in a T cell. In some embodiments, the fusion protein is secreted when expressed in a T cell.

In some embodiments, the cell further comprises a third nucleic acid sequence encoding a PD-1 polypeptide which is operably linked via its C-terminus to the N-terminus of an intracellular domain of a costimulatory polypeptide. In some embodiments, the cell further comprises a third nucleic acid sequence encoding an anti-PD-1 antibody, or antigen binding fragment thereof, which is operably linked via its C-terminus to the N-terminus of an intracellular domain of a costimulatory polypeptide. In some embodiments, the third nucleic acid sequence is included in the same nucleic acid molecule as the first and second nucleic acid sequences.

In some embodiments, the PD-1 polypeptide or anti-PD-1 antibody is linked to the intracellular domain of the costimulatory polypeptide via the transmembrane do-main of PD-1. In some embodiments, the costimulatory polypeptide is chosen from a group comprising OX40, CD2, CD27, CDS, ICAM-1, ICOS (CD278), 4-1BB (CD137), GITR, CD28, CD30, CD40, BAFFR, HVEM, CD7, LIGHT, NKG2C, SLAMF7, NKp80, CD160, CD226, FcγRI, FcγRII, and FcγRIII.

In some aspects, provided herein is a cell comprising a first nucleic acid sequence encoding a T cell receptor (TCR) fusion protein (TFP) wherein the TFP comprises a TCR subunit comprising at least a portion of a TCR extracellular domain, and a TCR transmembrane domain, and an antigen binding domain; and wherein the TCR subunit and the antigen binding domain are operatively linked, and a second nucleic acid sequence encoding an interleukin-15 receptor alpha (IL-15Ra) polypeptide or a fragment thereof.

In some embodiments, the first nucleic acid sequence and the second nucleic acid sequence are included in a single nucleic acid molecule. In some embodiments, the first nucleic acid sequence and the second nucleic acid sequence are included in two separate nucleic acid molecules. In some embodiments, the first nucleic acid sequence and the second nucleic acid sequence are operatively linked by a first linker. In some embodiments, the first linker comprises a protease cleavage site. In some embodiments, the protease cleavage site is a 2A cleavage site.

In some embodiments, the second nucleic acid sequence further encodes PD-1 or a fragment thereof. In some embodiments, the second nucleic acid sequence encodes the extracellular domain of PD-1. In some embodiments, the second nucleic acid sequence encodes the extracellular and trans-membrane domain of PD-1. In some embodiments, the second nucleic acid sequence further encodes CD28 or a fragment thereof. In some embodiments, the second nucleic acid sequence encodes the intracellular domain of CD28. In some embodiments, the second nucleic acid sequence encodes a fusion protein comprising the PD-1 extracellular domain and transmembrane domain linked to the CD28 intracellular domain linked to IL-15Ra. In some embodiments, the CD28 intracellular domain is linked to the intracellular domain of IL-15Ra. In some embodiments, the second nucleic acid sequence comprises a sequence of SEQ ID NO: 390.

In some embodiments, the cell further comprises a third nucleic acid sequence encoding an interleukin-15 (IL-15) polypeptide or a fragment thereof. In some embodiments, the IL-15 polypeptide or a fragment thereof is secreted when expressed in a T cell.

In some embodiments, the cell secretes the IL-15 polypeptide in response to a T cell activation agent. In some embodiments, IL-15 signaling is increased in response to a T cell activation agent. In some embodiments, the T cell activation agent comprises anti-CD3 antibody or a fragment thereof, anti-CD28 antibody or a fragment thereof, a cytokine, an antigen that binds the antigen binding domain of the TFP, or any combinations thereof. In some embodiments, the cell has enhanced survival rate compared with a cell not expressing the IL-15 or IL-15Rα polypeptide. In some embodiments, the cell has enhanced effector function compared with a cell not expressing the IL-15 or IL-15Rα polypeptide. In some embodiments, the cell has enhanced cytotoxicity compared with a cell not expressing the IL-15 or IL-15Rα polypeptide.

In some embodiments, the TFP functionally interacts with an endogenous TCR complex when expressed in a T cell. In some embodiments, the TCR intracellular domain comprises a stimulatory domain from an intracellular signaling domain of CD3 gamma, CD3 delta, or CD3 epsilon. In some embodiments, the TCR intracellular domain comprises an intracellular domain from TCR alpha, TCR beta, TCR gamma, or TCR delta. In some embodiments, the antigen binding domain is connected to the TCR extracellular domain by a third linker sequence. In some embodiments, the third linker is 120 amino acids in length or less. In some embodiments, the third linker sequence comprises (G₄S)ₙ, wherein G is glycine, S is serine, and n is an integer from 1 to 10. In some embodiments, n is an integer from 1 to 4.

In some embodiments, at least two of the TCR extracellular domain, the TCR transmembrane domain, and the TCR intracellular domain are from the same TCR subunit. In some embodiments, at least two of the TCR extracellular domain, the TCR transmembrane domain, and the TCR intracellular domain are from TCR alpha. In some embodiments, at least two of the TCR extracellular domain, the TCR transmembrane domain, and the TCR intracellular domain are from TCR beta. In some embodiments, at least two of the TCR extracellular domain, the TCR transmembrane domain, and the TCR intracellular domain are from TCR gamma. In some embodiments, at least two of the TCR extracellular domain, the TCR transmembrane domain, and the TCR intracellular domain are from TCR delta. In some embodiments, at least two of the TCR extracellular domain, the TCR transmembrane domain, and the TCR intracellular domain are from CD3 epsilon. In some embodiments, at least two of the TCR extracellular domain, the TCR transmembrane domain, and the TCR intracellular domain are from CD3 delta. In some embodiments, at least two of the TCR extracellular domain, the TCR transmembrane domain, and the TCR intracellular domain are from CD3 gamma.

In some embodiments, all three of the TCR extracellular domain, the TCR transmembrane domain, and the TCR intracellular domain are from the same TCR subunit. In some embodiments, the TCR extracellular domain, the TCR transmembrane domain, and the TCR intracellular domain are from CD3 epsilon. In some embodiments, the TCR extracellular domain, the TCR transmembrane domain, and the TCR intracellular domain are from CD3 delta. In some embodiments, the TCR extracellular domain, the TCR transmembrane domain, and the TCR intracellular domain are from CD3 gamma. In some embodiments, the TCR extracellular domain, the TCR transmembrane domain, and the TCR intracellular domain comprise or consist of the constant domain of TCR alpha. In some embodiments, the constant domain of TCR alpha is murine. In some embodiments, the TCR extracellular domain, the TCR transmembrane domain, and the TCR intracellular domain comprise or consist of the constant domain of TCR beta. In some embodiments, the constant domain of TCR beta is murine. In some embodiments, the TCR extracellular domain, the TCR transmembrane domain, and the TCR intracellular domain comprise or consist of the constant domain of TCR gamma. In some embodiments, the TCR extracellular domain, the TCR transmembrane domain, and the TCR intracellular domain comprise or consist of the constant domain of TCR delta.

In some embodiments, the antigen binding domain is a camelid antibody or binding fragment thereof. In some embodiments, the antigen binding domain is a murine antibody or binding fragment thereof. In some embodiments, the antigen binding domain is a human or humanized antibody or binding fragment thereof. In some embodiments, the antigen binding domain is a single-chain variable fragment (scFv) or a single domain antibody (sdAb) domain. In some embodiments, the antigen binding domain is a single domain antibody (sdAb). In some embodiments, the sdAb is a V_{HH}. In some embodiments, the antigen binding domain is selected from the group consisting of an anti-CD19 binding domain, an anti-B-cell maturation antigen (BCMA) binding do-main, and an anti-mesothelin (MSLN) binding domain, an anti-CD20 binding domain, an anti-CD70 binding domain, anti-MUC16 binding domain, an anti-Nectin-4 binding domain, an anti-GPC3 binding domain, and an anti-TROP-2 binding domain.

In some embodiments, the longevity of the cell is increased compared to a cell that does not comprise (i) a nucleic acid sequence encoding an interleukin-15 (IL-15) polypeptide or a fragment thereof or (ii) a nucleic acid sequence encoding an interleukin-15 receptor alpha (IL-15Ra) polypeptide or a fragment thereof. In some embodiments, the persistence of the cell is increased compared to a cell that does not comprise (i) a nucleic acid sequence encoding an interleukin-15 (IL-15) polypeptide or a fragment thereof or (ii) a nucleic acid sequence encoding an interleukin-15 receptor alpha (IL-15Ra) polypeptide or a fragment thereof. In some embodiments, the cytotoxicity of the cell is increased compared to a cell that does not comprise (i) a nucleic acid sequence encoding an interleukin-15 (IL-15) polypeptide or a fragment thereof or (ii) a nucleic acid sequence encoding an interleukin-15 receptor alpha (IL-15Ra) polypeptide or a fragment thereof. In some embodiments, the cytokine production of the cell is increased com-pared to a cell that does not comprise (i) a nucleic acid sequence encoding an interleukin-15 (IL-15) polypeptide or a fragment thereof or (ii) a nucleic acid sequence encoding an interleukin-15 receptor alpha (IL-15Ra) polypeptide or a fragment thereof. In some embodiments, the cell retains a naïve and/or central memory phenotype. In some embodiments, the cell has not differentiated into a terminal effector cell.

In some embodiments, proliferation of the cell is increased compared to a cell that does not comprise (i) a nucleic acid sequence encoding an interleukin-15 (IL-15) polypeptide or a fragment thereof or (ii) a nucleic acid sequence encoding an interleukin-15 receptor alpha (IL-15Ra) polypeptide or a fragment thereof. In some embodiments, the proliferation of the cell is increased for at least about 5%. In some embodiments, IL-15 is operatively linked to IL-15Ra.

In some embodiments, expression of an exhaustion marker of the cell is decreased compared to a cell that does not comprise (i) a nucleic acid sequence encoding an interleukin-15 (IL-15) polypeptide or a fragment thereof or (ii) a nucleic acid sequence encoding an interleukin-15 receptor alpha (IL-15Ra) polypeptide or a fragment thereof. In some embodiments, IL-15 is operatively linked to IL-15Ra. In some embodiments, the expression of the exhaustion marker of the cell is decreased for at least about 5%. In some embodiments, the exhaustion marker is PD-1, TIM-3 or LAG-3.

In some embodiments, tumor infiltration of the cell is increased compared to a cell that does not comprise (i) a nucleic acid sequence encoding an interleukin-15 (IL-15) polypeptide or a fragment thereof or (ii) a nucleic acid sequence encoding an interleukin-15 receptor alpha (IL-15Ra) polypeptide or a fragment thereof. In some embodiments, IL-15 is operatively linked to IL-15Ra. In some embodiments, the tumor infiltration of the cell is increased for at least about 2-fold.

In some embodiments, expression of TCF-1 of the cell is increased compared to a cell that does not comprise (i) a nucleic acid sequence encoding an interleukin-15 (IL-15) polypeptide or a fragment thereof or (ii) a nucleic acid sequence encoding an interleukin-15 receptor alpha (IL-15Ra) polypeptide or a fragment thereof. In some embodiments, IL-15 is operatively linked to IL-15Ra. In some embodiments, the expression of TCF-1 of the cell is increased for at least about 5%.

In some aspects, provided herein is a population of cells comprising the cell disclosed herein. In some embodiments, the population of cells has an increased proportion of cells having a central memory phenotype relative to a population of cells that do not comprise (i) a nucleic acid sequence encoding an interleukin-15 (IL-15) polypeptide or a fragment thereof or (ii) a nucleic acid sequence encoding interleukin-15 receptor alpha (IL-15Ra) polypeptide or a fragment thereof. In some embodiments, the population of cells has an increased proportion of cells having a naïve phenotype relative to a population of cells that do not comprise (i) a nucleic acid sequence encoding an interleukin-15 (IL-15) polypeptide or a fragment thereof or (ii) a nucleic acid sequence encoding an interleukin-15 receptor alpha (IL-15Ra) polypeptide or a fragment thereof. In some embodiments, the population of cells has a reduced proportion of cells having a terminal effector phenotype relative to a population of cells that do not comprise (i) a nucleic acid sequence encoding an interleukin-15 (IL-15) polypeptide or a fragment thereof or (ii) a nucleic acid sequence encoding an interleukin-15 receptor alpha (IL-15Ra) polypeptide or a fragment thereof. In some embodiments, the population of cells has an increased proportion of cells expressing TCF-1.

In some aspects, provided herein is a method of increasing the activity or persistence of a cell expressing a recombinant nucleic acid molecule comprising a sequence encoding a T cell receptor (TCR) fusion protein (TFP), the method comprising expressing an interleukin-15 (IL-15) polypeptide or a fragment thereof in the cell wherein the TFP comprises a TCR subunit comprising at least a portion of a TCR extracellular domain, and a TCR transmembrane domain, and an antigen binding domain; and wherein the TCR subunit and the antigen binding domain are operatively linked. In some embodiments, the cell is any one of the cells disclosed herein.

In some aspects, provided herein is a method of increasing the activity or persistence of a cell expressing a recombinant nucleic acid molecule comprising a sequence encoding a T cell receptor (TCR) fusion protein (TFP), the method comprising expressing an interleukin-15 receptor alpha (IL-15Rα) polypeptide or a fragment thereof in the cell wherein the TFP comprises a TCR subunit comprising at least a portion of a TCR extracellular domain, and a TCR transmembrane domain, and an antigen binding domain; and wherein the TCR subunit and the antigen binding domain are operatively linked. In some embodiments, the cell is any one of the cells disclosed herein.

In some aspects, provided herein is a method of increasing the proliferation of a cell expressing a recombinant nucleic acid molecule comprising a sequence encoding a T cell receptor (TCR) fusion protein (TFP), the method comprising expressing an interleukin-15 (IL-15) polypeptide or a fragment thereof in the cell: wherein the TFP comprises: (a) a TCR subunit comprising: (i) at least a portion of a TCR extracellular domain, and (ii) a TCR transmembrane domain, and (b) an antigen binding domain; and wherein the TCR subunit and the antigen binding domain are operatively linked. In some embodiments, IL-15 is operatively linked to IL-15Rα. In some embodiments, the cell is any one of the cells as described herein.

In some aspects, provided herein is a method of increasing the proliferation of a cell expressing a recombinant nucleic acid molecule comprising a sequence encoding a T cell receptor (TCR) fusion protein (TFP), the method comprising expressing an interleukin-15 receptor alpha (IL-15Rα) polypeptide or a fragment thereof in the cell: wherein the TFP comprises: (a) a TCR subunit comprising: (i) at least a portion of a TCR extracellular domain, and (ii) a TCR transmembrane domain, and (b) an antigen binding domain; and wherein the TCR subunit and the antigen binding domain are operatively linked. In some embodiments, the cell is any one of the cells as described herein.

In some aspects, provided herein is a method of increasing persistence or decreasing the expression of one or more exhaustion markers in a cell expressing a recombinant nucleic acid molecule comprising a sequence encoding a T cell receptor (TCR) fusion protein (TFP), the method comprising expressing an interleukin-15 (IL-15) polypeptide or a fragment thereof in the cell: wherein the TFP comprises: (a) a TCR subunit comprising: (i) at least a portion of a TCR extracellular domain, and (ii) a TCR transmembrane domain, and (b) an antigen binding domain; and wherein the TCR subunit and the antigen binding domain are operatively linked. In some embodiments, IL-15 is operatively linked to IL-15Rα. In some embodiments, the cell is any one of the cells as described herein.

In some aspects, provided herein is a method of decreasing the expression of one or more exhaustion markers in a cell expressing a recombinant nucleic acid molecule comprising a sequence encoding a T cell receptor (TCR) fusion protein (TFP), the method comprising expressing an interleukin-15 receptor alpha (IL-15Rα) polypeptide or a fragment thereof in the cell: wherein the TFP comprises: (a) a TCR subunit comprising: (i) at least a portion of a TCR extracellular domain, and (ii) a TCR transmembrane domain, and (b) an antigen binding domain; and wherein the TCR subunit and the antigen binding domain are operatively linked. In some embodiments, the cell is any one of the cells as described herein.

In some aspects, provided herein is a method of increasing the TCF-1+ T cell population among a population of a cells expressing a recombinant nucleic acid molecule comprising a sequence encoding a T cell receptor (TCR) fusion protein (TFP), the method comprising expressing an interleukin-15 (IL-15) polypeptide or a fragment thereof in the cell: wherein the TFP comprises: (a) a TCR subunit comprising: (i) at least a portion of a TCR extracellular domain, and (ii) a TCR transmembrane domain, and (b) an antigen binding domain; and wherein the TCR subunit and the antigen binding domain are operatively linked. In some embodiments, IL-15 is operatively linked to IL-15Rα. In some embodiments, the cell is any one of the cells as described herein.

In some aspects, provided herein is a method of increasing the TCF-1+ T cell population among a population of a cells expressing a recombinant nucleic acid molecule comprising a sequence encoding a T cell receptor (TCR) fusion protein (TFP), the method comprising expressing an interleukin-15 receptor alpha (IL-15Rα) polypeptide or a fragment thereof in the cell: wherein the TFP comprises: (a) a TCR subunit comprising: (i) at least a portion of a TCR extracellular domain, and (ii) a TCR transmembrane domain, and (b) an antigen binding domain; and wherein the TCR subunit and the antigen binding domain are operatively linked. In some embodiments, the cell is any one of the cells as described herein.

In some aspects, provided herein is a method of increasing the tumor infiltration of a cell expressing a recombinant nucleic acid molecule comprising a sequence encoding a T cell receptor (TCR) fusion protein (TFP), the method comprising expressing an interleukin-15 (IL-15) polypeptide or a fragment thereof in the cell: wherein the TFP comprises: (a) a TCR subunit comprising: (i) at least a portion of a TCR extracellular domain, and (ii) a TCR transmembrane domain, and (b) an antigen binding domain; and wherein the TCR subunit and the antigen binding domain are operatively linked. In some embodiments, the cell is any one of the cells as described herein. In some embodiments, IL-15 is operatively linked to IL-15Rα.

In some aspects, provided herein is a method of increasing the tumor infiltration of a cell expressing a recombinant nucleic acid molecule comprising a sequence encoding a T cell receptor (TCR) fusion protein (TFP), the method comprising expressing an interleukin-15 receptor alpha (IL-15Rα) polypeptide or a fragment thereof in the cell: wherein the TFP comprises: (a) a TCR subunit comprising: (i) at least a portion of a TCR extracellular domain, and (ii) a TCR transmembrane domain, and (b) an antigen binding domain; and wherein the TCR subunit and the antigen binding domain are operatively linked. In some embodiments, the cell is any one of the cells as described herein.

In some aspects, provided herein is a pharmaceutical composition comprising the cell or cells disclosed herein and a pharmaceutically acceptable carrier. In some aspects, provided herein is a method of treating a disease or a condition in a subject in need thereof, comprising administering to the subject a therapeutically effective amount of the pharmaceutical composition disclosed herein.

In some embodiments, the disease or the condition is a cancer or a disease or a condition associated with expression of CD19, B-cell maturation antigen (BCMA), mesothelin (MSLN), CD20, CD70, MUC16, Trop-2, Nectin-4, or GPC3. In some embodiments, the cancer is a hematologic cancer selected from the group consisting of B-cell acute lymphoid leukemia (B-ALL), T cell acute lymphoid leukemia (T-ALL), acute lymphoblastic leukemia (ALL), chronic myelogenous leukemia (CML), chronic lymphocytic leukemia (CLL), B cell prolymphocytic leukemia, blastic plasmacytoid dendritic cell neoplasm, Burkitt's lymphoma, diffuse large B cell lymphoma, follicular lymphoma, hairy cell leukemia, small cell-follicular lymphoma, large cell-follicular lymphoma, malignant lymphoproliferative conditions, MALT lymphoma, mantle cell lymphoma, Marginal zone lymphoma, multiple myeloma, myelodysplasia, myelodysplastic syndrome, non-Hodgkin's lymphoma, plasmablastic lymphoma, plasmacytoid dendritic cell neoplasm, Waldenstrom macroglobulinemia, and preleukemia. In some embodiments, the subject is a human.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 shows a schematic illustration of the constructs described in Example 1.

FIG. 2 is a series of graphs showing expansion of T cells having the TFP constructs shown generated according to the methods described in Example 2.

FIG. 3 is a series of plots showing the proportion of T cells having the TFP constructs shown generated according to the methods described in Example 2 having cell surface expression of IL-15-Rα, PD-1 and VHH after 10 days of expansion.

FIG. 4 is a series of plots showing the memory phenotype of T cells having the TFP constructs shown generated according to the methods described in Example 2 after 10 days of expansion.

FIG. 5 is a schematic illustration of the experiment described in Example 3.

FIGS. 6A-6C are a series of graphs showing the results of a CSFE assay of T cells having the TFP constructs shown in the assay described in Example 3. FIG. 6A and FIG. 6B show the in vitro persistence assays at day 7. FIG. 6C shows the in vitro persistence assay at day 21.

FIGS. 7A-7C are a series of graphs showing expansion of T cells having the TFP constructs shown in the assay described in Example 3. FIG. 7A shows expansion of T cells without stimulation. FIG. 7B shows expansion of T cells with sub-optimal TCR stimulation. FIG. 7C shows expansion of T cells at day 21 at various conditions (e.g., no stimulation, sub-optimal CD3/28 transactivation, or optimal CD3/28 dynabeads).

FIG. 8 is a schematic illustration of the experiment described in Example 4.

FIG. 9 is a series of graphs showing expansion of T cells having the TFP constructs shown in the assay described in Example 4.

FIG. 10 is a series of graphs showing the results of a CSFE assay of T cells having the TFP constructs shown at day 7 of the assay described in Example 4.

FIG. 11A and FIG. 11B are a series of graphs showing the number of VHH+ (11A) and CD3+ (11B) T cells having the TFP constructs shown at days 7, 14, and 21 of the assay described in Example 4.

FIG. 12 is a series of graphs showing the number of VHH+ T cells having the TFP constructs shown at days 7, 14, and 21 of the assay described in Example 4 relative to the number of VHH+ T cells expressing the MSLN TFP alone.

FIG. 13A and FIG. 13B are a series of graphs showing the memory phenotype of T cells having the TFP constructs shown at days 7 and 21 of the assay described in Example 4. FIG. 13A shows the memory phenotype at day 7. FIG. 13B shows the memory phenotype at day 21.

FIG. 14A and FIG. 14B are a series of graphs showing target lysis by T cells having the TFP constructs shown at days 8, 15, and 22 of the assay described in Example 4.

FIG. 15 is a series of graphs showing cytokine secretion by T cells having the TFP constructs shown at day 3 of the assay described in Example 4.

FIG. 16 is a series of graphs showing in vivo efficacy of T cells having the TFP constructs shown at reducing tumor volume in the murine xenograft model described in Example 5.

FIG. 17 shows schematic illustrations of the constructs described in Example 8.

FIG. 18A-18D is a series of graphs showing T cell expansion, transduction efficiency and memory phenotype after transduction with TRuC constructs. FIG. 18A shows T cell expansion. FIG. 18B shows representative co-expression of CD70 TRuC and mbIL-15fu proteins. FIG. 18C shows transduction summary of 3 donors and FIG. 18D shows the CD4+/CD8+ ratio for successfully transduced T cells (TRuC+).

FIG. 19A-19C is a series of graphs showing the T cell differentiation phenotypes of transduced cells. FIG. 19A shows characterization of T cell differentiation phenotype by flow cytometry for a representative donor. FIG. 19B and FIG. 19C show proportions for each T cell differentiation phenotype in TRuC+/CD4+ and TRuC+/CD8+ cells, respectively.

FIG. 20A-20E is a series of graphs showing cytotoxicity and cytokine production in a co-culture assay using TRuC-T cells. FIG. 20A shows 24 hour cytotoxicity against CD70^{High} target cell 786-O. FIG. 20B shows cytokine production against CD70^{High} target cell 786-O. FIG. 20C shows 24 hour cytotoxicity against CD70^{Moderate} target cell ACHN. FIG. 20D shows 24 hour cytokine production against CD70^{Moderate} target cell ACHN. FIG. 20E shows 24 hour cytotoxicity against CD70^{Negative} target cell K562.

FIG. 21A-21C is a series of graphs showing results of a set of persistence and expansion assays. FIG. 21A shows T cell persistence in the absence of stimulation. FIG. 21B shows T cell expansion after a single round of stimulation with CD70^{High} target cells 786-O. FIG. 21C shows T cell expansion after repeated stimulation with CD70^{High} target cell 786-0.

FIG. 22A-22J is a series of graphs showing results of in vivo efficacy studies in a mouse tumor model. FIG. 22A shows anti-tumor activity of T cells measured by BLI and FIG. 22B shows tumor volume as measured by caliper. FIG. 22C shows flow cytometry data for T cell accumulation in tumor and blood samples of a representative mouse. FIG. 22D shows percentage of human CD45+ cells in tumor. FIG. 22E shows percentage of human CD45+ cells in blood. FIG. 22F shows number of human CD45+ cells per tumor. FIG. 22G shows the number of human CD45+ cells per μL of blood. FIG. 22H shows the ratio of CD4+/CD8+ in TRuC+ cells in tumor. FIG. 22I shows the ratio of CD4+/CD8+ in TRuC+ cells in blood. FIG. 22J shows the ratio of CD4+/CD8+ in TRuC+ cells of the initial infusion.

FIG. 23A-23H is a series of graphs showing results of in vivo efficacy studies in a mouse tumor model. FIG. 23A shows tumor volume as measured by caliper. FIG. 23B shows percentage of human CD45+ cells in tumor. FIG. 23C shows percentage of human CD45+ cells in blood. FIG. 23D shows number of human CD45+ cells per tumor. FIG. 23E shows the number of human CD45+ cells per μL of blood. FIG. 23F shows the ratio of CD4+/CD8+ in TRuC+ cells in tumor. FIG. 23G shows the ratio of CD4+/CD8+ in TRuC+ cells in blood. FIG. 23H shows the ratio of CD4+/CD8+ in TRuC+ cells of the initial infusion.

FIGS. 24A-24D show graphs of quantification of T cell expansion (FIG. 24A), CD4/CD8 ratios (FIG. 24B), and transduction efficiency (Round 1—FIG. 24C; Round 2—FIG. 24D) for T cells transduced with various TFP and/or IL-15 constructs.

FIG. 25 shows surface expression of the indicated construct in transduced T cells as determined by flow cytometry.

FIG. 26 shows surface expression of the indicated construct in transduced T cells as determined by flow cytometry.

FIGS. 27A-27F show quantification of the differentiation state (Temra in FIG. 27A, Naïve in FIG. 27B, Tem in FIG. 27C and Tem in FIG. 27D) of CD4+ and CD8+ T cells transduced with the noted construct on day 10 post expansion. FIGS. 27E and 27F show the slight decrease in Temra and slight increase in Tem phenotypes in IL-15 expressing transduced T cells, respectively.

FIGS. 28A-28C show quantification of IL-15 production by transduced T cells across three donors (FIGS. 28A, 28B, and 28C each being from separate donors). The dotted line indicates the limit of detection for the assay.

FIGS. 29A and 29B show the results of a cytotoxicity assay wherein transduced T cells were co-cultured with MSTO-MSLN cells (FIG. 29A) or MSTO-MSLN-PDL1 cells (FIG. 29B).

FIGS. 30A-30D show the results of a cytokine response assay in MSTO-MSLN (FIG. 30A and FIG. 30C) or MSTO-MSLN-PDL1 (FIG. 30B and FIG. 30D) cells as measured by IFNγ (FIG. 30A and FIG. 30B) and IL-2 (FIG. 30C and FIG. 30D).

FIGS. 31A-31D show cell number quantifications as determined by CD45+ (FIG. 31A and FIG. 31C) or VHH+ (FIG. 31B and FIG. 31D) by FACs in a repeated stimulation assay conducted using transduced T cells and MSTO-MSLN cells (FIG. 31A and FIG. 31B) or MSTO-MSLN-PDL1 cells (FIG. 31C and FIG. 31D).

FIG. 32 shows cytokine release data from a repeated stimulation assay.

FIG. 33 shows the differentiation state of transduced T cells from a repeated stimulation assay. Flow cytometry plots are representative images from Day 4.

FIG. 34A and FIG. 34B show PD1 expression from a repeated stimulation assay conducted in MSTO-MSLN (FIG. 34A) or MSTO-MSLN-PDL1 (FIG. 34B) cells.

FIG. 35A and FIG. 35B show data from an antigen independent expansion (persistence) assay. FIG. 35A shows cell counts over time and FIG. 35B shows the cell counts on the final day of the experiment (Day 10).

FIG. 36A and FIG. 36B show data from an antigen independent expansion (persistence) assay. FIG. 36A shows percent VHH+ cells in culture over time, while FIG. 36B shows percent VHH+ cells in culture on the final day of the experiment (Day 10).

FIG. 37 shows quantifications of memory phenotypes of transduced T cells from an antigen independent expansion (persistence) assay.

FIG. 38A and FIG. 38B show data from an antigen independent expansion assay wherein PDL1-Fc was used to coat the bottom of the assay plate. FIG. 38A shows cell counts over time and FIG. 38B shows cell counts on Day 10.

FIG. 39A and FIG. 39B show cell count data from an antigen independent expansion assay when exogenous cytokines IL-2 (FIG. 39A) or IL-15 (FIG. 39B) were added.

FIGS. 40A-40D show data from an antigen independent expansion assay when exogenous cytokines IL-2 (FIG. 40A and FIG. 40B) and 11-15 (FIG. 40C and FIG. 40D) were added.

FIG. 41 shows a schematic and related data proposing a hypothesis for the decreased expansion of T cells co-expressing IL-15 in the presence of exogenous cytokines in an antigen independent expansion assay.

FIG. 42 shows flow cytometry plots of CD122 surface expression from transduced T cells on Day 4 of an antigen independent expansion assay.

FIG. 43 shows tumor volume quantification overtime in an in vivo efficacy study.

FIG. 44 shows tumor volume quantification overtime in an in vivo efficacy study split by treatment.

FIG. 45 shows data from an in vivo efficacy study at Day 65 and post re-challenge (Day 65).

FIG. 46 shows tumor volume quantifications overtime after re-challenge (Day 65) split by treatment.

FIG. 47 shows quantification of TCF7 across experimental conditions for transduced T cells derived from a single donor.

FIG. 48 shows quantification of TCF7 across experimental conditions for transduced T cells derived from a second donor.

FIG. 49 shows flow cytometry plots for CD8+ TCF7 (TCF-1) versus T-bet across experimental conditions and for transduced T cells derived from a single donor.

FIG. 50 shows flow cytometry plots for CD8+ TCF7 (TCF-1) versus T-bet across experimental conditions and for transduced T cells derived from a second donor.

FIG. 51 shows flow cytometry plots for CD8− TCF7 (TCF-1) versus T-bet across experimental conditions and for transduced T cells derived from a single donor.

FIG. 52 shows flow cytometry plots for CD8+ TCF7 (TCF-1) versus T-bet across experimental conditions and for transduced T cells derived from a second donor.

FIG. 53 shows flow cytometry plots for CD8+ TCF7 (TCF-1) versus Granzyme B across experimental conditions and for transduced T cells derived from a single donor.

FIG. 54 shows flow cytometry plots for CD8+ TCF7 (TCF-1) versus Granzyme B across experimental conditions and for transduced T cells derived from a second donor.

FIG. 55 shows flow cytometry plots for CD8− TCF7 (TCF-1) versus Granzyme B across experimental conditions and for transduced T cells derived from a single donor.

FIG. 56 shows flow cytometry plots for CD8− TCF7 (TCF-1) versus Granzyme B across experimental conditions and for transduced T cells derived from a second donor.

FIG. 57 shows data from an in vivo efficacy study. Tumors harvested from mice treated with transduced T cells were evaluated for CD45 expression and cell count.

FIG. 58 shows data from an in vivo efficacy study. Flow cytometry plots showing the surface expression of VHH in tumor tissues of mice treated with transduced T cells are shown.

FIG. 59 shows data from an in vivo efficacy study wherein Ki67 expression was assessed in tumor tissue as a measure of transduced T cell proliferation.

FIG. 60 shows data from an in vivo efficacy study wherein Ki67 expression was assessed in tumor tissue as a measure of transduced CD8+ or CD4+ transduced T cell proliferation.

FIG. 61 shows data from an in vivo efficacy study wherein CD4+ and CD8+ cell populations were measured by flow cytometry, quantified and plotted as a ratio in a histogram.

FIG. 62 shows flow cytometry plots of inhibitory marker (PD-1 and LAG-3) expression in tumor tissue collected from mice treated with transduced T cells.

FIG. 63 shows flow cytometry plots of inhibitory marker (PD-1 and TIGIT) expression in tumor tissue collected from mice treated with transduced T cells.

FIGS. 64A-64D show in vitro data for transduced T cells. FIG. 64A shows T cell expansion over a 10 day period. FIG. 64B shows flow cytometry plots measuring the surface presence of TC-210 and IL15Rα in transduced T cells. FIG. 64C shows flow cytometry plots for assessing memory phenotype by measuring CD45Ra and CCR7 surface expression in transduced T cells. FIG. 64D shows quantification of memory phenotypes in CD4+ or CD8+ transduced T cells.

FIGS. 65A-65C show data collected from in vitro studies of transduced T cells. FIG. 65A shows cytotoxicity and cytokine production after 24 hr co-culture with MSTO-MSLN cells. FIG. 65B shows flow cytometry plots of surface expression of CD27 and TCF-1 after a 96 hr activation assay. FIG. 65C shows flow cytometry plots of surface expression of TCF-1 and Granzyme B after a 96 hr activation assay.

FIG. 66 shows results of a persistence assay of transduced T cells grown under cytokine free culture conditions or cytokine (IL-2 or IL-15) supplemented culture conditions.

FIG. 67 shows the results of a repetitive stimulation assay using transduced T cells.

FIG. 68 shows data from an in vivo efficacy study of transduced T cells.

FIG. 69 shows data from tumor and blood samples collected during an in vivo efficacy study of transduced T cells.

### DETAILED DESCRIPTION OF THE INVENTION

Disclosed herein, in some embodiments, is a recombinant nucleic acid molecule comprising a first nucleic acid sequence encoding a T cell receptor (TCR) fusion protein (TFP) wherein the TFP comprises (a) a TCR subunit comprising (i) at least a portion of a TCR extracellular domain, and (ii) a TCR transmembrane domain, and (b) an antigen binding domain and wherein the TCR subunit and the antigen binding domain are operatively linked, and a second nucleic acid sequence encoding an interleukin-15 (IL-15) polypeptide or a fragment thereof.

Disclosed herein, in some embodiments, is a recombinant nucleic acid comprising a first nucleic acid sequence encoding a T cell receptor (TCR) fusion protein (TFP) wherein the TFP comprises (a) a TCR subunit comprising (i) at least a portion of a TCR extracellular domain, and (ii) a TCR transmembrane domain, and (b) an antigen binding domain and wherein the TCR subunit and the antigen binding domain are operatively linked, and a second nucleic acid sequence encoding an interleukin-15 receptor alpha (IL-15Rα) polypeptide or a fragment thereof.

Disclosed herein, in some embodiments, is a cell comprising a first nucleic acid sequence encoding a T cell receptor (TCR) fusion protein (TFP) wherein the TFP comprises (a) a TCR subunit comprising (i) at least a portion of a TCR extracellular domain, and (ii) a TCR transmembrane domain, and (b) an antigen binding domain and wherein the TCR subunit and the antigen binding domain are operatively linked, and a second nucleic acid sequence encoding an interleukin-15 (IL-15) polypeptide or a fragment thereof.

Disclosed herein, in some embodiment, is a cell comprising a first nucleic acid sequence encoding a T cell receptor (TCR) fusion protein (TFP) wherein the TFP comprises (a) a TCR subunit comprising (i) at least a portion of a TCR extracellular domain, and (ii) a TCR transmembrane domain, and (b) an antigen binding domain and wherein the TCR subunit and the antigen binding domain are operatively linked, and a second nucleic acid sequence encoding an interleukin-15 receptor alpha (IL-15Rα) polypeptide or a fragment thereof.

Disclosed herein, in some embodiments, is a method of increasing the activity or persistence of a cell expressing a recombinant nucleic acid molecule comprising a sequence encoding a T cell receptor (TCR) fusion protein (TFP), the method comprising expressing an interleukin-15 (IL-15) polypeptide or a fragment thereof in the cell wherein the TFP comprises (a) a TCR subunit comprising (i) at least a portion of a TCR extracellular domain, and (ii) a TCR transmembrane domain, and (b) an antigen binding domain and wherein the TCR subunit and the antigen binding domain are operatively linked.

Disclosed herein, in some embodiments, is a method of increasing the activity or persistence of a cell expressing a recombinant nucleic acid molecule comprising a sequence encoding a T cell receptor (TCR) fusion protein (TFP), the method comprising expressing an interleukin-15 receptor alpha (IL-15Rα) polypeptide or a fragment thereof in the cell wherein the TFP comprises (a) a TCR subunit comprising (i) at least a portion of a TCR extracellular domain, and (ii) a TCR transmembrane domain, and (b) an antigen binding domain and wherein the TCR subunit and the antigen binding domain are operatively linked.

Disclosed herein, in some embodiments, are polypeptides encoded by recombinant nucleic acid molecules described herein. Disclosed herein, in some embodiments, are vectors comprising recombinant nucleic acid molecules described herein. Disclosed herein, in some embodiments, are cells comprising the recombinant nucleic acid molecules described herein, the polypeptides described herein, or the vectors described herein. Disclosed herein, in some embodiments, is a population of cells comprising the cells described herein. Disclosed herein, in some embodiments, is a pharmaceutical composition comprising the cells described herein and a pharmaceutically acceptable carrier. Disclosed herein, in some embodiments, is a method of treating a disease or a condition in a subject in need thereof, comprising administering to the subject a therapeutically effective amount of the pharmaceutical compositions described herein.

### Certain Terminology

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

As used herein, “about” can mean plus or minus less than 1 or 1, 2, 3, 4, 5, 6, 7, 8, 9, 10, 11, 12, 13, 14, 15, 16, 17, 18, 19, 20, 25, 30, or greater than 30 percent, depending upon the situation and known or knowable by one skilled in the art.

As used herein the specification, “subject” or “subjects” or “individuals” may include, but are not limited to, mammals such as humans or non-human mammals, e.g., domesticated, agricultural or wild, animals, as well as birds, and aquatic animals. “Patients” are subjects suffering from or at risk of developing a disease, disorder or condition or otherwise in need of the compositions and methods provided herein.

As used herein, “treating” or “treatment” refers to any indicia of success in the treatment or amelioration of the disease or condition. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. As used herein, “treat or prevent” is sometimes used herein to refer to a method that results in some level of treatment or amelioration of the disease or condition and contemplates a range of results directed to that end, including but not restricted to prevention of the condition entirely.

As used herein, “preventing” refers to the prevention of the disease or condition, e.g., tumor formation, in the patient. For example, if an individual at risk of developing a tumor or other form of cancer is treated with the methods of the present disclosure and does not later develop the tumor or other form of cancer, then the disease has been prevented, at least over a period of time, in that individual.

As used herein, a “therapeutically effective amount” is the amount of a composition or an active component thereof sufficient to provide a beneficial effect or to otherwise reduce a detrimental non-beneficial event to the individual to whom the composition is administered. By “therapeutically effective dose” herein is meant a dose that produces one or more desired or desirable (e.g., beneficial) effects for which it is administered, such administration occurring one or more times over a given period of time. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999))

As used herein, a “T cell receptor (TCR) fusion protein” or “TFP” includes a recombinant polypeptide derived from the various polypeptides comprising the TCR that is generally capable of i) binding to a surface antigen on target cells and ii) interacting with other polypeptide components of the intact TCR complex, typically when co-located in or on the surface of a T cell. In the present disclosure, “TFP” and “TRuC” can be used interchangeably.

The term “stimulation” refers to a primary response induced by binding of a stimulatory domain or stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, and/or reorganization of cytoskeletal structures, and the like.

The term “stimulatory molecule” or “stimulatory domain” refers to a molecule or portion thereof expressed by a T cell that provides the primary cytoplasmic signaling sequence(s) that regulate primary activation of the TCR complex in a stimulatory way for at least some aspect of the T cell signaling pathway. In one aspect, the primary signal is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or “ITAM”. Examples of an ITAM containing primary cytoplasmic signaling sequence that is of particular use in the invention includes, but is not limited to, those derived from TCR zeta, FcR gamma, FcR beta, CD3 gamma, CD3 delta, CD3 epsilon, CD5, CD22, CD79a, CD79b, CD278 (also known as “ICOS”) and CD66d.

The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T cells may recognize these complexes using their T cell receptors (TCRs). APCs process antigens and present them to T cells.

“Major histocompatibility complex (MHC) molecules are typically bound by TCRs as part of peptide:MHC complex. The MHC molecule may be an MHC class I or II molecule. The complex may be on the surface of an antigen presenting cell, such as a dendritic cell or a B cell, or any other cell, including cancer cells, or it may be immobilized by, for example, coating on to a bead or plate.

The human leukocyte antigen system (HLA) is the name of the gene complex which encodes major histocompatibility complex (MHC) in humans and includes HLA class I antigens (A, B & C) and HLA class II antigens (DP, DQ, & DR). HLA alleles A, B and C present peptides derived mainly from intracellular proteins, e.g., proteins expressed within the cell.

During T cell development in vivo, T cells undergo a positive selection step to ensure recognition of self MHCs followed by a negative step to remove T cells that bind too strongly to MHC which present self-antigens. As a consequence, certain T cells and the TCRs they express will only recognize peptides presented by certain types of MHC molecules—i.e., those encoded by particular HLA alleles. This is known as HLA restriction.

One HLA allele of interest is HLA-A*0201, which is expressed in the vast majority (>50%) of the Caucasian population. Accordingly, TCRs which bind WT1 peptides presented by MHC encoded by HLA-A*0201 (i.e., are HLA-A*0201 restricted) are advantageous since an immunotherapy making use of such TCRs will be suitable for treating a large proportion of the Caucasian population.

Other HLA-A alleles of interest are HLA-A*0101, HLA-A*2402, and HLA-A*0301.

Widely expressed HLA-B alleles of interest are HLA-B*3501, HLA-B*0702 and HLA-B*3502.

An “intracellular signaling domain,” as the term is used herein, refers to an intracellular portion of a molecule. The intracellular signaling domain generates a signal that promotes an immune effector function of the TFP containing cell, e.g., a modified T-T cell. Examples of immune effector function, e.g., in a modified T-T cell, include cytolytic activity and T helper cell activity, including the secretion of cytokines. In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation.

A primary intracellular signaling domain can comprise an ITAM (“immunoreceptor tyrosine-based activation motif”). Examples of ITAM containing primary cytoplasmic signaling sequences include, but are not limited to, those derived from CD3 zeta, FcR gamma, FcR beta, CD3 gamma, CD3 delta, CD3 epsilon, CD5, CD22, CD79a, CD79b, and CD66d DAP10 and DAP12.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include but are not limited to an MHC class 1 molecule, BTLA and a Toll ligand receptor, as well as OX40, CD2, CD27, CD28, CDS, ICAM-1, LFA-1 (CD11a/CD18) and 4-1BB (CD137). A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H3, and a ligand that specifically binds with CD83, and the like. The intracellular signaling domain can comprise the entire intracellular portion, or the entire native intracellular signaling domain, of the molecule from which it is derived, or a functional fragment thereof. The term “4-1BB” refers to a member of the TNFR superfamily with an amino acid sequence provided as GenBank Acc. No. AAA62478.2, or the equivalent residues from a non-human species, e.g., mouse, rodent, monkey, ape and the like; and a “4-1BB costimulatory domain” is defined as amino acid residues 214-255 of GenBank Acc. No. AAA62478.2, or the equivalent residues from a non-human species, e.g., mouse, rodent, monkey, ape and the like.

The term “antibody,” as used herein, refers to a protein, or polypeptide sequences derived from an immunoglobulin molecule, which specifically binds to an antigen. Antibodies can be intact immunoglobulins of polyclonal or monoclonal origin, or fragments thereof and can be derived from natural or from recombinant sources.

The terms “antibody fragment” refers to at least one portion of an antibody, or recombinant variants thereof, that contains the antigen binding domain, i.e., an antigenic determining variable region of an intact antibody, that is sufficient to confer recognition and specific binding of the antibody fragment to a target, such as an antigen and its defined epitope. Examples of antibody fragments include, but are not limited to, Fab, Fab′, F(ab′)₂, and Fv fragments, single-chain (sc)Fv (“scFv”) antibody fragments, linear antibodies, single domain antibodies such as sdAb (either V_{L} or V_{H}), camelid V_{HH} domains, and multi-specific antibodies formed from antibody fragments.

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single polypeptide chain, and wherein the scFv retains the specificity of the intact antibody from which it is derived.

“Heavy chain variable region” or “V_{H}” with regard to an antibody refers to the fragment of the heavy chain that contains three CDRs interposed between flanking stretches known as framework regions, these framework regions are generally more highly conserved than the CDRs and form a scaffold to support the CDRs. A camelid “V_{HH}” domain is a heavy chain comprising a single variable antibody domain.

Unless specified, as used herein a scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}. In some embodiments, the linker may comprise SEQ ID NO: 401.

The portion of the TFP composition of the disclosure comprising an antibody or antibody fragment thereof may exist in a variety of forms where the antigen binding domain is expressed as part of a contiguous polypeptide chain including, for example, a single domain antibody fragment (sdAb), a single chain antibody (scFv) derived from a murine, humanized or human antibody (Harlow et al., 1999, In: Using Antibodies: A Laboratory Manual, Cold Spring Harbor Laboratory Press, N.Y.; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In one aspect, the antigen binding domain of a TFP composition of the disclosure comprises an antibody fragment. In a further aspect, the TFP comprises an antibody fragment that comprises a scFv or a sdAb.

The term “recombinant antibody” refers to an antibody that is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “Ag” refers to a molecule that is capable of being bound specifically by an antibody, or otherwise provokes an immune response. This immune response may involve either antibody production, or the activation of specific immunologically-competent cells, or both.

The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present disclosure includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components.

As used herein, the term “CD19” refers to the Cluster of Differentiation 19 protein, which is an antigenic determinant detectable on B cell leukemia precursor cells, other malignant B cells and most cells of the normal B cell lineage.

As used herein, the term “BCMA” refers to the B-cell maturation antigen also known as tumor necrosis factor receptor superfamily member 17 (TNFRSF17) and Cluster of Differentiation 269 protein (CD269) is a protein that in humans is encoded by the TNFRSF17 gene. TNFRSF17 is a cell surface receptor of the TNF receptor superfamily which recognizes B-cell activating factor (BAFF) (see, e.g., Laabi et al., EMBO 11 (11): 3897-904 (1992). This receptor is expressed in mature B lymphocytes and may be important for B-cell development and autoimmune response.

As used herein, the term “CD16” (also known as FcγRIII) refers to a cluster of differentiation molecule found on the surface of natural killer cells, neutrophil polymorphonuclear leukocytes, monocytes and macrophages. CD16 has been identified as Fc receptors FcγRIIIa (CD16a) and FcγRIIIb (CD16b), which participate in signal transduction. CD16 is a molecule of the immunoglobulin superfamily (IgSF) involved in antibody-dependent cellular cytotoxicity (ADCC).

“NKG2D,” as used herein, refers to a transmembrane protein belonging to the CD94/NKG2 family of C-type lectin-like receptors. In humans, NKG2D is expressed by NK cells, γδ T cells and CD8+ αβ T cells. NKG2D recognizes induced-self proteins from MIC and RAET1/ULBP families which appear on the surface of stressed, malignant transformed, and infected cells.

Mesothelin (MSLN) refers to a tumor differentiation antigen that is normally present on the mesothelial cells lining the pleura, peritoneum and pericardium. Mesothelin is over expressed in several human tumors, including mesothelioma and ovarian and pancreatic adenocarcinoma.

Tyrosine-protein kinase transmembrane receptor ROR1, also known as neurotrophic tyrosine kinase, receptor-related 1 (NTRKR1) is a member of the receptor tyrosine kinase-like orphan receptor (ROR) family. It plays a role in metastasis of cancer.

The term “MUC16”, also known as “mucin 16, cell-surface associated” or “ovarian cancer-related tumor marker CA125” is a membrane-tethered mucin that contains an extracellular domain at its amino terminus, a large tandem repeat domain, and a transmembrane domain with a short cytoplasmic domain. Products of this gene have been used as a marker for different cancers, with higher expression levels associated with poorer outcomes.

The term “CD22,” also known as sialic acid binding Ig-like lectin 2, SIGLEC-2, T cell surface antigen leu-14, and B cell receptor CD22, is a protein that mediates B cell/B cell interactions, and is thought to be involved in the localization of B cells in lymphoid tissues, and is associated with diseases including refractory hematologic cancer and hairy cell leukemia. A fully human anti-CD22 monoclonal antibody (“M971”) suitable for use with the methods disclosed herein is described, e.g., in Xiao et al., Mabs. 2009 May-June; 1(3): 297-303.

Programmed cell death protein 1, also known as “PD-1” and CD279 (cluster of differentiation 279), is a protein on the surface of cells that has a role in regulating the immune system's response to the cells of the human body by down-regulating the immune system and promoting self-tolerance by suppressing T cell inflammatory activity. This prevents autoimmune diseases, but it can also prevent the immune system from killing cancer cells. PD-1 is an immune checkpoint and guards against autoimmunity through two mechanisms. First, it promotes apoptosis (programmed cell death) of antigen-specific T-cells in lymph nodes. Second, it reduces apoptosis in regulatory T cells (anti-inflammatory, suppressive T cells). PD-1 is a cell surface receptor that belongs to the immunoglobulin superfamily and is expressed on T cells and pro-B cells. PD-1 binds two ligands, PD-L1 and PD-L2.

Programmed death-ligand 1 (“PD-L1”) is a 40 kDa type 1 transmembrane protein that has been speculated to play a major role in suppressing the adaptive arm of immune system during particular events such as pregnancy, tissue allografts, autoimmune disease and other disease states such as hepatitis. Normally the adaptive immune system reacts to antigens that are associated with immune system activation by exogenous or endogenous danger signals. In turn, clonal expansion of antigen-specific CD8+ T cells and/or CD4+ helper cells is propagated. The binding of PD-L1 to the inhibitory checkpoint molecule PD-1 transmits an inhibitory signal based on interaction with phosphatases (SHP-1 or SHP-2) via Immunoreceptor Tyrosine-Based Switch Motif (ITSM) motif. This reduces the proliferation of antigen-specific T-cells in lymph nodes, while simultaneously reducing apoptosis in regulatory T cells (anti-inflammatory, suppressive T cells)—further mediated by a lower regulation of the gene Bcl-2.

The “CD79α” and “CD79β” genes encode proteins that make up the B lymphocyte antigen receptor, a multimeric complex that includes the antigen-specific component, surface immunoglobulin (Ig). Surface Ig non-covalently associates with two other proteins, Ig-alpha and Ig-beta (encoded by CD79α and its paralog CD79β, respectively) which are necessary for expression and function of the B-cell antigen receptor. Functional disruption of this complex can lead to, e.g., human B-cell chronic lymphocytic leukemias.

B cell activating factor, or “BAFF” is a cytokine that belongs to the tumor necrosis factor (TNF) ligand family. This cytokine is a ligand for receptors TNFRSF13B/TACI, TNFRSF17/BCMA, and TNFRSF13C/BAFF-R. This cytokine is expressed in B cell lineage cells, and acts as a potent B cell activator. It has been also shown to play an important role in the proliferation and differentiation of B cells.

The term “interleukin 15” or “IL-15” refers to a pleiotropic cytokine that play important roles in maintenance and homeostatic expansion of various immune cells. IL-15 plays a critical role in the development of the NK lineage, and in survival, expansion, and function of NK cells. Local secretion of pleiotropic cytokines such as IL-15 in tumor microenvironment (TME) contributes to enhanced anti-tumor immunity. IL-15 is also involved in lymphocyte homeostasis as lymphocytes depend upon IL-15 for survival or expansion. IL-15 also plays multiple roles in peripheral innate and adaptive immune cell functions. IL-15 is trans-presented by antigen presenting cells and has a crucial role in the induction of central memory T cell subset and enhanced cytolytic effectors. It aids in T cell survival by reducing activation induced cell death (AICD). Human IL-15 precursor protein has two known isoforms based on the length of signal peptide. IL-15 (also referred to as IL-15-S48AA or IL-15LSP for “long signal peptide”) has a 48 amino acid signal peptide and propeptide while IL-15-S21AA or IL-15SSP (for “short signal peptide”), which is expressed from an alternatively spliced mRNA has a 21 amino acid signal peptide and propeptide. IL-15SSP has been shown not to be secreted, but rather stored intracellularly in the cytoplasm.

In some embodiments, IL-15 signal peptide comprises amino acids 1-29 of IL-15 protein sequence. In some embodiments, IL-15 signal peptide comprises a sequence of SEQ ID NO: 374. In some embodiments, IL-15 comprises amino acids 30-162 of IL-15 protein sequence. In some embodiments, IL-15 comprises a sequence of SEQ ID NO: 375.

(IL-15 protein sequence)
SEQ ID NO: 385
MRISKPHLRSISIQCYLCLLLNSHFLTEAGIHVFILGCFSAGLPKTEANW
VNVISDLKKIEDLIQSMHIDATLYTESDVHPSCKVTAMKCFLLELQVISL
ESGDASIHDTVENLIILANNSLSSNGNVTESGCKECEELEEKNIKEFLQS
FVHIVQMFINTS

The term “interleukin 15 receptor” or “IL-15R” refers to a receptor complex that IL-15 binds to and signals through. IL-15R is composed of three subunits, IL-15 receptor alpha chain (“IL-15Rα” or CD215), IL-2 receptor beta chain (“IL-2Rβ” or CD122) and IL-2 receptor gamma/the common gamma chain (“IL-2Rγ/γc” or CD132). Human IL-15Rα precursor protein has a 30 amino acid signal peptide, a 175 amino acid extracellular domain, a 23 amino acid single membrane-spanning transmembrane stretch, and a 39 amino acid cytoplasmic (or intracellular) domain and contains N- and O-linked glycosylation sites. IL-15Rα contains a Sushi domain (amino acid 31-95) which is essential for IL-15 binding. IL-15Rα also exists as a soluble form (sIL-15Rα). sIL-15Rα is constitutively generated from the transmembrane receptor through a defined proteolytic cleavage, and this process can be enhanced by certain chemical agents, such as PMA. The human sIL-15Rα, about 42 kDa in size, may could prolong the half-life of IL-15 or potentiate IL-15 signaling through IL-15 binding and IL-2Rβ/γc heterodimer. Although IL-15R shares subunits with IL-2R that contain the cytoplasmic motifs required for signal transduction, IL-15 signaling has separate biological effects in vivo apart from many biological activities overlapping with IL-2 signaling due to IL-15Rα subunit that is unique to IL-15R, availability and concentration of IL-15, the kinetics and affinity of IL-15-IL-15Rα binding. IL-15 binds to IL-15Rα specifically with high affinity, which then associates with a complex composed of IL-2Rβ and IL-2Rγ/γc subunits, expressed on the same cell (“cis-presentation”) or on a different cell (“trans-presentation”). The interaction between IL-15 and IL-15Rα is independent of the complex composed of IL-2Rβ and IL-2Rγ/γc subunits. IL-15 binding to the IL-2Rβ/γc heterodimeric receptor induces JAK1 activation that phosphorylates STAT3 via the beta chain, and JAK3 activation that phosphorylates STAT5 via the gamma chain. The IL-15/IL-15R interaction modulates not only T-cell development and homeostasis, but also in memory CD8+ T-cell and NK cell development, maintenance, expansion and activities.

In some embodiments, IL-15Rα cytoplasmic (or intracellular) domain comprises amino acids 229-267 of IL-15Rα protein. In some embodiments, IL-15Rα cytoplasmic (or intracellular) domain comprises a sequence of SEQ ID NO: 372. In some embodiments, IL-15Rα Sushi domain comprises amino acids 31-95 of IL-15Rα protein. In some embodiments, IL-15Rα Sushi domain comprises a sequence of SEQ ID NO: 382. In some embodiments, IL-15Rα comprises the transmembrane domain and the cytoplasmic (intracellular) domain of IL-15Rα protein. In some embodiments, IL-15Rα comprises amino acids 96-267 of IL-15Rα protein. In some embodiments, IL-15Rα comprises a sequence of SEQ ID NO: 383. In some embodiments, sIL-15Rα comprises amino acids 21-205 of IL-15Rα protein. In some embodiments, sIL-15Rα comprises a sequence of SEQ ID NO: 379. In some embodiments, IL-15Rα comprises a sequence of SEQ ID NO: 403.

(IL-15Rα protein sequence)
SEQ ID NO: 386
MAPRRARGCRTLGLPALLLLLLLRPPATRGITCPPPMSVEHADIWVKSYS
LYSRERYICNSGFKRKAGTSSLTECVLNKATNVAHWTTPSLKCIRDPALV
HQRPAPPSTVTTAGVTPQPESLSPSGKEPAASSPSSNNTAATTAAIVPGS
QLMPSKSPSTGTTEISSHESSHGTPSQTTAKNWELTASASHQPPGVYPQG
HSDTTVAISTSTVLLCGLSAVSLLACYLKSRQTPPLASVEMEAMEALPVT
WGTSSRDEDLENCSHHL

The term “anti-tumor effect” refers to a biological effect which can be manifested by various means, including but not limited to, e.g., a decrease in tumor volume, a decrease in the number of tumor cells, a decrease in the number of metastases, an increase in life expectancy, decrease in tumor cell proliferation, decrease in tumor cell survival, or amelioration of various physiological symptoms associated with the cancerous condition. An “anti-tumor effect” can also be manifested by the ability of the peptides, polynucleotides, cells and antibodies of the present disclosure in prevention of the occurrence of tumor in the first place.

The term “autologous” refers to any material derived from the same individual to whom it is later to be re-introduced into the individual.

The term “allogeneic” or, alternatively, “allogenic,” refers to any material derived from a different animal of the same species or different patient as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “xenogeneic” refers to a graft derived from an animal of a different species.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some versions contain one or more introns.

The term “effective amount” or “therapeutically effective amount” are used interchangeably herein, and refer to an amount of a compound, formulation, material, or composition, as described herein effective to achieve a particular biological or therapeutic result.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence driven by a promoter.

The term “functional disruption” refers to a physical or biochemical change to a specific (e.g., target) nucleic acid (e.g., gene, RNA transcript, of protein encoded thereby) that prevents its normal expression and/or behavior in the cell. In one embodiment, a functional disruption refers to a modification of the gene via a gene editing method. In one embodiment, a functional disruption prevents expression of a target gene (e.g., an endogenous gene).

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR™ gene delivery technology from Oxford BioMedica, the LENTIMAX™ vector system from Lentigen, and the like. Nonclinical types of lentiviral vectors are also available and would be known to one skilled in the art.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Human” or “fully human” refers to an immunoglobulin, such as an antibody or antibody fragment, where the whole molecule is of human origin or consists of an amino acid sequence identical to a human form of the antibody or immunoglobulin.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the present disclosure by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a TFP of the present disclosure can be replaced with other amino acid residues from the same side chain family and the altered TFP can be tested using the functional assays described herein.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “parenteral” administration of an immunogenic composition includes, e.g., subcutaneous (s.c.), intravenous (i.v.), intramuscular (i.m.), or intrasternal injection, intratumoral, or infusion techniques.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the transcription machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific” promoter refers to a nucleotide sequence which, when operably linked with a polynucleotide encodes or specified by a gene, causes the gene product to be produced in a cell substantially only if the cell is a cell of the tissue type corresponding to the promoter.

The terms “linker” and “flexible polypeptide linker” as used in the context of a scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Ser)ₙ, where n is a positive integer equal to or greater than 1. For example, n=1, n=2, n=3, n=4, n=5, n=6, n=7, n=8, n=9 and n=10. In one embodiment, the flexible polypeptide linkers include, but are not limited to, (Gly₄Ser)₄ or (Gly₄Ser)₃. In another embodiment, the linkers include multiple repeats of (Gly₂Ser), (GlySer) or (Gly₃Ser). Also included within the scope of the present disclosure are linkers described in WO2012/138475 (incorporated herein by reference). In some instances, the linker sequence comprises (G₄S)ₙ, wherein n=2 to 5. In some instances, the linker sequence comprises (G₄S)ₙ, wherein n=1 to 3.

As used herein, a 5′ cap (also termed an RNA cap, an RNA 7-methylguanosine cap or an RNA m7G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from Rnases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

As used herein, “in vitro transcribed RNA” refers to RNA, preferably mRNA, which has been synthesized in vitro. Generally, the in vitro transcribed RNA is generated from an in vitro transcription vector. The in vitro transcription vector comprises a template that is used to generate the in vitro transcribed RNA.

As used herein, a “poly(A)” is a series of adenosines attached by polyadenylation to the mRNA. In the preferred embodiment of a construct for transient expression, the polyA is between 50 and 5000, preferably greater than 64, more preferably greater than 100, most preferably greater than 300 or 400. Poly(A) sequences can be modified chemically or enzymatically to modulate mRNA functionality such as localization, stability or efficiency of translation.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals, human).

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

In the context of the present disclosure, “tumor antigen” or “hyperproliferative disorder antigen” or “antigen associated with a hyperproliferative disorder” refers to antigens that are common to specific hyperproliferative disorders. In certain aspects, the hyperproliferative disorder antigens of the present disclosure are derived from, cancers including but not limited to primary or metastatic melanoma, thymoma, lymphoma, sarcoma, lung cancer, liver cancer, NHL, leukemias, uterine cancer, cervical cancer, bladder cancer, kidney cancer and adenocarcinomas such as breast cancer, prostate cancer, ovarian cancer, pancreatic cancer, and the like.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to an antibody, an antibody fragment or a specific ligand, which recognizes and binds a cognate binding partner (e.g., CD19) present in a sample, but which does not necessarily and substantially recognize or bind other molecules in the sample.

As used herein, the term “meganuclease” refers to an endonuclease that binds double-stranded DNA at a recognition sequence that is greater than 12 base pairs. Preferably, the recognition sequence for a meganuclease of the present disclosure is 22 base pairs. A meganuclease can be an endonuclease that is derived from I-Crel and can refer to an engineered variant of I-Crel that has been modified relative to natural I-Crel with respect to, for example, DNA-binding specificity, DNA cleavage activity, DNA-binding affinity, or dimerization properties. Methods for producing such modified variants of I-Crel are known in the art (e.g., WO 2007/047859). A meganuclease as used herein binds to double-stranded DNA as a heterodimer or as a “single-chain meganuclease” in which a pair of DNA-binding domains are joined into a single polypeptide using a peptide linker. The term “homing endonuclease” is synonymous with the term “meganuclease.” Meganucleases of the present disclosure are substantially non-toxic when expressed in cells, particularly in human T cells, such that cells can be transfected and maintained at 37° C. without observing deleterious effects on cell viability or significant reductions in meganuclease cleavage activity when measured using the methods described herein.

As used herein, the term “single-chain meganuclease” refers to a polypeptide comprising a pair of nuclease subunits joined by a linker. A single-chain meganuclease has the organization: N-terminal subunit—Linker—C-terminal subunit. The two meganuclease subunits will generally be non-identical in amino acid sequence and will recognize non-identical DNA sequences. Thus, single-chain meganucleases typically cleave pseudo-palindromic or non-palindromic recognition sequences. A single-chain meganuclease may be referred to as a “single-chain heterodimer” or “single-chain heterodimeric meganuclease” although it is not, in fact, dimeric. For clarity, unless otherwise specified, the term “meganuclease” can refer to a dimeric or single-chain meganuclease.

As used herein, the term “TALEN” refers to an endonuclease comprising a DNA-binding domain comprising 16-22 TAL domain repeats fused to any portion of the Fok1 nuclease domain.

As used herein, the term “Compact TALEN” refers to an endonuclease comprising a DNA-binding domain with 16-22 TAL domain repeats fused in any orientation to any catalytically active portion of nuclease domain of the I-Tev1 homing endonuclease.

As used herein, the term “CRISPR” refers to a caspase-based endonuclease comprising a caspase, such as Cas9, and a guide RNA that directs DNA cleavage of the caspase by hybridizing to a recognition site in the genomic DNA.

As used herein, the term “megaTAL” refers to a single-chain nuclease comprising a transcription activator-like effector (TALE) DNA binding domain with an engineered, sequence-specific homing endonuclease.

As is used herein, the terms “T cell receptor” and “T cell receptor complex” are used interchangeably to refer to a molecule found on the surface of T cells that is, in general, responsible for recognizing antigens. The TCR comprises a heterodimer consisting of a TCR alpha and TCR beta chain in 95% of T cells, whereas 5% of T cells have TCRs consisting of TCR gamma and TCR delta chains. The TCR further comprises one or more of CD3ε, CD3γ, and CD3δ. In some embodiments, the TCR comprises CD3ε. In some embodiments, the TCR comprises CD3γ. In some embodiments, the TCR comprises CD3δ. In some embodiments, the TCR comprises CD3ζ. Engagement of the TCR with antigen, e.g., with antigen and MHC, results in activation of its T cells through a series of biochemical events mediated by associated enzymes, co-receptors, and specialized accessory molecules. In some embodiments, the constant domain of human TCR alpha has a sequence of SEQ ID NO: 142. In some embodiments, the constant domain of human TCR alpha has an IgC domain having a sequence of SEQ ID NO: 143, a transmembrane domain having a sequence of SEQ ID NO: 144, and an intracellular domain having a sequence of SS. In some embodiments, the constant domain of murine TCR alpha has a sequence of SEQ ID NO: 147. In some embodiments, the constant domain of murine TCR alpha has a transmembrane domain having a sequence of SEQ ID NO: 144, and an intracellular domain having a sequence of SS. In some embodiments, the constant domain of human TCR beta has a sequence of SEQ ID NO: 148. In some embodiments, the constant domain of human TCR beta has an IgC domain having a sequence of SEQ ID NO: 149, a transmembrane domain having a sequence of SEQ ID NO: 150, and an intracellular domain having a sequence of SEQ ID NO: 151. In some embodiments, the constant domain of murine TCR beta has a sequence of SEQ ID NO: 152. In some embodiments, the constant domain of murine TCR beta has a transmembrane domain having a sequence of SEQ ID NO: 152, and an intracellular domain having a sequence of SEQ ID NO: 153. In some embodiments, the constant domain of human TCR delta has a sequence of SEQ ID NO: 243. In some embodiments, the constant domain of human TCR delta has an IgC domain having a sequence of SEQ ID NO: 265, a transmembrane domain having a sequence of SEQ ID NO: 158, and an intracellular domain having a sequence of L. In some embodiments, the constant domain of human TCR gamma has a sequence of SEQ ID NO: 21. In some embodiments, the constant domain of human TCR gamma has an IgC domain having a sequence of SEQ ID NO: 155, a transmembrane domain having a sequence of SEQ ID NO: 156, and an intracellular domain having a sequence of SEQ ID NO: 157.

In some embodiments, human CD3 epsilon has a sequence of SEQ ID NO: 364. In some embodiments, human CD3 epsilon has an extracellular domain having a sequence of SEQ ID NO: 126, a transmembrane domain having a sequence of SEQ ID NO: 127, and an intracellular domain, e.g., an intracellular signaling domain, having a sequence of SEQ ID NO: 128. In some embodiments, human CD3 delta has a sequence of SEQ ID NO: 136. In some embodiments, human CD3 delta has an extracellular domain having a sequence of SEQ ID NO: 138, a transmembrane domain having a sequence of SEQ ID NO: 139, and an intracellular domain, e.g., an intracellular signaling domain, having a sequence of SEQ ID NO: 140. In some embodiments, human CD3 gamma has a sequence of SEQ ID NO: 130. In some embodiments, human CD3 gamma has an extracellular domain having a sequence of SEQ ID NO: 132, a transmembrane domain having a sequence of SEQ ID NO: 133, and an intracellular domain, e.g., an intracellular signaling domain, having a sequence of SEQ ID NO: 134.

Ranges: throughout this disclosure, various aspects of the present disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

Provided herein are compositions of matter and methods of use for the treatment of a disease such as cancer, using modified T cells comprising a T cell receptors (TCR) fusion protein (TFP) in combination with an IL-15 and/or IL-15Rα polypeptide. Advantageously, when these IL-15 and/or IL-15-Rα proteins are expressed in combination with the TCR fusion proteins, they can confer increased persistence, prolonged activity, and increased efficacy on the T cells for treating the malignancies described herein. As used herein, a “T cell receptor (TCR) fusion protein” or “TFP” includes a recombinant polypeptide derived from the various polypeptides comprising the TCR that is generally capable of i) binding to a surface antigen on target cells and ii) interacting with other polypeptide components of the intact TCR complex, typically when co-located in or on the surface of a T cell. As provided herein, TFPs provide substantial benefits as compared to Chimeric Antigen Receptors. The term “Chimeric Antigen Receptor” or alternatively a “CAR” refers to a recombinant polypeptide comprising an extracellular antigen binding domain in the form of, e.g., a single domain antibody or scFv, a transmembrane domain, and cytoplasmic signaling domains (also referred to herein as “intracellular signaling domains”) comprising a functional signaling domain derived from a stimulatory molecule as defined below. Generally, the central intracellular signaling domain of a CAR is derived from the CD3 zeta chain that is normally found associated with the TCR complex. The CD3 zeta signaling domain can be fused with one or more functional signaling domains derived from at least one co-stimulatory molecule such as 4-1BB (i.e., CD137), CD27 and/or CD28.

### T Cell Receptor (TCR) Fusion Proteins (TFPs)

The present disclosure encompasses recombinant DNA constructs encoding TFPs, wherein the TFP comprises a binding domain, e.g., an antibody or antibody fragment, a ligand, or a ligand binding protein, wherein the sequence of the binding domain is contiguous with and in the same reading frame as a nucleic acid sequence encoding a TCR subunit or portion thereof. The present disclosure encompasses recombinant DNA constructs encoding TFPs, wherein the TFP comprises an antibody fragment that binds specifically to a tumor associated antigen (TAA) wherein the sequence of the antibody fragment is contiguous with and in the same reading frame as a nucleic acid sequence encoding a TCR subunit or portion thereof. The present disclosure encompasses recombinant DNA constructs encoding TFPs, wherein the TFP comprises an antibody fragment that binds specifically to CD19, e.g., human CD19, wherein the sequence of the antibody fragment is contiguous with and in the same reading frame as a nucleic acid sequence encoding a TCR subunit or portion thereof. The present disclosure encompasses recombinant DNA constructs encoding TFPs, wherein the TFP comprises an antibody fragment that binds specifically to mesothelin, e.g., human mesothelin, wherein the sequence of the antibody fragment is contiguous with and in the same reading frame as a nucleic acid sequence encoding a TCR subunit or portion thereof. The present disclosure encompasses recombinant DNA constructs encoding TFPs, wherein the TFP comprises an antibody fragment that binds specifically to MUC16, e.g., human MUC16, wherein the sequence of the antibody fragment is contiguous with and in the same reading frame as a nucleic acid sequence encoding a TCR subunit or portion thereof. The present disclosure encompasses recombinant DNA constructs encoding TFPs, wherein the TFP comprises an antibody fragment that binds specifically to CD20, e.g., human CD20, wherein the sequence of the antibody fragment is contiguous with and in the same reading frame as a nucleic acid sequence encoding a TCR subunit or portion thereof. The present disclosure encompasses recombinant DNA constructs encoding TFPs, wherein the TFP comprises an antibody fragment that binds specifically to CD70, e.g., human CD70, wherein the sequence of the antibody fragment is contiguous with and in the same reading frame as a nucleic acid sequence encoding a TCR subunit or portion thereof. The present disclosure encompasses recombinant DNA constructs encoding TFPs, wherein the TFP comprises an antibody fragment that binds specifically to CD79B, e.g., human CD79B, wherein the sequence of the antibody fragment is contiguous with and in the same reading frame as a nucleic acid sequence encoding a TCR subunit or portion thereof. The present disclosure encompasses recombinant DNA constructs encoding TFPs, wherein the TFP comprises an antibody fragment that binds specifically to HER2, e.g., human HER2, wherein the sequence of the antibody fragment is contiguous with and in the same reading frame as a nucleic acid sequence encoding a TCR subunit or portion thereof. The present disclosure encompasses recombinant DNA constructs encoding TFPs, wherein the TFP comprises an antibody fragment that binds specifically to PSMA, e.g., human PSMA, wherein the sequence of the antibody fragment is contiguous with and in the same reading frame as a nucleic acid sequence encoding a TCR subunit or portion thereof. The present disclosure encompasses recombinant DNA constructs encoding TFPs, wherein the TFP comprises an antibody fragment that binds specifically to BCMA, e.g., human BCMA, wherein the sequence of the antibody fragment is contiguous with and in the same reading frame as a nucleic acid sequence encoding a TCR subunit or portion thereof. The present disclosure encompasses recombinant DNA constructs encoding TFPs, wherein the TFP comprises an antibody fragment that binds specifically to ROR1, e.g., human ROR1, wherein the sequence of the antibody fragment is contiguous with and in the same reading frame as a nucleic acid sequence encoding a TCR subunit or portion thereof. The present disclosure encompasses recombinant DNA constructs encoding TFPs, wherein the TFP comprises an antibody fragment that binds specifically to CD22, e.g., human CD22, wherein the sequence of the antibody fragment is contiguous with and in the same reading frame as a nucleic acid sequence encoding a TCR subunit or portion thereof. The present disclosure encompasses recombinant DNA constructs encoding TFPs, wherein the TFP comprises an antibody fragment that binds specifically to GPC3, e.g., human GPC3, wherein the sequence of the antibody fragment is contiguous with and in the same reading frame as a nucleic acid sequence encoding a TCR subunit or portion thereof. The present disclosure encompasses recombinant DNA constructs encoding TFPs, wherein the TFP comprises an antibody fragment that binds specifically to Nectin-4, e.g., human Nectin-4, wherein the sequence of the antibody fragment is contiguous with and in the same reading frame as a nucleic acid sequence encoding a TCR subunit or portion thereof. The present disclosure encompasses recombinant DNA constructs encoding TFPs, wherein the TFP comprises an antibody fragment that binds specifically to Trop-2, e.g., human Trop-2, wherein the sequence of the antibody fragment is contiguous with and in the same reading frame as a nucleic acid sequence encoding a TCR subunit or portion thereof. The TFPs provided herein are able to associate with one or more endogenous (or alternatively, one or more exogenous, or a combination of endogenous and exogenous) TCR subunits in order to form a functional TCR complex.

In one aspect, the TFP of the present disclosure comprises a target-specific binding element otherwise referred to as an antigen binding domain. The choice of moiety depends upon the type and number of target antigen that define the surface of a target cell. For example, the antigen binding domain may be chosen to recognize a target antigen that acts as a cell surface marker on target cells associated with a particular disease state. Thus, examples of cell surface markers that may act as target antigens for the antigen binding domain in a TFP of the present disclosure include those associated with viral, bacterial and parasitic infections; autoimmune diseases; and cancerous diseases (e.g., malignant diseases).

In one aspect, the TFP-mediated T cell response can be directed to an antigen of interest by way of engineering an antigen-binding domain into the TFP that specifically binds a desired antigen.

The antigen binding domain can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (V_{H}), a light chain variable domain (V_{L}) and a variable domain (V_{HH}) of a camelid derived nanobody, and to an alternative scaffold known in the art to function as antigen binding domain, such as a recombinant fibronectin domain, anticalin, DARPIN and the like. Likewise, a natural or synthetic ligand specifically recognizing and binding the target antigen can be used as antigen binding domain for the TFP. In some instances, it is beneficial for the antigen binding domain to be derived from the same species in which the TFP will ultimately be used in. For example, for use in humans, it may be beneficial for the antigen binding domain of the TFP to comprise human or humanized residues for the antigen binding domain of an antibody or antibody fragment.

Thus, in one aspect, the antigen-binding domain comprises a murine, humanized or human antibody or an antibody fragment, or a murine antibody or antibody fragment. In one embodiment, the murine, humanized or human anti-TAA binding domain comprises one or more (e.g., all three) light chain complementary determining region 1 (LC CDR1), light chain complementary determining region 2 (LC CDR2), and light chain complementary determining region 3 (LC CDR3) of a murine, humanized or human anti-TAA binding domain described herein, and/or one or more (e.g., all three) heavy chain complementary determining region 1 (HC CDR1), heavy chain complementary determining region 2 (HC CDR2), and heavy chain complementary determining region 3 (HC CDR3) of a murine, humanized or human anti-CD19 binding domain described herein, e.g., a murine, humanized or human anti-TAA binding domain comprising one or more, e.g., all three, LC CDRs and one or more, e.g., all three, HC CDRs. In one embodiment, the murine, humanized or human anti-CD19 binding domain comprises one or more (e.g., all three) heavy chain complementary determining region 1 (HC CDR1), heavy chain complementary determining region 2 (HC CDR2), and heavy chain complementary determining region 3 (HC CDR3) of a murine, humanized or human anti-TAA binding domain described herein, e.g., the murine, humanized or human anti-TAA binding domain has two variable heavy chain regions, each comprising a HC CDR1, a HC CDR2 and a HC CDR3 described herein. In one embodiment, the murine, humanized or human anti-TAA binding domain comprises a humanized or human light chain variable region described herein and/or a murine, humanized or human heavy chain variable region described herein. In one embodiment, the murine, humanized or human anti-TAA binding domain comprises a murine, humanized or human heavy chain variable region described herein, e.g., at least two murine, humanized or human heavy chain variable regions described herein. In one embodiment, the anti-TAA binding domain is a scFv comprising a light chain and a heavy chain of an amino acid sequence provided herein. In an embodiment, the anti-TAA binding domain (e.g., a scFv) comprises: a light chain variable region comprising an amino acid sequence having at least one, two or three modifications (e.g., substitutions) but not more than 30, 20 or 10 modifications (e.g., substitutions) of an amino acid sequence of a light chain variable region provided herein, or a sequence with 95-99% identity with an amino acid sequence provided herein; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided herein, or a sequence with 95-99% identity to an amino acid sequence provided herein. In one embodiment, the murine, humanized or human anti-TAA binding domain is a scFv, and a light chain variable region comprising an amino acid sequence described herein, is attached to a heavy chain variable region comprising an amino acid sequence described herein, via a linker, e.g., a linker described herein. In one embodiment, the murine, humanized, or human anti-TAA binding domain includes a (Gly₄-Ser)˜ linker, wherein n is 1, 2, 3, 4, 5, or 6, preferably 3 or 4. The light chain variable region and heavy chain variable region of a scFv can be, e.g., in any of the following orientations: light chain variable region-linker-heavy chain variable region or heavy chain variable region-linker-light chain variable region. In some instances, the linker sequence comprises a long linker (LL) sequence. In some instances, the long linker sequence comprises (G₄S)ₙ, wherein n=2 to 4. In some instances, the linker sequence comprises a short linker (SL) sequence. In some instances, the short linker sequence comprises (G₄S)ₙ, wherein n=1 to 3.

In some embodiments, the antigen-binding domain comprises an anti-CD19 murine, humanized or human antibody or an antibody fragment, or a murine antibody or antibody fragment having a light chain CDR1 of SEQ ID NO:73, a CDR2 of SEQ ID NO:75, and a CDR3 of SEQ ID NO:77 and a heavy chain CDR1 of SEQ ID NO:79, a CDR2 of SEQ ID NO:81, and a CDR3 of SEQ ID NO:83. In some embodiments, the anti-CD19 antibody is a murine scFv. In some embodiments, the anti-CD-19 antibody comprises a VL of SEQ ID NO:85 and a VH of SEQ ID NO:87

In some embodiments, the antigen-binding domain comprises an anti-mesothelin murine, humanized or human single domain antibody or an antibody fragment having a CDR1 of SEQ ID NO:60, a CDR2 of SEQ ID NO:61, and a CDR3 of SEQ ID NO:62 or a CDR1 of SEQ ID NO:63, a CDR2 of SEQ ID NO:64, and a CDR3 of SEQ ID NO:65 or a CDR1 of SEQ ID NO:66, a CDR2 of SEQ ID NO:67, and a CDR3 of SEQ ID NO:68. In some embodiments, the anti-mesothelin antibody has a variable domain of SEQ ID NO:69, SEQ ID NO:70, or SEQ ID NO:71.

In some embodiments, the antigen-binding domain comprises an anti-CD70 murine, humanized or human single domain antibody or an antibody fragment having a CDR1 of SEQ ID NO:88, a CDR2 of SEQ ID NO:89, and a CDR3 of SEQ ID NO:90, or a CDR1 of SEQ ID NO:92, a CDR2 of SEQ ID NO:93, and a CDR3 of SEQ ID NO:94, or a CDR1 of SEQ ID NO:96, a CDR2 of SEQ ID NO:97, and a CDR3 of SEQ ID NO:98, or a CDR1 of SEQ ID NO:100, a CDR2 of SEQ ID NO:101, and a CDR3 of SEQ ID NO:102, or a CDR1 of SEQ ID NO: 104, a CDR2 of SEQ ID NO: 105, and a CDR3 of SEQ ID NO: 106, or a CDR1 of SEQ ID NO:108, a CDR2 of SEQ ID NO:109, and a CDR3 of SEQ ID NO:110, or a CDR1 of SEQ ID NO:112, a CDR2 of SEQ ID NO:113, and a CDR3 of SEQ ID NO:114, or a CDR1 of SEQ ID NO116, a CDR2 of SEQ ID NO: 117, and a CDR3 of SEQ ID NO:118, or a CDR1 of SEQ ID NO:120, a CDR2 of SEQ ID NO:121, and a CDR3 of SEQ ID NO:122.

In some embodiments, the antigen-binding domain comprises an anti-CD70 murine, humanized or human single domain antibody or antibody fragment have a CDR1 of SEQ ID NO: 392, a CDR2 of SEQ ID NO: 393, a CDR3 of SEQ ID NO: 394, or a CDR1 of SEQ ID NO: 396, a CDR2 of SEQ ID NO: 397, a CDR3 of SEQ ID NO: 398. In some embodiments, the antigen binding domain comprises CDR sequences selected from the group consisting of SEQ ID NO: 392-394 and 396-398. In some embodiments, the antigen binding domain comprises SEQ ID Nos: 392-394 and 396-398. In some embodiments, the antigen-binding domain comprises an anti-CD70 heavy chain variable domain region comprising SEQ ID NO: 392 as CDR1, a SEQ ID NO: 393 as CDR2, and SEQ ID NO: 394 as CDR3. In some embodiments, the antigen binding domain comprises an anti-CD70 light chain variable domain region comprising SEQ ID NO: 396 as CDR1, SEQ ID NO: 397 as CDR2, and SEQ ID NO: 398 as CDR3. In some embodiments, the antigen binding domain comprises an anti-CD70 heavy chain variable domain region comprising SEQ ID NO: 392 as CDR1, SEQ ID NO: 393 as CDR2, and SEQ ID NO: 394 as CDR3 and a light chain variable domain region comprising SEQ ID NO: 396 as CDR1, SEQ ID NO: 397 as CDR2, and SEQ ID NO: 398 as CDR3. In some embodiments, the antigen binding domain comprises SEQ ID NO: 395. In some embodiments, the antigen binding domain comprises SEQ ID NO: 399. In some embodiments, the antigen binding domain comprises SEQ ID NO: 395 and SEQ ID NO: 399. In some embodiments, the antigen binding domain comprises a linker sequence. In some embodiments, the antigen binding domain linker sequence comprises SEQ ID NO: 401. In some embodiments, the antigen binding domain comprises SEQ ID Nos: 399, 401, and 395. In some embodiments, the antigen binding domain comprises SEQ ID Nos: 399, 401, and 395 when read in 5′ to 3′ order.

In some aspects, a non-human antibody is humanized, where specific sequences or regions of the antibody are modified to increase similarity to an antibody naturally produced in a human or fragment thereof. In one aspect, the antigen binding domain is humanized.

A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J Mol. Biol., 235(3):959-73 (1994), each of which is incorporated herein in its entirety by reference. Often, framework residues in the framework regions will be substituted with the corresponding residue from the CDR donor antibody to alter, for example improve, antigen binding. These framework substitutions are identified by methods well-known in the art, e.g., by modeling of the interactions of the CDR and framework residues to identify framework residues important for antigen binding and sequence comparison to identify unusual framework residues at particular positions (see, e.g., Queen et al., U.S. Pat. No. 5,585,089; and Riechmann et al., 1988, Nature, 332:323, which are incorporated herein by reference in their entireties.)

A humanized antibody or antibody fragment has one or more amino acid residues remaining in it from a source which is nonhuman. These nonhuman amino acid residues are often referred to as “import” residues, which are typically taken from an “import” variable domain. As provided herein, humanized antibodies or antibody fragments comprise one or more CDRs from nonhuman immunoglobulin molecules and framework regions wherein the amino acid residues comprising the framework are derived completely or mostly from human germline. Multiple techniques for humanization of antibodies or antibody fragments are well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference in their entirety). In such humanized antibodies and antibody fragments, substantially less than an intact human variable domain has been substituted by the corresponding sequence from a nonhuman species. Humanized antibodies are often human antibodies in which some CDR residues and possibly some framework (FR) residues are substituted by residues from analogous sites in rodent antibodies. Humanization of antibodies and antibody fragments can also be achieved by veneering or resurfacing (EP 592,106; EP 519,596; Padlan, 1991, Molecular Immunology, 28(4/5):489-498; Studnicka et al., Protein Engineering, 7(6):805-814 (1994); and Roguska et al., Proc. Natl. Acad. Sci. USA, 91:969-973 (1994)) or chain shuffling (U.S. Pat. No. 5,565,332), the contents of which are incorporated herein by reference in their entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J Immunol., 151:2296 (1993); Chothia et al., J. Mol. Biol., 196:901 (1987), the contents of which are incorporated herein by reference herein in their entirety). Another method uses a particular framework derived from the consensus sequence of all human antibodies of a particular subgroup of light or heavy chains. The same framework may be used for several different humanized antibodies (see, e.g., Nicholson et al., Mol. Immun. 34 (16-17): 1157-1165 (1997); Carter et al., Proc. Natl. Acad. Sci. USA, 89:4285 (1992); Presta et al., J Immunol., 151:2623 (1993), the contents of which are incorporated herein by reference herein in their entirety). In some embodiments, the framework region, e.g., all four framework regions, of the heavy chain variable region are derived from a V_{H}4-4-59 germline sequence. In one embodiment, the framework region can comprise, one, two, three, four or five modifications, e.g., substitutions, e.g., from the amino acid at the corresponding murine sequence. In one embodiment, the framework region, e.g., all four framework regions of the light chain variable region are derived from a VK3-1.25 germline sequence. In one embodiment, the framework region can comprise, one, two, three, four or five modifications, e.g., substitutions, e.g., from the amino acid at the corresponding murine sequence.

In some aspects, the portion of a TFP composition of the present disclosure that comprises an antibody fragment is humanized with retention of high affinity for the target antigen and other favorable biological properties. According to one aspect of the present disclosure, humanized antibodies and antibody fragments are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, e.g., the analysis of residues that influence the ability of the candidate immunoglobulin to bind the target antigen. In this way, FR residues can be selected and combined from the recipient and import sequences so that the desired antibody or antibody fragment characteristic, such as increased affinity for the target antigen, is achieved. In general, the CDR residues are directly and most substantially involved in influencing antigen binding.

A humanized antibody or antibody fragment may retain a similar antigenic specificity as the original antibody, e.g., in the present disclosure, the ability to bind human a tumor associated antigen (TAA). In some embodiments, a humanized antibody or antibody fragment may have improved affinity and/or specificity of binding to, e.g., human CD19, human BCMA, or another tumor associated antigen.

In one aspect, the binding domain is characterized by particular functional features or properties of an antibody or antibody fragment. For example, in one aspect, the portion of a TFP composition of the present disclosure that comprises an antigen binding domain specifically binds human CD19. In one aspect, the antigen binding domain has the same or a similar binding specificity to human CD19 as the FMC63 scFv described in Nicholson et al., Mol. Immun. 34 (16-17): 1157-1165 (1997). In one aspect, the present disclosure relates to an antigen binding domain comprising an antibody or antibody fragment, wherein the antibody binding domain specifically binds to a CD19 or BCMA protein or fragment thereof, wherein the antibody or antibody fragment comprises a variable light chain and/or a variable heavy chain that includes an amino acid sequence provided herein. In certain aspects, the scFv is contiguous with and in the same reading frame as a leader sequence.

In one aspect, the anti-tumor-associated antigen binding domain is a fragment, e.g., a single chain variable fragment (scFv). In one aspect, the anti-TAA binding domain is a Fv, a Fab, a (Fab′)₂, or a bi-functional (e.g., bi-specific) hybrid antibody (e.g., Lanzavecchia et al., Eur. J. Immunol. 17, 105 (1987)). In one aspect, the antibodies and fragments thereof of the present disclosure binds a CD19 protein with wild-type or enhanced affinity. In another aspect, the anti-TAA binding domain comprises a single domain antibody (sdAb or V_{HH}).

Also provided herein are methods for obtaining an antibody antigen binding domain specific for a target antigen (e.g., CD19, BCMA or any target antigen described elsewhere herein for targets of fusion moiety binding domains), the method comprising providing by way of addition, deletion, substitution or insertion of one or more amino acids in the amino acid sequence of a V_{H} domain set out herein a V_{H} domain which is an amino acid sequence variant of the V_{H} domain, optionally combining the V_{H} domain thus provided with one or more V_{L} domains, and testing the V_{H} domain or V_{H}/V_{L} combination or combinations to identify a specific binding member or an antibody antigen binding domain specific for a target antigen of interest (e.g., MSLN, CD79B, etc.) and optionally with one or more desired properties.

In some instances, V_{H} domains and scFvs can be prepared according to method known in the art (see, for example, Bird et al., (1988) Science 242:423-426 and Huston et al., (1988) Proc. Natl. Acad. Sci. USA 85:5879-5883). scFv molecules can be produced by linking V_{H} and V_{L} regions together using flexible polypeptide linkers. The scFv molecules comprise a linker (e.g., a Ser-Gly linker) with an optimized length and/or amino acid composition. The linker length can greatly affect how the variable regions of a scFv fold and interact. In fact, if a short polypeptide linker is employed (e.g., between 5-10 amino acids) intra-chain folding is prevented. Inter-chain folding is also required to bring the two variable regions together to form a functional epitope binding site. In some instances, the linker sequence comprises a linker sequence. In some instances, the long linker sequence comprises (G₄S)ₙ, wherein n=2 to 4. In some instances, the linker sequence comprises (G₄S)ₙ, wherein n=1 to 3. For examples of linker orientation and size see, e.g., Hollinger et al., 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, each of which is incorporated herein by reference.

An scFv can comprise a linker of about 10, 11, 12, 13, 14, 15 or greater than 15 residues between its V_{L} and V_{H} regions. The linker sequence may comprise any naturally occurring amino acid. In some embodiments, the linker sequence comprises amino acids glycine and serine. In another embodiment, the linker sequence comprises sets of glycine and serine repeats such as (Gly₄Ser)ₙ, where n is a positive integer equal to or greater than 1. In one embodiment, the linker can be (Gly₄Ser)₄ or (Gly₄Ser)₃. Variation in the linker length may retain or enhance activity, giving rise to superior efficacy in activity studies. In some instances, the linker sequence comprises (G₄S)ₙ, wherein n=2 to 4. In some instances, the linker sequence comprises (G₄S)ₙ, wherein n=1 to 3. In some embodiments, the linker sequence may comprise SEQ ID NO: 401.

### Stability and Mutations

The stability of a tumor associated antigen binding domain, e.g., scFv molecules (e.g., soluble scFv) can be evaluated in reference to the biophysical properties (e.g., thermal stability) of a conventional control scFv molecule or a full-length antibody. In one embodiment, the humanized or human scFv has a thermal stability that is greater than about 0.1, about 0.25, about 0.5, about 0.75, about 1, about 1.25, about 1.5, about 1.75, about 2, about 2.5, about 3, about 3.5, about 4, about 4.5, about 5, about 5.5, about 6, about 6.5, about 7, about 7.5, about 8, about 8.5, about 9, about 9.5, about 10 degrees, about 11 degrees, about 12 degrees, about 13 degrees, about 14 degrees, or about 15 degrees Celsius than a parent scFv in the described assays.

The improved thermal stability of the anti-TAA binding domain, e.g., scFv is subsequently conferred to the entire TAA-TFP construct, leading to improved therapeutic properties of the anti-TAA TFP construct. The thermal stability of the binding domain, e.g., scFv or sdAb, can be improved by at least about 2° C. or 3° C. as compared to a conventional antibody. In one embodiment, the binding domain, has a 1° C. improved thermal stability as compared to a conventional antibody. In another embodiment, the binding domain, has a 2° C. improved thermal stability as compared to a conventional antibody. In another embodiment, the scFv has a 4° C., 5° C., 6° C., 7° C., 8° C., 9° C., 10° C., 11° C., 12° C., 13° C., 14° C., or 15° C. improved thermal stability as compared to a conventional antibody. Comparisons can be made, for example, between the scFv molecules disclosed herein and scFv molecules or Fab fragments of an antibody from which the scFv V_{H} and V_{L} were derived. Thermal stability can be measured using methods known in the art. For example, in one embodiment, T_{M} can be measured. Methods for measuring T_{M} and other methods of determining protein stability are described in more detail below.

Mutations in antibody sequences (arising through humanization or direct mutagenesis of the soluble scFv) alter the stability of the antibody or fragment thereof and improve the overall stability of the antibody and the TFP construct. Stability of the humanized antibody or fragment thereof is compared against the murine antibody or fragment thereof using measurements such as T_{M}, temperature denaturation and temperature aggregation. In one embodiment, the binding domain, e.g., a scFv or sdAb, comprises at least one mutation arising from the humanization process such that the mutated scFv confers improved stability to the anti-TAA TFP construct. In another embodiment, the anti-TAA binding domain, e.g., scFv or sdAb, comprises at least 1, 2, 3, 4, 5, 6, 7, 8, 9, 10 mutations arising from the humanization process such that the mutated scFv or sdAb confers improved stability to the TAA-TFP construct.

In one aspect, the antigen binding domain of the TFP comprises an amino acid sequence that is homologous to an antigen binding domain amino acid sequence described herein, and the antigen binding domain retains the desired functional properties of the anti-tumor-associated antigen antibody fragments described herein. In one specific aspect, the TFP composition of the present disclosure comprises an antibody fragment. In a further aspect, that antibody fragment comprises a scFv.

In various aspects, the antigen binding domain of the TFP is engineered by modifying one or more amino acids within one or both variable regions (e.g., V_{H} and/or V_{L}), for example within one or more CDR regions and/or within one or more framework regions. In one specific aspect, the TFP composition of the present disclosure comprises an antibody fragment. In a further aspect, that antibody fragment comprises a scFv.

It will be understood by one of ordinary skill in the art that the antibody or antibody fragment of the present disclosure may further be modified such that they vary in amino acid sequence (e.g., from wild-type), but not in desired activity. For example, additional nucleotide substitutions leading to amino acid substitutions at “non-essential” amino acid residues may be made to the protein. For example, a nonessential amino acid residue in a molecule may be replaced with another amino acid residue from the same side chain family. In another embodiment, a string of amino acids can be replaced with a structurally similar string that differs in order and/or composition of side chain family members, e.g., a conservative substitution, in which an amino acid residue is replaced with an amino acid residue having a similar side chain, may be made.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Percent identity in the context of two or more nucleic acids or polypeptide sequences refers to two or more sequences that are the same. Two sequences are “substantially identical” if two sequences have a specified percentage of amino acid residues or nucleotides that are the same (e.g., 60% identity, optionally 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known in the art. Optimal alignment of sequences for comparison can be conducted, e.g., by the local homology algorithm of Smith and Waterman, (1970) Adv. Appl. Math. 2:482c, by the homology alignment algorithm of Needleman and Wunsch, (1970) J. Mol. Biol. 48:443, by the search for similarity method of Pearson and Lipman, (1988) Proc. Natl. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., (2003) Current Protocols in Molecular Biology). Two examples of algorithms that are suitable for determining percent sequence identity and sequence similarity are the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information.

In one aspect, the present disclosure contemplates modifications of the starting antibody or fragment (e.g., scFv) amino acid sequence that generate functionally equivalent molecules. For example, the V_{H} or V_{L} of a binding domain, e.g., scFv, comprised in the TFP can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity of the starting V_{H} or V_{L} framework region of the anti-CD19 binding domain, e.g., scFv. The present disclosure contemplates modifications of the entire TFP construct, e.g., modifications in one or more amino acid sequences of the various domains of the TFP construct in order to generate functionally equivalent molecules. The TFP construct can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity of the starting TFP construct.

### Extracellular Domain

The extracellular domain may be derived either from a natural or from a recombinant source. Where the source is natural, the domain may be derived from any protein, but in particular a membrane-bound or transmembrane protein. In one aspect the extracellular domain is capable of associating with the transmembrane domain. An extracellular domain of particular use in this present disclosure may include at least the extracellular region(s) of e.g., the alpha, beta or zeta chain of the T cell receptor, or CD3 epsilon, CD3 gamma, or CD3 delta, or in alternative embodiments, CD28, CD45, CD4, CD5, CD8, CD9, CD16, CD22, CD33, CD37, CD64, CD80, CD86, CD134, CD137, or CD154. In some embodiments, the extracellular domain is a TCR extracellular domain. In some instances, the TCR extracellular domain comprises an extracellular domain or portion thereof of a protein selected from the group consisting of a TCR alpha chain, a TCR beta chain, a TCR gamma chain, a TCR delta chain, a CD3 epsilon TCR subunit, a CD3 gamma TCR subunit, a CD3 delta TCR subunit, functional fragments thereof, and amino acid sequences thereof having at least one but not more than 20 modifications.

In some embodiments, the TCR extracellular domain comprises an extracellular domain or portion thereof of a TCR alpha chain, a TCR beta chain, a TCR delta chain, or a TCR gamma chain. In some embodiments, the TCR extracellular domain comprises an IgC domain of a TCR alpha chain, a TCR beta chain, a TCR delta chain, or a TCR gamma chain.

In some embodiments, the extracellular domain comprises, or comprises at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more consecutive amino acid residues of the extracellular domain of a TCR alpha chain, a TCR beta chain, a TCR delta chain, or a TCR gamma chain. In some embodiments, the extracellular domain comprises a sequence having at least about 50%, 55%, 60%, 65%, 70%, 75%, 80%, 85%, 90%, 95%, 98%, 99% or more sequence identity to a sequence encoding the extracellular domain of a TCR alpha chain, a TCR beta chain, a TCR delta chain, or a TCR gamma chain. In some embodiments, the extracellular domain comprises a sequence encoding the extracellular domain of a TCR alpha chain, a TCR beta chain, a TCR delta chain, or a TCR gamma chain having a truncation of at least 1, 2, 3, 4, 5, 6, 7, 8, 9, 10, 11, 12, 13, 14, 15, 16, 17, 18, 19, 20, 21, 22, 23, 24, 25 or more amino acids at the N- or C-terminus or at both the N- and C-terminus.

In some embodiments, the extracellular domain comprises, or comprises at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more consecutive amino acid residues of an IgC domain of TCR alpha, a TCR beta, a TCR delta, or a TCR gamma. In some embodiments, the extracellular domain comprises a sequence having at least about 50%, 55%, 60%, 65%, 70%, 75%, 80%, 85%, 90%, 95%, 98%, 99% or more sequence identity to a sequence encoding an IgC domain of TCR alpha, a TCR beta, a TCR delta, or a TCR gamma. In some embodiments, the extracellular domain comprises a sequence encoding an IgC domain of TCR alpha, TCR beta, TCR delta, or TCR gamma having a truncation of at least 1, 2, 3, 4, 5, 6, 7, 8, 9, 10, 11, 12, 13, 14, 15, 16, 17, 18, 19, 20, 21, 22, 23, 24, 25 or more amino acids at the N- or C-terminus or at both the N- and C-terminus.

In some embodiments, the extracellular domain comprises, or comprises at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more consecutive amino acid residues of the extracellular domain of a CD3 epsilon TCR subunit, a CD3 gamma TCR subunit, or a CD3 delta TCR subunit. In some embodiments, the extracellular domain comprises a sequence having at least about 50%, 55%, 60%, 65%, 70%, 75%, 80%, 85%, 90%, 95%, 98%, 99% or more sequence identity to a sequence encoding the extracellular domain of a CD3 epsilon TCR subunit, a CD3 gamma TCR subunit, or a CD3 delta TCR subunit. In some embodiments, the extracellular domain comprises a sequence encoding the extracellular domain of a CD3 epsilon TCR subunit, a CD3 gamma TCR subunit, or a CD3 delta TCR subunit having a truncation of at least 1, 2, 3, 4, 5, 6, 7, 8, 9, 10, 11, 12, 13, 14, 15, 16, 17, 18, 19, 20, 21, 22, 23, 24, 25 or more amino acids at the N- or C-terminus or at both the N- and C-terminus.

The extracellular domain can be a TCR extracellular domain. The TCR extracellular domain can be derived from a TCR alpha chain, a TCR beta chain, a TCR gamma chain, a TCR delta chain, a CD3 epsilon TCR subunit, a CD3 gamma TCR subunit or a CD3 delta TCR subunit. The extracellular domain can be a full-length TCR extracellular domain or fragment (e.g., functional fragment) thereof. The extracellular domain can comprise a variable domain of a TCR alpha chain, a TCR beta chain, a TCR gamma chain or a TCR delta chain. The extracellular domain can comprise a variable domain and a constant domain of a TCR alpha chain, a TCR beta chain, a TCR gamma chain or a TCR delta chain. In some cases, the extracellular domain may not comprise a variable domain.

The extracellular domain can comprise a constant domain of a TCR alpha chain, a TCR beta chain, a TCR gamma chain or a TCR delta chain. The extracellular domain can comprise a full-length constant domain of a TCR alpha chain, a TCR beta chain, a TCR gamma chain or a TCR delta chain. The extracellular domain can comprise a fragment (e.g., functional fragment) of the full-length constant domain of a TCR alpha chain, a TCR beta chain, a TCR gamma chain or a TCR delta chain. For example, the extracellular domain can comprise at least about 5, 10, 15, 20, 25, 30, 35, 40, 45, 50, 55, 60, 65, 70, 75, 80, 85, 90, 95, 100, 150 or more amino acid residues of the constant domain of a TCR alpha chain, a TCR beta chain, a TCR gamma chain or a TCR delta chain.

The TCR alpha chain, a TCR beta chain, a TCR gamma chain or a TCR delta chain described herein can be derived from various species. The TCR chain can be a murine or human TCR chain. For example, the extracellular domain can comprise a constant domain of a murine TCR alpha chain, a murine TCR beta chain, a human TCR gamma chain or a human TCR delta chain.

### Transmembrane Domain

In general, a TFP sequence contains an extracellular domain and a transmembrane domain encoded by a single genomic sequence. In alternative embodiments, a TFP can be designed to comprise a transmembrane domain that is heterologous to the extracellular domain of the TFP. A transmembrane domain can include one or more additional amino acids adjacent to the transmembrane region, e.g., one or more amino acid associated with the extracellular region of the protein from which the transmembrane was derived (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids of the extracellular region) and/or one or more additional amino acids associated with the intracellular region of the protein from which the transmembrane protein is derived (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids of the intracellular region). In some cases, the transmembrane domain can include at least 30, 35, 40, 45, 50, 55, 60 or more amino acids of the extracellular region. In some cases, the transmembrane domain can include at least 30, 35, 40, 45, 50, 55, 60 or more amino acids of the intracellular region. In one aspect, the transmembrane domain is one that is associated with one of the other domains of the TFP is used. In some instances, the transmembrane domain can be selected or modified by amino acid substitution to avoid binding of such domains to the transmembrane domains of the same or different surface membrane proteins, e.g., to minimize interactions with other members of the receptor complex. In one aspect, the transmembrane domain is capable of homodimerization with another TFP on the TFP-T cell surface. In a different aspect the amino acid sequence of the transmembrane domain may be modified or substituted so as to minimize interactions with the binding domains of the native binding partner present in the same TFP.

The transmembrane domain may be derived either from a natural or from a recombinant source. Where the source is natural, the domain may be derived from any membrane-bound or transmembrane protein. In one aspect the transmembrane domain is capable of signaling to the intracellular domain(s) whenever the TFP has bound to a target. In some instances, the TCR-integrating subunit comprises a transmembrane domain comprising a transmembrane domain of a protein selected from the group consisting of a TCR alpha chain, a TCR beta chain, a TCR gamma chain, a TCR delta chain, a TCR zeta chain, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, CD154, functional fragments thereof, and amino acid sequences thereof having at least one but not more than 20 modifications.

In some embodiments, the transmembrane domain comprises, or comprises at least 5, 6, 7, 8, 9, 10, 11, 12, 13, 14, 15, 16, 17, 18, 19, 20, 21, 22, 23, 24, 25, 26, 27, 28, 29, or 30 or more consecutive amino acid residues of the transmembrane domain of a TCR alpha chain, a TCR beta chain, a TCR gamma chain, a TCR delta chain, a CD3 epsilon TCR subunit, a CD3 gamma TCR subunit, or a CD3 delta TCR subunit. In some embodiments, the transmembrane domain comprises a sequence having at least about 50%, 55%, 60%, 65%, 70%, 75%, 80%, 85%, 90%, 95%, 98%, 99% or more sequence identity to a sequence encoding the transmembrane domain of a TCR alpha chain, a TCR beta chain, a TCR gamma chain, a TCR delta chain, a CD3 epsilon TCR subunit, a CD3 gamma TCR subunit, or a CD3 delta TCR subunit. In some embodiments, the transmembrane domain comprises a sequence encoding the transmembrane domain of a TCR alpha chain, a TCR beta chain, a TCR gamma chain, a TCR delta chain, a CD3 epsilon TCR subunit, a CD3 gamma TCR subunit, or a CD3 delta TCR subunit having a truncation of at least 1, 2, 3, 4, 5, 6, 7, 8, 9, or 10 or more amino acids at the N- or C-terminus or at both the N- and C-terminus.

In some instances, the transmembrane domain can be attached to the extracellular region of the TFP, e.g., the antigen binding domain of the TFP, via a hinge, e.g., a hinge from a human protein. For example, in one embodiment, the hinge can be a human immunoglobulin (Ig) hinge, e.g., an IgG4 hinge, or a CD8a hinge.

### Linkers

Optionally, a short oligo- or polypeptide linker, between 2 and 10 amino acids in length may form the linkage between the binding element and the TCR extracellular domain of the TFP. A glycine-serine doublet provides a particularly suitable linker. In some cases, the linker may be at least about 5, 6, 7, 8, 9, 10, 11, 12, 13, 14, 15, 16, 17, 18, 19, 20, or more in length. For example, in one aspect, the linker comprises the amino acid sequence of GGGGSGGGGS or a sequence (GGGGS)x or (G₄S)ₙ, wherein X or n is 1, 2, 3, 4, 5, 6, 7, 8, 9, or 10 or more. In some embodiments, X or n is an integer from 1 to 10. In some embodiments, X or n is an integer from 1 to 4. In some embodiments, X or n is 2. In some embodiments, X or n is 4. In some embodiments, the linker is encoded by a nucleotide sequence of

GGTGGCGGAGGTTCTGGAGGTGGAGGTTCC.

In some embodiments, a linker sequence may comprise SEQ ID NO: 387. In some embodiments, the linker sequence may comprise SEQ ID NO: 388. In some embodiments, the linker sequence may comprise SEQ ID NO: 378. In some embodiments, the linker sequence may comprise SEQ ID NO: 405. In some embodiments, the linker sequence may comprise SEQ ID NO: 23. In some embodiments, the linker sequence may comprise SEQ ID NO: 365. In some embodiments, the linker sequence may comprise SEQ ID NO: 401.

### Cytoplasmic Domain

The cytoplasmic domain of the TFP can include an intracellular domain. In some embodiments, the intracellular domain is from CD3 gamma, CD3 delta, CD3 epsilon, TCR alpha, TCR beta, TCR gamma, or TCR delta. In some embodiments, the intracellular domain comprises a signaling domain, if the TFP contains CD3 gamma, delta or epsilon polypeptides; TCR alpha, TCR beta, TCR gamma, and TCR delta subunits generally have short (e.g., 1-19 amino acids in length) intracellular domains and are generally lacking in a signaling domain. An intracellular signaling domain is generally responsible for activation of at least one of the normal effector functions of the immune cell in which the TFP has been introduced. While the intracellular domains of TCR alpha, TCR beta, TCR gamma, and TCR delta do not have signaling domains, they are able to recruit proteins having a primary intracellular signaling domain described herein, e.g., CD3 zeta, which functions as an intracellular signaling domain. The term “effector function” refers to a specialized function of a cell. Effector function of a T cell, for example, may be cytolytic activity or helper activity including the secretion of cytokines. Thus, the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Examples of intracellular domains for use in the TFP of the present disclosure include the cytoplasmic sequences of the T cell receptor (TCR) and co-receptors that are able to act in concert to initiate signal transduction following antigen receptor engagement, as well as any derivative or variant of these sequences and any recombinant sequence that has the same functional capability. In some embodiments, the intracellular domain comprises the intracellular domain of a TCR alpha chain, a TCR beta chain, a TCR gamma chain, a TCR delta chain, a CD3 epsilon TCR subunit, a CD3 gamma TCR subunit, or a CD3 delta TCR subunit. In some embodiments, the intracellular domain comprises, or comprises at least 1, 2, 3, 4, 5, 6, 7, 8, 9, 10, 11, 12, 13, 14, 15, 16, 17, 18, or 19 or more consecutive amino acid residues of the intracellular domain of a TCR alpha chain, a TCR beta chain, a TCR gamma chain, or a TCR delta chain. In some embodiments, the intracellular domain comprises a sequence having at least about 50%, 55%, 60%, 65%, 70%, 75%, 80%, 85%, 90%, 95%, 98%, 99% or more sequence identity to a sequence encoding the intracellular domain of a TCR alpha chain, a TCR beta chain, a TCR gamma chain, or a TCR delta chain. In some embodiments, the transmembrane domain comprises a sequence encoding the intracellular domain of a TCR alpha chain, a TCR beta chain, a TCR gamma chain, or a TCR delta chain having a truncation of at least 1, 2, 3, 4, 5, 6, 7, 8, 9, or 10 or more amino acids at the N- or C-terminus or at both the N- and C-terminus.

In some embodiments, the intracellular domain comprises, or comprises at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, or 62 or more consecutive amino acid residues of the intracellular domain of a CD3 epsilon TCR subunit, a CD3 gamma TCR subunit, or a CD3 delta TCR subunit. In some embodiments, the intracellular domain comprises a sequence having at least about 50%, 55%, 60%, 65%, 70%, 75%, 80%, 85%, 90%, 95%, 98%, 99% or more sequence identity to a sequence encoding the intracellular domain of a CD3 epsilon TCR subunit, a CD3 gamma TCR subunit, or a CD3 delta TCR subunit. In some embodiments, the intracellular domain comprises a sequence encoding the intracellular domain of a CD3 epsilon TCR subunit, a CD3 gamma TCR subunit, or a CD3 delta TCR subunit having a truncation of at least 1, 2, 3, 4, 5, 6, 7, 8, 9, 10, 11, 12, 13, 14, 15, 16, 17, 18, 19, 20, 21, 22, 23, 24, 25 or more amino acids at the N- or C-terminus or at both the N- and C-terminus.

It is known that signals generated through the TCR alone are insufficient for full activation of naive T cells and that a secondary and/or costimulatory signal is required. Thus, naïve T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs (ITAMs).

Examples of ITAMs containing primary intracellular signaling domains that are of particular use in the present disclosure include those of CD3 zeta, FcR gamma, FcR beta, CD3 gamma, CD3 delta, CD3 epsilon, CD5, CD22, CD79a, CD79b, and CD66d. In one embodiment, a TFP of the present disclosure comprises an intracellular signaling domain, e.g., a primary signaling domain of CD3-epsilon. In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs.

The intracellular signaling domain of the TFP can comprise a CD3 signaling domain, e.g., CD3 epsilon, CD3 delta, CD3 gamma, or CD3 zeta, by itself or it can be combined with any other desired intracellular signaling domain(s) useful in the context of a TFP of the present disclosure. For example, the intracellular signaling domain of the TFP can comprise a CD3 epsilon chain portion and a costimulatory signaling domain. The costimulatory signaling domain refers to a portion of the TFP comprising the intracellular domain of a costimulatory molecule. A costimulatory molecule is a cell surface molecule other than an antigen receptor or its ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like. For example, CD27 costimulation has been demonstrated to enhance expansion, effector function, and survival of human TFP-T cells in vitro and augments human T cell persistence and antitumor activity in vivo (Song et al., Blood. 2012; 119(3):696-706).

The intracellular signaling sequences within the cytoplasmic portion of the TFP of the present disclosure may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequences.

In one embodiment, a glycine-serine doublet can be used as a suitable linker. In one embodiment, a single amino acid, e.g., an alanine, a glycine, can be used as a suitable linker.

In one aspect, the TFPs described herein may comprise a TCR extracellular domain, a TCR transmembrane domain, and a TCR intracellular domain, wherein at least two of the TCR extracellular domain, the TCR transmembrane domain, and the TCR intracellular domain are from the same TCR subunit. In some embodiments, at least two of the TCR extracellular domain, the TCR transmembrane domain, and the TCR intracellular domain can be from TCR alpha. In some embodiments, at least two of the TCR extracellular domain, the TCR transmembrane domain, and the TCR intracellular domain can be from TCR beta. In some embodiments, at least two of the TCR extracellular domain, the TCR transmembrane domain, and the TCR intracellular domain can be from TCR gamma. In some embodiments, at least two of the TCR extracellular domain, the TCR transmembrane domain, and the TCR intracellular domain can be from TCR delta. In some embodiments, at least two of the TCR extracellular domain, the TCR transmembrane domain, and the TCR intracellular domain can be from CD3 epsilon. In some embodiments, at least two of the TCR extracellular domain, the TCR transmembrane domain, and the TCR intracellular domain can be from CD3 delta. In some embodiments, at least two of the TCR extracellular domain, the TCR transmembrane domain, and the TCR intracellular domain can be from CD3 gamma.

In one aspect, the TFPs described herein may comprise a TCR extracellular domain, a TCR transmembrane domain, and a TCR intracellular domain, wherein all three of the TCR extracellular domain, the TCR transmembrane domain, and the TCR intracellular domain can be from the same TCR subunit. In some embodiments, the TCR extracellular domain, the TCR transmembrane domain, and the TCR intracellular domain can be from CD3 epsilon. In some embodiments, the TCR extracellular domain, the TCR transmembrane domain, and the TCR intracellular domain can be from CD3 delta. In some embodiments, the TCR extracellular domain, the TCR transmembrane domain, and the TCR intracellular domain can be from CD3 gamma. In some embodiments, the TCR extracellular domain, the TCR transmembrane domain, and the TCR intracellular domain may comprise the constant domain of TCR alpha. In some embodiments, the TCR extracellular domain, the TCR transmembrane domain, and the TCR intracellular domain may comprise the constant domain of TCR beta. In some embodiments, the TCR extracellular domain, the TCR transmembrane domain, and the TCR intracellular domain may comprise the constant domain of TCR gamma. In some embodiments, the TCR extracellular domain, the TCR transmembrane domain, and the TCR intracellular domain may comprise the constant domain of TCR delta. In some embodiments, the constant domain of TCR alpha or the constant domain of TCR beta may be murine.

In one aspect, the TFP-expressing cell described herein can further comprise a second TFP, e.g., a second TFP that includes a different antigen binding domain, e.g., to the same target (e.g., MSLN) or a different target (e.g., CD70, CD19, or MUC16). In one embodiment, when the TFP-expressing cell comprises two or more different TFPs, the antigen binding domains of the different TFPs can be such that the antigen binding domains do not interact with one another. For example, a cell expressing a first and second TFP can have an antigen binding domain of the first TFP, e.g., as a fragment, e.g., a scFv, that does not form an association with the antigen binding domain of the second TFP, e.g., the antigen binding domain of the second TFP is a V_{HH}.

### IL-15 and IL-15 Receptor Alpha Polypeptides

In some aspects, the TFP-expressing cells described herein can further express another agent, for example, an agent that can enhance longevity or activity of TFP-expressing cells described herein. In some embodiments, the agent is a cytokine such as a pleiotropic cytokine that plays important roles in maintenance and homeostatic expansion of immune cells. In some embodiments, local secretion of a pleiotropic cytokine in tumor microenvironment (TME) can contribute to enhanced anti-tumor immunity. In some embodiments, the agent activates a cytokine signaling. In some embodiments the agent activates interleukin-15 (IL-15) signaling. In some embodiments the agent comprises interleukin-15 (IL-15) and/or interleukin-15 receptor (IL-15R). In some embodiments, the IL-15R is an IL-15R alpha (IL-15Rα) subunit.

The present disclosure provides compositions and methods to enhance TFP-T cell persistence by engineering constitutive IL-15 signaling. The increased TFP persistence can lead to enhanced duration of response (DOR) in patients. IL-15 may have a crucial role in the maintenance of naïve and central memory CD8+ T cells and enhancing their survival by reducing activation induced cell death (AICD). IL-15 expressing TFP-T cells, in some cases, can show increased in vitro and in vivo persistence. IL-15 or variants thereof can be a T cell intrinsic enhancement so that they can be used for TFPs targeting various tumor types. As described herein, various constructs containing the IL-15 or IL-15R alpha or fragment thereof can be referred to as enhancements.

The T cells engineered with a TFP (e.g., TFP containing an antigen binding domain targeting a TAA) and IL-15 enhancements described herein can show high transduction efficiency and co-expression. For example, in some cases, T cells engineered with anti-mesothelin TFP and IL-15 enhancements described herein can show high transduction efficiency and co-expression. For another example, in some cases, T cells engineered with anti-CD70 TFP and IL-15 enhancements described herein can show high transduction efficiency and co-expression. In various embodiments, a cell (e.g., a TFP-T cell or transduced T cell) described herein can express a IL-15 polypeptide or fragment thereof or a IL-15R subunit or fragment thereof constitutively.

The present disclosure encompasses recombinant nucleic acid molecules encoding an interleukin-15 (IL-15) polypeptide or a fragment thereof. In some embodiments, the IL-15 polypeptide or a fragment thereof comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or more consecutive amino acid residues of IL-15. In some embodiments, the IL-15 polypeptide or a fragment thereof comprises a sequence having at least about 50%, 55%, 60%, 65%, 70%, 75%, 80%, 85%, 90%, 95%, 98%, 99% or more sequence identity to a sequence encoding IL-15. In some embodiments, the IL-15 polypeptide or a fragment thereof comprises a sequence encoding IL-15 having a truncation of at least 1, 2, 3, 4, 5, 6, 7, 8, 9, 10, 11, 12, 13, 14, 15, 16, 17, 18, 19, 20, 21, 22, 23, 24, 25 or more amino acids at the N- or C-terminus or at both the N- and C-terminus.

In some embodiments, the IL-15 polypeptide or a fragment thereof may comprise an IL-15 signal peptide. In some embodiments, the IL-15 polypeptide or a fragment thereof may comprise amino acids 1-29 of IL-15. In some embodiments, the IL-15 polypeptide or a fragment thereof may comprise amino acids 1-29 of SEQ ID NO: 385. In some embodiments, the IL-15 polypeptide or a fragment thereof may comprise a sequence of SEQ ID NO: 374. In some embodiments, the IL-15 polypeptide or a fragment thereof may comprise amino acids 30-162 of IL-15. In some embodiments, the IL-15 polypeptide or a fragment thereof may comprise amino acids 30-162 of SEQ ID NO: 385. In some embodiments, the IL-15 polypeptide or a fragment thereof may comprise a sequence of SEQ ID NO: 375. In some embodiments, the IL-15 polypeptide or a fragment thereof may comprise amino acids 1-162 of SEQ ID NO: 385. In some embodiments, the IL-15 polypeptide or a fragment thereof may comprise a sequence of SEQ ID NO: 374 and a sequence of SEQ ID NO: 375. In some embodiments, the IL-15 polypeptide or a fragment thereof may comprise a sequence of SEQ ID NO: 380. In some embodiments, IL-15 polypeptide is secreted when expressed in a cell, such as a T cell.

The present disclosure further encompasses recombinant nucleic acid molecules encoding an interleukin-15 receptor (IL-15R) subunit polypeptide or a fragment thereof. For example, the IL-15R subunit may be IL-15 receptor alpha chain (“IL-15Rα” or CD215), IL-2 receptor beta chain (“IL-2Rβ” or CD122) and IL-2 receptor gamma/the common gamma chain (“IL-2Rγ/γc” or CD132). In some embodiments, the IL-15R subunit is an IL-15Rα or a fragment thereof. In some embodiments, the IL-15Rα polypeptide or a fragment thereof comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, or more consecutive amino acid residues of IL-15Rα. In some embodiments, the IL-15Rα polypeptide or a fragment thereof comprises a sequence having at least about 50%, 55%, 60%, 65%, 70%, 75%, 80%, 85%, 90%, 95%, 98%, 99% or more sequence identity to a sequence encoding IL-15Rα. In some embodiments, the IL-15Rα polypeptide or a fragment thereof comprises a sequence encoding IL-15Rα having a truncation of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more amino acids at the N- or C-terminus or at both the N- and C-terminus.

In some embodiments, the IL-15Rα polypeptide or a fragment thereof may comprise IL-15Rα signal peptide. In some embodiments, the IL-15Rα polypeptide or a fragment thereof may comprise amino acids 1-30 of IL-15Rα. In some embodiments, the IL-15Rα polypeptide or a fragment thereof may comprise amino acids 1-30 of SEQ ID NO: 386. In some embodiments, the IL-15Rα polypeptide or a fragment thereof does not comprise IL-15Rα signal peptide. In some embodiments, the IL-15Rα polypeptide or a fragment thereof does not comprise amino acids 1-30 of IL-15Rα. In some embodiments, the IL-15Rα polypeptide or a fragment thereof does not comprise amino acids 1-30 of SEQ ID NO: 386.

In some embodiments, the IL-15Rα polypeptide or a fragment thereof may comprise IL-15Rα Sushi domain. In some embodiments, the IL-15Rα polypeptide or a fragment thereof may comprise amino acids 31-95 of IL-15Rα. In some embodiments, the IL-15Rα polypeptide or a fragment thereof may comprise amino acids 31-95 of SEQ ID NO: 386. In some embodiments, the IL-15Rα polypeptide or a fragment thereof may comprise a sequence of SEQ ID NO: 382.

In some embodiments, the IL-15Rα polypeptide or a fragment thereof may comprise an intracellular domain of IL-15Rα. In some embodiments, the IL-15Rα polypeptide or a fragment thereof may comprise amino acids 229-267 of IL-15Rα. In some embodiments, the IL-15Rα polypeptide or a fragment thereof may comprise amino acids 229-267 of a sequence of SEQ ID NO: 386. In some embodiments, the IL-15Rα polypeptide or a fragment thereof may comprise a sequence of SEQ ID NO: 372.

In some embodiments, the IL-15Rα polypeptide or a fragment thereof may comprise IL-15Rα Sushi domain, transmembrane domain, and intracellular domain. In some embodiments, the IL-15Rα polypeptide or a fragment thereof may comprise amino acids 31-267 of IL-15Rα. In some embodiments, the IL-15Rα polypeptide or a fragment thereof may comprise amino acids 31-267 of SEQ ID NO: 386. In some embodiments, the IL-15Rα polypeptide or a fragment thereof may comprise a sequence of SEQ ID NO: 382. In some embodiments, the IL-15Rα polypeptide or a fragment thereof may comprise a sequence of SEQ ID NO: 383. In some embodiments, the IL-15Rα polypeptide or a fragment thereof may comprise amino acids 96-267 of SEQ ID NO: 386. In some embodiments, the IL-15Rα polypeptide or a fragment thereof may comprise a sequence of SEQ ID NO: 382 and a sequence of SEQ ID NO: 383. In some embodiments, IL-15Rα comprises a sequence of SEQ ID NO: 403.

In some embodiments, the IL-15Rα polypeptide or a fragment thereof may be a soluble IL-15Rα (sIL-15Rα). In some embodiments, the IL-15Rα polypeptide or a fragment thereof may comprise amino acids 21-205 of IL-15Rα. In some embodiments, the IL-15Rα polypeptide or a fragment thereof may comprise amino acids 21-205 of a sequence of SEQ ID NO: 386. In some embodiments, the IL-15Rα polypeptide or a fragment thereof may comprise a sequence of SEQ ID NO: 379.

The present disclosure encompasses recombinant nucleic acid molecules encoding a fusion protein comprising an IL-15 polypeptide linked to an IL-15R subunit. In some embodiments, IL-15 and IL-15R subunit are operatively linked by a linker. In some embodiments, the IL-15R subunit is IL-15R alpha (IL-15Rα). For example, IL-15 polypeptide may be linked to N-terminus of IL-15Rα subunit. For example, IL-15 polypeptide may be linked to C-terminus of IL-15Rα subunit. In some embodiments, IL-15 and IL-15Rα are operatively linked by a linker. In some embodiments, the linker is not a cleavable linker. For example, the linker may comprise a sequence comprising (G₄S)ₙ, wherein G is glycine, S is serine, and n is an integer from 1 to 10. In some embodiments, n is an integer from 1 to 4. In some embodiments, n is 3. In some embodiments, the linker comprises a sequence of SEQ ID NO: 378. In some embodiments, the linker comprises a sequence of SEQ ID NO: 405.

In some embodiments, the fusion protein may comprise amino acids 30-162 of IL-15. In some embodiments, the fusion protein may comprise amino acids 30-162 of a sequence of SEQ ID NO: 385. In some embodiments, the fusion protein may comprise a sequence of SEQ ID NO: 375. In some embodiments, the fusion protein does not comprise IL-15 signal peptide. In some embodiments, the fusion protein does not comprise amino acids 1-29 of IL-15. In some embodiments, the fusion protein does not comprise amino acids 1-29 of a sequence of SEQ ID NO: 385. In some embodiments, the fusion protein does not comprise a sequence of SEQ ID NO: 374.

In some embodiments, the fusion protein may comprise a Sushi domain. In some embodiments, the fusion protein may comprise amino acids 31-95 of IL-15Rα. In some embodiments, the fusion protein may comprise amino acids 31-95 of a sequence of SEQ ID NO: 386. In some embodiments, the fusion protein may comprise a sequence of SEQ ID NO: 382.

In some embodiments, the fusion protein may comprise the intracellular domain of IL-15Rα. In some embodiments, the fusion protein may comprise amino acids 229-267 of IL-15Rα. In some embodiments, the fusion protein may comprise amino acids 229-267 of a sequence of SEQ ID NO: 386. In some embodiments, the fusion protein may comprise a sequence of SEQ ID NO: 372.

In some embodiments, the fusion protein may comprise a soluble IL-15Rα (sIL-15Rα). In some embodiments, the fusion protein may comprise amino acids 21-205 of IL-15Rα. In some embodiments, the fusion protein may comprise amino acids 21-205 of a sequence of SEQ ID NO: 386. In some embodiments, the fusion protein may comprise a sequence of SEQ ID NO: 379.

In some embodiments, the fusion protein may comprise the transmembrane domain and the intracellular domain of IL-15Rα. In some embodiments, the fusion protein may comprise amino acids 96-267 of IL-15Rα. In some embodiments, the fusion protein may comprise amino acids 96-267 of a sequence of SEQ ID NO: 386. In some embodiments, the fusion protein may comprise a sequence of SEQ ID NO: 383.

In some embodiments, the fusion protein may comprise the Sushi domain, the transmembrane domain, and the intracellular domain of IL-15Rα. In some embodiments, the fusion protein may comprise amino acids 31-267 of IL-15Rα. In some embodiments, the fusion protein may comprise amino acids 31-267 of a sequence of SEQ ID NO: 386. In some embodiments, the fusion protein may comprise a sequence of SEQ ID NO: 382 and a sequence of SEQ ID NO: 383. In some embodiments, IL-15Rα comprises a sequence of SEQ ID NO: 403.

In some embodiments, the fusion protein further comprises an epitope tag. An epitope tag as described herein can be a peptide epitope tag or a protein epitope tag. Examples of a peptide epitope tag includes, but are not limited to, 6×His (also known as His-tag or hexahistidine tag), FLAG (e.g., 3×FLAG), HA, Myc, and V5. Examples of a protein epitope tag include, but are not limited to, green fluorescent protein (GFP), glutathione-S-transferase (GST), β-galactosidase (β-GAL), Luciferase, Maltose Binding Protein (MBP), Red Fluorescence Protein (RFP), and Vesicular Stomatitis Virus Glycoprotein (VSV-G). In some embodiments, the fusion protein further comprises a FLAG tag. In some embodiments, the fusion protein further comprises a 3×FLAG tag. In some embodiments, the fusion protein further comprises a sequence of SEQ ID NO: 384.

In some embodiments, the fusion protein comprises a sequence of SEQ ID NO: 377. In some embodiments, the fusion protein comprises a sequence of SEQ ID NO: 381. In some embodiments, the fusion protein is expressed on cell surface when expressed in a T cell. In some embodiments, the fusion protein is secreted when expressed in a T cell.

In some aspects, cells expressing TFPs, an IL-15 polypeptide or a fragment thereof, an IL-15Rα polypeptide or a fragment thereof, and/or a fusion protein comprising an IL-15 polypeptide and an IL-15Rα polypeptide described herein can yet further express another agent that can enhance the activity of a modified T cell expressing TFPs. For example, in one embodiment, the agent that can enhance the activity of a modified T cell can be a PD-1 polypeptide. In these embodiments, the PD-1 polypeptide may be operably linked to the N-terminus of an intracellular domain of a costimulatory polypeptide via the C-terminus of the PD-1 polypeptide. For example, in another embodiment, the agent that can enhance the activity of a modified T cell expressing TFPs can be an anti-PD-1 antibody, or antigen binding fragment thereof. In this embodiment, the anti-PD-1 antibody or antigen binding fragment thereof may be operably linked to the N-terminus of an intracellular domain of a costimulatory polypeptide via the C-terminus of the anti-PD-1 antibody, or antigen binding fragment thereof. In some embodiments, the PD-1 polypeptide or anti-PD-1 antibody is linked to the intracellular domain of the costimulatory polypeptide via the transmembrane domain of PD-1. In some embodiments, the costimulatory polypeptide is selected from the group consisting of OX40, CD2, CD27, CDS, ICAM-1, ICOS (CD278), 4-1BB (CD137), GITR, CD28, CD30, CD40, BAFFR, HVEM, CD7, LIGHT, NKG2C, SLAMF7, NKp80, CD160, CD226, FcγRI, FcγRII, and FcγRIII.

In some aspects, the agent that can enhance the activity of a modified T cell expressing TFPs can be linked to an IL-15Rα polypeptide or a fragment thereof. For example, the agent can be an agent that can inhibit an inhibitory molecule that can decrease the ability of a T cell expressing a TFP to mount an immune effector response. In some embodiments, the agent which inhibits an inhibitory molecule comprises a first polypeptide, e.g., an inhibitory molecule, associated with a second polypeptide that provides a positive signal to the cell, e.g., an intracellular signaling domain described herein. In one embodiment, the agent may comprise a first polypeptide, e.g., of an inhibitory molecule such as PD-1, LAG3, CTLA4, CD160, BTLA, LAIR1, TIM3, 2B4, and TIGIT, or a fragment of any of these (e.g., at least a portion of an extracellular domain of any of these), and a second polypeptide which is an intracellular signaling domain described herein (e.g., comprising a costimulatory domain (e.g., 4-1BB, CD27, or CD28, as described herein)) and/or a primary signaling domain (e.g., IL-15Rα described herein). In some embodiments, the agent may be PD-1 or a fragment thereof. For example, the agent may comprise the extracellular domain of PD-1. In some embodiments, the agent may comprise the extracellular domain and transmembrane domain of PD-1. In some embodiments, the agent may further comprise CD28 or a fragment thereof. In some embodiments, the agent may comprise the intracellular domain of CD28. In some embodiments, the agent may comprise a fusion protein comprising the PD-1 extracellular domain and transmembrane domain linked to the CD28 intracellular domain linked to IL-15Rα. In some embodiments, the CD28 intracellular domain is linked to the intracellular domain of IL-15Rα.

In some embodiments, the PD-1 or a fragment thereof may comprise a sequence of SEQ ID NO: 366. In some embodiments, the PD-1 or a fragment thereof may comprise a sequence of SEQ ID NO: 367. In some embodiments, the PD-1 or a fragment thereof may comprise a sequence of SEQ ID NO: 368. In some embodiments, the PD-1 or a fragment thereof may comprise a sequence of SEQ ID NO: 369. In some embodiments, the transmembrane domain of PD-1 may comprise a sequence of SEQ ID NO: 370. In some embodiments, the intracellular domain of CD28 may comprise a sequence of SEQ ID NO: 371. In some embodiments, the intracellular domain of IL-15Rα comprises amino acids 229-267 of IL-15Rα. In some embodiments, the intracellular domain of IL-15Rα comprises amino acids 229-267 of a sequence of SEQ ID NO: 386. In some embodiments, the fusion protein comprises a sequence of SEQ ID NO: 372. In some embodiments, the fusion protein comprises a sequence of SEQ ID NO: 376.

In some aspects, the agent that can enhance the activity of a modified T cell expressing TFPs can be linked to a fusion protein comprising an IL-15 polypeptide and an IL-15Rα polypeptide. In some embodiments, the agent may be PD-1 or a fragment thereof. For example, the agent may comprise the extracellular domain of PD-1. In some embodiments, the agent may comprise the extracellular domain and transmembrane domain of PD-1. In some embodiments, the agent may further comprise CD28 or a fragment thereof. In some embodiments, the agent may comprise the intracellular domain of CD28. In some embodiments, the agent may comprise a fusion protein comprising the PD-1 extracellular domain and transmembrane domain linked to the CD28 intracellular domain linked to the fusion protein comprising an IL-15 polypeptide and an IL-15Rα polypeptide. In some embodiments, the CD28 intracellular domain is linked to the intracellular domain of IL-15Rα. In some embodiments, the intracellular domain of IL-15Rα is linked to the IL-15 polypeptide by a linker described herein. In some embodiments, the linker comprises a cleavage site. The cleavage site can be a self-cleaving peptide such as a T2A, P2A, E2A or F2A cleavage site. In some embodiments, the cleavage site can comprise a sequence of SEQ ID NO: 373. In some embodiments, the cleavage site may comprise SEQ ID NO: 23. In some embodiments, the cleavage site may comprise SEQ ID NO: 365.

In some embodiments, the fusion protein may comprise a PD-1 or a fragment thereof comprising a sequence of SEQ ID NO: 366. In some embodiments, the fusion protein may comprise a PD-1 or a fragment thereof comprising a sequence of SEQ ID NO: 367. In some embodiments, the fusion protein may comprise a PD-1 or a fragment thereof comprising a sequence of SEQ ID NO: 368. In some embodiments, the fusion protein may comprise a PD-1 or a fragment thereof comprising a sequence of SEQ ID NO: 369. In some embodiments, the fusion protein may comprise a PD-1 or a fragment thereof comprising a transmembrane domain of PD-1 comprising a sequence of SEQ ID NO: 370. In some embodiments, the fusion protein may comprise a CD28 or a fragment comprising the intracellular domain of CD28 comprising a sequence of SEQ ID NO: 371. In some embodiments, the intracellular domain of IL-15Rα comprises amino acids 229-267 of IL-15Rα. In some embodiments, the intracellular domain of IL-15Rα comprises amino acids 229-267 of a sequence of SEQ ID NO: 386. In some embodiments, the fusion protein comprises a sequence of SEQ ID NO: 372. In some embodiments, the IL-15 polypeptide comprises IL-15 signal peptide. In some embodiments, the IL-15 polypeptide comprises amino acids 1-29 of IL-15. In some embodiments, the IL-15 polypeptide comprises amino acids 1-29 of a sequence of SEQ ID NO: 385. In some embodiments, the IL-15 polypeptide comprises a sequence of SEQ ID NO: 374. In some embodiments, the IL-15 polypeptide comprises amino acids 30-162 of IL-15. In some embodiments, the IL-15 polypeptide comprises amino acids 30-162 of a sequence of SEQ ID NO: 385. In some embodiments, the IL-15 polypeptide comprises a sequence of SEQ ID NO: 375. In some embodiments, the fusion protein comprises a sequence of SEQ ID NO: 361.

Disclosed herein, in some embodiments, are polypeptides encoded by any of recombinant nucleic acid molecules described herein.

### Other Agents

In another aspect, the cells expressing TFP, IL-15, and/or IL-15Rα described herein can further express another agent, e.g., an agent which enhances the activity of a modified T cell. For example, in one embodiment, the agent can be an agent which inhibits an inhibitory molecule. Inhibitory molecules, e.g., PD-1, can, in some embodiments, decrease the ability of a modified T cell to mount an immune effector response. Examples of inhibitory molecules include PD-1, PD-L1, CTLA4, TIM3, LAG3, VISTA, BTLA, TIGIT, LAIR1, CD160, 2B4 and TGFR beta. In one embodiment, the agent which inhibits an inhibitory molecule comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD-1, LAG3, CTLA4, CD160, BTLA, LAIR1, TIM3, 2B4 and TIGIT, or a fragment of any of these (e.g., at least a portion of an extracellular domain of any of these), and a second polypeptide which is an intracellular signaling domain described herein (e.g., comprising a costimulatory domain (e.g., 4-1BB, CD27 or CD28, e.g., as described herein) and/or a primary signaling domain (e.g., a CD3 zeta signaling domain described herein). In one embodiment, the agent comprises a first polypeptide of PD-1 or a fragment thereof (e.g., at least a portion of an extracellular domain of PD-1), and a second polypeptide of an intracellular signaling domain described herein (e.g., a CD28 signaling domain described herein and/or a CD3 zeta signaling domain described herein). PD-1 is an inhibitory member of the CD28 family of receptors that also includes CD28, CTLA-4, ICOS, and BTLA. PD-1 is expressed on activated B cells, T cells and myeloid cells (Agata et al., 1996, Int. Immunol 8:765-75). Two ligands for PD-1, PD-L1 and PD-L2, have been shown to downregulate T cell activation upon binding to PD-1 (Freeman et al., 2000 J. Exp. Med. 192:1027-34; Latchman et al., 2001 Nat. Immunol. 2:261-8; Carter et al., 2002 Eur. J. Immunol. 32:634-43). PD-L1 is abundant in human cancers (Dong et al., 2003 J. Mol. Med. 81:281-7; Blank et al., 2005 Cancer Immunol. Immunother. 54:307-314; Konishi et al., 2004 Clin. Cancer Res. 10:5094). Immune suppression can be reversed by inhibiting the local interaction of PD-1 with PD-L1.

In one embodiment, the agent comprises the extracellular domain (ECD) of an inhibitory molecule, e.g., Programmed Death 1 (PD-1) can be fused to a transmembrane domain and optionally an intracellular signaling domain such as 41BB and CD3 zeta (also referred to herein as a PD-1 TFP). In one embodiment, the PD-1 TFP, when used in combinations with an anti-TAA TFP described herein, improves the persistence of the T cell. In one embodiment, the TFP is a PD-1 TFP comprising the extracellular domain of PD-1. Alternatively, provided are TFPs containing an antibody or antibody fragment such as a scFv that specifically binds to the Programmed Death-Ligand 1 (PD-L1) or Programmed Death-Ligand 2 (PD-L2).

In another aspect, the present disclosure provides a population of TFP-expressing T cells, e.g., TFP-T cells. In some embodiments, the population of TFP-expressing T cells comprises a mixture of cells expressing different TFPs. For example, in one embodiment, the population of TFP-T cells can include a first cell expressing a TFP having a binding domain described herein, and a second cell expressing a TFP having a different anti-TAA binding domain, e.g., a binding domain described herein that differs from the binding domain in the TFP expressed by the first cell. As another example, the population of TFP-expressing cells can include a first cell expressing a TFP that includes a first binding domain binding domain, e.g., as described herein, and a second cell expressing a TFP that includes an antigen binding domain to a target other than the binding domain of the first cell (e.g., another tumor-associated antigen).

In another aspect, the present disclosure provides a population of cells wherein at least one cell in the population expresses a TFP having a domain described herein, and a second cell expressing another agent, e.g., an agent which enhances the activity of a modified T cell. For example, in one embodiment, the agent can be an agent which inhibits an inhibitory molecule. Inhibitory molecules, e.g., can, in some embodiments, decrease the ability of a modified T cell to mount an immune effector response. Examples of inhibitory molecules include PD-1, PD-L1, PD-L2, CTLA4, TIM3, LAG3, VISTA, BTLA, TIGIT, LAIR1, CD160, 2B4 and TGFR beta. In one embodiment, the agent that inhibits an inhibitory molecule comprises a first polypeptide, e.g., an inhibitory molecule, associated with a second polypeptide that provides a positive signal to the cell, e.g., an intracellular signaling domain described herein.

### Recombinant Nucleic Acid Molecules

Disclosed herein are recombinant nucleic acid molecules comprising a first nucleic acid sequence encoding a T cell receptor (TCR) fusion protein (TFP) described herein and a second nucleic acid sequence encoding an Interleukin-15 (IL-15) polypeptide or a fragment thereof. Disclosed herein are recombinant nucleic acid molecules comprising a first nucleic acid sequence encoding a T cell receptor (TCR) fusion protein (TFP) and a second nucleic acid sequence encoding an Interleukin-15 receptor alpha (IL-15Rα) polypeptide or a fragment thereof. Also disclosed herein are recombinant nucleic acid molecules a first nucleic acid sequence encoding a T cell receptor (TCR) fusion protein (TFP) and a second nucleic acid sequence encoding a fusion protein comprising an IL-15 polypeptide or a fragment thereof linked to an IL-15Rα polypeptide or a fragment thereof. Further disclosed herein are recombinant nucleic acid molecules a first nucleic acid sequence encoding a T cell receptor (TCR) fusion protein (TFP) and a second nucleic acid sequence encoding a fusion protein comprising a fusion protein comprising an IL-15Rα polypeptide or a fragment thereof linked to PD-1 or a fragment thereof and/or CD28 or a fragment thereof.

### Recombinant Nucleic Acid Encoding a TFP and a TCR Constant Domain

Disclosed herein, in some embodiments, are recombinant nucleic acid molecules comprising a sequence encoding a T cell receptor (TCR) fusion protein (TFP). The TFP can comprise a TCR subunit comprising at least a portion of a TCR extracellular domain. The TCR subunit can further comprise a transmembrane domain. The TCR subunit can further comprise an intracellular domain of TCR gamma, TCR delta, TCR alpha or TCR beta or an intracellular domain comprising a stimulatory domain from an intracellular signaling domain of CD3 epsilon, CD3 gamma, CD3 delta. The TFP can further comprise an antibody (e.g., a human, humanized, or murine antibody) comprising an antigen binding domain. The recombinant nucleic acid molecule can further comprise a sequence encoding a TCR constant domain, wherein the TCR constant domain is a TCR alpha constant domain, a TCR beta constant domain, a TCR alpha constant domain and a TCR beta constant domain, a TCR gamma constant domain, a TCR delta constant domain, or a TCR gamma constant domain and a TCR delta constant domain. The TCR subunit and the antibody can be operatively linked. The TFP can functionally incorporate into a TCR complex (e.g., an endogenous TCR complex) when expressed in a T cell.

The constant domain can comprise a constant domain of a TCR alpha chain, a TCR beta chain, a TCR gamma chain or a TCR delta chain. The constant domain can comprise a full-length constant domain of a TCR alpha chain, a TCR beta chain, a TCR gamma chain or a TCR delta chain. The constant domain can comprise a fragment (e.g., functional fragment) of the full-length constant domain of a TCR alpha chain, a TCR beta chain, a TCR gamma chain or a TCR delta chain. For example, the constant domain can comprise at least about 5, 10, 15, 20, 25, 30, 35, 40, 45, 50, 55, 60, 65, 70, 75, 80, 85, 90, 95, 100, 150 or more amino acid residues of the constant domain of a TCR alpha chain, a TCR beta chain, a TCR gamma chain or a TCR delta chain. The sequence encoding the TCR constant domain can further encode the transmembrane domain and/or intracellular region of a TCR alpha chain, a TCR beta chain, a TCR gamma chain or a TCR delta chain. The sequence encoding the TCR constant domain can encode a full-length constant region of a TCR alpha chain, a TCR beta chain, a TCR gamma chain or a TCR delta chain. The constant region of a TCR chain can comprise a constant domain, a transmembrane domain, and an intracellular region. The constant region of a TCR chain can also exclude the transmembrane domain and the intracellular region of the TCR alpha chain, a TCR beta chain, a TCR gamma chain or a TCR delta chain.

The TCR alpha chain, a TCR beta chain, a TCR gamma chain or a TCR delta chain described herein can be derived from various species. The TCR chain can be a murine or human TCR chain. For example, the constant domain can comprise a constant domain of a murine or human TCR alpha chain, TCR beta chain, TCR gamma chain or TCR delta chain.

The constant domain can comprise truncations, additions, or substitutions of a sequence of a constant domain described herein. For example, the constant domain can comprise a truncated version of a constant domain described herein having at least about 5, 10, 15, 20, 25, 30, 35, 40, 45, 50, 55, 60, 65, 70, 75, 80, 85, 90, 95, 100, 150 or more amino acid residues of SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 142, SEQ ID NO: 143, SEQ ID NO: 146, SEQ ID NO: 148, SEQ ID NO: 149, SEQ ID NO: 152, SEQ ID NO: 155, SEQ ID NO:207, SEQ ID NO:209, SEQ ID NO:243 or SEQ ID NO:265. For example, the constant domain can comprise a sequence having at least about 5, 10, 15, 20, 25, 30, 35, 40, 45, 50, 55, 60, 65, 70, 75, 80, 85, 90, 95, 100, 150 or more additional amino acid residues of SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 142, SEQ ID NO: 143, SEQ ID NO: 146, SEQ ID NO: 148, SEQ ID NO: 149, SEQ ID NO: 152, SEQ ID NO: 155, SEQ ID NO:207, SEQ ID NO:209, SEQ ID NO:243 or SEQ ID NO:265. For example, the constant domain can comprise a sequence having at least about 5, 10, 15, 20, 25, 30, 35, 40, 45, 50, 55, 60, 65, 70, 75, 80, 85, 90, 95, 100, 150 or more amino acid substitutions of SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 142, SEQ ID NO: 143, SEQ ID NO: 146, SEQ ID NO: 148, SEQ ID NO: 149, SEQ ID NO: 152, SEQ ID NO: 155, SEQ ID NO:207, SEQ ID NO:209, SEQ ID NO:243 or SEQ ID NO:265. The constant domain can comprise a sequence or fragment thereof of SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 142, SEQ ID NO: 143, SEQ ID NO: 146, SEQ ID NO: 148, SEQ ID NO: 149, SEQ ID NO: 152, SEQ ID NO: 155, SEQ ID NO:207, SEQ ID NO:209, SEQ ID NO:243 or SEQ ID NO:265. The constant domain can comprise at least 1, 2, 3, 4, 5, 6, 7, 8, 9, 10 or more modifications, mutations or deletions of the sequence of SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 142, SEQ ID NO: 143, SEQ ID NO: 146, SEQ ID NO: 148, SEQ ID NO: 149, SEQ ID NO: 152, SEQ ID NO: 155, SEQ ID NO:207, SEQ ID NO:209, SEQ ID NO:243 or SEQ ID NO:265. The constant domain can comprise at most 20, 19, 18, 17, 16, 15, 14, 13, 12, 11, 10, 9, 8, 7, 6, 5, 4, 3, 2 or 1 modification, mutations or deletions of the sequence of SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 142, SEQ ID NO: 143, SEQ ID NO: 146, SEQ ID NO:148, SEQ ID NO: 149, SEQ ID NO: 152, SEQ ID NO: 155, SEQ ID NO:207, SEQ ID NO:209, SEQ ID NO:243 or SEQ ID NO:265. The constant domain can comprise a sequence having a sequence identity of at least about 40%, 45%, 50%, 55%, 60%, 65%, 70%, 75%, 80%, 85%, 90%, 95%, 99% or 100% to the sequence of SEQ ID NO: 16, SEQ ID NO: 17, SEQ ID NO: 18, SEQ ID NO: 19, SEQ ID NO: 20, SEQ ID NO: 21, or SEQ ID NO: 22, SEQ ID NO: 142, SEQ ID NO: 143, SEQ ID NO: 146, SEQ ID NO: 148, SEQ ID NO: 149, SEQ ID NO: 152, SEQ ID NO: 155, SEQ ID NO:207, SEQ ID NO:209, SEQ ID NO:243 or SEQ ID NO:265.

The murine TCR alpha constant domain can comprise positions 2-137 of SEQ ID NO: 146. The murine TCR alpha constant domain can comprise truncations, additions, or substitutions of a sequence of a constant domain described herein. For example, the constant domain can comprise a truncated version of a constant domain described herein having at least about 5, 10, 15, 20, 25, 30, 35, 40, 45, 50, 55, 60, 65, 70, 75, 80, 85, 90, 95, 100, 150 or more amino acid residues of positions 2-137 of SEQ ID NO: 146. For example, the constant domain can comprise a sequence having at least about 5, 10, 15, 20, 25, 30, 35, 40, 45, 50, 55, 60, 65, 70, 75, 80, 85, 90, 95, 100, 150 or more additional amino acid residues of positions 2-137 of SEQ ID NO: 146. For example, the constant domain can comprise a sequence having at least about 5, 10, 15, 20, 25, 30, 35, 40, 45, 50, 55, 60, 65, 70, 75, 80, 85, 90, 95, 100, 150 or more amino acid substitutions of positions 2-137 of SEQ ID NO:146. The constant domain can comprise a sequence or fragment thereof of positions 2-137 of SEQ ID NO: 146. The constant domain can comprise at least 1, 2, 3, 4, 5, 6, 7, 8, 9, 10 or more modifications, mutations or deletions of the sequence of positions 2-137 of SEQ ID NO: 146. The constant domain can comprise at most 20, 19, 18, 17, 16, 15, 14, 13, 12, 11, 10, 9, 8, 7, 6, 5, 4, 3, 2 or 1 modification, mutations or deletions of the sequence of positions 2-137 of SEQ ID NO: 146. The constant domain can comprise a sequence having a sequence identity of at least about 40%, 45%, 50%, 55%, 60%, 65%, 70%, 75%, 80%, 85%, 90%, 95%, 99% or 100% to the sequence of positions 2-137 of SEQ ID NO: 146.

The murine TCR beta constant domain can comprise positions 2-173 of SEQ ID NO: 152. The murine TCR beta constant domain can comprise truncations, additions, or substitutions of a sequence of a constant domain described herein. For example, the constant domain can comprise a truncated version of a constant domain described herein having at least about 5, 10, 15, 20, 25, 30, 35, 40, 45, 50, 55, 60, 65, 70, 75, 80, 85, 90, 95, 100, 150 or more amino acid residues of positions 2-173 of SEQ ID NO: 152. For example, the constant domain can comprise a sequence having at least about 5, 10, 15, 20, 25, 30, 35, 40, 45, 50, 55, 60, 65, 70, 75, 80, 85, 90, 95, 100, 150 or more additional amino acid residues of positions 2-173 of SEQ ID NO: 152. For example, the constant domain can comprise a sequence having at least about 5, 10, 15, 20, 25, 30, 35, 40, 45, 50, 55, 60, 65, 70, 75, 80, 85, 90, 95, 100, 150 or more amino acid substitutions of positions 2-173 of SEQ ID NO:152. The constant domain can comprise a sequence or fragment thereof of positions 22-173 of SEQ ID NO:152. The constant domain can comprise at least 1, 2, 3, 4, 5, 6, 7, 8, 9, 10 or more modifications, mutations or deletions of the sequence of positions 2-173 of SEQ ID NO: 152. The constant domain can comprise at most 20, 19, 18, 17, 16, 15, 14, 13, 12, 11, 10, 9, 8, 7, 6, 5, 4, 3, 2 or 1 modification, mutations or deletions of the sequence of positions 2-173 of SEQ ID NO: 152. The constant domain can comprise a sequence having a sequence identity of at least about 40%, 45%, 50%, 55%, 60%, 65%, 70%, 75%, 80%, 85%, 90%, 95%, 99% or 100% to the sequence of positions 2-173 of SEQ ID NO: 152.

In some instances, the TCR constant domain is a TCR delta constant domain. The TCR delta constant domain can comprise SEQ ID NO:20, SEQ ID NO:22, SEQ ID NO:243 or SEQ ID NO:265, functional fragments thereof, and amino acid sequences thereof having at least one but not more than 20 modification. In some embodiments, the TCR delta constant domain can comprise SEQ ID NO:243. The TCR delta constant domain can comprise truncations, additions, or substitutions of a sequence of a constant domain described herein. For example, the constant domain can comprise a truncated version of a constant domain described herein having at least about 5, 10, 15, 20, 25, 30, 35, 40, 45, 50, 55, 60, 65, 70, 75, 80, 85, 90, 95, 100, 150 or more amino acid residues of SEQ ID NO:243. For example, the constant domain can comprise a sequence having at least about 5, 10, 15, 20, 25, 30, 35, 40, 45, 50, 55, 60, 65, 70, 75, 80, 85, 90, 95, 100, 150 or more additional amino acid residues of SEQ ID NO:243. For example, the constant domain can comprise a sequence having at least about 5, 10, 15, 20, 25, 30, 35, 40, 45, 50, 55, 60, 65, 70, 75, 80, 85, 90, 95, 100, 150 or more amino acid substitutions of SEQ ID NO:243. The constant domain can comprise a sequence or fragment thereof of SEQ ID NO:243. The constant domain can comprise at least 1, 2, 3, 4, 5, 6, 7, 8, 9, 10 or more modifications, mutations or deletions of the sequence of SEQ ID NO:243. The constant domain can comprise at most 20, 19, 18, 17, 16, 15, 14, 13, 12, 11, 10, 9, 8, 7, 6, 5, 4, 3, 2 or 1 modification, mutations or deletions of the sequence of SEQ ID NO:243. The constant domain can comprise a sequence having a sequence identity of at least about 40%, 45%, 50%, 55%, 60%, 65%, 70%, 75%, 80%, 85%, 90%, 95%, 99% or 100% to the sequence of SEQ ID NO:243.

The TCR delta constant domain can comprise SEQ ID NO:20, SEQ ID NO:22, SEQ ID NO:243 or SEQ ID NO:265, functional fragments thereof, or amino acid sequences thereof having at least one but not more than 20 modifications. In some cases, the sequence encoding a TCR delta constant domain further encodes a TCR delta variable domain, thereby encoding a full TCR delta domain. The full TCR delta domain can be delta 2 or delta 1. The full TCR delta constant domain can comprise SEQ ID NO:256, functional fragments thereof, and amino acid sequences thereof having at least one but not more than 20 modifications.

The full TCR delta domain can comprise truncations, additions, or substitutions of a sequence of a constant domain described herein. For example, the delta domain can comprise a truncated version of a delta domain described herein having at least about 5, 10, 15, 20, 25, 30, 35, 40, 45, 50, 55, 60, 65, 70, 75, 80, 85, 90, 95, 100, 150 or more amino acid residues of SEQ ID NO:256. For example, the delta domain can comprise a sequence having at least about 5, 10, 15, 20, 25, 30, 35, 40, 45, 50, 55, 60, 65, 70, 75, 80, 85, 90, 95, 100, 150 or more additional amino acid residues of SEQ ID NO:256. For example, the delta domain can comprise a sequence having at least about 5, 10, 15, 20, 25, 30, 35, 40, 45, 50, 55, 60, 65, 70, 75, 80, 85, 90, 95, 100, 150 or more amino acid substitutions of SEQ ID NO:256. The delta domain can comprise a sequence or fragment thereof of SEQ ID NO:256. The delta domain can comprise at least 1, 2, 3, 4, 5, 6, 7, 8, 9, 10 or more modifications, mutations or deletions of the sequence of SEQ ID NO:256. The delta domain can comprise at most 20, 19, 18, 17, 16, 15, 14, 13, 12, 11, 10, 9, 8, 7, 6, 5, 4, 3, 2 or 1 modification, mutations or deletions of the sequence of SEQ ID NO:256. The delta domain can comprise a sequence having a sequence identity of at least about 40%, 45%, 50%, 55%, 60%, 65%, 70%, 75%, 80%, 85%, 90%, 95%, 99% or 100% to the sequence of SEQ ID NO:256.

The TCR gamma constant domain can comprise SEQ ID NO:21. The TCR gamma constant domain can comprise truncations, additions, or substitutions of a sequence of a constant domain described herein. For example, the constant domain can comprise a truncated version of a constant domain described herein having at least about 5, 10, 15, 20, 25, 30, 35, 40, 45, 50, 55, 60, 65, 70, 75, 80, 85, 90, 95, 100, 150 or more amino acid residues of SEQ ID NO:21. For example, the constant domain can comprise a sequence having at least about 5, 10, 15, 20, 25, 30, 35, 40, 45, 50, 55, 60, 65, 70, 75, 80, 85, 90, 95, 100, 150 or more additional amino acid residues of SEQ ID NO:21. For example, the constant domain can comprise a sequence having at least about 5, 10, 15, 20, 25, 30, 35, 40, 45, 50, 55, 60, 65, 70, 75, 80, 85, 90, 95, 100, 150 or more amino acid substitutions of SEQ ID NO:21. The constant domain can comprise a sequence or fragment thereof of SEQ ID NO:21. The constant domain can comprise at least 1, 2, 3, 4, 5, 6, 7, 8, 9, 10 or more modifications, mutations or deletions of the sequence of SEQ ID NO:21. The constant domain can comprise at most 20, 19, 18, 17, 16, 15, 14, 13, 12, 11, 10, 9, 8, 7, 6, 5, 4, 3, 2 or 1 modification, mutations or deletions of the sequence of SEQ ID NO:21. The constant domain can comprise a sequence having a sequence identity of at least about 40%, 45%, 50%, 55%, 60%, 65%, 70%, 75%, 80%, 85%, 90%, 95%, 99% or 100% to the sequence of SEQ ID NO:243.

The TCR gamma constant domain can comprise SEQ ID NO:21 or SEQ ID NO: 155, functional fragments thereof, and amino acid sequences thereof having at least one but not more than 20 modifications. In some cases, the sequence encoding the TCR gamma constant domain further encodes a TCR gamma variable domain, thereby encoding a full TCR gamma domain. The full TCR gamma domain can be gamma 9 or gamma 4. The full TCR gamma domain can comprise SEQ ID NO:255, functional fragments thereof, and amino acid sequences thereof having at least one but not more than 20 modifications.

The full TCR gamma domain can comprise truncations, additions, or substitutions of a sequence of a constant domain described herein. For example, the gamma domain can comprise a truncated version of a gamma domain described herein having at least about 5, 10, 15, 20, 25, 30, 35, 40, 45, 50, 55, 60, 65, 70, 75, 80, 85, 90, 95, 100, 150 or more amino acid residues of SEQ ID NO:255. For example, the gamma domain can comprise a sequence having at least about 5, 10, 15, 20, 25, 30, 35, 40, 45, 50, 55, 60, 65, 70, 75, 80, 85, 90, 95, 100, 150 or more additional amino acid residues of SEQ ID NO:255. For example, the gamma domain can comprise a sequence having at least about 5, 10, 15, 20, 25, 30, 35, 40, 45, 50, 55, 60, 65, 70, 75, 80, 85, 90, 95, 100, 150 or more amino acid substitutions of SEQ ID NO:255. The gamma domain can comprise a sequence or fragment thereof of SEQ ID NO:255. The gamma domain can comprise at least 1, 2, 3, 4, 5, 6, 7, 8, 9, 10 or more modifications, mutations or gamma of the sequence of SEQ ID NO:255. The gamma domain can comprise at most 20, 19, 18, 17, 16, 15, 14, 13, 12, 11, 10, 9, 8, 7, 6, 5, 4, 3, 2 or 1 modification, mutations or deletions of the sequence of SEQ ID NO:255. The gamma domain can comprise a sequence having a sequence identity of at least about 40%, 45%, 50%, 55%, 60%, 65%, 70%, 75%, 80%, 85%, 90%, 95%, 99% or 100% to the sequence of SEQ ID NO:255.

TCR beta chain (Homo sapiens):
(SEQ ID NO: 16)
VEDLNKVFPPEVAVFEPSEAEISHTQKATLVCLATGFFPDHVELSWWVNG
KEVHSGVSTDPQPLKEQPALNDSRYCLSSRLRVSATFWQNPRNHFRCQVQ
FYGLSENDEWTQDRAKPVTQIVSAEAWGRADCGFTSVSYQQGVLSATILY
EILLGKATLYAVLVSALVLMAMVKRKDF.

The murine TCR beta chain constant region canonical sequence is:

(SEQ ID NO: 152)
EDLRNVTPPKVSLFEPSKAEIANKQKATLVCLARGFFPDHVELSWWVNGK
EVHSGVSTDPQAYKESNYSYCLSSRLRVSATFWHNPRNHFRCQVQFHGLS
EEDKWPEGSPKPVTQNISAEAWGRADCGITSASYQQGVLSATILYEILLG
KATLYAVLVSTLVVMAMVKRKNS.
TCR alpha constant region (Mus musculus)
(or [mm]TRAC(82-137)):
(SEQ ID NO: 17)
ATYPSSDVPCDATLTEKSFETDMNLNFQNLSVMGLRILLLKVAGFNLLMT
LRLWSS.

The murine TCR alpha chain constant (mTRAC) region canonical sequence is:

(SEQ ID NO: 146)
XIQNPEPAVYQLKDPRSQDSTLCLFTDFDSQINVPKTMESGTFITDKTVL
DMKAMDSKSNGAIAWSNQTSFTCQDIFKETNATYPSSDVPCDATLTEKSF
ETDMNLNFQNLSVMGLRILLLKVAGFNLLMTLRLWSS.
TCR beta constant region (Mus musculus)
(or [mm]TRBC1(123-173)):
(SEQ ID NO: 18)
GRADCGITSASYQQGVLSATILYEILLGKATLYAVLVSTLVVMAMVKRKN
S

The murine TCR beta chain constant region canonical sequence is:

(SEQ ID NO: 152)
EDLRNVTPPKVSLFEPSKAEIANKQKATLVCLARGFFPDHVELSWWVNGK
EVHSGVSTDPQAYKESNYSYCLSSRLRVSATFWHNPRNHFRCQVQFHGLS
EEDKWPEGSPKPVTQNISAEAWGRADCGITSASYQQGVLSATILYEILLG
KATLYAVLVSTLVVMAMVKRKNS.
TCR beta chain (Homo sapiens):
(SEQ ID NO: 19)
PVDSGVTQTPKHLITATGQRVTLRCSPRSGDLSVSWYQQSLDQGLQFLIQ
YYNGEERAKGNILERFSAQQFPDLHSELNLSSLELGDSALYFCASSPRTG
LNTEAFFGQGTRLTVVEDLNKVFPPEVAVFEPSEAEISHTQKATLVCLAT
GFFPDHVELSWWVNGKEVHSGVSTDPQPLKEQPALNDSRYCLSSRLRVSA
TFWQNPRNHFRCQVQFYGLSENDEWTQDRAKPVTQIVSAEAWGRADCGFT
SVSYQQGVLSATILYEILLGKATLYAVLVSALVLMAMVKRKDF.
TCR delta constant region version 1
(Homo sapiens):
(SEQ ID NO: 20)
SQPHTKPSVFVMKNGTNVACLVKEFYPKDIRINLVSSKKITEFDPAIVIS
PSGKYNAVKLGKYEDSNSVTCSVQHDNKTVHSTDFEVKTDSTDHVKPKET
ENTKQPSKSCHKPKAIVHTEKVNMMSLTVLGLRMLFAKTVAVNFLLTAKL
FF.
TCR gamma constant region (Homo sapiens)
(or [hs]TRGC(1-173)):
(SEQ ID NO: 21)
DKQLDADVSPKPTIFLPSIAETKLQKAGTYLCLLEKFFPDVIKIHWQEKK
SNTILGSQEGNTMKTNDTYMKFSWLTVPEKSLDKEHRCIVRHENNKNGVD
QEIIFPPIKTDVITMDPKDNCSKDANDTLLLQLTNTSAYYMYLLLLLKSV
VYFAIITCCLLRRTAFCCNGEKS.
TCR delta constant region version 2
(Homo sapiens):
(SEQ ID NO: 22)
SQPHTKPSVFVMKNGTNVACLVKEFYPKDIRINLVSSKKITEFDPAIVIS
PSGKYNAVKLGKYEDSNSVTCSVQHDNKTVHSTDFEVKTDSTDHVKPKET
ENTKQPSKSCHKPKAIVHTEKVNMMSLTVLGLRMLFAKTVAVNFLLTAK.

In some instances, the TCR constant domain is a TCR delta constant domain. The sequence encoding the TCR delta constant domain can further encode a second antigen binding domain or ligand binding domain that is operatively linked to the sequence encoding the TCR delta constant domain. The second antigen binding domain or ligand binding domain can be the same or different as the antigen binding domain or ligand binding domain of the TFP.

In some instances, the TCR constant domain is a TCR gamma constant domain. The sequence encoding the TCR gamma constant domain can further encode a second antigen binding domain or ligand binding domain that is operatively linked to the sequence encoding the TCR gamma constant domain. The second antigen binding domain or ligand binding domain can be the same or different as the antigen binding domain or ligand binding domain of the TFP.

In some instances, the recombinant nucleic acid comprises a sequence encoding a TCR gamma constant domain and a TCR delta constant domain. The TCR gamma constant domain can comprise SEQ ID NO:21 or SEQ ID NO: 155, functional fragments thereof, and amino acid sequences thereof having at least one but not more than 20 modifications. The sequence encoding the TCR gamma constant domain can further encode a TCR gamma variable domain, thereby encoding a full TCR gamma domain. The TCR gamma domain can be gamma 9 or gamma 4. The full TCR gamma domain comprises SEQ ID NO:255, functional fragments thereof, and amino acid sequences thereof having at least one but not more than 20 modifications. The TCR delta constant domain can comprise SEQ ID NO:20, SEQ ID NO:22, SEQ ID NO:243 or SEQ ID NO:265, functional fragments thereof, and amino acid sequences thereof having at least one but not more than 20 modifications. The sequence encoding the TCR delta constant domain can further encode a TCR delta variable domain, thereby encoding a full TCR delta domain. The TCR delta domain can be delta 2 or delta 1. The full TCR delta domain can comprise SEQ ID NO:256, functional fragments thereof, and amino acid sequences thereof having at least one but not more than 20 modifications.

In some instances, the TCR constant domain incorporates into a functional TCR complex when expressed in a T cell. In some instances, the TCR constant domain incorporates into a same functional TCR complex as the functional TCR complex that incorporates the TFP when expressed in a T cell. In some instances, the sequence encoding the TFP and the sequence encoding the TCR constant domain are contained within a same nucleic acid molecule. In some instances, the sequence encoding the TFP and the sequence encoding the TCR constant domain are contained within different nucleic acid molecules. The sequence can further encode a cleavage site (e.g., a protease cleavage site) between the encoded TFP and the TCR constant domain. The cleavage site can be a protease cleavage site. The cleavage site can be a self-cleaving peptide such as a T2A, P2A, E2A or F2A cleavage site. The cleavage site can comprise a sequence of SEQ ID NO: 23.

T2A cleavage site: EGRGSLLTCGDVEENPGP (SEQ ID NO: 23).

The TCR subunit of the TFP and the constant domain can comprise a sequence derived from a same TCR chain or a different TCR chain. In some cases, the TCR subunit of the TFP and the constant domain are derived from different TCR chains. For example, the TCR subunit can comprise (1) at least a portion of a TCR extracellular domain, (2) a transmembrane domain, and (3) an intracellular domain, where the TCR extracellular domain, the transmembrane domain and the intracellular domain are derived from a TCR alpha chain, and the constant domain can comprise a constant domain of a TCR beta chain. For another example, the TCR subunit can comprise (1) at least a portion of a TCR extracellular domain, (2) a transmembrane domain, and (3) an intracellular domain, where the TCR extracellular domain, the transmembrane domain and the intracellular domain are derived from a TCR beta chain, and the constant domain can comprise a constant domain of a TCR alpha chain. For another example, the TCR subunit can comprise (1) at least a portion of a TCR extracellular domain, (2) a transmembrane domain, and (3) an intracellular domain, where the TCR extracellular domain, the transmembrane domain and the intracellular domain are derived from a TCR gamma chain, and the constant domain can comprise a constant domain of a TCR delta chain. For yet another example, the TCR subunit can comprise (1) at least a portion of a TCR extracellular domain, (2) a transmembrane domain, and (3) an intracellular domain, where the TCR extracellular domain, the transmembrane domain and the intracellular domain are derived from a TCR delta chain, and the constant domain can comprise a constant domain of a TCR gamma chain.

In some instances, the TCR subunit and the antibody domain, the antigen domain or the binding ligand or fragment thereof are operatively linked by a linker sequence. In some instances, the linker sequence comprises (G₄S)ₙ, wherein n=1 to 4.

In some instances, the transmembrane domain is a TCR transmembrane domain from CD3 epsilon, CD3 gamma, CD3 delta, TCR gamma, TCR delta, TCR alpha or TCR beta. In some instances, the intracellular domain is derived from only CD3 epsilon, only CD3 gamma, only CD3 delta, only TCR gamma, only TCR delta, only TCR alpha or only TCR beta.

In some instances, the TCR subunit comprises (i) at least a portion of a TCR extracellular domain, (ii) a TCR transmembrane domain, and (iii) a TCR intracellular domain, wherein at least two or all of (i), (ii), and (iii) are from the same TCR subunit.

In some instances, the TCR extracellular domain comprises an extracellular domain or portion thereof of a protein selected from the group consisting of a TCR alpha chain, a TCR beta chain, a TCR gamma chain, a TCR delta chain, a CD3 epsilon TCR subunit, a CD3 gamma TCR subunit, a CD3 delta TCR subunit, functional fragments thereof, and amino acid sequences thereof having at least one but not more than 20 modifications.

In some instances, the TCR subunit comprises a transmembrane domain comprising a transmembrane domain of a protein selected from the group consisting of a TCR alpha chain, a TCR beta chain, a TCR zeta chain, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, CD154, functional fragments thereof, and amino acid sequences thereof having at least one but not more than 20 modifications.

In some instances, the TCR subunit comprises a TCR intracellular domain of TCR alpha chain, a TCR beta chain, a TCR gamma chain, a TCR delta chain, or a fragment thereof. In some instances, the TCR subunit comprises an intracellular domain comprising a stimulatory domain of a protein selected from an intracellular signaling domain of CD3 epsilon, CD3 gamma or CD3 delta, or an amino acid sequence having at least one modification thereto.

In some instances, the TCR subunit can comprise (i) at least a portion of a TCR extracellular domain, (ii) a TCR transmembrane domain, and (iii) a TCR intracellular domain of a TCR gamma chain or a TCR delta chain. The TCR extracellular domain can comprise the extracellular portion of a constant domain of a TCR gamma chain or a TCR delta chain, functional fragments thereof, and amino acid sequences thereof having at least one but not more than 20 modifications. In some embodiments, the TCR subunit comprising (i) at least a portion of a TCR extracellular domain, (ii) a TCR transmembrane domain, and (iii) a TCR intracellular domain is or comprises a delta constant domain, or a fragment thereof, e.g., a delta constant domain described herein. The delta constant domain can have the sequence of SEQ ID NO:20, SEQ ID NO:22, SEQ ID NO:243 or SEQ ID NO:265, functional fragments thereof, and amino acid sequences thereof having at least one but not more than 20 modifications. In some embodiments, the TCR subunit comprising (i) at least a portion of a TCR extracellular domain, (ii) a TCR transmembrane domain, and (iii) a TCR intracellular domain is or comprises a gamma constant domain, e.g., a gamma constant domain described herein. The gamma constant domain can have the sequence of SEQ ID NO:21 or SEQ ID NO: 155, functional fragments thereof, and amino acid sequences thereof having at least one but not more than 20 modifications. The extracellular domain of the TFP may not comprise the variable domain of a gamma chain or a delta chain.

The TCR subunit of the TFP can comprise the extracellular, transmembrane and intracellular domain of CD3 epsilon, CD3 gamma, or CD3 delta. In some embodiments, recombinant nucleic acid comprises a TFP comprising the extracellular, transmembrane and intracellular domain of CD3 epsilon, CD3 gamma, or CD3 delta and the constant domains of TCR beta and TCR alpha. In some embodiments, recombinant nucleic acid comprises a TFP comprising the extracellular, transmembrane and intracellular domain of CD3 epsilon and the constant domains of TCR gamma and TCR delta. In some embodiments, recombinant nucleic acid comprises a TFP comprising the extracellular, transmembrane and intracellular domain of CD3 epsilon and full length TCF gamma and full length TCR delta. In some embodiments, the TCR subunit of the TFP comprises CD3 epsilon. The TCR subunit of CD3 epsilon can comprise the sequence of SEQ ID NO:364 functional fragments thereof, and amino acid sequences thereof having at least one but not more than 20 modifications.

In some instances, the TCR subunit comprising at least a portion of a murine TCR alpha or murine TCR beta extracellular domain and a murine TCR alpha or murine TCR beta transmembrane domain is or comprises a TCR alpha constant domain or a TCR beta constant domain. The TCR subunit can comprise an intracellular domain of murine TCR alpha or murine TCR beta. The TCR constant domain can be a TCR alpha constant domain, e.g., a TCR alpha constant domain described herein. The TCR alpha constant domain can comprise SEQ ID NO: 17, SEQ ID NO: 142, SEQ ID NO: 143, SEQ ID NO: 146, or SEQ ID NO:207, functional fragments thereof, and amino acid sequences thereof having at least one but not more than 20 modifications. The sequence encoding the TCR alpha constant domain can further encode a second antigen binding domain or ligand binding domain that is operatively linked to the sequence encoding the TCR alpha constant domain. The second antigen binding domain or ligand binding domain can be the same or different as the antigen binding domain or ligand binding domain of the TFP. The TCR alpha constant domain can comprise a murine TCR alpha constant domain. The murine TCR alpha constant domain can comprise amino acids 2-137 of the murine TCR alpha constant domain. The murine TCR alpha constant domain can comprise amino acids 2-137 of SEQ ID NO: 146. The murine TCR alpha constant domain can comprise a sequence of SEQ ID NO:207. The murine TCR alpha constant domain can comprise amino acids 82-137 of SEQ ID NO: 146. The murine TCR alpha constant domain comprises a sequence of SEQ ID NO: 17. The TCR constant domain can be a TCR beta constant domain, e.g., a TCR beta constant domain described herein. The TCR beta constant domain can comprise SEQ ID NO: 18, SEQ ID NO: 148, SEQ ID NO: 149, SEQ ID NO: 152, or SEQ ID NO:209, functional fragments thereof, and amino acid sequences thereof having at least one but not more than 20 modifications. The sequence encoding the TCR beta constant domain can further encode a second antigen binding domain or ligand binding domain that is operatively linked to the sequence encoding the TCR beta constant domain. The second antigen binding domain or ligand binding domain can be the same or different as the antigen binding domain or ligand binding domain of the TFP. TCR beta constant domain can comprise a murine TCR beta constant domain. The murine TCR beta constant domain can comprise amino acids 2-173 of the murine TCR beta constant domain. The murine TCR beta constant domain can comprise amino acids 2-173 of SEQ ID NO: 152. The murine TCR beta constant domain can comprise SEQ ID NO:209. The TCR beta constant domain can comprise amino acids 123-173 of SEQ ID NO: 152. The TCR beta constant domain can comprise SEQ ID NO: 18

The recombinant nucleic acid can comprise sequence encoding a TCR alpha constant domain and a TCR beta constant domain. The TCR alpha constant domain can comprise SEQ ID NO: 17, SEQ ID NO: 142, SEQ ID NO: 143, SEQ ID NO: 146, or SEQ ID NO:207, functional fragments thereof, and amino acid sequences thereof having at least one but not more than 20 modifications. The TCR beta constant domain can comprise SEQ ID NO: 18, SEQ ID NO: 148, SEQ ID NO: 149, SEQ ID NO: 152, or SEQ ID NO:209, functional fragments thereof, and amino acid sequences thereof having at least one but not more than 20 modifications. The intracellular signaling domain can be CD3 epsilon, CD3 gamma, or CD3 delta. The intracellular signaling domain can be CD3 epsilon.

The sequence encoding the TCR constant domain can comprise from 5′ to 3′, a first leader sequence, an antigen binding domain sequence, a linker, a TRAC gene sequence, a cleavable linker sequence, a second leader sequence, and a TRBC gene sequence. The sequence encoding the TCR constant domain can comprise, from 5′ to 3′, a first leader sequence, an antigen binding domain sequence, a linker, a TRAC gene sequence, a cleavable linker sequence, a second leader sequence, and a TRBC gene sequence. The sequence encoding the TCR constant domain can comprise, from 5′ to 3′, a first leader sequence, a TRAC gene sequence, a cleavable linker sequence, a second leader sequence, an antigen binding domain sequence, a linker, and a TRBC gene sequence. The sequence encoding the TCR constant domain can comprise, from 5′ to 3′, a first leader sequence, an antigen binding domain sequence, a linker, a TRAC gene sequence, a cleavable linker sequence, a second leader sequence, an antigen binding domain sequence, a linker, and a TRBC gene sequence. The sequence encoding the TCR constant domain can comprise, from 5′-3′, a first leader sequence, a TRAC gene sequence, a first cleavable linker sequence, a second leader sequence, a TRBC gene sequence, a second cleavable linker sequence, a third leader sequence, an antigen binding domain sequence, a linker sequence, and a CD3 epsilon gene sequence.

As described herein, the at least one but not more than 20 modifications thereto of a sequence described herein can comprise a modification of an amino acid that mediates cell signaling or a modification of an amino acid that is phosphorylated in response to a ligand binding to the TFP.

In some instances, the TCR subunit comprises an intracellular domain comprising a stimulatory domain of a protein selected from a functional signaling domain of 4-1BB and/or a functional signaling domain of CD3 zeta, or an amino acid sequence having at least one modification thereto.

In some instances, the recombinant nucleic acid further comprises a sequence encoding a costimulatory domain. In some instances, the costimulatory domain comprises a functional signaling domain of a protein selected from the group consisting of OX40, CD2, CD27, CD28, CDS, ICAM-1, LFA-1 (CD11a/CD18), ICOS (CD278), and 4-1BB (CD137), and amino acid sequences thereof having at least one but not more than 20 modifications thereto.

In some instances, the TCR subunit comprises an immunoreceptor tyrosine-based activation motif (ITAM) of a TCR subunit that comprises an ITAM or portion thereof of a protein selected from the group consisting of CD3 zeta TCR subunit, CD3 epsilon TCR subunit, CD3 gamma TCR subunit, CD3 delta TCR subunit, TCR zeta chain, Fc epsilon receptor 1 chain, Fc epsilon receptor 2 chain, Fc gamma receptor 1 chain, Fc gamma receptor 2a chain, Fc gamma receptor 2b1 chain, Fc gamma receptor 2b2 chain, Fc gamma receptor 3a chain, Fc gamma receptor 3b chain, Fc beta receptor 1 chain, TYROBP (DAP12), CD5, CD16a, CD16b, CD22, CD23, CD32, CD64, CD79a, CD79b, CD89, CD278, CD66d, functional fragments thereof, and amino acid sequences thereof having at least one but not more than 20 modifications thereto. In some instances, the ITAM replaces an ITAM of CD3 gamma, CD3 delta, or CD3 epsilon. In some instances, the ITAM is selected from the group consisting of CD3 zeta TCR subunit, CD3 epsilon TCR subunit, CD3 gamma TCR subunit, and CD3 delta TCR subunit and replaces a different ITAM selected from the group consisting of CD3 zeta TCR subunit, CD3 epsilon TCR subunit, CD3 gamma TCR subunit, and CD3 delta TCR subunit.

In some instances, the TFP, the TCR gamma constant domain, the TCR delta constant domain, and any combination thereof is capable of functionally interacting with an endogenous TCR complex and/or at least one endogenous TCR polypeptide. In some instances, (a) the TCR constant domain is a TCR gamma constant domain and the TFP functionally integrates into a TCR complex comprising an endogenous subunit of TCR delta, CD3 epsilon, CD3 gamma, CD3 delta, or a combination thereof, (b) the TCR constant domain is a TCR delta constant domain and the TFP functionally integrates into a TCR complex comprising an endogenous subunit of TCR gamma, CD3 epsilon, CD3 gamma, CD3 delta, or a combination thereof, or (c) the TCR constant domain is a TCR gamma constant domain and a TCR delta constant domain and the TFP functionally integrates into a TCR complex comprising an endogenous subunit of CD3 epsilon, CD3 gamma, CD3 delta, or a combination thereof.

In some instances, the at least one but not more than 20 modifications thereto comprise a modification of an amino acid that mediates cell signaling or a modification of an amino acid that is phosphorylated in response to a ligand binding to the TFP.

The antibody or antigen binding domain can be an antibody fragment. The antibody or antigen binding domain can be murine, human or humanized. In some instances, the murine, human or humanized antibody is an antibody fragment. In some instances, the antibody fragment is a scFv, a single domain antibody domain, a VH domain or a VL domain. In some instances, murine, human or humanized antibody comprising an antigen binding domain is selected from a group consisting of an anti-CD19 binding domain, anti-B-cell maturation antigen (BCMA) binding domain, anti-mesothelin (MSLN) binding domain, anti-CD22 binding domain, anti-PD-1 binding domain, anti-BAFF or BAFF receptor binding domain, and anti-ROR-1 binding domain.

An antigen binding domain described herein can be selected from a group consisting of an anti-CD19 binding domain, an anti-B-cell maturation antigen (BCMA) binding domain, an anti-mesothelin (MSLN) binding domain, an anti-CD20 binding domain, an anti-CD70 binding domain, an anti-79b binding domain, an anti-HER2 binding domain, an anti-PMSA binding domain, an anti-MUC16 binding domain, an anti-CD22 binding domain, an anti-PD-L1 binding domain, an anti BAFF or BAFF receptor binding domain, an anti-Nectin-4 binding domain, an anti-TROP-2 binding domain, an anti-GPC3 binding domain, and anti-ROR-1 binding domain.

In some instances, the nucleic acid is selected from the group consisting of a DNA and an RNA. In some instances, the nucleic acid is an mRNA. In some instances, the recombinant nucleic acid comprises a nucleic acid analog, wherein the nucleic acid analog is not in an encoding sequence of the recombinant nucleic acid. In some instances, the nucleic analog is selected from the group consisting of 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), 2′-O—N-methylacetamido (2′-O-NMA) modified, a locked nucleic acid (LNA), an ethylene nucleic acid (ENA), a peptide nucleic acid (PNA), a 1′,5′-anhydrohexitol nucleic acid (HNA), a morpholino, a methylphosphonate nucleotide, a thiolphosphonate nucleotide, and a 2′-fluoro N3-P5′-phosphoramidite.

In some instances, the recombinant nucleic acid further comprises a leader sequence. In some instances, the recombinant nucleic acid further comprises a promoter sequence. In some instances, the recombinant nucleic acid further comprises a sequence encoding a poly(A) tail. In some instances, the recombinant nucleic acid further comprises a 3′UTR sequence. In some instances, the nucleic acid is an isolated nucleic acid or a non-naturally occurring nucleic acid. In some instances, the nucleic acid is an in vitro transcribed nucleic acid.

In some instances, the recombinant nucleic acid further comprises a sequence encoding a TCR alpha transmembrane domain. In some instances, the recombinant nucleic acid further comprises a sequence encoding a TCR beta transmembrane domain. In some instances, the recombinant nucleic acid further comprises a sequence encoding a TCR alpha transmembrane domain and a sequence encoding a TCR beta transmembrane domain.

In some instances, the TCR subunit comprises an immunoreceptor tyrosine-based activation motif (ITAM) of a TCR subunit that comprises an ITAM or portion thereof of a protein selected from the group consisting of CD3 zeta TCR subunit, CD3 epsilon TCR subunit, CD3 gamma TCR subunit, CD3 delta TCR subunit, TCR zeta chain, Fc epsilon receptor 1 chain, Fc epsilon receptor 2 chain, Fc gamma receptor 1 chain, Fc gamma receptor 2a chain, Fc gamma receptor 2b1 chain, Fc gamma receptor 2b2 chain, Fc gamma receptor 3a chain, Fc gamma receptor 3b chain, Fc beta receptor 1 chain, TYROBP (DAP12), CD5, CD16a, CD16b, CD22, CD23, CD32, CD64, CD79a, CD79b, CD89, CD278, CD66d, functional fragments thereof, and amino acid sequences thereof having at least one but not more than 20 modifications thereto. In some instances, the ITAM replaces an ITAM of CD3 gamma, CD3 delta, or CD3 epsilon. In some instances, the ITAM is selected from the group consisting of CD3 zeta TCR subunit, CD3 epsilon TCR subunit, CD3 gamma TCR subunit, and CD3 delta TCR subunit and replaces a different ITAM selected from the group consisting of CD3 zeta TCR subunit, CD3 epsilon TCR subunit, CD3 gamma TCR subunit, and CD3 delta TCR subunit.

In some instances, the TFP, the TCR gamma constant domain, the TCR delta constant domain, the TCR alpha constant domain, the TCR beta constant domain, and any combination thereof is capable of functionally interacting with an endogenous TCR complex and/or at least one endogenous TCR polypeptide. In some instances, (a) the TCR constant domain is a TCR gamma constant domain and the TFP functionally integrates into a TCR complex comprising an endogenous subunit of TCR beta, CD3 epsilon, CD3 gamma, CD3 delta, or a combination thereof, (b) the TCR constant domain is a TCR delta constant domain and the TFP functionally integrates into a TCR complex comprising an endogenous subunit of TCR alpha, CD3 epsilon, CD3 gamma, CD3 delta, or a combination thereof, (c) the TCR constant domain is a TCR gamma constant domain and a TCR delta constant domain and the TFP functionally integrates into a TCR complex comprising an endogenous subunit of CD3 epsilon, CD3 gamma, CD3 delta, or a combination thereof, (d) the TCR constant domain is a TCR alpha constant domain and the TFP functionally integrates into a TCR complex comprising an endogenous subunit of TCR beta, CD3 epsilon, CD3 gamma, CD3 delta, or a combination thereof, or (e) the TCR constant domain is a TCR beta constant domain and the TFP functionally integrates into a TCR complex comprising an endogenous subunit of TCR alpha, CD3 epsilon, CD3 gamma, CD3 delta, or a combination thereof.

In some instances, the at least one but not more than 20 modifications thereto comprise a modification of an amino acid that mediates cell signaling or a modification of an amino acid that is phosphorylated in response to a ligand binding to the TFP.

In some instances, the murine, human or humanized antibody is an antibody fragment. In some instances, the antibody fragment is a scFv, a single domain antibody domain (sdAb), a VH domain or a VL domain. In some instances, murine, human or humanized antibody comprising an antigen binding domain is selected from a group consisting of an anti-CD19 binding domain, anti-B-cell maturation antigen (BCMA) binding domain, anti-mesothelin (MSLN) binding domain, anti-CD22 binding domain, anti-PD-1 binding domain, anti PD-L1 binding domain, anti IL13Rα2 binding domain, anti-BAFF or BAFFR binding domain, and anti-ROR-1 binding domain.

In some instances, the antigen domain comprises a ligand. In some instances, the ligand binds to the receptor of a cell. In some instances, the ligand binds to the polypeptide expressed on a surface of a cell. In some instances, the receptor or polypeptide expressed on a surface of a cell comprises a stress response receptor or polypeptide. In some instances, the receptor or polypeptide expressed on a surface of a cell is an MHC class I-related glycoprotein. In some instances, the MHC class I-related glycoprotein is selected from the group consisting of MICA, MICB, RAET1E, RAET1G, ULBP1, ULBP2, ULBP3, ULBP4 and combinations thereof. In some instances, the antigen domain comprises a monomer, a dimer, a trimer, a tetramer, a pentamer, a hexamer, a heptamer, an octomer, a nonamer, or a decamer. In some instances, the antigen domain comprises a monomer or a dimer of the ligand or fragment thereof. In some instances, the ligand or fragment thereof is a monomer, a dimer, a trimer, a tetramer, a pentamer, a hexamer, a heptamer, an octomer, a nonamer, or a decamer. In some instances, the ligand or fragment thereof is a monomer or a dimer. In some instances, the antigen domain does not comprise an antibody or fragment thereof. In some instances, the antigen domain does not comprise a variable region. In some instances, the antigen domain does not comprise a CDR. In some instances, the ligand or fragment thereof is a Natural Killer Group 2D (NKG2D) ligand or a fragment thereof.

In some instances, the TCR subunit and the antibody domain, the antigen domain or the binding ligand or fragment thereof are operatively linked by a linker sequence. In some instances, the linker sequence comprises (G₄S)ₙ, wherein n=1 to 4.

In some instances, the transmembrane domain is a TCR transmembrane domain from CD3 epsilon, CD3 gamma, CD3 delta, TCR alpha, TCR beta, TCR delta, or TCR gamma. In some instances, the intracellular domain is derived from only CD3 epsilon, only CD3 gamma, only CD3 delta, only TCR alpha, only TCR beta, only TCR delta, or only TCR gamma.

In some instances, the TCR subunit comprises (i) at least a portion of a TCR extracellular domain, (ii) a TCR transmembrane domain, and (iii) a TCR intracellular domain, wherein at least two of (i), (ii), and (iii) are from the same TCR subunit.

In some instances, the TCR extracellular domain comprises an extracellular domain or portion thereof of a protein selected from the group consisting of a TCR alpha chain, a TCR beta chain, a TCR delta chain, a TCR gamma chain, a CD3 epsilon TCR subunit, a CD3 gamma TCR subunit, a CD3 delta TCR subunit, functional fragments thereof, and amino acid sequences thereof having at least one but not more than 20 modifications.

In some instances, the TCR subunit comprises a transmembrane domain comprising a transmembrane domain of a protein selected from the group consisting of a TCR alpha chain, a TCR beta chain, a TCR delta chain, a TCR gamma chain, a TCR zeta chain, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, CD154, functional fragments thereof, and amino acid sequences thereof having at least one but not more than 20 modifications.

In some instances, the TFP, the TCR gamma constant domain, the TCR delta constant domain, the TCR alpha constant domain, the TCR beta constant domain, and any combination thereof is capable of functionally interacting with an endogenous TCR complex and/or at least one endogenous TCR polypeptide. In some instances, (a) the TCR constant domain is a TCR gamma constant domain and the TFP functionally integrates into a TCR complex comprising an endogenous subunit of TCR beta, CD3 epsilon, CD3 gamma, CD3 delta, or a combination thereof, (b) the TCR constant domain is a TCR delta constant domain and the TFP functionally integrates into a TCR complex comprising an endogenous subunit of TCR gamma, CD3 epsilon, CD3 gamma, CD3 delta, or a combination thereof, (c) the TCR constant domain is a TCR gamma constant domain and a TCR delta constant domain and the TFP functionally integrates into a TCR complex comprising an endogenous subunit of CD3 epsilon, CD3 gamma, CD3 delta, or a combination thereof, (d) the TCR constant domain is a TCR alpha constant domain and the TFP functionally integrates into a TCR complex comprising an endogenous subunit of TCR beta, CD3 epsilon, CD3 gamma, CD3 delta, or a combination thereof, or (e) the TCR constant domain is a TCR beta constant domain and the TFP functionally integrates into a TCR complex comprising an endogenous subunit of TCR alpha, CD3 epsilon, CD3 gamma, CD3 delta, or a combination thereof.

In some instances, the at least one but not more than 20 modifications thereto comprise a modification of an amino acid that mediates cell signaling or a modification of an amino acid that is phosphorylated in response to a ligand binding to the TFP.

In some instances, the murine, human or humanized antibody is an antibody fragment. In some instances, the antibody fragment is a scFv, a single domain antibody domain, a VH domain or a VL domain. In some instances, murine, human or humanized antibody comprising an antigen binding domain is selected from a group consisting of an anti-CD19 binding domain, anti-CD20 binding domain, anti-mesothelin binding domain, anti-PMSA binding domain, anti-CD70 binding domain, anti-CD79b binding domain, anti-MUC16 binding domain, anti-anti-B-cell maturation antigen (BCMA) binding domain, anti-mesothelin (MSLN) binding domain, anti-IL13Rα2 binding domain, anti-CD22 binding domain, anti-BAFF or anti-BAFFR binding domain, anti-PD-1 binding domain, anti-PD-L1 binding domain, and anti-ROR-1 binding domain.

In some embodiments, a sequence encoding the antigen binding domain or ligand binding domain is operatively linked to a sequence encoding a delta constant domain. In some embodiments, the intracellular domain is an intracellular domain of TCR gamma. In some embodiments, a sequence encoding the antigen binding domain or ligand binding domain is operatively linked to a sequence encoding a gamma constant domain. In some embodiments, the intracellular domain is an intracellular domain of TCR delta. In some embodiments, a sequence encoding the antigen binding domain or ligand binding domain is operatively linked to both a sequence encoding a TCR delta constant domain or fragment thereof and a TCR gamma constant domain or fragment thereof. In some embodiments, the intracellular signaling domain is CD3 epsilon, CD3 gamma, or CD3 delta. In some embodiments, the intracellular signaling domain is CD3 epsilon. In some embodiments, the recombinant nucleic acid further comprises at least one leader sequence and at least one linker. In some embodiments, the recombinant nucleic acid further comprises a portion of a TCR alpha constant domain, a portion of a TCR beta domain, or both. In some embodiments, the sequence comprises, from 5′ to 3′, a first leader sequence, an antigen binding domain sequence, a linker, a TRDC gene sequence, a cleavable linker sequence, a second leader sequence, and a TRGC gene sequence. In some embodiments, the sequence comprises, from 5′-3′, a first leader sequence, a TRDC gene sequence, a cleavable linker sequence, a second leader sequence, an antigen binding domain sequence, a linker sequence, and a TRGC gene sequence. In some embodiments, the sequence comprises, from 5′-3′, a first leader sequence, an antigen binding domain sequence, a first linker sequence, a TRDC gene sequence, a cleavable linker, a second leader sequence, a second antigen binding domain sequence, a second linker sequence, and a TRGC gene sequence. In some embodiments, the sequence comprises, from 5′-3′, a first leader sequence, a TRDC gene sequence, a first cleavable linker sequence, a second leader sequence, a TRGC gene sequence, a second cleavable linker sequence, a third leader sequence, an antigen binding domain sequence, a linker sequence, and a CD3 epsilon gene sequence. In some embodiments, the sequence comprises, from 5′-3′, a first leader sequence, a first antigen binding domain sequence, a first linker sequence, a TRDC gene sequence or fragment thereof, a TRAC gene sequence or fragment thereof, a cleavable linker sequence, a second leader sequence, a second antigen binding domain sequence, a second linker sequence, a TRGC gene sequence or fragment thereof, and a TRBC gene sequence or fragment thereof. In some embodiments, the binding ligand is capable of binding an Fc domain of the antibody. In some embodiments, the binding ligand is capable of selectively binding an IgG1 antibody. In some embodiments, the binding ligand is capable of specifically binding an IgG4 antibody. In some embodiments, the antibody or fragment thereof binds to a cell surface antigen. In some embodiments, the antibody or fragment thereof is murine, human or humanized. In some embodiments, the antibody or fragment thereof binds to a cell surface antigen on the surface of a tumor cell. In some embodiments, the binding ligand comprises a monomer, a dimer, a trimer, a tetramer, a pentamer, a hexamer, a heptamer, an octomer, a nonamer, or a decamer. In some embodiments, the binding ligand does not comprise an antibody or fragment thereof. In some embodiments, the binding ligand comprises a CD16 polypeptide or fragment thereof. In some embodiments, the binding ligand comprises a CD16-binding polypeptide. In some embodiments, the binding ligand is human or humanized. In some embodiments, the recombinant nucleic acid further comprises a nucleic acid sequence encoding an antibody or fragment thereof capable of being bound by the binding ligand. In some embodiments, the antibody or fragment thereof is capable of being secreted from a cell.

Recombinant Nucleic Acid Encoding IL-15 and/or IL-15Rα

Disclosed herein are recombinant nucleic acid molecules comprising a first nucleic acid sequence encoding a TFP described herein and a second nucleic acid sequence encoding an IL-15 polypeptide or a fragment thereof. Any recombinant nucleic acid molecules comprising a nucleic acid sequence encoding a TFP described herein may further comprise a second nucleic acid sequence encoding an IL-15 polypeptide or a fragment thereof. Further disclosed herein are recombinant nucleic acid molecules comprising a first nucleic acid sequence encoding a TFP described herein and a second nucleic acid sequence encoding an IL-15Rα polypeptide or a fragment thereof. Any recombinant nucleic acid molecules comprising a nucleic acid sequence encoding a TFP described herein may further comprise a second nucleic acid sequence encoding an IL-15Rα polypeptide or a fragment thereof.

Disclosed herein, in some embodiments, are recombinant nucleic acid molecules comprising a first nucleic acid sequence encoding a TFP described herein and a second nucleic acid sequence encoding an IL-15 polypeptide or a fragment thereof, wherein the first nucleic acid sequence and the second nucleic acid sequence are included in two separate nucleic acid molecules. Disclosed herein, in some embodiments, are recombinant nucleic acid molecules comprising a first nucleic acid sequence encoding a TFP described herein and a second nucleic acid sequence encoding an IL-15 polypeptide or a fragment thereof, wherein the first nucleic acid sequence and the second nucleic acid sequence are included in a single nucleic acid molecule. In some embodiments, the first nucleic acid sequence and the second nucleic acid sequence are operatively linked by a first linker. Further disclosed herein, in some embodiments, are recombinant nucleic acid molecules comprising a first nucleic acid sequence encoding a TFP described herein and a second nucleic acid sequence encoding an IL-15Rα polypeptide or a fragment thereof, wherein the first nucleic acid sequence and the second nucleic acid sequence are included in two separate nucleic acid molecules. Further disclosed herein, in some embodiments, are recombinant nucleic acid molecules comprising a first nucleic acid sequence encoding a TFP described herein and a second nucleic acid sequence encoding an IL-15Rα polypeptide or a fragment thereof, wherein the first nucleic acid sequence and the second nucleic acid sequence are included in a single nucleic acid molecule. In some embodiments, the first nucleic acid sequence and the second nucleic acid sequence are operatively linked by a first linker. For example, the first linker may be a cleavable linker. In some embodiments, the first linker may comprise a protease cleavage site. The cleavage site can be a self-cleaving peptide, for example, a 2A cleavage site such as a T2A, P2A, E2A or F2A cleavage site. In some embodiments, the protease cleavage site is a T2A cleavage site. The cleavage site can comprise a sequence of SEQ ID NO: 365, when expressed. In some embodiments, the first linker comprises a sequence of SEQ ID NO: 365, when expressed.

In some embodiments, the nucleic acid sequence encoding the IL-15 polypeptide, or a fragment thereof may comprise a sequence encoding IL-15 signal peptide. In some embodiments, IL-15 signal peptide comprises amino acids 1-29 of SEQ ID NO: 385, when expressed. In some embodiments, IL-15 signal peptide comprises a sequence of SEQ ID NO: 374, when expressed. In some embodiments, the nucleic acid sequence encoding the IL-15 polypeptide, or a fragment thereof may comprise a sequence encoding amino acids 30-162 of SEQ ID NO: 385. In some embodiments, the nucleic acid sequence encoding the IL-15 polypeptide, or a fragment thereof may comprise a sequence encoding a sequence of SEQ ID NO: 375. In some embodiments, the nucleic acid sequence encoding the IL-15 polypeptide, or a fragment thereof may comprise a sequence encoding amino acids 1-162 of SEQ ID NO: 385. In some embodiments, the nucleic acid sequence encoding the IL-15 polypeptide, or a fragment thereof may comprise a sequence encoding a sequence of SEQ ID NO: 374 and a sequence of SEQ ID NO: 375. In some embodiments, the IL-15 polypeptide or a fragment thereof is secreted when expressed in a T cell. In some embodiments, the IL-15 polypeptide comprises a sequence of SEQ ID NO: 375, when expressed.

Disclosed herein, in some embodiments, are recombinant nucleic acid molecules comprising a first nucleic acid sequence encoding a TFP described herein and a second nucleic acid sequence encoding an IL-15 polypeptide or a fragment thereof and an IL-15R subunit or a fragment thereof, wherein the first nucleic acid sequence and the second nucleic acid sequence are included in two separate nucleic acid molecules. Disclosed herein, in some embodiments, are recombinant nucleic acid molecules comprising a first nucleic acid sequence encoding a TFP described herein and a second nucleic acid sequence encoding an IL-15 polypeptide or a fragment thereof and an IL-15R subunit or a fragment thereof, wherein the first nucleic acid sequence and the second nucleic acid sequence are included in a single nucleic acid molecule. In some embodiments, the first nucleic acid sequence and the second nucleic acid sequence are operatively linked by a first linker described herein. An IL-15R subunit may be an IL-15R alpha (IL-15Rα), an IL-2R beta (IL-20), or an IL-2R gamma/the common gamma chain (IL-2Rγ/γc). In some embodiments, the IL-15R subunit is IL-15R alpha (IL-15Rα). In some embodiments, IL-15 and IL-15R subunit are operatively linked by a second linker. In some embodiments, IL-15 and IL-15Rα are operatively linked by a second linker. In some embodiments, the second linker is not a cleavable linker. For example, the second linker may comprise a sequence comprising (G₄S)ₙ, wherein G is glycine, S is serine, and n is an integer from 1 to 10. In some embodiments, n is an integer from 1 to 4. In some embodiments, n is 3. In some embodiments, the second linker comprises a sequence of SEQ ID NO: 378. In some embodiments, the second linker comprises a sequence of SEQ ID NO: 405.

In some embodiments, the nucleic acid sequence encoding the IL-15Rα polypeptide or a fragment thereof may comprise a sequence encoding the intracellular domain of IL-15Rα. In some embodiments, the nucleic acid sequence encoding the IL-15Rα polypeptide or a fragment thereof may comprise a sequence encoding amino acids 229-267 of IL-15Rα. In some embodiments, the nucleic acid sequence encoding the IL-15Rα polypeptide or a fragment thereof may comprise a sequence encoding amino acids 229-267 of SEQ ID NO: 386. In some embodiments, the nucleic acid sequence encoding the IL-15Rα polypeptide or a fragment thereof may comprise a sequence encoding a sequence of SEQ ID NO: 372.

In some embodiments, the nucleic acid sequence encoding the IL-15Rα polypeptide or a fragment thereof may comprise a sequence encoding IL-15Rα Sushi domain. In some embodiments, the nucleic acid sequence encoding the IL-15Rα polypeptide or a fragment thereof may comprise a sequence encoding amino acids 31-95 of IL-15Rα. In some embodiments, the nucleic acid sequence encoding the IL-15Rα polypeptide or a fragment thereof may comprise a sequence encoding amino acids 31-95 of SEQ ID NO: 386. In some embodiments, the nucleic acid sequence encoding the IL-15Rα polypeptide or a fragment thereof may comprise a sequence encoding a sequence of SEQ ID NO: 382.

In some embodiments, the nucleic acid sequence encoding the IL-15Rα polypeptide or a fragment thereof may comprise a sequence encoding the transmembrane domain and the intracellular domain of IL-15Rα. In some embodiments, the nucleic acid sequence encoding the IL-15Rα polypeptide or a fragment thereof may comprise a sequence encoding amino acids 96-267 of IL-15Rα. In some embodiments, the nucleic acid sequence encoding the IL-15Rα polypeptide or a fragment thereof may comprise a sequence encoding amino acids 96-267 of SEQ ID NO: 386. In some embodiments, the nucleic acid sequence encoding the IL-15Rα polypeptide or a fragment thereof may comprise a sequence encoding a sequence of SEQ ID NO: 383.

In some embodiments, the nucleic acid sequence encoding the IL-15Rα polypeptide or a fragment thereof may comprise a sequence encoding the Sushi domain, the transmembrane domain, and the intracellular domain of IL-15Rα. In some embodiments, the nucleic acid sequence encoding the IL-15Rα polypeptide or a fragment thereof may comprise a sequence encoding amino acids 31-267 of IL-15Rα. In some embodiments, the nucleic acid sequence encoding the IL-15Rα polypeptide or a fragment thereof may comprise a sequence encoding amino acids 31-267 of SEQ ID NO: 386. In some embodiments, the nucleic acid sequence encoding the IL-15Rα polypeptide or a fragment thereof may comprise a sequence encoding a sequence of SEQ ID NO: 382 and a sequence of SEQ ID NO: 383. In some embodiments, IL-15Rα comprises a sequence of SEQ ID NO: 403.

In some embodiments, the nucleic acid sequence encoding the IL-15Rα polypeptide or a fragment thereof may comprise a sequence encoding a soluble IL-15Rα (sIL-15Rα). In some embodiments, the nucleic acid sequence encoding the IL-15Rα polypeptide or a fragment thereof may comprise a sequence encoding amino acids 21-205 of IL-15Rα. In some embodiments, the nucleic acid sequence encoding the IL-15Rα polypeptide or a fragment thereof may comprise a sequence encoding amino acids 21-205 of SEQ ID NO: 386. In some embodiments, the nucleic acid sequence encoding the IL-15Rα polypeptide or a fragment thereof may comprise a sequence encoding a sequence of SEQ ID NO: 379.

Disclosed herein, in some embodiments, are recombinant nucleic acid molecules comprising a first nucleic acid sequence encoding a TFP described herein and a second nucleic acid sequence encoding a fusion protein comprising an IL-15 polypeptide linked to an IL-15Rα subunit, wherein the first nucleic acid sequence and the second nucleic acid sequence are included in two separate nucleic acid molecules. Disclosed herein, in some embodiments, are recombinant nucleic acid molecules comprising a first nucleic acid sequence encoding a TFP described herein and a second nucleic acid sequence encoding a fusion protein comprising an IL-15 polypeptide linked to an IL-15Rα subunit, wherein the first nucleic acid sequence and the second nucleic acid sequence are included in a single nucleic acid molecule. In some embodiments, the first nucleic acid sequence and the second nucleic acid sequence are operatively linked by a first linker described herein. For example, IL-15 polypeptide may be linked to N-terminus of IL-15Rα subunit. For example, IL-15 polypeptide may be linked to C-terminus of IL-15Rα subunit.

In some embodiments, the nucleic acid sequence encoding the fusion protein may comprise a sequence encoding amino acids 1-29 of IL-15. In some embodiments, the nucleic acid sequence encoding the fusion protein may comprise a sequence encoding amino acids 1-29 of SEQ ID NO: 385. In some embodiments, the nucleic acid sequence encoding the fusion protein may comprise a sequence encoding a sequence of SEQ ID NO: 374. In some embodiments, the nucleic acid sequence encoding the fusion protein may comprise a sequence encoding amino acids 30-162 of IL-15. In some embodiments, the nucleic acid sequence encoding the fusion protein may comprise a sequence encoding amino acids 30-162 of SEQ ID NO: 385. In some embodiments, the nucleic acid sequence encoding the fusion protein may comprise a sequence encoding a sequence of SEQ ID NO: 375. In some embodiments, the nucleic acid sequence encoding the fusion protein may comprise a sequence encoding amino acids 1-162 of IL-15. In some embodiments, the nucleic acid sequence encoding the fusion protein may comprise a sequence encoding amino acids 1-162 of SEQ ID NO: 385. In some embodiments, the nucleic acid sequence encoding the fusion protein may comprise a sequence encoding a sequence of SEQ ID NO: 374 and a sequence encoding a sequence of SEQ ID NO: 375.

In some embodiments, the nucleic acid sequence encoding the fusion protein may comprise a sequence encoding the intracellular domain of IL-15Rα. In some embodiments, the nucleic acid sequence encoding the fusion protein may comprise a sequence encoding amino acids 229-267 of IL-15Rα. In some embodiments, the nucleic acid sequence encoding the fusion protein may comprise a sequence encoding amino acids 229-267 of SEQ ID NO: 386. In some embodiments, the nucleic acid sequence encoding the fusion protein may comprise a sequence encoding a sequence of SEQ ID NO: 372.

In some embodiments, the nucleic acid sequence encoding the fusion protein may further comprise a sequence encoding IL-15Rα Sushi domain. In some embodiments, the nucleic acid sequence encoding the fusion protein may comprise a sequence encoding amino acids 31-95 of IL-15Rα. In some embodiments, the nucleic acid sequence encoding the fusion protein may comprise a sequence encoding amino acids 31-95 of SEQ ID NO: 386. In some embodiments, the nucleic acid sequence encoding the fusion protein may comprise a sequence encoding a sequence of SEQ ID NO: 382.

In some embodiments, the nucleic acid sequence encoding the fusion protein may comprise a sequence encoding the transmembrane domain and the intracellular domain of IL-15Rα. In some embodiments, the nucleic acid sequence encoding the fusion protein may comprise a sequence encoding amino acids 96-267 of IL-15Rα. In some embodiments, the nucleic acid sequence encoding the fusion protein may comprise a sequence encoding amino acids 96-267 of SEQ ID NO: 386. In some embodiments, the nucleic acid sequence encoding the fusion protein may comprise a sequence encoding a sequence of SEQ ID NO: 383.

In some embodiments, the nucleic acid sequence encoding the fusion protein may comprise a sequence encoding the Sushi domain, the transmembrane domain, and the intracellular domain of IL-15Rα. In some embodiments, the nucleic acid sequence encoding the fusion protein may comprise a sequence encoding amino acids 31-267 of IL-15Rα. In some embodiments, the nucleic acid sequence encoding the fusion protein may comprise a sequence encoding amino acids 31-267 of SEQ ID NO: 386. In some embodiments, the nucleic acid sequence encoding the fusion protein may comprise a sequence encoding a sequence of SEQ ID NO: 382 and a sequence of SEQ ID NO: 383. In some embodiments, IL-15Rα comprises a sequence of SEQ ID NO: 403.

In some embodiments, the nucleic acid sequence encoding the fusion protein may comprise a sequence encoding a soluble IL-15Rα (sIL-15Rα). In some embodiments, the nucleic acid sequence encoding the fusion protein may comprise a sequence encoding amino acids 21-205 of IL-15Rα. In some embodiments, the nucleic acid sequence encoding the fusion protein may comprise a sequence encoding amino acids 21-205 of SEQ ID NO: 386. In some embodiments, the nucleic acid sequence encoding the fusion protein may comprise a sequence encoding a sequence of SEQ ID NO: 379.

In some embodiments, the nucleic acid sequence encoding the fusion protein may further comprise a sequence encoding an epitope tag. An epitope tag as described herein can be a peptide epitope tag or a protein epitope tag. Examples of a peptide epitope tag includes, but are not limited to, 6×His (also known as His-tag or hexahistidine tag), FLAG (e.g., 3×FLAG), HA, Myc, and V5. Examples of a protein epitope tag include, but are not limited to, green fluorescent protein (GFP), glutathione-S-transferase (GST), β-galactosidase (β-GAL), Luciferase, Maltose Binding Protein (MBP), Red Fluorescence Protein (RFP), and Vesicular Stomatitis Virus Glycoprotein (VSV-G). In some embodiments, the nucleic acid sequence encoding the fusion protein further comprises a sequence encoding a FLAG tag. In some embodiments, the nucleic acid sequence encoding the fusion protein further comprises a sequence encoding a 3×FLAG tag. In some embodiments, the nucleic acid sequence encoding the fusion protein further comprises a sequence encoding a sequence of SEQ ID NO: 384.

In some embodiments, the fusion protein is expressed on cell surface when expressed from the recombinant nucleic acid molecule described herein in a T cell. In some embodiments, the fusion protein is secreted when expressed from the recombinant nucleic acid molecule described herein in a T cell.

Disclosed herein, in some embodiments, are recombinant nucleic acid molecules comprising a first nucleic acid sequence encoding a TFP described herein, a second nucleic acid sequence encoding an IL-15 polypeptide or a fragment thereof, and a third nucleic acid sequence encoding an agent that can enhance the activity of a modified T cell expressing the TFP. In some embodiments, the third nucleic acid sequence is included in a separate nucleic acid sequence. In some embodiments, the third nucleic acid sequence is included in the same nucleic acid molecule as the first nucleic acid sequence or the second nucleic acid sequence, or the first and the second nucleic acid sequences. For example, in one embodiment, the agent that can enhance the activity of a modified T cell can be a PD-1 polypeptide. In these embodiments, the PD-1 polypeptide may be operably linked to the N-terminus of an intracellular domain of a costimulatory polypeptide via the C-terminus of the PD-1 polypeptide. For example, in another embodiment, the agent that can enhance the activity of a modified T cell can be an anti-PD-1 antibody, or antigen binding fragment thereof. In this embodiment, the anti-PD-1 antibody or antigen binding fragment thereof may be operably linked to the N-terminus of an intracellular domain of a costimulatory polypeptide via the C-terminus of the anti-PD-1 antibody, or antigen binding fragment thereof. In some embodiments, the PD-1 polypeptide or anti-PD-1 antibody is linked to the intracellular domain of the costimulatory polypeptide via the transmembrane domain of PD-1. In some embodiments, the costimulatory polypeptide is selected from the group consisting of OX40, CD2, CD27, CDS, ICAM-1, ICOS (CD278), 4-1BB (CD137), GITR, CD28, CD30, CD40, BAFFR, HVEM, CD7, LIGHT, NKG2C, SLAMF7, NKp80, CD160, CD226, FcγRI, FcγRII, and FcγRIII.

Disclosed herein, in some embodiments, are recombinant nucleic acid molecules comprising a first nucleic acid sequence encoding a TFP described herein and a second nucleic acid sequence encoding an IL-15Rα polypeptide or a fragment thereof, wherein the first nucleic acid sequence and the second nucleic acid sequence are operatively linked by a first linker described herein, and wherein the second nucleic acid sequence further encodes an agent that can enhance the activity of a modified T cell expressing the TFP. For example, the agent can be an agent that can inhibit an inhibitory molecule that can decrease the ability of a T cell expressing a TFP to mount an immune effector response. In some embodiments, the agent which inhibits an inhibitory molecule comprises a first polypeptide, e.g., an inhibitory molecule, associated with a second polypeptide that provides a positive signal to the cell, e.g., an intracellular signaling domain described herein. In one embodiment, the agent may comprise a first polypeptide, e.g., of an inhibitory molecule such as PD-1, LAG3, CTLA4, CD160, BTLA, LAIR1, TIM3, 2B4, and TIGIT, or a fragment of any of these (e.g., at least a portion of an extracellular domain of any of these), and a second polypeptide which is an intracellular signaling domain described herein (e.g., comprising a costimulatory domain (e.g., 4-1BB, CD27, or CD28, as described herein)) and/or a primary signaling domain (e.g., IL-15Rα described herein). In some embodiments, the second nucleic acid sequence further comprises a sequence encoding PD-1 or a fragment thereof. In some embodiments, the second nucleic acid sequence comprises a sequence encoding the extracellular domain of PD-1. In some embodiments, the second nucleic acid sequence comprises a sequence encoding the extracellular domain and transmembrane domain of PD-1. In some embodiments, the second nucleic acid sequence may further comprise a sequence encoding CD28 or a fragment thereof. In some embodiments, the second nucleic acid sequence comprises a sequence encoding the intracellular domain of CD28. In some embodiments, the second nucleic acid sequence comprises a sequence encoding a fusion protein comprising the PD-1 extracellular domain and transmembrane domain linked to the CD28 intracellular domain linked to IL-15Rα. In some embodiments, the CD28 intracellular domain is linked to the intracellular domain of IL-15Rα. In some embodiments, the intracellular domain of IL-15Rα comprises amino acids 229-267 of IL-15Rα. In some embodiments, the intracellular domain of IL-15Rα comprises amino acids 229-267 of SEQ ID NO: 386. In some embodiments the intracellular domain of IL-15Rα comprises a sequence of SEQ ID NO: 372.

In some embodiments, the second nucleic acid sequence encoding PD-1, or a fragment thereof may comprise a nucleic acid sequence encoding a sequence of SEQ ID NO: 366. In some embodiments, the second nucleic acid sequence encoding PD-1, or a fragment thereof may comprise a nucleic acid sequence encoding a sequence of SEQ ID NO: 367. In some embodiments, the second nucleic acid sequence encoding PD-1, or a fragment thereof may comprise a nucleic acid sequence encoding a sequence of SEQ ID NO: 368. In some embodiments, the second nucleic acid sequence encoding PD-1, or a fragment thereof may comprise a nucleic acid sequence encoding a sequence of SEQ ID NO: 369. In some embodiments, the nucleic acid sequence encoding the transmembrane domain of PD-1 may comprise a nucleic acid sequence encoding a sequence of SEQ ID NO: 370. In some embodiments, the nucleic acid sequence encoding the intracellular domain of CD28 may comprise a nucleic acid sequence encoding a sequence of SEQ ID NO: 371. In some embodiments, the intracellular domain of IL-15Rα comprises amino acids 229-267 of IL-15Rα. In some embodiments, the nucleic acid encoding the intracellular domain of IL-15Rα comprises a nucleic acid encoding amino acids 229-267 of SEQ ID NO: 386. In some embodiments, the nucleic acid encoding the intracellular domain of IL-15Rα comprises a nucleic acid encoding a sequence of SEQ ID NO: 372.

Disclosed herein, in some embodiments, are recombinant nucleic acid molecules comprising a first nucleic acid sequence encoding a TFP described herein, a second nucleic acid sequence encoding an IL-15Rα polypeptide or a fragment thereof and an agent that can enhance the activity of a modified T cell expressing the TFP described herein, and a third nucleic acid sequence encoding an IL-15 polypeptide or a fragment thereof. In some embodiments, the first nucleic acid sequence and the second nucleic acid sequence are included in two separate nucleic acid sequences. In some embodiments, the first nucleic acid sequence and the second nucleic acid sequence are included in a single nucleic acid sequence. In some embodiments, the first nucleic acid sequence and the second nucleic acid sequence are operatively linked by a first linker described herein. In some embodiments, the third nucleic acid sequence is included in a separate nucleic acid sequence. In some embodiments, the third nucleic acid sequence is included in the same nucleic acid molecule as the first nucleic acid sequence or the second nucleic acid sequence, or the first and the second nucleic acid sequences. In some embodiments, the third nucleic acid sequence encoding the IL-15 polypeptide may comprise a sequence encoding amino acids 1-29 of IL-15. In some embodiments, the third nucleic acid sequence encoding the IL-15 polypeptide may comprise a sequence encoding amino acids 1-29 of SEQ ID NO: 385. In some embodiments, the third nucleic acid sequence encoding the IL-15 polypeptide may comprise a sequence encoding a sequence of SEQ ID NO: 374. In some embodiments, the third nucleic acid sequence encoding the IL-15 polypeptide may comprise a sequence encoding amino acids 30-162 of IL-15. In some embodiments, the third nucleic acid sequence encoding the IL-15 polypeptide may comprise a sequence encoding amino acids 30-162 of SEQ ID NO: 385. In some embodiments, the third nucleic acid sequence encoding the IL-15 polypeptide may comprise a sequence encoding a sequence of SEQ ID NO: 375. In some embodiments, the IL-15 polypeptide is secreted when expressed in a T cell. In some embodiments, the third nucleic acid sequence encoding the IL-15 polypeptide may comprise a sequence encoding amino acids 1-162 of IL-15. In some embodiments, the third nucleic acid sequence encoding the IL-15 polypeptide may comprise a sequence encoding amino acids 1-162 of SEQ ID NO: 385. In some embodiments, the third nucleic acid sequence encoding the IL-15 polypeptide may comprise a sequence encoding a sequence of SEQ ID NO: 374 and a sequence of SEQ ID NO: 375.

Disclosed herein in some embodiments, are recombinant nucleic acid molecules comprising a nucleic acid sequence encoding a TFP described herein, a nucleic acid sequence encoding a PD-1 polypeptide or a fragment thereof, a nucleic acid sequence encoding CD28 polypeptide or a fragment thereof, a nucleic acid sequence encoding an IL-15Rα or a fragment thereof described herein, and a nucleic acid sequence encoding an IL-15 polypeptide, or a fragment thereof described herein. In some embodiments, the nucleic acid sequence encoding a TFP may comprise a sequence encoding CSF2RA signal peptide. In some embodiments, the nucleic acid sequence encoding a TFP may comprise a nucleic acid sequence encoding a sequence of SEQ ID NO: 362. In some embodiments, the nucleic acid sequence encoding a TFP may comprise a sequence encoding anti-MSLN antibody. In some embodiments, the nucleic acid sequence encoding a TFP may comprise a nucleic acid sequence encoding a sequence of SEQ ID NO: 363. In some embodiments, the nucleic acid sequence encoding a TFP may comprise a sequence encoding CD3a. In some embodiments, the nucleic acid sequence encoding a TFP may comprise a nucleic acid sequence encoding a sequence of SEQ ID NO: 364. In some embodiments, the nucleic acid sequence encoding the PD-1 polypeptide, or a fragment thereof comprises a sequence encoding PD-1 signal peptide. In some embodiments, the nucleic acid sequence encoding the PD-1 polypeptide, or a fragment thereof comprises a nucleic acid sequence encoding a sequence of SEQ ID NO: 366. In some embodiments, the nucleic acid sequence encoding the PD-1 polypeptide, or a fragment thereof comprises a sequence encoding PD-1 N-Loop. In some embodiments, the nucleic acid sequence encoding the PD-1 polypeptide, or a fragment thereof comprises a nucleic acid sequence encoding a sequence of SEQ ID NO: 367. In some embodiments, the nucleic acid sequence encoding the PD-1 polypeptide, or a fragment thereof comprises a sequence encoding PD-1 IgV. In some embodiments, the nucleic acid sequence encoding the PD-1 polypeptide, or a fragment thereof comprises a nucleic acid sequence encoding a sequence of SEQ ID NO: 368. In some embodiments, the nucleic acid sequence encoding the PD-1 polypeptide, or a fragment thereof comprises a sequence encoding PD-1 Stalk. In some embodiments, the nucleic acid sequence encoding the PD-1 polypeptide, or a fragment thereof comprises a nucleic acid sequence encoding a sequence of SEQ ID NO: 369. In some embodiments, the nucleic acid sequence encoding the PD-1 polypeptide, or a fragment thereof comprises a sequence encoding PD-1 transmembrane domain. In some embodiments, the nucleic acid sequence encoding the PD-1 polypeptide, or a fragment thereof comprises a nucleic acid sequence encoding a sequence of SEQ ID NO: 370. In some embodiments, the nucleic acid sequence encoding the CD28 polypeptide or a fragment thereof comprises a sequence encoding CD28 intracellular domain. In some embodiments, the nucleic acid sequence encoding the PD-1 polypeptide, or a fragment thereof comprises a nucleic acid sequence encoding a sequence of SEQ ID NO: 371. In some embodiments, the nucleic acid sequence encoding IL-15Rα polypeptide or fragment thereof comprise a sequence encoding amino acids 229-267 of IL-15Rα. In some embodiments, the nucleic acid sequence encoding IL-15Rα polypeptide or fragment thereof may comprise a nucleic acid sequence encoding a sequence of SEQ ID NO: 372. In some embodiments, the nucleic acid sequence encoding IL-15 polypeptide or fragment thereof comprise a sequence encoding amino acids 1-29 of IL-15. In some embodiments, the nucleic acid sequence encoding IL-15 polypeptide or fragment thereof comprise a nucleic acid sequence encoding a sequence of SEQ ID NO: 374. In some embodiments, the nucleic acid sequence encoding IL-15 polypeptide or fragment thereof may comprise a sequence encoding amino acids 30-162 of IL-15. In some embodiments, the nucleic acid sequence encoding IL-15 polypeptide or fragment thereof may comprise a nucleic acid sequence encoding a sequence of SEQ ID NO: 375. In some embodiments, the nucleic acid sequence encoding the TFP and the nucleic acid sequence encoding the PD-1 polypeptide, or a fragment thereof are operatively linked by a T2A linker. In some embodiments, the T2A linker may comprise a sequence of SEQ ID NO: 365. In some embodiments, the nucleic acid sequence encoding the IL-15Rα or a fragment thereof and the nucleic acid sequence encoding the IL-15 polypeptide, or a fragment thereof are operatively linked by a P2A linker. In some embodiments, the P2A linker may comprise a sequence of SEQ ID NO: 373. In some embodiments, the recombinant nucleic acid molecule comprising a nucleic acid sequence encoding a TFP described herein, a nucleic acid sequence encoding a PD-1 polypeptide or a fragment thereof, a nucleic acid sequence encoding CD28 polypeptide or a fragment thereof, a nucleic acid sequence encoding an IL-15Rα, and a nucleic acid sequence encoding an IL-15 polypeptide, or a fragment thereof may comprise a nucleic acid sequence encoding a sequence of SEQ ID NO: 361.

Disclosed herein in some embodiments, are recombinant nucleic acid molecules comprising a nucleic acid sequence encoding a TFP described herein, a nucleic acid sequence encoding a PD-1 polypeptide or a fragment thereof, a nucleic acid sequence encoding CD28 polypeptide or a fragment thereof, and a nucleic acid sequence encoding an IL-15Rα or a fragment thereof described herein. In some embodiments, the nucleic acid sequence encoding a TFP may comprise a sequence encoding CSF2RA signal peptide. In some embodiments, the nucleic acid sequence encoding a TFP may comprise a nucleic acid sequence encoding a sequence of SEQ ID NO: 362. In some embodiments, the nucleic acid sequence encoding a TFP may comprise a sequence encoding anti-MSLN antibody. In some embodiments, the nucleic acid sequence encoding a TFP may comprise a nucleic acid sequence encoding a sequence of SEQ ID NO: 363. In some embodiments, the nucleic acid sequence encoding a TFP may comprise a sequence encoding CD3ε. In some embodiments, the nucleic acid sequence encoding a TFP may comprise a nucleic acid sequence encoding a sequence of SEQ ID NO: 364. In some embodiments, the nucleic acid sequence encoding the PD-1 polypeptide, or a fragment thereof comprises a sequence encoding PD-1 signal peptide. In some embodiments, the nucleic acid sequence encoding the PD-1 polypeptide, or a fragment thereof comprises a nucleic acid sequence encoding a sequence of SEQ ID NO: 366. In some embodiments, the nucleic acid sequence encoding the PD-1 polypeptide, or a fragment thereof comprises a sequence encoding PD-1 N-Loop. In some embodiments, the nucleic acid sequence encoding the PD-1 polypeptide, or a fragment thereof comprises a nucleic acid sequence encoding a sequence of SEQ ID NO: 367. In some embodiments, the nucleic acid sequence encoding the PD-1 polypeptide, or a fragment thereof comprises a sequence encoding PD-1 IgV. In some embodiments, the nucleic acid sequence encoding the PD-1 polypeptide, or a fragment thereof comprises a nucleic acid sequence encoding a sequence of SEQ ID NO: 368. In some embodiments, the nucleic acid sequence encoding the PD-1 polypeptide, or a fragment thereof comprises a sequence encoding PD-1 Stalk. In some embodiments, the nucleic acid sequence encoding the PD-1 polypeptide, or a fragment thereof comprises a nucleic acid sequence encoding a sequence of SEQ ID NO: 369. In some embodiments, the nucleic acid sequence encoding the PD-1 polypeptide, or a fragment thereof comprises a sequence encoding PD-1 transmembrane domain. In some embodiments, the nucleic acid sequence encoding the PD-1 polypeptide, or a fragment thereof comprises a nucleic acid sequence encoding a sequence of SEQ ID NO: 370. In some embodiments, the nucleic acid sequence encoding the CD28 polypeptide or a fragment thereof comprises a sequence encoding CD28 intracellular domain. In some embodiments, the nucleic acid sequence encoding the PD-1 polypeptide, or a fragment thereof comprises a nucleic acid sequence encoding a sequence of SEQ ID NO: 371. In some embodiments, the nucleic acid sequence encoding IL-15Rα polypeptide or fragment thereof comprise a sequence encoding amino acids 229-267 of IL-15Rα. In some embodiments, the nucleic acid sequence encoding IL-15Rα polypeptide or fragment thereof may comprise a nucleic acid sequence encoding a sequence of SEQ ID NO: 372. In some embodiments, the nucleic acid sequence encoding the TFP and the nucleic acid sequence encoding the PD-1 polypeptide, or a fragment thereof are operatively linked by a T2A linker. In some embodiments, the T2A linker may comprise a sequence of SEQ ID NO: 365. In some embodiments, the recombinant nucleic acid molecule comprising a nucleic acid sequence encoding a TFP described herein, a nucleic acid sequence encoding a PD-1 polypeptide or a fragment thereof, a nucleic acid sequence encoding CD28 polypeptide or a fragment thereof, and a nucleic acid sequence encoding an IL-15Rα may comprise a nucleic acid sequence encoding a sequence of SEQ ID NO: 376.

Disclosed herein in some embodiments, are recombinant nucleic acid molecules comprising a nucleic acid sequence encoding a TFP described herein, a nucleic acid sequence encoding an IL-15 polypeptide, or a fragment thereof described herein, and a nucleic acid sequence encoding an IL-15Rα or a fragment thereof described herein. In some embodiments, the nucleic acid sequence encoding a TFP may comprise a sequence encoding CSF2RA signal peptide. In some embodiments, the nucleic acid sequence encoding a TFP may comprise a nucleic acid sequence encoding a sequence of SEQ ID NO: 362. In some embodiments, the nucleic acid sequence encoding a TFP may comprise a sequence encoding anti-MSLN antibody. In some embodiments, the nucleic acid sequence encoding a TFP may comprise a nucleic acid sequence encoding a sequence of SEQ ID NO: 363. In some embodiments, the nucleic acid sequence encoding a TFP may comprise a sequence encoding CD3ε. In some embodiments, the nucleic acid sequence encoding a TFP may comprise a nucleic acid sequence encoding a sequence of SEQ ID NO: 364. In some embodiments, the nucleic acid sequence encoding IL-15 polypeptide or fragment thereof comprise a sequence encoding amino acids 1-29 of IL-15. In some embodiments, the nucleic acid sequence encoding IL-15 polypeptide or fragment thereof comprise a nucleic acid sequence encoding a sequence of SEQ ID NO: 374. In some embodiments, the nucleic acid sequence encoding IL-15 polypeptide or fragment thereof may comprise a sequence encoding amino acids 30-162 of IL-15. In some embodiments, the nucleic acid sequence encoding IL-15 polypeptide or fragment thereof may comprise a nucleic acid sequence encoding a sequence of SEQ ID NO: 375. In some embodiments, the nucleic acid sequence encoding IL-15Rα polypeptide or fragment thereof comprise a sequence encoding amino acids 21-205 of IL-15Rα. In some embodiments, the nucleic acid sequence encoding IL-15Rα polypeptide or fragment thereof may comprise a nucleic acid sequence encoding a sequence of SEQ ID NO: 379. In some embodiments, the nucleic acid sequence encoding the TFP and the nucleic acid sequence encoding the IL-15 polypeptide, or a fragment thereof are operatively linked by a T2A linker. In some embodiments, the T2A linker may comprise a sequence of SEQ ID NO: 365. In some embodiments, the nucleic acid sequence encoding the IL-15 polypeptide or a fragment thereof and the nucleic acid sequence encoding the IL-15Rα or a fragment thereof are operatively linked by a non-cleavable linker. In some embodiments, the non-cleavable linker may comprise a sequence of SEQ ID NO: 378. In some embodiments, the non-cleavable linker comprises a sequence of SEQ ID NO: 405. In some embodiments, the recombinant nucleic acid molecule comprising a nucleic acid sequence encoding a TFP described herein, a nucleic acid sequence encoding an IL-15 polypeptide or a fragment thereof, and a nucleic acid sequence encoding an IL-15Rα may comprise a nucleic acid sequence encoding a sequence of SEQ ID NO: 377.

Disclosed herein in some embodiments, are recombinant nucleic acid molecules comprising a nucleic acid sequence encoding a TFP described herein and a nucleic acid sequence encoding an IL-15 polypeptide, or a fragment thereof described herein. In some embodiments, the nucleic acid sequence encoding a TFP may comprise a sequence encoding CSF2RA signal peptide. In some embodiments, the nucleic acid sequence encoding a TFP may comprise a nucleic acid sequence encoding a sequence of SEQ ID NO: 362. In some embodiments, the nucleic acid sequence encoding a TFP may comprise a sequence encoding anti-MSLN antibody. In some embodiments, the nucleic acid sequence encoding a TFP may comprise a nucleic acid sequence encoding a sequence of SEQ ID NO: 363. In some embodiments, the nucleic acid sequence encoding a TFP may comprise a sequence encoding CD3ε. In some embodiments, the nucleic acid sequence encoding a TFP may comprise a nucleic acid sequence encoding a sequence of SEQ ID NO: 364. In some embodiments, the nucleic acid sequence encoding IL-15 polypeptide or fragment thereof comprise a sequence encoding amino acids 1-29 of IL-15. In some embodiments, the nucleic acid sequence encoding IL-15 polypeptide or fragment thereof comprise a nucleic acid sequence encoding a sequence of SEQ ID NO: 374. In some embodiments, the nucleic acid sequence encoding IL-15 polypeptide or fragment thereof may comprise a sequence encoding amino acids 30-162 of IL-15. In some embodiments, the nucleic acid sequence encoding IL-15 polypeptide or fragment thereof may comprise a nucleic acid sequence encoding a sequence of SEQ ID NO: 375. In some embodiments, the nucleic acid sequence encoding the TFP and the nucleic acid sequence encoding the IL-15 polypeptide, or a fragment thereof are operatively linked by a T2A linker. In some embodiments, the T2A linker may comprise a sequence of SEQ ID NO: 365. In some embodiments, the recombinant nucleic acid molecule comprising a nucleic acid sequence encoding a TFP described herein and a nucleic acid sequence encoding an IL-15 polypeptide, or a fragment thereof may comprise a nucleic acid sequence encoding a sequence of SEQ ID NO: 380.

Disclosed herein in some embodiments, are recombinant nucleic acid molecules comprising a nucleic acid sequence encoding a TFP described herein, a nucleic acid sequence encoding an IL-15 polypeptide, or a fragment thereof described herein, and a nucleic acid sequence encoding an IL-15Rα or a fragment thereof described herein. In some embodiments, the nucleic acid sequence encoding a TFP may comprise a sequence encoding CSF2RA signal peptide. In some embodiments, the nucleic acid sequence encoding a TFP may comprise a nucleic acid sequence encoding a sequence of SEQ ID NO: 362. In some embodiments, the nucleic acid sequence encoding a TFP may comprise a sequence encoding anti-MSLN antibody. In some embodiments, the nucleic acid sequence encoding a TFP may comprise a nucleic acid sequence encoding a sequence of SEQ ID NO: 363. In some embodiments, the nucleic acid sequence encoding a TFP may comprise a sequence encoding CD3ε. In some embodiments, the nucleic acid sequence encoding a TFP may comprise a nucleic acid sequence encoding a sequence of SEQ ID NO: 364. In some embodiments, the nucleic acid sequence encoding IL-15 polypeptide or fragment thereof comprise a sequence encoding amino acids 1-29 of IL-15. In some embodiments, the nucleic acid sequence encoding IL-15 polypeptide or fragment thereof comprise a nucleic acid sequence encoding a sequence of SEQ ID NO: 374. In some embodiments, the nucleic acid sequence encoding IL-15 polypeptide or fragment thereof may comprise a sequence encoding amino acids 30-162 of IL-15. In some embodiments, the nucleic acid sequence encoding IL-15 polypeptide or fragment thereof may comprise a nucleic acid sequence encoding a sequence of SEQ ID NO: 375. In some embodiments, the nucleic acid sequence encoding IL-15Rα polypeptide or fragment thereof comprise a sequence encoding amino acids 31-95 of IL-15Rα. In some embodiments, the nucleic acid sequence encoding IL-15Rα polypeptide or fragment thereof may comprise a nucleic acid sequence encoding a sequence of SEQ ID NO: 382. In some embodiments, the nucleic acid sequence encoding IL-15Rα polypeptide or fragment thereof comprise a sequence encoding amino acids 96-267 of IL-15Rα. In some embodiments, the nucleic acid sequence encoding IL-15Rα polypeptide or fragment thereof may comprise a nucleic acid sequence encoding a sequence of SEQ ID NO: 383. In some embodiments, the nucleic acid sequence encoding IL-15Rα polypeptide or fragment thereof may comprise a nucleic acid sequence encoding SEQ ID NO: 403. In some embodiments, the nucleic acid sequence encoding the TFP and the nucleic acid sequence encoding the IL-15 polypeptide, or a fragment thereof are operatively linked by a T2A linker. In some embodiments, the T2A linker may comprise a sequence of SEQ ID NO: 365. In some embodiments, the nucleic acid sequence encoding the IL-15 polypeptide or a fragment thereof and the nucleic acid sequence encoding the IL-15Rα or a fragment thereof are operatively linked by a non-cleavable linker. In some embodiments, the non-cleavable linker may comprise a sequence of SEQ ID NO: 378. In some embodiments, the non-cleavable linker may comprise a sequence of SEQ ID NO: 405. In some embodiments, the recombinant nucleic acid molecule may further comprise a sequence encoding a 3×FLAG tag. In some embodiments, the 3×FLAG comprises a sequence of SEQ ID NO: 384. In some embodiments, the recombinant nucleic acid molecule comprising a nucleic acid sequence encoding a TFP described herein, a nucleic acid sequence encoding an IL-15 polypeptide or a fragment thereof, and a nucleic acid sequence encoding an IL-15Rα may comprise a nucleic acid sequence encoding a sequence of SEQ ID NO: 381.

Disclosed herein in some embodiments, are recombinant nucleic acid molecules comprising a nucleic acid sequence encoding a TFP described herein and a nucleic acid sequence encoding an anti-CD70 antibody or a fragment thereof (CD70 CD3ε). In some embodiments, the nucleic acid sequence encoding a TFP may comprise a sequence encoding CSF2RA signal peptide. In some embodiments, the nucleic acid sequence encoding a TFP may comprise a nucleic acid sequence encoding a sequence of SEQ ID NO: 362. In some embodiments, the nucleic acid sequence encoding a TFP may comprise a sequence encoding an anti-CD70 antibody or a fragment thereof. In some embodiments, the nucleic acid sequence encoding a TFP may comprise a nucleic acid sequence encoding a sequence of SEQ ID NO: 399. In some embodiments, the nucleic acid sequence encoding a TFP may comprise a nucleic acid sequence encoding a sequence of SEQ ID NO: 395. In some embodiments, a linker sequence may be used in the antibody sequence region. In some embodiments, the linker sequence may comprise a sequence encoding SEQ ID NO: 401. In some embodiments, the nucleic acid sequence encoding a TFP may comprise a sequence encoding CD3ε. In some embodiments, the nucleic acid sequence encoding a TFP may comprise a nucleic acid sequence encoding a sequence of SEQ ID NO: 364. In some embodiments, the nucleic acid sequence encoding a TFP may comprise a nucleic acid sequence encoding a sequence of SEQ ID NO: 364. In some embodiments, the nucleic acid sequence encoding the anti-CD70 antibody or a fragment thereof and the nucleic acid sequence encoding the CD3ε or a fragment thereof are operatively linked by a linker. In some embodiments, the linker may comprise a sequence of SEQ ID NO: 387. In some embodiments, the recombinant nucleic acid molecule comprising a nucleic acid sequence encoding a CD70 CD3ε TFP as described herein may comprise a nucleic acid sequence encoding a sequence of SEQ ID NO: 400.

Disclosed herein in some embodiments, are recombinant nucleic acid molecules comprising a nucleic acid sequence encoding a TFP described herein, a nucleic acid sequence encoding an anti-CD70 antibody or a fragment thereof, a nucleic acid sequence encoding an IL-15 polypeptide, or a fragment thereof, and a nucleic acid sequence encoding an IL-15Rα or a fragment thereof (CD70+mbIL-15Rα). In some embodiments, the nucleic acid sequence encoding a TFP may comprise a sequence encoding CSF2RA signal peptide. In some embodiments, the nucleic acid sequence encoding a TFP may comprise a nucleic acid sequence encoding a sequence of SEQ ID NO: 362. In some embodiments, the nucleic acid sequence encoding a TFP may comprise a sequence encoding an anti-CD70 antibody or a fragment thereof. In some embodiments, the nucleic acid sequence encoding a TFP may comprise a nucleic acid sequence encoding a sequence of SEQ ID NO: 399. In some embodiments, the nucleic acid sequence encoding a TFP may comprise a nucleic acid sequence encoding a sequence of SEQ ID NO: 395. In some embodiments, a linker sequence may be used in the antibody sequence region. In some embodiments, the linker sequence may comprise a sequence encoding SEQ ID NO: 401. In some embodiments, the nucleic acid sequence encoding a TFP may comprise a sequence encoding CD3ε. In some embodiments, the nucleic acid sequence encoding a TFP may comprise a nucleic acid sequence encoding a sequence of SEQ ID NO: 364. In some embodiments, the nucleic acid sequence encoding a TFP may comprise a nucleic acid sequence encoding a sequence of SEQ ID NO: 364. In some embodiments, the nucleic acid sequence encoding the anti-CD70 antibody or a fragment thereof and the nucleic acid sequence encoding the CD3ε or a fragment thereof are operatively linked by a linker. In some embodiments, the linker may comprise a sequence of SEQ ID NO: 387. In some embodiments, the recombinant nucleic acid molecule may comprise a nucleic acid sequence encoding an IL-15 polypeptide or fragment thereof. In some embodiments, the nucleic acid sequence encoding IL-15 polypeptide or fragment thereof comprises a nucleic acid sequence encoding a sequence of SEQ ID NO: 385. In some embodiments, the recombinant nucleic acid molecule may comprise a nucleic acid sequence encoding IL-15Rα polypeptide or fragment thereof. In some embodiments, the nucleic acid sequence encoding IL-15Rα polypeptide or fragment thereof may comprise a nucleic acid sequence encoding a sequence of SEQ ID NO: 403. In some embodiments, the nucleic acid sequence encoding the TFP and the nucleic acid sequence encoding the IL-15 polypeptide, or a fragment thereof are operatively linked by a T2A linker. In some embodiments, the T2A linker may comprise a sequence of SEQ ID NO: 365. In some embodiments, the nucleic acid sequence encoding the IL-15 polypeptide or a fragment thereof and the nucleic acid sequence encoding the IL-15Rα or a fragment thereof are operatively linked by a non-cleavable linker. In some embodiments, the non-cleavable linker may comprise a sequence of SEQ ID NO: 378. In some embodiments, the non-cleavable linker comprises a sequence of SEQ ID NO: 405. In some embodiments, the recombinant nucleic acid molecule comprising a nucleic acid sequence encoding a TFP described herein, a nucleic acid sequence encoding an IL-15 polypeptide or a fragment thereof, and a nucleic acid sequence encoding an IL-15Rα may comprise a nucleic acid sequence encoding a sequence of SEQ ID NO: 402. In some embodiments, the recombinant nucleic acid molecule may comprise a sequence encoding SEQ ID NO: 404.

In some embodiments, recombinant nucleic acid molecules described herein further comprise a leader sequence. In some embodiments, the recombinant nucleic acid molecule is selected from the group consisting of a DNA and an RNA. In some embodiments, the recombinant nucleic acid molecule is an mRNA. In some embodiments, the recombinant nucleic acid molecule is a circRNA. In some embodiments, the recombinant nucleic acid molecule comprises a nucleic acid analog. In some embodiments, the nucleic acid analog is not in an encoding sequence of the recombinant nucleic acid. In some embodiments, the nucleic analog is selected from the group consisting of 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), 2′-O—N-methylacetamido (2′-O-NMA) modified, a locked nucleic acid (LNA), an ethylene nucleic acid (ENA), a peptide nucleic acid (PNA), a 1′,5′-anhydrohexitol nucleic acid (HNA), a morpholino, a methylphosphonate nucleotide, a thiolphosphonate nucleotide, and a 2′-fluoro N3-P5′-phosphoramidite. In some embodiments, the recombinant nucleic acid molecule further comprises a leader sequence. In some embodiments, the recombinant nucleic acid molecule further comprises a promoter sequence. In some embodiments, the recombinant nucleic acid molecule further comprises a sequence encoding a poly(A) tail. In some embodiments, the recombinant nucleic acid molecule further comprises a 3′UTR sequence. In some embodiments, the recombinant nucleic acid molecule is an isolated nucleic acid or a non-naturally occurring nucleic acid. In some embodiments, the nucleic acid is an in vitro transcribed nucleic acid.

### Vectors

Further disclosed herein, in some embodiments, are vectors comprising the recombinant nucleic acid molecules disclosed herein. In some instances, the vector is selected from the group consisting of a DNA, a RNA, a plasmid, a lentivirus vector, adenoviral vector, an adeno-associated viral vector (AAV), a Rous sarcoma viral (RSV) vector, or a retrovirus vector. In some instances, the vector is an AAV6 vector. In some instances, the vector further comprises a promoter. In some instances, the vector is an in vitro transcribed vector.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The present disclosure also provides vectors in which a DNA of the present disclosure is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

In another embodiment, the vector comprising the nucleic acid encoding the desired TFP, IL-15 polypeptide, and/or IL-15Rα polypeptide of the present disclosure is an adenoviral vector (A5/35). In another embodiment, the expression of nucleic acids encoding TFPs, IL-15 polypeptide, and/or IL-15Rα polypeptide can be accomplished using of transposons such as sleeping beauty, crisper, CAS9, and zinc finger nucleases. See below June et al. 2009 Nature Reviews Immunology 9.10: 704-716, is incorporated herein by reference.

The expression constructs of the present disclosure may also be used for nucleic acid immunization and gene therapy, using standard gene delivery protocols. Methods for gene delivery are known in the art (see, e.g., U.S. Pat. Nos. 5,399,346, 5,580,859, 5,589,466, incorporated by reference herein in their entireties). In another embodiment, the present disclosure provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of virally based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

An example of a promoter that is capable of expressing a TFP transgene, IL-15 transgene, and/or IL-15Rα transgene in a mammalian T cell is the EF1a promoter. The native EF1a promoter drives expression of the alpha subunit of the elongation factor-1 complex, which is responsible for the enzymatic delivery of aminoacyl tRNAs to the ribosome. The EF1a promoter has been extensively used in mammalian expression plasmids and has been shown to be effective in driving TFP, IL-15, and/or IL-15Rα expression from transgenes cloned into a lentiviral vector (see, e.g., Milone et al., Mol. Ther. 17(8): 1453-1464 (2009)). Another example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter. Further, the present disclosure should not be limited to the use of constitutive promoters. Inducible promoters are also contemplated as part of the present disclosure. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline-regulated promoter.

In order to assess the expression of a TFP polypeptide, IL-15 polypeptide, and/or IL-15Rα polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, Molecular Cloning: A Laboratory Manual, volumes 1-4, Cold Spring Harbor Press, NY). A preferred method for the introduction of a polynucleotide into a host cell is calcium phosphate transfection

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like (see, e.g., U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present disclosure, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and western blots) or by assays described herein to identify agents falling within the scope of the present disclosure.

The present disclosure further provides a vector comprising a nucleic acid molecule encoding a TFP described herein, an IL-15 polypeptide or a fragment described herein, and/or IL-15Rα polypeptide or a fragment described herein. In one aspect, a vector encoding a TFP described herein, an IL-15 polypeptide or a fragment described herein, and/or IL-15Rα polypeptide or a fragment described herein can be directly transduced into a cell, e.g., a T cell. In one aspect, the vector is a cloning or expression vector, e.g., a vector including, but not limited to, one or more plasmids (e.g., expression plasmids, cloning vectors, minicircles, minivectors, double minute chromosomes), retroviral and lentiviral vector constructs. In one aspect, the vector is capable of expressing the TFP construct, an IL-15 construct, and/or an IL-15Rα construct in mammalian T cells. In one aspect, the mammalian T cell is a human T cell.

### Recombinant RNAs

Disclosed herein are methods for producing in vitro transcribed RNA encoding TFPs, IL-15 or IL-15Rα described herein. The present disclosure also includes a TFP encoding RNA construct, a IL-15 encoding RNA construct, and/or IL-15Rα encoding RNA construct that can be directly transfected into a cell. A method for generating mRNA for use in transfection can involve in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the nucleic acid to be expressed, and a polyA tail, typically 50-2000 bases in length. RNA so produced can efficiently transfect different kinds of cells. In one aspect, the template includes sequences for the TFP, IL-15 polypeptide or a fragment thereof, and/or IL-15Rα polypeptide or a fragment thereof described herein.

In one aspect the anti-TAA TFP, IL-15 polypeptide or a fragment thereof, and/or IL-15Rα polypeptide or a fragment thereof described herein is encoded by a messenger RNA (mRNA). In one aspect the mRNA encoding the anti-TAA TFP, IL-15 polypeptide or a fragment thereof, or IL-15Rα polypeptide and/or a fragment thereof described herein is introduced into a T cell for production of a T cell expressing the TFP, IL-15 polypeptide or a fragment thereof, and/or IL-15Rα polypeptide or a fragment thereof described herein. In one embodiment, the in vitro transcribed RNA encoding a TFP, IL-15 polypeptide or a fragment thereof, or IL-15Rα polypeptide or a fragment thereof described herein can be introduced to a cell as a form of transient transfection. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired template for in vitro transcription is a TFP of the present disclosure. In one embodiment, the DNA to be used for PCR contains an open reading frame. The DNA can be from a naturally occurring DNA sequence from the genome of an organism. In one embodiment, the nucleic acid can include some or all of the 5′ and/or 3′ untranslated regions (UTRs). The nucleic acid can include exons and introns. In one embodiment, the DNA to be used for PCR is a human nucleic acid sequence. In another embodiment, the DNA to be used for PCR is a human nucleic acid sequence including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

PCR is used to generate a template for in vitro transcription of mRNA which is used for transfection. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a nucleic acid that is normally transcribed in cells (the open reading frame), including 5′ and 3′ UTRs. The primers can also be designed to amplify a portion of a nucleic acid that encodes a particular domain of interest. In one embodiment, the primers are designed to amplify the coding region of a human cDNA, including all or portions of the 5′ and 3′ UTRs. Primers useful for PCR can be generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Any DNA polymerase useful for PCR can be used in the methods disclosed herein. The reagents and polymerase are commercially available from a number of sources.

Chemical structures with the ability to promote stability and/or translation efficiency may also be used. The RNA preferably has 5′ and 3′ UTRs. In one embodiment, the 5′ UTR is between one and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the nucleic acid of interest. Alternatively, UTR sequences that are not endogenous to the nucleic acid of interest can be added by incorporating the UTR sequences into the forward and reverse primers or by any other modifications of the template. The use of UTR sequences that are not endogenous to the nucleic acid of interest can be useful for modifying the stability and/or translation efficiency of the RNA. For example, it is known that AU-rich elements in 3′UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous nucleic acid. Alternatively, when a 5′ UTR that is not endogenous to the nucleic acid of interest is being added by PCR as described above, a consensus Kozak sequence can be redesigned by adding the 5′ UTR sequence. Kozak sequences can increase the efficiency of translation of some RNA transcripts but do not appear to be required for all RNAs to enable efficient translation. The requirement for Kozak sequences for many mRNAs is known in the art. In other embodiments the 5′ UTR can be 5′UTR of an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one preferred embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In some embodiments, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatemeric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However, polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a poly-T tail, such as 100 T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps can also provide stability to RNA molecules. In some embodiments, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

RNA can be introduced into target cells using any of a number of different methods, for instance, commercially available methods which include, but are not limited to, electroporation (Amaxa Nucleofector®-II (Amaxa Biosystems, Cologne, Germany)), ECM 830 (BTX) (Harvard Instruments, Boston, Mass.) or the Gene Pulser® II (BioRad, Denver, Colo.), Multiporator® (Eppendorf, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001).

### Modified Cells

Disclosed herein, in some embodiments, are cells comprising the recombinant nucleic acid disclosed herein, the polypeptide disclosed herein, or the vectors disclosed herein. Disclosed herein, in some embodiments, are cells comprising the recombinant nucleic acid disclosed herein, the polypeptide disclosed herein, or the vectors disclosed herein; wherein cells comprising the sequence encoding a TFP disclosed herein, an IL-15 polypeptide or a fragment disclosed herein, and/or an IL-15Rα polypeptide or a fragment disclosed herein. The modified cells described herein can be engineered cells expressing an anti-mesothelin TFP and IL-15 enhancements described herein. The engineered cells can show high transduction efficiency and co-expression. The anti-mesothelin TFP-T cells expressing the IL-15 enhancement can be highly cytotoxic and produce cytokines in response to mesothelin-expressing tumor cells. The TFP-T cells expressing the IL-15 enhancements described herein can have a favorable phenotype enriched for CD8+T_{CSM}/T_{CM} cells and show enhanced stemness following activation. The IL-15 enhancements described herein can autonomously increase TFP-T cell persistence in vitro and in vivo in the absence of external activating stimuli. The TFP-T cells bearing IL-15 enhancements can show increased expansion and/or persistence in vivo (e.g., protecting mice from tumor rechallenge as shown in Examples). The IL-15 enhancements described herein can have the potential to increase TFP-T cells persistence in cancer patients for improved efficacy against solid tumors.

In some embodiments, the cell is a T cell. In some embodiments, the T cell is a human T cell. In some embodiments, the T cell is a CD8+ or CD4+ T cell. In some embodiments, the T cell is a human αβ T cell. In some embodiments, the T cell is a human γδ T cell. In some embodiments, the cell is a human NKT cell. In some embodiments, the cell is an allogeneic cell or an autologous cell. In some embodiments, the T cell is modified to comprise a functional disruption of the TCR. In some embodiments, the modified T cells are γδ T cells and do not comprise a functional disruption of an endogenous TCR. In some embodiments, the γδ T cells are Vδ1+Vδ2−γδ T cells. In some embodiments, the γδ T cells are Vδ1−Vδ2+γδ T cells. In some embodiments, the γδ T cells are Vδ1−Vδ2−γδ T cells.

Disclosed herein, in some embodiments, are cells comprising the recombinant nucleic acid disclosed herein, the polypeptide disclosed herein, or the vectors disclosed herein wherein cells comprising the sequence encoding TFP disclosed herein, IL-15 polypeptide or a fragment disclosed herein, and/or IL-15Rα polypeptide or a fragment disclosed herein. In some embodiments, the IL-15 polypeptide or a fragment thereof is secreted when expressed in a cell. For example, cells disclosed herein may secrete IL-15 polypeptide expressed from the recombinant nucleic acid molecules disclosed herein in response to a cell activation agent. In some embodiments, IL-15 signaling is increased in response to a cell activation agent. In some embodiment, the cell activation agent comprises a T cell activation agent. A T cell activation agent, as described herein, may include, but is not limited to, an anti-CD3 antibody or a fragment thereof, an anti-CD28 antibody or a fragment thereof, a cytokine, an antigen that binds the antigen binding domain of the TFP described herein, or any combinations thereof.

Disclosed herein, in some embodiments, are cells comprising the sequence encoding TFP disclosed herein, IL-15 polypeptide or a fragment disclosed herein, and/or IL-15Rα polypeptide or a fragment disclosed herein may have enhanced survival rate, enhanced effector function, and/or enhanced cytotoxicity compared to cells that do not comprise the sequence encoding TFP disclosed herein, IL-15 polypeptide or a fragment disclosed herein, and/or IL-15Rα polypeptide or a fragment disclosed herein. In some embodiments, the cell has enhanced survival rate compared to a cell that does not have IL-15 signaling. In some embodiments, the cell has enhanced survival rate compared to a cell that does not express the IL-15 polypeptide or a fragment thereof and/or IL-15Rα polypeptide or a fragment thereof. In some embodiments, the cell has enhanced effector function compared to a cell that does not have IL-15 signaling. In some embodiments, the cell has enhanced effector function compared to a cell that does not express the IL-15 polypeptide or a fragment thereof and/or IL-15Rα polypeptide or a fragment thereof. In some embodiments, the cell has enhanced cytotoxicity compared to a cell that does not have IL-15 signaling. In some embodiments, the cell has enhanced cytotoxicity compared to a cell that does not express the IL-15 polypeptide or a fragment thereof and/or IL-15Rα polypeptide or a fragment thereof.

Disclosed herein, in some embodiments, are cells comprising the sequence encoding TFP disclosed herein, IL-15 polypeptide or a fragment disclosed herein, and/or IL-15Rα polypeptide or a fragment disclosed herein may have increased longevity compared to cells that do not comprise the sequence encoding TFP disclosed herein, IL-15 polypeptide or a fragment disclosed herein, and/or IL-15Rα polypeptide or a fragment disclosed herein. In some embodiments, the longevity of the cell is increased compared to a cell that does not comprise (i) a nucleic acid sequence encoding an interleukin-15 (IL-15) polypeptide or a fragment thereof or (ii) a nucleic acid sequence encoding an interleukin-15 receptor alpha (IL-15Rα) polypeptide or a fragment thereof.

Disclosed herein, in some embodiments, are cells comprising the sequence encoding TFP disclosed herein, IL-15 polypeptide or a fragment disclosed herein, and/or IL-15Rα polypeptide or a fragment disclosed herein may have increased persistence compared to cells that do not comprise the sequence encoding TFP disclosed herein, IL-15 polypeptide or a fragment disclosed herein, and/or IL-15Rα polypeptide or a fragment disclosed herein. In some embodiments, the persistence of the cell is increased compared to a cell that does not comprise (i) a nucleic acid sequence encoding an interleukin-15 (IL-15) polypeptide or a fragment thereof or (ii) a nucleic acid sequence encoding an interleukin-15 receptor alpha (IL-15Rα) polypeptide or a fragment thereof.

Disclosed herein, in some embodiments, are cells comprising the sequence encoding TFP disclosed herein, IL-15 polypeptide or a fragment disclosed herein, and/or IL-15Rα polypeptide or a fragment disclosed herein may have increased cytotoxicity compared to cells that do not comprise the sequence encoding TFP disclosed herein, IL-15 polypeptide or a fragment disclosed herein, and/or IL-15Rα polypeptide or a fragment disclosed herein. In some embodiments, the cytotoxicity of the cell is increased compared to a cell that does not comprise (i) a nucleic acid sequence encoding an interleukin-15 (IL-15) polypeptide or a fragment thereof or (ii) a nucleic acid sequence encoding an interleukin-15 receptor alpha (IL-15Rα) polypeptide or a fragment thereof.

Disclosed herein, in some embodiments, are cells comprising the sequence encoding TFP disclosed herein, IL-15 polypeptide or a fragment disclosed herein, and/or IL-15Rα polypeptide or a fragment disclosed herein may have increased cytokine production compared to cells that do not comprise the sequence encoding TFP disclosed herein, IL-15 polypeptide or a fragment disclosed herein, and/or IL-15Rα polypeptide or a fragment disclosed herein. In some embodiments, the cytokine production of the cell is increased compared to a cell that does not comprise (i) a nucleic acid sequence encoding an interleukin-15 (IL-15) polypeptide or a fragment thereof or (ii) a nucleic acid sequence encoding an interleukin-15 receptor alpha (IL-15Rα) polypeptide or a fragment thereof.

In some embodiments, cells disclosed herein retains naïve and/or central memory phenotypes. In some embodiments, cells disclosed herein have not differentiated into terminal effector cells.

Disclosed herein, in some embodiments, is a population of cells comprising any of the cell described herein. Disclosed herein, in some embodiments, is a population of cells comprising any of the cell described herein, wherein the population of cells has an increased proportion of cells having a central memory phenotype relative to a population of cells that do not comprise the sequence encoding TFP disclosed herein, IL-15 polypeptide or a fragment disclosed herein, and/or IL-15Rα polypeptide or a fragment disclosed herein. In some embodiments, the population of cells has an increased proportion of cells having a central memory phenotype relative to a population of cells that do not comprise (i) a nucleic acid sequence encoding an interleukin-15 (IL-15) polypeptide or a fragment thereof or (ii) a nucleic acid sequence encoding interleukin-15 receptor alpha (IL-15Rα) polypeptide or a fragment thereof.

Disclosed herein, in some embodiments, is population of cells comprising any of the cell described herein, wherein the population of cells has an increased proportion of cells having a naïve phenotype relative to a population of cells that do not comprise the sequence encoding TFP disclosed herein, IL-15 polypeptide or a fragment disclosed herein, and/or IL-15Rα polypeptide or a fragment disclosed herein. In some embodiments, the population of cells has an increased proportion of cells having a naïve phenotype relative to a population of cells that do not comprise (i) a nucleic acid sequence encoding an interleukin-15 (IL-15) polypeptide or a fragment thereof or (ii) a nucleic acid sequence encoding an interleukin-15 receptor alpha (IL-15Rα) polypeptide or a fragment thereof.

Disclosed herein, in some embodiments, is population of cells comprising any of the cell described herein, wherein the population of cells has a reduced proportion of cells having a terminal effector phenotype relative to a population of cells that do not comprise the sequence encoding TFP disclosed herein, IL-15 polypeptide or a fragment disclosed herein, and/or IL-15Rα polypeptide or a fragment disclosed herein. In some embodiments, the population of cells has a reduced proportion of cells having a terminal effector phenotype relative to a population of cells that do not comprise (i) a nucleic acid sequence encoding an interleukin-15 (IL-15) polypeptide or a fragment thereof or (ii) a nucleic acid sequence encoding an interleukin-15 receptor alpha (IL-15Rα) polypeptide or a fragment thereof.

Disclosed herein, in some embodiments, are modified T cells comprising the recombinant nucleic acid disclosed herein, or the vectors disclosed herein; wherein the modified T cell comprises a functional disruption of an endogenous TCR. Also disclosed herein, in some embodiments, are modified T cells comprising the sequence encoding the TFP of the nucleic acid disclosed herein or a TFP encoded by the sequence of the nucleic acid disclosed herein, wherein the modified T cell comprises a functional disruption of an endogenous TCR. Further disclosed herein, in some embodiments, are modified allogenic T cells comprising the sequence encoding the TFP disclosed herein or a TFP encoded by the sequence of the nucleic acid disclosed herein.

In some instances, the T cell further comprises a heterologous sequence encoding a TCR constant domain, wherein the TCR constant domain is a TCR alpha constant domain, a TCR beta constant domain, a TCR alpha constant domain and a TCR beta constant domain, a TCR gamma constant domain, a TCR delta constant domain or a TCR gamma constant domain and a TCR delta constant domain. In some instances, the endogenous TCR that is functionally disrupted is an endogenous TCR alpha chain, an endogenous TCR beta constant domain, an endogenous TCR alpha constant domain and an endogenous TCR beta constant domain, an endogenous TCR gamma chain, an endogenous TCR delta chain, or an endogenous TCR gamma chain and an endogenous TCR delta chain. In some instances, the endogenous TCR that is functionally disrupted has reduced binding to MHC-peptide complex compared to that of an unmodified control T cell. In some instances, the functional disruption is a disruption of a gene encoding the endogenous TCR. In some instances, the disruption of a gene encoding the endogenous TCR is a removal of a sequence of the gene encoding the endogenous TCR from the genome of a T cell. In some instances, the T cell is a human T cell. In some instances, the T cell is a CD8+ or CD4+ T cell. In some instances, the T cell is an allogenic T cell. In some instances, the modified T cells further comprise a nucleic acid encoding an inhibitory molecule that comprises a first polypeptide comprising at least a portion of an inhibitory molecule, associated with a second polypeptide comprising a positive signal from an intracellular signaling domain. In some instances, the inhibitory molecule comprises the first polypeptide comprising at least a portion of PD-1 and the second polypeptide comprising a costimulatory domain and primary signaling domain. In some embodiments, a T cell expressing the TFP descried herein can inhibit tumor growth when expressed in a T cell.

In some embodiments, proliferation of the cell is increased compared to a cell that does not comprise (i) a nucleic acid sequence encoding an interleukin-15 (IL-15) polypeptide or a fragment thereof or (ii) a nucleic acid sequence encoding an interleukin-15 receptor alpha (IL-15Rα) polypeptide or a fragment thereof. For example, the proliferation of the cell can be increased for at least about 5%. In some embodiments, IL-15 is operatively linked to IL-15Rα. In some embodiments, the activity or persistence of the cell expressing a recombinant nucleic acid molecule comprising a sequence encoding TFP as described herein and a sequence encoding an IL-15 polypeptide or a fragment thereof as described herein is increased. In some embodiments, IL-15 is operatively linked to IL-15Rα. In some embodiments, the activity or persistence of the cell expressing a recombinant nucleic acid molecule comprising a sequence encoding TFP as described herein and a sequence encoding an IL-15 polypeptide or a fragment thereof as described herein is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, at least 1000%, at least 2000%, at least 3000%, at least 4000%, at least 5000%, at least 6000%, at least 7000%, at least 8000%, at least 9000%, at least 10000%, at least 20000%, at least 30000%, at least 40000%, at least 50000%, at least 60000%, at least 70000%, at least 80000%, at least 90000%, or at least 100000% as compared with the cells expressing a recombinant nucleic acid molecule comprising a sequence encoding TFP as described herein, but do not express a recombinant nucleic acid molecule comprising a sequence encoding an IL-15 polypeptide or a fragment thereof as described herein. In some embodiments, the activity or persistence of the cell expressing a recombinant nucleic acid molecule comprising a sequence encoding TFP as described herein and a sequence encoding an IL-15 polypeptide or a fragment thereof as described herein is increased by at least 2 fold, at least 3 fold, at least 4 fold, at least 5 fold, at least 6 fold, at least 7 fold, at least 8 fold, at least 9 fold, at least 10 fold, at least 15 fold, at least 20 fold, at least 25 fold, at least 30 fold, at least 35 fold, at least 40 fold, at least 45 fold, at least 50 fold, at least 55 fold, at least 60 fold, at least 65 fold, at least 70 fold, at least 75 fold, at least 80 fold, at least 85 fold, at least 90 fold, at least 95 fold, at least 100 fold, at least 150 fold, at least 200 fold, at least 250 fold, at least 300 fold, at least 350 fold, at least 400 fold, at least 450 fold, at least 500 fold, at least 550 fold, at least 600 fold, at least 650 fold, at least 700 fold, at least 750 fold, at least 800 fold, at least 850 fold, at least 900 fold, at least 950 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared with the cells expressing a recombinant nucleic acid molecule comprising a sequence encoding TFP as described herein, but do not express a recombinant nucleic acid molecule comprising a sequence encoding an IL-15 polypeptide or a fragment thereof as described herein.

In some embodiments, the activity or persistence of the cell expressing a recombinant nucleic acid molecule comprising a sequence encoding TFP as described herein and a sequence encoding an IL-15Rα polypeptide or a fragment thereof as described herein is increased. In some embodiments, the activity or persistence of the cell expressing a recombinant nucleic acid molecule comprising a sequence encoding TFP as described herein and a sequence encoding an IL-15Rα polypeptide or a fragment thereof as described herein is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, at least 1000%, at least 2000%, at least 3000%, at least 4000%, at least 5000%, at least 6000%, at least 7000%, at least 8000%, at least 9000%, at least 10000%, at least 20000%, at least 30000%, at least 40000%, at least 50000%, at least 60000%, at least 70000%, at least 80000%, at least 90000%, or at least 100000% as compared with the cells expressing a recombinant nucleic acid molecule comprising a sequence encoding TFP as described herein, but do not express a recombinant nucleic acid molecule comprising a sequence encoding an IL-15Rα polypeptide or a fragment thereof as described herein. In some embodiments, the activity or persistence of the cell expressing a recombinant nucleic acid molecule comprising a sequence encoding TFP as described herein and a sequence encoding an IL-15Rα polypeptide or a fragment thereof as described herein is increased by at least 2 fold, at least 3 fold, at least 4 fold, at least 5 fold, at least 6 fold, at least 7 fold, at least 8 fold, at least 9 fold, at least 10 fold, at least 15 fold, at least 20 fold, at least 25 fold, at least 30 fold, at least 35 fold, at least 40 fold, at least 45 fold, at least 50 fold, at least 55 fold, at least 60 fold, at least 65 fold, at least 70 fold, at least 75 fold, at least 80 fold, at least 85 fold, at least 90 fold, at least 95 fold, at least 100 fold, at least 150 fold, at least 200 fold, at least 250 fold, at least 300 fold, at least 350 fold, at least 400 fold, at least 450 fold, at least 500 fold, at least 550 fold, at least 600 fold, at least 650 fold, at least 700 fold, at least 750 fold, at least 800 fold, at least 850 fold, at least 900 fold, at least 950 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared with the cells expressing a recombinant nucleic acid molecule comprising a sequence encoding TFP as described herein, but do not express a recombinant nucleic acid molecule comprising a sequence encoding an IL-15Rα polypeptide or a fragment thereof as described herein.

In some embodiments, the proliferation of the cell expressing a recombinant nucleic acid molecule comprising a sequence encoding TFP as described herein and a sequence encoding an IL-15 polypeptide or a fragment thereof as described herein is increased. In some embodiments, IL-15 is operatively linked to IL-15Rα. In some embodiments, the proliferation of the cell expressing a recombinant nucleic acid molecule comprising a sequence encoding TFP as described herein and a sequence encoding an IL-15 polypeptide or a fragment thereof as described herein is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, at least 1000%, at least 2000%, at least 3000%, at least 4000%, at least 5000%, at least 6000%, at least 7000%, at least 8000%, at least 9000%, at least 10000%, at least 20000%, at least 30000%, at least 40000%, at least 50000%, at least 60000%, at least 70000%, at least 80000%, at least 90000%, or at least 100000% as compared with the cells expressing a recombinant nucleic acid molecule comprising a sequence encoding TFP as described herein, but do not express a recombinant nucleic acid molecule comprising a sequence encoding an IL-15 polypeptide or a fragment thereof as described herein. In some embodiments, the proliferation of the cell expressing a recombinant nucleic acid molecule comprising a sequence encoding TFP as described herein and a sequence encoding an IL-15 polypeptide or a fragment thereof as described herein is increased by at least 2 fold, at least 3 fold, at least 4 fold, at least 5 fold, at least 6 fold, at least 7 fold, at least 8 fold, at least 9 fold, at least 10 fold, at least 15 fold, at least 20 fold, at least 25 fold, at least 30 fold, at least 35 fold, at least 40 fold, at least 45 fold, at least 50 fold, at least 55 fold, at least 60 fold, at least 65 fold, at least 70 fold, at least 75 fold, at least 80 fold, at least 85 fold, at least 90 fold, at least 95 fold, at least 100 fold, at least 150 fold, at least 200 fold, at least 250 fold, at least 300 fold, at least 350 fold, at least 400 fold, at least 450 fold, at least 500 fold, at least 550 fold, at least 600 fold, at least 650 fold, at least 700 fold, at least 750 fold, at least 800 fold, at least 850 fold, at least 900 fold, at least 950 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared with the cells expressing a recombinant nucleic acid molecule comprising a sequence encoding TFP as described herein, but do not express a recombinant nucleic acid molecule comprising a sequence encoding an IL-15 polypeptide or a fragment thereof as described herein.

In some embodiments, the proliferation of the cell expressing a recombinant nucleic acid molecule comprising a sequence encoding TFP as described herein and a sequence encoding an IL-15Rα polypeptide or a fragment thereof as described herein is increased. In some embodiments, the proliferation of the cell expressing a recombinant nucleic acid molecule comprising a sequence encoding TFP as described herein and a sequence encoding an IL-15Rα polypeptide or a fragment thereof as described herein is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, at least 1000%, at least 2000%, at least 3000%, at least 4000%, at least 5000%, at least 6000%, at least 7000%, at least 8000%, at least 9000%, at least 10000%, at least 20000%, at least 30000%, at least 40000%, at least 50000%, at least 60000%, at least 70000%, at least 80000%, at least 90000%, or at least 100000% as compared with the cells expressing a recombinant nucleic acid molecule comprising a sequence encoding TFP as described herein, but do not express a recombinant nucleic acid molecule comprising a sequence encoding an IL-15Rα polypeptide or a fragment thereof as described herein. In some embodiments, the proliferation of the cell expressing a recombinant nucleic acid molecule comprising a sequence encoding TFP as described herein and a sequence encoding an IL-15Rα polypeptide or a fragment thereof as described herein is increased by at least 2 fold, at least 3 fold, at least 4 fold, at least 5 fold, at least 6 fold, at least 7 fold, at least 8 fold, at least 9 fold, at least 10 fold, at least 15 fold, at least 20 fold, at least 25 fold, at least 30 fold, at least 35 fold, at least 40 fold, at least 45 fold, at least 50 fold, at least 55 fold, at least 60 fold, at least 65 fold, at least 70 fold, at least 75 fold, at least 80 fold, at least 85 fold, at least 90 fold, at least 95 fold, at least 100 fold, at least 150 fold, at least 200 fold, at least 250 fold, at least 300 fold, at least 350 fold, at least 400 fold, at least 450 fold, at least 500 fold, at least 550 fold, at least 600 fold, at least 650 fold, at least 700 fold, at least 750 fold, at least 800 fold, at least 850 fold, at least 900 fold, at least 950 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared with the cells expressing a recombinant nucleic acid molecule comprising a sequence encoding TFP as described herein, but do not express a recombinant nucleic acid molecule comprising a sequence encoding an IL-15Rα polypeptide or a fragment thereof as described herein.

In some embodiments, expression of an exhaustion marker of the cell is decreased compared to a cell that does not comprise (i) a nucleic acid sequence encoding an interleukin-15 (IL-15) polypeptide or a fragment thereof or (ii) a nucleic acid sequence encoding an interleukin-15 receptor alpha (IL-15Rα) polypeptide or a fragment thereof. For example, expression of the exhaustion marker of the cell can be decreased for at least about 5%. The exhaustion marker can be PD-1, TIM-3 or LAG-3. In some embodiments, IL-15 is operatively linked to IL-15Rα.

In some embodiments, the expression of one or more exhaustion markers in the cell expressing a recombinant nucleic acid molecule comprising a sequence encoding TFP as described herein and a sequence encoding an IL-15 polypeptide or a fragment thereof as described herein is decreased. In some embodiments, IL-15 is operatively linked to IL-15Rα. In some embodiments, the expression of one or more exhaustion markers in the cell expressing a recombinant nucleic acid molecule comprising a sequence encoding TFP as described herein and a sequence encoding an IL-15 polypeptide or a fragment thereof as described herein is decreased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, at least 99.5%, at least 99.9%, or 100% as compared with the cells expressing a recombinant nucleic acid molecule comprising a sequence encoding TFP as described herein, but do not express a recombinant nucleic acid molecule comprising a sequence encoding an IL-15 polypeptide or a fragment thereof as described herein. In some embodiments, the expression of one or more exhaustion markers in the cell expressing a recombinant nucleic acid molecule comprising a sequence encoding TFP as described herein and a sequence encoding an IL-15 polypeptide or a fragment thereof as described herein is decreased by at least 2 fold, at least 3 fold, at least 4 fold, at least 5 fold, at least 6 fold, at least 7 fold, at least 8 fold, at least 9 fold, at least 10 fold, at least 15 fold, at least 20 fold, at least 25 fold, at least 30 fold, at least 35 fold, at least 40 fold, at least 45 fold, at least 50 fold, at least 55 fold, at least 60 fold, at least 65 fold, at least 70 fold, at least 75 fold, at least 80 fold, at least 85 fold, at least 90 fold, at least 95 fold, at least 100 fold, at least 150 fold, at least 200 fold, at least 250 fold, at least 300 fold, at least 350 fold, at least 400 fold, at least 450 fold, at least 500 fold, at least 550 fold, at least 600 fold, at least 650 fold, at least 700 fold, at least 750 fold, at least 800 fold, at least 850 fold, at least 900 fold, at least 950 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared with the cells expressing a recombinant nucleic acid molecule comprising a sequence encoding TFP as described herein, but do not express a recombinant nucleic acid molecule comprising a sequence encoding an IL-15 polypeptide or a fragment thereof as described herein.

In some embodiments, the expression of one or more exhaustion markers in the cell expressing a recombinant nucleic acid molecule comprising a sequence encoding TFP as described herein and a sequence encoding an IL-15Rα polypeptide or a fragment thereof as described herein is decreased. In some embodiments, the expression of one or more exhaustion markers in the cell expressing a recombinant nucleic acid molecule comprising a sequence encoding TFP as described herein and a sequence encoding an IL-15Rα polypeptide or a fragment thereof as described herein is decreased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, at least 99.5%, at least 99.9%, or 100% as compared with the cells expressing a recombinant nucleic acid molecule comprising a sequence encoding TFP as described herein, but do not express a recombinant nucleic acid molecule comprising a sequence encoding an IL-15Rα polypeptide or a fragment thereof as described herein. In some embodiments, the expression of one or more exhaustion markers in the cell expressing a recombinant nucleic acid molecule comprising a sequence encoding TFP as described herein and a sequence encoding an IL-15Rα polypeptide or a fragment thereof as described herein is decreased by at least 2 fold, at least 3 fold, at least 4 fold, at least 5 fold, at least 6 fold, at least 7 fold, at least 8 fold, at least 9 fold, at least 10 fold, at least 15 fold, at least 20 fold, at least 25 fold, at least 30 fold, at least 35 fold, at least 40 fold, at least 45 fold, at least 50 fold, at least 55 fold, at least 60 fold, at least 65 fold, at least 70 fold, at least 75 fold, at least 80 fold, at least 85 fold, at least 90 fold, at least 95 fold, at least 100 fold, at least 150 fold, at least 200 fold, at least 250 fold, at least 300 fold, at least 350 fold, at least 400 fold, at least 450 fold, at least 500 fold, at least 550 fold, at least 600 fold, at least 650 fold, at least 700 fold, at least 750 fold, at least 800 fold, at least 850 fold, at least 900 fold, at least 950 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared with the cells expressing a recombinant nucleic acid molecule comprising a sequence encoding TFP as described herein, but do not express a recombinant nucleic acid molecule comprising a sequence encoding an IL-15Rα polypeptide or a fragment thereof as described herein.

In some embodiments, expression of TCF-1 of the cell is increased compared to a cell that does not comprise (i) a nucleic acid sequence encoding an interleukin-15 (IL-15) polypeptide or a fragment thereof or (ii) a nucleic acid sequence encoding an interleukin-15 receptor alpha (IL-15Rα) polypeptide or a fragment thereof. For example, the expression of TCF-1 of the cell is increased for at least about 5%. In some embodiments, IL-15 is operatively linked to IL-15Rα.

In some embodiments, the TCF-1+ T cell population is increased in a population of the cells expressing a recombinant nucleic acid molecule comprising a sequence encoding TFP as described herein and a sequence encoding an IL-15 polypeptide or a fragment thereof as described herein. In some embodiments, IL-15 is operatively linked to IL-15Rα. In some embodiments, the TCF-1+ T cell population is increased in a population of the cell expressing a recombinant nucleic acid molecule comprising a sequence encoding TFP as described herein and a sequence encoding an IL-15 polypeptide or a fragment thereof as described herein by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, at least 1000%, at least 2000%, at least 3000%, at least 4000%, at least 5000%, at least 6000%, at least 7000%, at least 8000%, at least 9000%, at least 10000%, at least 20000%, at least 30000%, at least 40000%, at least 50000%, at least 60000%, at least 70000%, at least 80000%, at least 90000%, or at least 100000% as compared with a population of the cells expressing a recombinant nucleic acid molecule comprising a sequence encoding TFP as described herein, but do not express a recombinant nucleic acid molecule comprising a sequence encoding an IL-15 polypeptide or a fragment thereof as described herein. In some embodiments, the TCF-1+ T cell population is increased in a population of the cells expressing a recombinant nucleic acid molecule comprising a sequence encoding TFP as described herein and a sequence encoding an IL-15 polypeptide or a fragment thereof as described herein by at least 2 fold, at least 3 fold, at least 4 fold, at least 5 fold, at least 6 fold, at least 7 fold, at least 8 fold, at least 9 fold, at least 10 fold, at least 15 fold, at least 20 fold, at least 25 fold, at least 30 fold, at least 35 fold, at least 40 fold, at least 45 fold, at least 50 fold, at least 55 fold, at least 60 fold, at least 65 fold, at least 70 fold, at least 75 fold, at least 80 fold, at least 85 fold, at least 90 fold, at least 95 fold, at least 100 fold, at least 150 fold, at least 200 fold, at least 250 fold, at least 300 fold, at least 350 fold, at least 400 fold, at least 450 fold, at least 500 fold, at least 550 fold, at least 600 fold, at least 650 fold, at least 700 fold, at least 750 fold, at least 800 fold, at least 850 fold, at least 900 fold, at least 950 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared with a population of the cells expressing a recombinant nucleic acid molecule comprising a sequence encoding TFP as described herein, but do not express a recombinant nucleic acid molecule comprising a sequence encoding an IL-15 polypeptide or a fragment thereof as described herein.

In some embodiments, the TCF-1+ T cell population is increased in a population of the cells expressing a recombinant nucleic acid molecule comprising a sequence encoding TFP as described herein and a sequence encoding an IL-15Rα polypeptide or a fragment thereof as described herein. In some embodiments, the TCF-1+ T cell population is increased in a population of the cell expressing a recombinant nucleic acid molecule comprising a sequence encoding TFP as described herein and a sequence encoding an IL-15Rα polypeptide or a fragment thereof as described herein by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, at least 1000%, at least 2000%, at least 3000%, at least 4000%, at least 5000%, at least 6000%, at least 7000%, at least 8000%, at least 9000%, at least 10000%, at least 20000%, at least 30000%, at least 40000%, at least 50000%, at least 60000%, at least 70000%, at least 80000%, at least 90000%, or at least 100000% as compared with a population of the cells expressing a recombinant nucleic acid molecule comprising a sequence encoding TFP as described herein, but do not express a recombinant nucleic acid molecule comprising a sequence encoding an IL-15Rα polypeptide or a fragment thereof as described herein. In some embodiments, the TCF-1+ T cell population is increased in a population of the cell expressing a recombinant nucleic acid molecule comprising a sequence encoding TFP as described herein and a sequence encoding an IL-15Rα polypeptide or a fragment thereof as described herein by at least 2 fold, at least 3 fold, at least 4 fold, at least 5 fold, at least 6 fold, at least 7 fold, at least 8 fold, at least 9 fold, at least 10 fold, at least 15 fold, at least 20 fold, at least 25 fold, at least 30 fold, at least 35 fold, at least 40 fold, at least 45 fold, at least 50 fold, at least 55 fold, at least 60 fold, at least 65 fold, at least 70 fold, at least 75 fold, at least 80 fold, at least 85 fold, at least 90 fold, at least 95 fold, at least 100 fold, at least 150 fold, at least 200 fold, at least 250 fold, at least 300 fold, at least 350 fold, at least 400 fold, at least 450 fold, at least 500 fold, at least 550 fold, at least 600 fold, at least 650 fold, at least 700 fold, at least 750 fold, at least 800 fold, at least 850 fold, at least 900 fold, at least 950 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared with a population of the cells expressing a recombinant nucleic acid molecule comprising a sequence encoding TFP as described herein, but do not express a recombinant nucleic acid molecule comprising a sequence encoding an IL-15Rα polypeptide or a fragment thereof as described herein.

In some embodiments, tumor infiltration of the cell is increased compared to a cell that does not comprise (i) a nucleic acid sequence encoding an interleukin-15 (IL-15) polypeptide or a fragment thereof or (ii) a nucleic acid sequence encoding an interleukin-15 receptor alpha (IL-15Rα) polypeptide or a fragment thereof. For example, the tumor infiltration of the cell can be increased for at least about 2-fold. In some embodiments, IL-15 is operatively linked to IL-15Rα. In some embodiments, the tumor infiltration of the cell expressing a recombinant nucleic acid molecule comprising a sequence encoding TFP as described herein and a sequence encoding an IL-15 polypeptide or a fragment thereof as described herein is increased. In some embodiments, IL-15 is operatively linked to IL-15Rα. In some embodiments, the tumor infiltration of the cell expressing a recombinant nucleic acid molecule comprising a sequence encoding TFP as described herein and a sequence encoding an IL-15 polypeptide or a fragment thereof as described herein is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, at least 1000%, at least 2000%, at least 3000%, at least 4000%, at least 5000%, at least 6000%, at least 7000%, at least 8000%, at least 9000%, at least 10000%, at least 20000%, at least 30000%, at least 40000%, at least 50000%, at least 60000%, at least 70000%, at least 80000%, at least 90000%, or at least 100000% as compared with the cells expressing a recombinant nucleic acid molecule comprising a sequence encoding TFP as described herein, but do not express a recombinant nucleic acid molecule comprising a sequence encoding an IL-15 polypeptide or a fragment thereof as described herein. In some embodiments, the tumor infiltration of the cell expressing a recombinant nucleic acid molecule comprising a sequence encoding TFP as described herein and a sequence encoding an IL-15 polypeptide or a fragment thereof as described herein is increased by at least 2 fold, at least 3 fold, at least 4 fold, at least 5 fold, at least 6 fold, at least 7 fold, at least 8 fold, at least 9 fold, at least 10 fold, at least 15 fold, at least 20 fold, at least 25 fold, at least 30 fold, at least 35 fold, at least 40 fold, at least 45 fold, at least 50 fold, at least 55 fold, at least 60 fold, at least 65 fold, at least 70 fold, at least 75 fold, at least 80 fold, at least 85 fold, at least 90 fold, at least 95 fold, at least 100 fold, at least 150 fold, at least 200 fold, at least 250 fold, at least 300 fold, at least 350 fold, at least 400 fold, at least 450 fold, at least 500 fold, at least 550 fold, at least 600 fold, at least 650 fold, at least 700 fold, at least 750 fold, at least 800 fold, at least 850 fold, at least 900 fold, at least 950 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared with the cells expressing a recombinant nucleic acid molecule comprising a sequence encoding TFP as described herein, but do not express a recombinant nucleic acid molecule comprising a sequence encoding an IL-15 polypeptide or a fragment thereof as described herein.

In some embodiments, the tumor infiltration of the cell expressing a recombinant nucleic acid molecule comprising a sequence encoding TFP as described herein and a sequence encoding an IL-15Rα polypeptide or a fragment thereof as described herein is increased. In some embodiments, the tumor infiltration of the cell expressing a recombinant nucleic acid molecule comprising a sequence encoding TFP as described herein and a sequence encoding an IL-15Rα polypeptide or a fragment thereof as described herein is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, at least 1000%, at least 2000%, at least 3000%, at least 4000%, at least 5000%, at least 6000%, at least 7000%, at least 8000%, at least 9000%, at least 10000%, at least 20000%, at least 30000%, at least 40000%, at least 50000%, at least 60000%, at least 70000%, at least 80000%, at least 90000%, or at least 100000% as compared with the cells expressing a recombinant nucleic acid molecule comprising a sequence encoding TFP as described herein, but do not express a recombinant nucleic acid molecule comprising a sequence encoding an IL-15Rα polypeptide or a fragment thereof as described herein. In some embodiments, the tumor infiltration of the cell expressing a recombinant nucleic acid molecule comprising a sequence encoding TFP as described herein and a sequence encoding an IL-15Rα polypeptide or a fragment thereof as described herein is increased by at least 2 fold, at least 3 fold, at least 4 fold, at least 5 fold, at least 6 fold, at least 7 fold, at least 8 fold, at least 9 fold, at least 10 fold, at least 15 fold, at least 20 fold, at least 25 fold, at least 30 fold, at least 35 fold, at least 40 fold, at least 45 fold, at least 50 fold, at least 55 fold, at least 60 fold, at least 65 fold, at least 70 fold, at least 75 fold, at least 80 fold, at least 85 fold, at least 90 fold, at least 95 fold, at least 100 fold, at least 150 fold, at least 200 fold, at least 250 fold, at least 300 fold, at least 350 fold, at least 400 fold, at least 450 fold, at least 500 fold, at least 550 fold, at least 600 fold, at least 650 fold, at least 700 fold, at least 750 fold, at least 800 fold, at least 850 fold, at least 900 fold, at least 950 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared with the cells expressing a recombinant nucleic acid molecule comprising a sequence encoding TFP as described herein, but do not express a recombinant nucleic acid molecule comprising a sequence encoding an IL-15Rα polypeptide or a fragment thereof as described herein.

### Sources of T Cells

Prior to expansion and genetic modification, a source of T cells is obtained from a subject. The term “subject” is intended to include living organisms in which an immune response can be elicited (e.g., mammals). Examples of subjects include humans, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain aspects of the present disclosure, any number of T cell lines available in the art, may be used. In certain aspects of the present disclosure, T cells can be obtained from a unit of blood collected from a subject using any number of techniques known to the skilled artisan, such as Ficoll™ separation. In one preferred aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and to place the cells in an appropriate buffer or media for subsequent processing steps. In one aspect of the present disclosure, the cells are washed with phosphate buffered saline (PBS). In an alternative aspect, the wash solution lacks calcium and may lack magnesium or may lack many if not all divalent cations. Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe® 2991 cell processor, the Baxter Oncology CytoMate™, or the Haemonetics® Cell Saver® 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed, and the cells directly resuspended in culture media.

In one aspect, T cells are isolated from peripheral blood lymphocytes by lysing the red blood cells and depleting the monocytes, for example, by centrifugation through a PERCOLL® gradient or by counterflow centrifugal elutriation. A specific subpopulation of T cells, such as CD3+, CD28+, CD4+, CD8+, CD45RA+, and CD45RO+ T cells, can be further isolated by positive or negative selection techniques. For example, in one aspect, T cells are isolated by incubation with anti-CD3/anti-CD28 (e.g., 3×28)-conjugated beads, such as DYNABEADS® M-450 CD3/CD28 T, for a time period sufficient for positive selection of the desired T cells. In one aspect, the time period is about 30 minutes. In a further aspect, the time period ranges from 30 minutes to 36 hours or longer and all integer values there between. In a further aspect, the time period is at least 1, 2, 3, 4, 5, or 6 hours. In yet another preferred aspect, the time period is 10 to 24 hours. In one aspect, the incubation time period is 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this present disclosure. In certain aspects, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain aspects, it may be desirable to enrich for or positively select for regulatory T cells which typically express CD4+, CD25+, CD62Lhi, GITR+, and FoxP3+. Alternatively, in certain aspects, T regulatory cells are depleted by anti-C25 conjugated beads or other similar method of selection.

In one embodiment, a T cell population can be selected that expresses one or more of IFN-γ TNF-alpha, IL-17A, IL-2, IL-3, IL-4, GM-CSF, IL-10, IL-13, granzyme B, and perforin, or other appropriate molecules, e.g., other cytokines. Methods for screening for cell expression can be determined, e.g., by the methods described in PCT Publication No.: WO 2013/126712.

For isolation of a desired population of cells by positive or negative selection, the concentration of cells and surface (e.g., particles such as beads) can be varied. In certain aspects, it may be desirable to significantly decrease the volume in which beads and cells are mixed together (e.g., increase the concentration of cells), to ensure maximum contact of cells and beads. For example, in one aspect, a concentration of 2 billion cells/mL is used. In one aspect, a concentration of 1 billion cells/mL is used. In a further aspect, greater than 100 million cells/mL is used. In a further aspect, a concentration of cells of 10, 15, 20, 25, 30, 35, 40, 45, or 50 million cells/mL is used. In yet one aspect, a concentration of cells from 75, 80, 85, 90, 95, or 100 million cells/mL is used. In further aspects, concentrations of 125 or 150 million cells/mL can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (e.g., leukemic blood, tumor tissue, etc.). Such populations of cells may have therapeutic value and would be desirable to obtain. For example, using high concentration of cells allows more efficient selection of CD8+ T cells that normally have weaker CD28 expression.

In a related aspect, it may be desirable to use lower concentrations of cells. By significantly diluting the mixture of T cells and surface (e.g., particles such as beads), interactions between the particles and cells are minimized. This selects for cells that express high amounts of desired antigens to be bound to the particles. For example, CD4+ T cells express higher levels of CD28 and are more efficiently captured than CD8+ T cells in dilute concentrations. In one aspect, the concentration of cells used is 5×10⁶/mL. In other aspects, the concentration used can be from about 1×10⁵/mL to 1×10⁶/mL, and any integer value in between. In other aspects, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1 per minute and stored in the vapor phase of a liquid nitrogen storage tank. Other methods of controlled freezing may be used as well as uncontrolled freezing immediately at −20° C. or in liquid nitrogen. In certain aspects, cryopreserved cells are thawed and washed as described herein and allowed to rest for one hour at room temperature prior to activation using the methods of the present disclosure.

Also contemplated in the context of the present disclosure is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one aspect a blood sample or an apheresis is taken from a generally healthy subject. In certain aspects, a blood sample or an apheresis is taken from a generally healthy subject who is at risk of developing a disease, but who has not yet developed a disease, and the cells of interest are isolated and frozen for later use. In certain aspects, the T cells may be expanded, frozen, and used at a later time. In certain aspects, samples are collected from a patient shortly after diagnosis of a particular disease as described herein but prior to any treatments. In a further aspect, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents such as cyclosporin, azathioprine, methotrexate, and mycophenolate, antibodies, or other immunoablative agents such as alemtuzumab, anti-CD3 antibodies, cytoxan, fludarabine, cyclosporin, tacrolimus, rapamycin, mycophenolic acid, steroids, romidepsin, and irradiation.

In a further aspect of the present disclosure, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present disclosure to collect blood cells, including T cells, dendritic cells, or other cells of the hematopoietic lineage, during this recovery phase. Further, in certain aspects, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

### Activation and Expansion of T Cells

T cells may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and 7,572,631.

Generally, the T cells of the present disclosure may be expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells, CD8+ T cells, or CD4+CD8+ T cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol. Meth. 227(1-2):53-63, 1999). In some embodiments, T cells are activated by incubation with anti-CD3/anti-CD28-conjugated beads, such as DYNABEADS® or Trans-Act® beads, for a time period sufficient for activation of the T cells. In one aspect, the time period is at least 1, 2, 3, 4, 5, or 6 hours. In yet another preferred aspect, the time period is 10 to 24 hours, e.g., 24 hours. In some embodiments, T cells are activated by stimulation with an anti-CD3 antibody and an anti-CD28 antibody in combination with cytokines that bind the common gamma-chain (e.g., IL-2, IL-7, IL-12, IL-15, IL-21, and others). In some embodiments, T cells are activated by stimulation with an anti-CD3 antibody and an anti-CD28 antibody in combination with 10, 20, 30, 40, 50, 60, 70, 80, 90, 100, 200, 300, 400, 500, 600, 700, 800, 900, or 100 U/mL of IL-2, IL-7, and/or IL-15. In some embodiments, the cells are activated for 24 hours. In some embodiments, after transduction, the cells are expanded in the presence of anti-CD3 antibody, anti-CD28 antibody in combination with the same cytokines. In some embodiments, cells activated in the presence of an anti-CD3 antibody and an anti-CD28 antibody in combination with cytokines that bind the common gamma-chain are expanded in the presence of the same cytokines in the absence of the anti-CD3 antibody and anti-CD28 antibody after transduction. In some embodiments, after transduction, the cells are expanded in the presence of anti-CD3 antibody, anti-CD28 antibody in combination with the same cytokines up to a first washing step, when the cells are sub-cultured in media that includes the cytokines but does not include the anti-CD3 antibody and anti-CD28 antibody. In some embodiments, the cells are subcultured every 1, 2, 3, 4, 5, or 6 days. In some embodiments, cells are expanded for 4, 5, 6, 7, 8, 9, 10, 11, 12, 13, 14, 15, 16, 17, 18, 19, 20, 21, 22, 23, 24, 25, 26, 27, 28, 29, or 30 days.

The expansion of T cells may be stimulated with zoledronic acid (Zometa), alendronic acid (Fosamax) or other related bisphosphonate drugs at concentrations of 0.1, 0.25, 0.5, 1.0, 2.0, 3.0, 4.0, 5.0, 7.5, 10, or 100 μM in the presence of feeder cells (irradiated cancer cells, PBMCs, artificial antigen presenting cells). The expansion of T cells may be stimulated with isopentyl pyrophosphate (IPP), (E)-4-Hydroxy-3-methyl-but-2-enyl pyrophosphate (HMBPP or HMB-PP) or other structurally related compounds at concentrations of 0.1, 0.25, 0.5, 1.0, 2.0, 3.0, 4.0, 5.0, 7.5, 10, or 100 μM in the presence of feeder cells (irradiated cancer cells, PBMCs, artificial antigen presenting cells). In some embodiments, the expansion of T cells may be stimulated with synthetic phosphoantigens (e.g., bromohydrin pyrophosphate; BrHPP), 2M3B1 PP, or 2-methyl-3-butenyl-1-pyrophosphate in the presence of IL-2 for one-to-two weeks. In some embodiments, the expansion of T cells may be stimulated with immobilized anti-TCRyd (e.g., pan TCRY6) in the presence of IL-2, e.g., for approximately 14 days. In some embodiments, the expansion of T cells may be stimulated with culture of immobilized anti-CD3 antibodies (e.g., OKT3) in the presence of IL-2. In some embodiments, the aforementioned culture is maintained for about seven days prior to subculture in soluble anti-CD3, and IL-2.

T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population (TC, CD8+). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

Once an anti-CD19, anti-BCMA, anti-CD22, anti-ROR1, anti-PD-1, or anti-BAFF, anti-MUC16, anti-mesothelin, anti-HER2, anti-PMSA, anti-CD20, anti-CD70, anti-GPC3, anti-Nectin-4, anti-Trop2, or antiCD79b TFP is constructed, various assays can be used to evaluate the activity of the molecule, such as but not limited to, the ability to expand T cells following antigen stimulation, sustain T cell expansion in the absence of re-stimulation, and anti-cancer activities in appropriate in vitro and animal models. Assays to evaluate the effects of an anti-CD19, anti-BCMA, anti-GPC3, anti-Nectin-4, anti-Trop2, anti-CD22, anti-MSLN, anti-CD79B, anti-ROR1, anti-PD-1, anti-IL13Ra2, anti-PD-L1, anti-CD20, anti-CD70, or anti-BAFF or BAFFR TFP are described in further detail below.

Western blot analysis of TFP expression in primary T cells can be used to detect the presence of monomers and dimers (see, e.g., Milone et al., Molecular Therapy 17(8): 1453-1464 (2009)). Very briefly, T cells (1:1 mixture of CD4⁺ and CD8⁺ T cells) expressing the TFPs are expanded in vitro for more than 10 days followed by lysis and SDS-PAGE under reducing conditions. TFPs are detected by western blotting using an antibody to a TCR chain. The same T cell subsets are used for SDS-PAGE analysis under non-reducing conditions to permit evaluation of covalent dimer formation.

In vitro expansion of TFP⁺ T cells following antigen stimulation can be measured by flow cytometry. For example, a mixture of CD4⁺ and CD8⁺ T cells are stimulated with alphaCD3/alphaCD28 and APCs followed by transduction with lentiviral vectors expressing GFP under the control of the promoters to be analyzed. Exemplary promoters include the CMV IE gene, EF-1alpha, ubiquitin C, or phosphoglycerokinase (PGK) promoters. GFP fluorescence is evaluated on day 6 of culture in the CD4+ and/or CD8+ T cell subsets by flow cytometry (see, e.g., Milone et al., Molecular Therapy 17(8): 1453-1464 (2009)). Alternatively, a mixture of CD4+ and CD8+ T cells are stimulated with alphaCD3/alphaCD28 coated magnetic beads on day 0 and transduced with TFP on day 1 using a bicistronic lentiviral vector expressing TFP along with eGFP using a 2A ribosomal skipping sequence. Cultures are re-stimulated with either TAA+K562 cells (K562-TAA), wild-type K562 cells (K562 wild type) or K562 cells expressing hCD32 and 4-1BBL in the presence of anti-CD3 and anti-CD28 antibody (K562-BBL-3/28) following washing. Exogenous IL-2 is added to the cultures every other day at 100 IU/mL. GFP+ T cells are enumerated by flow cytometry using bead-based counting (see, e.g., Milone et al., Molecular Therapy 17(8): 1453-1464 (2009)).

Sustained TFP+ T cell expansion in the absence of re-stimulation can also be measured (see, e.g., Milone et al., Molecular Therapy 17(8): 1453-1464 (2009)). Briefly, mean T cell volume (fl) is measured on day 8 of culture using a Coulter Multisizer III particle counter following stimulation with alphaCD3/alphaCD28 coated magnetic beads on day 0, and transduction with the indicated TFP on day 1.

Animal models can also be used to measure a TFP-T activity. For example, xenograft model using, e.g., human CD19-specific TFP+ T cells to treat a primary human pre-B ALL in immunodeficient mice can be used (see, e.g., Milone et al., Molecular Therapy 17(8): 1453-1464 (2009)). After establishment of ALL, mice are randomized as to treatment groups. Different numbers of engineered T cells are coinjected at a 1:1 ratio into NOD/SCID/γ−/− mice bearing B-ALL. The number of copies of each vector in spleen DNA from mice is evaluated at various times following T cell injection. Animals are assessed for leukemia at weekly intervals. Peripheral blood CD19+ B-ALL blast cell counts are measured in mice that are injected with alphaCD19-zeta TFP+ T cells or mock-transduced T cells. Survival curves for the groups are compared using the log-rank test. In addition, absolute peripheral blood CD4+ and CD8+ T cell counts 4 weeks following T cell injection in NOD/SCID/γ−/− mice can also be analyzed. Mice are injected with leukemic cells and 3 weeks later are injected with T cells engineered to express TFP by a bicistronic lentiviral vector that encodes the TFP linked to eGFP. T cells are normalized to 45-50% input GFP+ T cells by mixing with mock-transduced cells prior to injection and confirmed by flow cytometry. Animals are assessed for leukemia at 1-week intervals. Survival curves for the TFP+ T cell groups are compared using the log-rank test.

Dose dependent TFP treatment response can be evaluated (see, e.g., Milone et al., Molecular Therapy 17(8): 1453-1464 (2009)). For example, peripheral blood is obtained 35-70 days after establishing leukemia in mice injected on day 21 with TFP T cells, an equivalent number of mock-transduced T cells, or no T cells. Mice from each group are randomly bled for determination of peripheral blood CD19+ ALL blast counts and then killed on days 35 and 49. The remaining animals are evaluated on days 57 and 70.

Assessment of cell proliferation and cytokine production has been previously described, e.g., at Milone et al., Molecular Therapy 17(8): 1453-1464 (2009). Briefly, assessment of TFP-mediated proliferation is performed in microtiter plates by mixing washed T cells with K562 cells expressing the tumor associated antigen (TAA, e.g., CD19) CD19 (K19) or CD32 and CD137 (KT32-BBL) for a final T cell:K562 ratio of 2:1. K562 cells are irradiated with gamma-radiation prior to use. Anti-CD3 (clone OKT3) and anti-CD28 (clone 9.3) monoclonal antibodies are added to cultures with KT32-BBL cells to serve as a positive control for stimulating T cell proliferation since these signals support long-term CD8+ T cell expansion ex vivo. T cells are enumerated in cultures using CountBright™ fluorescent beads (Invitrogen) and flow cytometry as described by the manufacturer. TFP+ T cells are identified by GFP expression using T cells that are engineered with eGFP-2A linked TFP-expressing lentiviral vectors. For TFP+ T cells not expressing GFP, the TFP+ T cells are detected with biotinylated recombinant CD19 protein and a secondary avidin-PE conjugate. CD4+ and CD8+ expression on T cells are also simultaneously detected with specific monoclonal antibodies (BD Biosciences). Cytokine measurements are performed on supernatants collected 24 hours following re-stimulation using the human TH1/TH2 cytokine cytometric bead array kit (BD Biosciences) according the manufacturer's instructions. Fluorescence is assessed using a FACScalibur™ flow cytometer (BD Biosciences), and data are analyzed according to the manufacturer's instructions.

Cytotoxicity can be assessed by a standard ⁵¹Cr-release assay (see, e.g., Milone et al., Molecular Therapy 17(8): 1453-1464 (2009)). Target cells (K562 lines and primary pro-B-ALL cells) are loaded with ⁵¹Cr (as NaCrO₄, New England Nuclear) at 37° C. for 2 hours with frequent agitation, washed twice in complete RPMI and plated into microtiter plates. Effector T cells are mixed with target cells in the wells in complete RPMI at varying ratios of effector cell:target cell (E:T). Additional wells containing media only (spontaneous release, SR) or a 1% solution of Triton-X 100 detergent (total release, TR) are also prepared. After 4 hours of incubation at 37° C., supernatant from each well is harvested. Released ⁵¹Cr is then measured using a gamma particle counter (Packard Instrument Co., Waltham, Mass.). Each condition is performed in at least triplicate, and the percentage of lysis is calculated using the formula: % Lysis=(ER−SR)/(TR−SR), where ER represents the average ⁵¹Cr released for each experimental condition.

Imaging technologies can be used to evaluate specific trafficking and proliferation of TFPs in tumor-bearing animal models. Such assays have been described, e.g., in Barrett et al., Human Gene Therapy 22:1575-1586 (2011). NOD/SCID/γc−/− (NSG) mice are injected IV with Nalm-6 cells (ATCC® CRL-3273™) followed 7 days later with T cells 4 hour after electroporation with the TFP constructs. The T cells are stably transfected with a lentiviral construct to express firefly luciferase, and mice are imaged for bioluminescence. Alternatively, therapeutic efficacy and specificity of a single injection of TFP+ T cells in Nalm-6 xenograft model can be measured as the following: NSG mice are injected with Nalm-6 transduced to stably express firefly luciferase, followed by a single tail-vein injection of T cells electroporated with a TAA-TFP 7 days later. Animals are imaged at various time points post injection. For example, photon-density heat maps of firefly luciferase positive leukemia in representative mice at day 5 (2 days before treatment) and day 8 (24 hours post TFP+ PBLs) can be generated.

Other assays, including those described in the Example section herein as well as those that are known in the art can also be used to evaluate the anti-CD19, anti-BCMA, anti-CD22, anti-MSLN, anti-CD79B, anti-GPC3, anti-Nectin-4, anti-Trop2, anti-IL13Ra2, anti-PD-1, anti-ROR1, anti-PD-L1, or anti-BAFF or BAFFR TFP constructs disclosed herein.

### Pharmaceutical Compositions

Disclosed herein, in some embodiments, are pharmaceutical compositions comprising: (a) the cells of the disclosure; and (b) a pharmaceutically acceptable carrier. Disclosed herein, in some embodiments, are pharmaceutical compositions comprising: (a) the modified T cells of the disclosure; and (b) a pharmaceutically acceptable carrier. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are in one aspect formulated for intravenous administration.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

In one embodiment, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In one embodiment, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

When “an immunologically effective amount,” “an anti-tumor effective amount,” “a tumor-inhibiting effective amount,” or “therapeutic amount” is indicated, the precise amount of the compositions of the present disclosure to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, in some instances 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. Med. 319:1676, 1988).

In certain aspects, it may be desired to administer activated T cells to a subject and then subsequently redraw blood (or have an apheresis performed), activate T cells therefrom according to the present disclosure, and reinfuse the patient with these activated and expanded T cells. This process can be carried out multiple times every few weeks. In certain aspects, T cells can be activated from blood draws of from 10 cc to 400 cc. In certain aspects, T cells are activated from blood draws of 20 cc, 30 cc, 40 cc, 50 cc, 60 cc, 70 cc, 80 cc, 90 cc, or 100 cc.

The administration of the subject compositions may be carried out in any convenient manner, including by aerosol inhalation, injection, ingestion, transfusion, implantation or transplantation. The compositions described herein may be administered to a patient trans arterially, subcutaneously, intradermally, intratumorally, intranodally, intramedullary, intramuscularly, by intravenous (i.v.) injection, or intraperitoneally. In one aspect, the T cell compositions of the present disclosure are administered to a patient by intradermal or subcutaneous injection. In one aspect, the T cell compositions of the present disclosure are administered by i.v. injection. The compositions of T cells may be injected directly into a tumor, lymph node, or site of infection.

In a particular exemplary aspect, subjects may undergo leukapheresis, wherein leukocytes are collected, enriched, or depleted ex vivo to select and/or isolate the cells of interest, e.g., T cells. These T cell isolates may be expanded by methods known in the art and treated such that one or more TFP constructs of the present disclosure may be introduced, thereby creating a modified T-T cell of the present disclosure. Subjects in need thereof may subsequently undergo standard treatment with high dose chemotherapy followed by peripheral blood stem cell transplantation. In certain aspects, following or concurrent with the transplant, subjects receive an infusion of the expanded modified T cells of the present disclosure. In an additional aspect, expanded cells are administered before or following surgery.

The dosage of the above treatments to be administered to a patient will vary with the precise nature of the condition being treated and the recipient of the treatment. The scaling of dosages for human administration can be performed according to art-accepted practices. The dose for alemtuzumab, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

In one embodiment, the TFP is introduced into T cells, e.g., using in vitro transcription, and the subject (e.g., human) receives an initial administration of TFP T cells of the present disclosure, and one or more subsequent administrations of the TFP T cells of the present disclosure, wherein the one or more subsequent administrations are administered less than 15 days, e.g., 14, 13, 12, 11, 10, 9, 8, 7, 6, 5, 4, 3, or 2 days after the previous administration. In one embodiment, more than one administration of the TFP T cells of the present disclosure are administered to the subject (e.g., human) per week, e.g., 2, 3, or 4 administrations of the TFP T cells of the present disclosure are administered per week. In one embodiment, the subject (e.g., human subject) receives more than one administration of the TFP T cells per week (e.g., 2, 3 or 4 administrations per week) (also referred to herein as a cycle), followed by a week of no TFP T cells administrations, and then one or more additional administration of the TFP T cells (e.g., more than one administration of the TFP T cells per week) is administered to the subject. In another embodiment, the subject (e.g., human subject) receives more than one cycle of TFP T cells, and the time between each cycle is less than 10, 9, 8, 7, 6, 5, 4, or 3 days. In one embodiment, the TFP T cells are administered every other day for 3 administrations per week. In one embodiment, the TFP T cells of the present disclosure are administered for at least two, three, four, five, six, seven, eight or more weeks.

In one aspect, CD19 TFP T cells are generated using lentiviral viral vectors, such as lentivirus. TFP-T cells generated that way will have stable TFP expression.

In one aspect, TFP T cells transiently express TFP vectors for 4, 5, 6, 7, 8, 9, 10, 11, 12, 13, 14, 15 days after transduction. Transient expression of TFPs can be affected by RNA TFP vector delivery. In one aspect, the TFP RNA is transduced into the T cell by electroporation.

A potential issue that can arise in patients being treated using transiently expressing TFP T cells (particularly with murine scFv bearing TFP T cells) is anaphylaxis after multiple treatments.

Without being bound by this theory, it is believed that such an anaphylactic response might be caused by a patient developing humoral anti-TFP response, i.e., anti-TFP antibodies having an anti-IgE isotype. It is thought that a patient's antibody producing cells undergo a class switch from IgG isotype (that does not cause anaphylaxis) to IgE isotype when there is a ten to fourteen day break in exposure to antigen.

If a patient is at high risk of generating an anti-TFP antibody response during the course of transient TFP therapy (such as those generated by RNA transductions), TFP T cell infusion breaks should not last more than ten to fourteen days.

### Methods of Producing Modified T Cells

Disclosed herein, in some embodiments, are methods of producing the modified T cell of the disclosure, the method comprising (a) disrupting an endogenous TCR gene encoding a TCR alpha chain, a TCR beta chain, a TCR gamma chain, a TCR delta chain, or any combination thereof, thereby producing a T cell containing a functional disruption of an endogenous TCR gene; and (b) transducing the T cell containing a functional disruption of an endogenous TCR gene with the recombinant nucleic acid of the disclosure, or the vectors disclosed herein. In some instances, disrupting comprises transducing the T cell with a nuclease protein or a nucleic acid sequence encoding a nuclease protein that targets the endogenous gene encoding a TCR alpha chain, a TCR beta chain, or a TCR alpha chain and a TCR beta chain.

Further disclosed herein, in some embodiments, are methods of producing the modified T cell of the disclosure, the method comprising transducing a T cell containing a functional disruption of an endogenous TCR gene with the recombinant nucleic acid disclosed herein, or the vectors disclosed herein. In some instances, the T cell containing a functional disruption of an endogenous TCR gene is a T cell containing a functional disruption of an endogenous TCR gene encoding a TCR alpha chain, a TCR beta chain, or a TCR alpha chain and a TCR beta chain.

In some instances, the T cell is a human T cell. In some instances, the T cell containing a functional disruption of an endogenous TCR gene has reduced binding to MHC-peptide complex compared to that of an unmodified control T cell.

In some instances, the nuclease is a meganuclease, a zinc-finger nuclease (ZFN), a transcription activator-like effector nuclease (TALEN), a CRISPR/Cas nuclease, CRISPR/Cas nickase, or a megaTAL nuclease. In some instances, the sequence comprised by the recombinant nucleic acid or the vector is inserted into the endogenous TCR subunit gene at the cleavage site, and wherein the insertion of the sequence into the endogenous TCR subunit gene functionally disrupts the endogenous TCR subunit. In some instances, the nuclease is a meganuclease. In some instances, the meganuclease comprises a first subunit and a second subunit, wherein the first subunit binds to a first recognition half-site of the recognition sequence, and wherein the second subunit binds to a second recognition half-site of the recognition sequence. In some instances, the meganuclease is a single-chain meganuclease comprising a linker, wherein the linker covalently joins the first subunit and the second subunit.

### Gene Editing Technologies

In some embodiments, the modified T cells disclosed herein are engineered using a gene editing technique such as clustered regularly interspaced short palindromic repeats (CRISPR®, see, e.g., U.S. Pat. No. 8,697,359), transcription activator-like effector (TALE) nucleases (TALENs, see, e.g., U.S. Pat. No. 9,393,257), meganucleases (endodeoxyribonucleases having large recognition sites comprising double-stranded DNA sequences of 12 to 40 base pairs), zinc finger nuclease (ZFN, see, e.g., Urnov et al., Nat. Rev. Genetics (2010) v11, 636-646), or megaTAL nucleases (a fusion protein of a meganuclease to TAL repeats) methods. In this way, a chimeric construct may be engineered to combine desirable characteristics of each subunit, such as conformation or signaling capabilities. See also Sander & Joung, Nat. Biotech. (2014) v32, 347-55; and June et al., 2009 Nature Reviews Immunol. 9.10: 704-716, each incorporated herein by reference. In some embodiments, one or more of the extracellular domain, the transmembrane domain, or the cytoplasmic domain of a TFP subunit are engineered to have aspects of more than one natural TCR subunit domain (e.g., are chimeric).

Recent developments of technologies to permanently alter the human genome and to introduce site-specific genome modifications in disease relevant genes lay the foundation for therapeutic applications. These technologies are now commonly known as “genome editing.

The endogenous TCR gene encoding a TCR alpha chain, a TCR beta chain, or a TCR alpha chain and a TCR beta chain can be inactivated in the modified cell (e.g., modified T cell) described herein. The inactivation can include disruption of genomic gene locus, gene silencing, inhibition or reduction of transcription, or inhibition or reduction of translation. The endogenous TCR gene can be silenced, for example, by inhibitory nucleic acids such as siRNA and shRNA. The translation of the endogenous TCR gene can be inhibited by inhibitory nucleic acids such as microRNA. In some embodiments, gene editing techniques are employed to disrupt an endogenous TCR gene. In some embodiments, mentioned endogenous TCR gene encodes a TCR alpha chain, a TCR beta chain, or a TCR alpha chain and a TCR beta chain. In some embodiments, gene editing techniques pave the way for multiplex genomic editing, which allows simultaneous disruption of multiple genomic loci in endogenous TCR gene. In some embodiments, multiplex genomic editing techniques are applied to generate gene-disrupted T cells that are deficient in the expression of endogenous TCR, and/or human leukocyte antigens (HLAs), and/or programmed cell death protein 1 (PD-1), and/or other genes.

Current gene editing technologies comprise meganucleases, zinc-finger nucleases (ZFN), TAL effector nucleases (TALEN), and clustered regularly interspaced short palindromic repeats (CRISPR)/CRISPR-associated (Cas) system. These four major classes of gene-editing techniques share a common mode of action in binding a user-defined sequence of DNA and mediating a double-stranded DNA break (DSB). DSB may then be repaired by either non-homologous end joining (NHEJ) or—when donor DNA is present—homologous recombination (HR), an event that introduces the homologous sequence from a donor DNA fragment. Additionally, nickase nucleases generate single-stranded DNA breaks (SSB). DSBs may be repaired by single strand DNA incorporation (ssDI) or single strand template repair (ssTR), an event that introduces the homologous sequence from a donor DNA.

Genetic modification of genomic DNA can be performed using site-specific, rare-cutting endonucleases that are engineered to recognize DNA sequences in the locus of interest. Methods for producing engineered, site-specific endonucleases are known in the art. For example, zinc-finger nucleases (ZFNs) can be engineered to recognize and cut predetermined sites in a genome. ZFNs are chimeric proteins comprising a zinc finger DNA-binding domain fused to the nuclease domain of the Fok1 restriction enzyme. The zinc finger domain can be redesigned through rational or experimental means to produce a protein that binds to a pre-determined DNA sequence −18 base pairs in length. By fusing this engineered protein domain to the Fok1 nuclease, it is possible to target DNA breaks with genome-level specificity. ZFNs have been used extensively to target gene addition, removal, and substitution in a wide range of eukaryotic organisms (reviewed in Durai et al. (2005), Nucleic Acids Res 33, 5978). Likewise, TAL-effector nucleases (TALENs) can be generated to cleave specific sites in genomic DNA. Like a ZFN, a TALEN comprises an engineered, site-specific DNA-binding domain fused to the Fok1 nuclease domain (reviewed in Mak et al. (2013), Curr Opin Struct Biol. 23:93-9). In this case, however, the DNA binding domain comprises a tandem array of TAL-effector domains, each of which specifically recognizes a single DNA base pair. Compact TALENs have an alternative endonuclease architecture that avoids the need for dimerization (Beurdeley et al. (2013), Nat Commun. 4: 1762). A Compact TALEN comprises an engineered, site-specific TAL-effector DNA-binding domain fused to the nuclease domain from the I-TevI homing endonuclease. Unlike Fok1, I-TevI does not need to dimerize to produce a double-strand DNA break so a Compact TALEN is functional as a monomer.

Engineered endonucleases based on the CRISPR/Cas9 system are also known in the art (Ran et al. (2013), Nat Protoc. 8:2281-2308; Mali et al. (2013), Nat Methods 10:957-63). The CRISPR gene-editing technology is composed of an endonuclease protein whose DNA-targeting specificity and cutting activity can be programmed by a short guide RNA or a duplex crRNA/TracrRNA. A CRISPR endonuclease comprises two components: (1) a caspase effector nuclease, typically microbial Cas9; and (2) a short “guide RNA” or an RNA duplex comprising an 18 to 20 nucleotide targeting sequence that directs the nuclease to a location of interest in the genome. By expressing multiple guide RNAs in the same cell, each having a different targeting sequence, it is possible to target DNA breaks simultaneously to multiple sites in the genome (multiplex genomic editing).

There are two classes of CRISPR systems known in the art (Adli (2018) Nat. Commun. 9:1911), each containing multiple CRISPR types. Class 1 contains type I and type III CRISPR systems that are commonly found in Archaea. And, Class II contains type II, IV, V, and VI CRISPR systems. Although the most widely used CRISPR/Cas system is the type II CRISPR-Cas9 system, CRISPR/Cas systems have been repurposed by researchers for genome editing. More than 10 different CRISPR/Cas proteins have been remodeled within last few years (Adli (2018) Nat. Commun. 9:1911). Among these, such as Cas12a (Cpf1) proteins from Acid-aminococcus sp (AsCpf1) and Lachnospiraceae bacterium (LbCpf1), are particularly interesting.

Homing endonucleases are a group of naturally-occurring nucleases that recognize 15-40 base-pair cleavage sites commonly found in the genomes of plants and fungi. They are frequently associated with parasitic DNA elements, such as group 1 self-splicing introns and inteins. They naturally promote homologous recombination or gene insertion at specific locations in the host genome by producing a double-stranded break in the chromosome, which recruits the cellular DNA-repair machinery (Stoddard (2006), Q. Rev. Biophys. 38: 49-95). Specific amino acid substations could reprogram DNA cleavage specificity of homing nucleases (Niyonzima (2017), Protein Eng Des Sel. 30(7): 503-522). Meganucleases (MN) are monomeric proteins with innate nuclease activity that are derived from bacterial homing endonucleases and engineered for a unique target site (Gersbach (2016), Molecular Therapy. 24: 430-446). In some embodiments, meganuclease is engineered I-CreI homing endonuclease. In other embodiments, meganuclease is engineered I-SceI homing endonuclease.

In addition to mentioned four major gene editing technologies, chimeric proteins comprising fusions of meganucleases, ZFNs, and TALENs have been engineered to generate novel monomeric enzymes that take advantage of the binding affinity of ZFNs and TALENs and the cleavage specificity of meganucleases (Gersbach (2016), Molecular Therapy 24: 430-446). For example, A megaTAL is a single chimeric protein, which is the combination of the easy-to-tailor DNA binding domains from TALENs with the high cleavage efficiency of meganucleases.

In order to perform the gene editing technique, the nucleases, and in the case of the CRISPR/Cas9 system, a gRNA, may need to be efficiently delivered to the cells of interest. Delivery methods such as physical, chemical, and viral methods are also know in the art (Mali (2013). Indian J. Hum. Genet. 19: 3-8.). In some instances, physical delivery methods can be selected from the methods but not limited to electroporation, microinjection, or use of ballistic particles. On the other hand, chemical delivery methods require use of complex molecules such calcium phosphate, lipid, or protein. In some embodiments, viral delivery methods are applied for gene editing techniques using viruses such as but not limited to adenovirus, lentivirus, and retrovirus.

As an example, the endogenous TCR gene (e.g., a TRAC locus or a TRBC locus) encoding a TCR alpha chain, a TCR beta chain, or a TCR alpha chain and a TCR beta chain can be inactivated by CRISPR/Cas9 system. The gRNA used to inactivate (e.g., disrupt) the TRAC locus can comprise a sequence of SEQ ID: 406. The gRNA used to disrupt the TRBC locus can comprise a sequence of SEQ ID: 197.

(SEQ ID NO: 406)
CTCGACCAGCTTGACATCAC.
(SEQ ID NO: 197)
ACACTGGTGTGCCTGGCCAC.

### Methods of Treatment

Disclosed herein, in some embodiments, is a method of treating a disease or a condition in a subject in need thereof, comprising administering to the subject a therapeutically effective amount of the pharmaceutical compositions described herein. Further disclosed herein, in some embodiments, are methods of treating a disease or a condition in a subject in need thereof, the method comprising administering to the subject a therapeutically effective amount of a pharmaceutical composition comprising (a) a cell produced according to the methods disclosed herein; and (b) a pharmaceutically acceptable carrier. In some embodiments, the disease or the condition is a cancer or a disease or a condition associated with expression of CD19, B-cell maturation antigen (BCMA), mesothelin (MSLN), CD20, CD70, MUC16, Trop-2, Nectin-4, or GPC3. In some embodiments, the cancer is a hematologic cancer. Examples of a hematologic cancer include, but are not limited to, B-cell acute lymphoid leukemia (B-ALL), T cell acute lymphoid leukemia (T-ALL), acute lymphoblastic leukemia (ALL), chronic myelogenous leukemia (CML), chronic lymphocytic leukemia (CLL), B cell prolymphocytic leukemia, blastic plasmacytoid dendritic cell neoplasm, Burkitt's lymphoma, diffuse large B cell lymphoma, follicular lymphoma, hairy cell leukemia, small cell-follicular lymphoma, large cell-follicular lymphoma, malignant lymphoproliferative conditions, MALT lymphoma, mantle cell lymphoma, Marginal zone lymphoma, multiple myeloma, myelodysplasia, myelodysplastic syndrome, non-Hodgkin's lymphoma, plasmablastic lymphoma, plasmacytoid dendritic cell neoplasm, Waldenstrom macroglobulinemia, and preleukemia. In some embodiments, the subject is a mammal. In some embodiments, the subject is a human.

Disclosed herein, in some embodiments, are methods of increasing the activity or persistence of a cell expressing a recombinant nucleic acid molecule comprising a sequence encoding the TFP disclosed herein, the method comprising expressing an interleukin-15 (IL-15) polypeptide or a fragment thereof in the cell. Further disclosed herein, in some embodiments, are methods of increasing the activity or persistence of a cell expressing a recombinant nucleic acid molecule comprising a sequence encoding the TFP disclosed herein, the method comprising expressing an interleukin-15 receptor alpha (IL-15Rα) polypeptide or a fragment thereof in the cell. In some embodiments, the cell is any one of cells described herein.

Disclosed herein, in some embodiments, are methods of treating cancer in a subject in need thereof, the method comprising administering to the subject a therapeutically effective amount of the pharmaceutical compositions disclosed herein. Further disclosed herein, in some embodiments, are methods of treating cancer in a subject in need thereof, the method comprising administering to the subject a pharmaceutical composition comprising (a) a modified T cell produced according to the methods disclosed herein; and (b) a pharmaceutically acceptable carrier.

In some instances, the modified T cell is an autologous T cell. In some embodiments, the T cell is an allogeneic T cell. In some instances, less cytokines are released in the subject compared a subject administered an effective amount of an unmodified control T cell. In some instances, less cytokines are released in the subject compared a subject administered an effective amount of a modified T cell comprising the recombinant nucleic acid disclosed herein, or the vector disclosed herein.

In some instances, the method comprises administering the pharmaceutical composition in combination with an agent that increases the efficacy of the pharmaceutical composition. In some instances, the method comprises administering the pharmaceutical composition in combination with an agent that ameliorates one or more side effects associated with the pharmaceutical composition.

In some instances, the cancer is a solid cancer, a lymphoma or a leukemia. In some instances, the cancer is selected from the group consisting of renal cell carcinoma, breast cancer, lung cancer, ovarian cancer, prostate cancer, colon cancer, cervical cancer, brain cancer, liver cancer, pancreatic cancer, kidney and stomach cancer.

The present disclosure includes a type of cellular therapy where T cells are genetically modified to express a TFP and an IL-15 and/or IL-15Rα and the modified T cell is infused to a recipient in need thereof. The infused cell is able to kill tumor cells in the recipient. Unlike antibody therapies, modified T cells are able to replicate in vivo resulting in long-term persistence that can lead to sustained tumor control. In various aspects, the T cells administered to the patient, or their progeny, persist in the patient for at least four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the T cell to the patient.

The present disclosure also includes a type of cellular therapy where T cells are modified, e.g., by in vitro transcribed RNA, to transiently express a TFP and an IL-15 and/or IL-15Rα and the modified T cell is infused to a recipient in need thereof. The infused cell is able to kill tumor cells in the recipient. Thus, in various aspects, the T cells administered to the patient, is present for less than one month, e.g., three weeks, two weeks, or one week, after administration of the T cell to the patient.

Without wishing to be bound by any particular theory, the anti-tumor immunity response elicited by the modified T cells may be an active or a passive immune response, or alternatively may be due to a direct vs indirect immune response.

In one aspect, the human modified T cells of the disclosure may be a type of vaccine for ex vivo immunization and/or in vivo therapy in a mammal. In one aspect, the mammal is a human.

With respect to ex vivo immunization, at least one of the following occurs in vitro prior to administering the cell into a mammal: i) expansion of the cells, ii) introducing a nucleic acid encoding a TFP and an IL-15 and/or IL-15Rα to the cells or iii) cryopreservation of the cells.

Ex vivo procedures are well known in the art and are discussed more fully below. Briefly, cells are isolated from a mammal (e.g., a human) and genetically modified (e.g., transduced or transfected in vitro) with a vector disclosed herein. The modified T cell can be administered to a mammalian recipient to provide a therapeutic benefit. The mammalian recipient may be a human and the modified cell can be autologous with respect to the recipient. Alternatively, the cells can be allogeneic, syngeneic or xenogeneic with respect to the recipient.

The procedure for ex vivo expansion of hematopoietic stem and progenitor cells is described in U.S. Pat. No. 5,199,942, incorporated herein by reference, can be applied to the cells of the present disclosure. Other suitable methods are known in the art; therefore, the present disclosure is not limited to any particular method of ex vivo expansion of the cells. Briefly, ex vivo culture and expansion of T cells comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

In addition to using a cell-based vaccine in terms of ex vivo immunization, the present disclosure also provides compositions and methods for in vivo immunization to elicit an immune response directed against an antigen in a patient.

Generally, the cells activated and expanded as described herein may be utilized in the treatment and prevention of diseases that arise in individuals who are immunocompromised.

The modified T cells of the present disclosure may be administered either alone, or as a pharmaceutical composition in combination with diluents and/or with other components such as IL-2 or other cytokines or cell populations.

### Combination Therapies

A modified T cell described herein may be used in combination with other known agents and therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In some embodiments, the “at least one additional therapeutic agent” includes a modified T cell. Also provided are T cells that express multiple TFPs, which bind to the same or different target antigens, or same or different epitopes on the same target antigen.

A modified T cell described herein and the at least one additional therapeutic agent can be administered simultaneously, in the same or in separate compositions, or sequentially. For sequential administration, the modified T cell described herein can be administered first, and the additional agent can be administered second, or the order of administration can be reversed.

In further aspects, a modified T cell described herein may be used in a treatment regimen in combination with surgery, chemotherapy, radiation, immunosuppressive agents, such as cyclosporin, azathioprine, methotrexate, mycophenolate, and tacrolimus, antibodies, or other immunoablative agents such as alemtuzumab, anti-CD3 antibodies or other antibody therapies, cytoxin, fludarabine, cyclosporin, tacrolimus, rapamycin, mycophenolic acid, steroids, romidepsin, cytokines, and irradiation. peptide vaccine, such as that described in Izumoto et al., 2008 J. Neurosurg. 108:963-971.

In one embodiment, the subject can be administered an agent which reduces or ameliorates a side effect associated with the administration of a modified T cell. Side effects associated with the administration of a modified T cell include but are not limited to cytokine release syndrome (CRS), and hemophagocytic lymphohistiocytosis (HLH), also termed Macrophage Activation Syndrome (MAS). Symptoms of CRS include high fevers, nausea, transient hypotension, hypoxia, and the like. Accordingly, the methods disclosed herein can comprise administering a modified T cell described herein to a subject and further administering an agent to manage elevated levels of a soluble factor resulting from treatment with a modified T cell. In one embodiment, the soluble factor elevated in the subject is one or more of IFN-γ, TNFα, IL-2 and IL-6. Therefore, an agent administered to treat this side effect can be an agent that neutralizes one or more of these soluble factors. Such agents include, but are not limited to a steroid, an inhibitor of TNFα, and an inhibitor of IL-6. An example of a TNFα inhibitor is entanercept. An example of an IL-6 inhibitor is tocilizumab (toc).

In one embodiment, the subject can be administered an agent which enhances the activity of a modified T cell. For example, in one embodiment, the agent can be an agent which inhibits an inhibitory molecule. Inhibitory molecules, e.g., Programmed Death 1 (PD-1), can, in some embodiments, decrease the ability of a modified T cell to mount an immune effector response. Examples of inhibitory molecules include PD-1, PD-L1, CTLA4, TIM3, LAG3, VISTA, BTLA, TIGIT, LAIR1, CD160, 2B4 and TGFR beta. Inhibition of an inhibitory molecule, e.g., by inhibition at the DNA, RNA or protein level, can optimize a modified T cell performance. In embodiments, an inhibitory nucleic acid, e.g., an inhibitory nucleic acid, e.g., a dsRNA, e.g., an siRNA or shRNA, can be used to inhibit expression of an inhibitory molecule in the TFP-expressing cell. In an embodiment the inhibitor is a shRNA. In an embodiment, the inhibitory molecule is inhibited within a modified T cell. In these embodiments, a dsRNA molecule that inhibits expression of the inhibitory molecule is linked to the nucleic acid that encodes a component, e.g., all of the components, of the TFP. In one embodiment, the inhibitor of an inhibitory signal can be, e.g., an antibody or antibody fragment that binds to an inhibitory molecule. For example, the agent can be an antibody or antibody fragment that binds to PD-1, PD-L1, PD-L2 or CTLA4 (e.g., ipilimumab (also referred to as MDX-010 and MDX-101 and marketed as Yervoy®; Bristol-Myers Squibb; tremelimumab (IgG2 monoclonal antibody available from Pfizer, formerly known as ticilimumab, CP-675,206)). In an embodiment, the agent is an antibody or antibody fragment that binds to TIM3. In an embodiment, the agent is an antibody or antibody fragment that binds to LAG3.

In some embodiments, the agent which enhances the activity of a modified T cell can be, e.g., a fusion protein comprising a first domain and a second domain, wherein the first domain is an inhibitory molecule, or fragment thereof, and the second domain is a polypeptide that is associated with a positive signal, e.g., a polypeptide comprising an intracellular signaling domain as described herein. In some embodiments, the polypeptide that is associated with a positive signal can include a costimulatory domain of CD28, CD27, ICOS, e.g., an intracellular signaling domain of CD28, CD27 and/or ICOS, and/or a primary signaling domain, e.g., of CD3 zeta, e.g., described herein. In one embodiment, the fusion protein is expressed by the same cell that expressed the TFP. In another embodiment, the fusion protein is expressed by a cell, e.g., a T cell that does not express an anti-TAA TFP.

### EXAMPLES

### Example 1. TFP Constructs

TFP constructs can be generated as previously described. An anti-MSLN or CD19 binder can be linked to a CD3 or TCR DNA fragment by either a DNA sequence encoding a short linker (SL): AAAGGGGSGGGGSGGGGSLE (SEQ ID NO: 387) or a long linker (LL): AAAIEVMYPPPYLGGGGSGGGGSGGGGSLE (SEQ ID NO: 388) into pLRPO or p510 vector. In some embodiments, the TFP used is TC-210 (e.g., an anti-MSLN MH1e VHH antibody linked to CD3 epsilon) having the sequence of SEQ ID NO: 195. In some embodiments, the TFP used is TC-110 (e.g., an anti-CD19 FMC63 scFv antibody linked to CD3 epsilon) having the sequence of SEQ ID NO: 196.

### Source of TCR Subunits

A TCR complex contains the CD3-epsilon polypeptide, the CD3-gamma poly peptide, the CD3-delta polypeptide, and the TCR alpha chain polypeptide and the TCR beta chain polypeptide or the TCR delta chain polypeptide and the TCR gamma chain polypeptide. TCR alpha, TCR beta, TCR gamma, and TCR delta recruit the CD3 zeta polypeptide. The human CD3-epsilon polypeptide canonical sequence is Uniprot Accession No. P07766. The human CD3-gamma polypeptide canonical sequence is Uniprot Accession No. P09693. The human CD3-delta polypeptide canonical sequence is Uniprot Accession No. P043234. The human CD3-zeta polypeptide canonical sequence is Uniprot Accession No. P20963. The human TCR alpha chain canonical sequence is Uniprot Accession No. Q6ISU1. The murine TCR alpha chain canonical sequence is Uniprot Accession No. A0A075B662. The human TCR beta chain constant region canonical sequence is Uniprot Accession No. P01850. The murine TCR beta chain constant region canonical sequence is Uniprot Accession No. P01852.

The human CD3-epsilon polypeptide canonical sequence is:

(SEQ ID NO: 124)
MQSGTHWRVLGLCLLSVGVWGQDGNEEMGGITQTPYKVSISGTTVILTCP
QYPGSEILWQHNDKNIGGDEDDKNIGSDEDHLSLKEFSELEQSGYYVCYP
RGSKPEDANFYLYLRARVCENCMEMDVMSVATIVIVDICITGGLLLLVYY
WSKNRKAKAKPVTRGAGAGGRQRGQNKERPPPVPNPDYEPIRKGQRDLYS
GLNQRRI.

The mature human CD3-epsilon polypeptide sequence is:

(SEQ ID NO: 364)
DGNEEMGGITQTPYKVSISGTTVILTCPQYPGSEILWQHNDKNIGGDEDD
KNIGSDEDHLSLKEFSELEQSGYYVCYPRGSKPEDANFYLYLRARVCENC
MEMDVMSVATIVIVDICITGGLLLLVYYWSKNRKAKAKPVTRGAGAGGRQ
RGQNKERPPPVPNPDYEPIRKGQRDLYSGLNQRRI.

The signal peptide of human CD3ε is: MQSGTHWRVLGLCLLSVGVWGQ (SEQ ID NO: 125).

The extracellular domain of human CD3ε is:

(SEQ ID NO: 126)
DGNEEMGGITQTPYKVSISGTTVILTCPQYPGSEILWQHNDKNIGGDEDD
KNIGSDEDHLSLKEFSELEQSGYYVCYPRGSKPEDANFYLYLRARVCENC
MEMD.

The transmembrane domain of human CD3ε is: VMSVATIVIVDICITGGLLLLVYYWS (SEQ ID NO: 127).

The intracellular domain of human CD3ε is:

(SEQ ID NO: 128)
KNRKAKAKPVTRGAGAGGRQRGQNKERPPPVPNPDYEPIRKGQRDLYSGL
NQRRI.

The human CD3-gamma polypeptide canonical sequence is:

(SEQ ID NO: 129)
MEQGKGLAVLILAIILLQGTLAQSIKGNHLVKVYDYQEDGSVLLTCDAEA
KNITWFKDGKMIGFLTEDKKKWNLGSNAKDPRGMYQCKGSQNKSKPLQVY
YRMCQNCIELNAATISGFLFAEIVSIFVLAVGVYFIAGQDGVRQSRASDK
QTLLPNDQLYQPLKDREDDQYSHLQGNQLRRN.

The mature human CD3-gamma polypeptide sequence is:

(SEQ ID NO: 130)
QSIKGNHLVKVYDYQEDGSVLLTCDAEAKNITWFKDGKMIGFLTEDKKKW
NLGSNAKDPRGMYQCKGSQNKSKPLQVYYRMCQNCIELNAATISGFLFAE
IVSIFVLAVGVYFIAGQDGVRQSRASDKQTLLPNDQLYQPLKDREDDQYS
HLQGNQLRRN.

The signal peptide of human CD3γ is: MEQGKGLAVLILAIILLQGTLA (SEQ ID NO:131).

The extracellular domain of human CD3γ is:

(SEQ ID NO: 132)
QSIKGNHLVKVYDYQEDGSVLLTCDAEAKNITWFKDGKMIGFLTEDKKK
WNLGSNAKDPRGMYQCKGSQNKSKPLQVYYRMCQNCIELNAATIS.

The transmembrane domain of human CD3γ is: GFLFAEIVSIFVLAVGVYFIA (SEQ ID NO: 133).

The intracellular domain of human CD3γ is:

(SEQ ID NO: 134)
GQDGVRQSRASDKQTLLPNDQLYQPLKDREDDQYSHLQGNQLRRN.

The human CD3-delta polypeptide canonical sequence is:

(SEQ ID NO: 135)
MEHSTFLSGLVLATLLSQVSPFKIPIEELEDRVFVNCNTSITWVEGTVG
TLLSDITRLDLGKRILDPRGIYRCNGTDIYKDKESTVQVHYRMCQSCVE
LDPATVAGIIVTDVIATLLLALGVFCFAGHETGRLSGAADTQALLRNDQ
VYQPLRDRDDAQYSHLGGNWARNKS.

The mature human CD3-delta polypeptide sequence is:

(SEQ ID NO: 136)
FKIPIEELEDRVFVNCNTSITWVEGTVGTLLSDITRLDLGKRILDPRGI
YRCNGTDIYKDKESTVQVHYRMCQSCVELDPATVAGIIVTDVIATLLLA
LGVFCFAGHETGRLSGAADTQALLRNDQVYQPLRDRDDAQYSHLGGNWA
RNKS.

The signal peptide of human CD3δ is: MEHSTFLSGLVLATLLSQVSP (SEQ ID NO:137).

The extracellular domain of human CD3δ is:

(SEQ ID NO: 138)
FKIPIEELEDRVFVNCNTSITWVEGTVGTLLSDITRLDLGKRILDPRGI
YRCNGTDIYKDKESTVQVHYRMCQSCVELDPATVA.

The transmembrane domain of human CD3δ is: GIIVTDVIATLLLALGVFCFA (SEQ ID NO: 139).

The intracellular domain of human CD3δ is:

(SEQ ID NO: 140)
GHETGRLSGAADTQALLRNDQVYQPLRDRDDAQYSHLGGNWARNK.

The human CD3-zeta polypeptide canonical sequence is:

(SEQ ID NO: 141)
MKWKALFTAAILQAQLPITEAQSFGLLDPKLCYLLDGILFIYGVILTAL
FLRVKFSRSADAPAYQQGQNQLYNELNLGRREEYDVLDKRRGRDPEMGG
KPQRRKNPQEGLYNELQKDKMAEAYSEIGMKGERRRGKGHDGLYQGLST
ATKDTYDALHMQALPPR.

The human TCR alpha chain constant region canonical sequence is:

(SEQ ID NO: 142)
IQNPDPAVYQLRDSKSSDKSVCLFTDFDSQTNVSQSKDSDVYITDKTV
LDMRSMDFKSNSAVAWSNKSDFACANAFNNSIIPEDTFFPSPESSCDV
KLVEKSFETDTNLNFQNLSVIGFRILLLKVAGFNLLMTLRLWSS.

The human TCR alpha chain human IgC sequence is:

(SEQ ID NO: 143)
IQNPDPAVYQLRDSKSSDKSVCLFTDFDSQTNVSQSKDSDVYITDKTV
LDMRSMDFKSNSAVAWSNKSDFACANAFNNSIIPEDTFFPSPESSCDVK
LVEKSFETDTNLNFQNLS

The transmembrane domain of the human TCR alpha chain is:

(SEQ ID NO: 144)
VIGFRILLLKVAGFNLLMTLRLW.

The intracellular domain of the human TCR alpha chain is: SS (SEQ ID NO: 145)

The murine TCR alpha chain constant (mTRAC) region canonical sequence is:

(SEQ ID NO: 146)
XIQNPEPAVYQLKDPRSQDSTLCLFTDFDSQINVPKTMESGTFITDKTV
LDMKAMDSKSNGAIAWSNQTSFTCQDIFKETNATYPSSDVPCDATLTEK
SFETDMNLNFQNLSVMGLRILLLKVAGFNLLMTLRLWSS.

The transmembrane domain of the murine TCR alpha chain is: MGLRILLLKVAGFNLLMTLRLW (SEQ ID NO: 147).

The intracellular domain of the murine TCR alpha chain is: SS (SEQ ID NO: 145)

The human TCR beta chain constant region (mTRBC) canonical sequence is:

(SEQ ID NO: 148)
EDLNKVFPPEVAVFEPSEAEISHTQKATLVCLATGFFPDHVELSWWVNG
KEVHSGVSTDPQPLKEQPALNDSRYCLSSRLRVSATFWQNPRNHFRCQV
QFYGLSENDEWTQDRAKPVTQIVSAEAWGRADCGFTSVSYQQGVLSATI
LYEILLGKATLYAVLVSALVLMAMVKRKDF.

The human TCR beta chain human IgC sequence is:

(SEQ ID NO: 149)
EDLNKVFPPEVAVFEPSEAEISHTQKATLVCLATGFFPDHVELSWWVNG
KEVHSGVSTDPQPLKEQPALNDSRYCLSSRLRVSATFWQNPRNHFRCQV
QFYGLSENDEWTQDRAKPVTQIVSAEAWGRADCGFTSVSYQQGVLSATI
LYE

The transmembrane domain of the human TCR beta chain is: ILLGKATLYAVLVSALVLMAM (SEQ ID NO: 150).

The intracellular domain of the human TCR beta chain is: VKRKDF (SEQ ID NO: 151)

The murine TCR beta chain constant region canonical sequence is:

(SEQ ID NO: 152)
EDLRNVTPPKVSLFEPSKAEIANKQKATLVCLARGFFPDHVELSWWVNG
KEVHSGVSTDPQAYKESNYSYCLSSRLRVSATFWHNPRNHFRCQVQFHG
LSEEDKWPEGSPKPVTQNISAEAWGRADCGITSASYQQGVLSATILYEI
LLGKATLYAVLVSTLVVMAMVKRKNS.

The transmembrane domain of the murine TCR beta chain is:

(SEQ ID NO: 153)
ILYEILLGKATLYAVLVSTLVVMAMVK.

The intracellular domain of the murine TCR beta chain is: KRKNS (SEQ ID NO: 154)

The human TCR gamma chain constant region canonical sequence is:

(SEQ ID NO: 21)
DKQLDADVSPKPTIFLPSIAETKLQKAGTYLCLLEKFFPDVIKIHWQEK
KSNTILGSQEGNTMKTNDTYMKFSWLTVPEKSLDKEHRCIVRHENNKNG
VDQEIIFPPIKTDVITMDPKDNCSKDANDTLLLQLTNTSAYYMYLLLLL
KSVVYFAIITCCLLRRTAFCCNGEKS.

The human TCR gamma human IgC sequence is: PG,

(SEQ ID NO: 155)
DKQLDADVSPKPTIFLPSIAETKLQKAGTYLCLLEKFFPDVIKIHWQEK
KSNTILGSQEGNTMKTNDTYMKFSWLTVPEKSLDKEHRCIVRHENNKNG
VDQEIIFPPIKTDVITMDPKDNCSKDANDTLLLQLTNTSA

The transmembrane domain of the human TCR gamma chain is: YYMYLLLLLKSVVYFAIITCCLL (SEQ ID NO: 156).

The intracellular domain of the human TCR gamma chain is: RRTAFCCNGEKS (SEQ ID NO: 157)

The human TCR delta chain C region canonical sequence is:

(SEQ ID NO: 243)
SQPHTKPSVFVMKNGTNVACLVKEFYPKDIRINLVSSKKITEFDPAIVI
SPSGKYNAVKLGKYEDSNSVTCSVQHDNKTVHSTDFEVKTDSTDHVKPK
ETENTKQPSKSCHKPKAIVHTEKVNMMSLTVLGLRMLFAKTVAVNFLLT
AKLFFL.

The human TCR delta human IgC sequence is:

(SEQ ID NO: 265)
SQPHTKPSVFVMKNGTNVACLVKEFYPKDIRINLVSSKKITEFDPAIVI
SPSGKYNAVKLGKYEDSNSVTCSVQHDNKTVHSTDFEVKTDSTDHVKPK
ETENTKQPSKSCHKPKAIVHTEKVNMMSLTV

The transmembrane domain of the human TCR delta chain is:

(SEQ ID NO: 158)
LGLRMLFAKTVAVNFLLTAKLFF.

The intracellular domain of the human TCR delta chain is: L

### IL-15 Peptides and Other Peptides

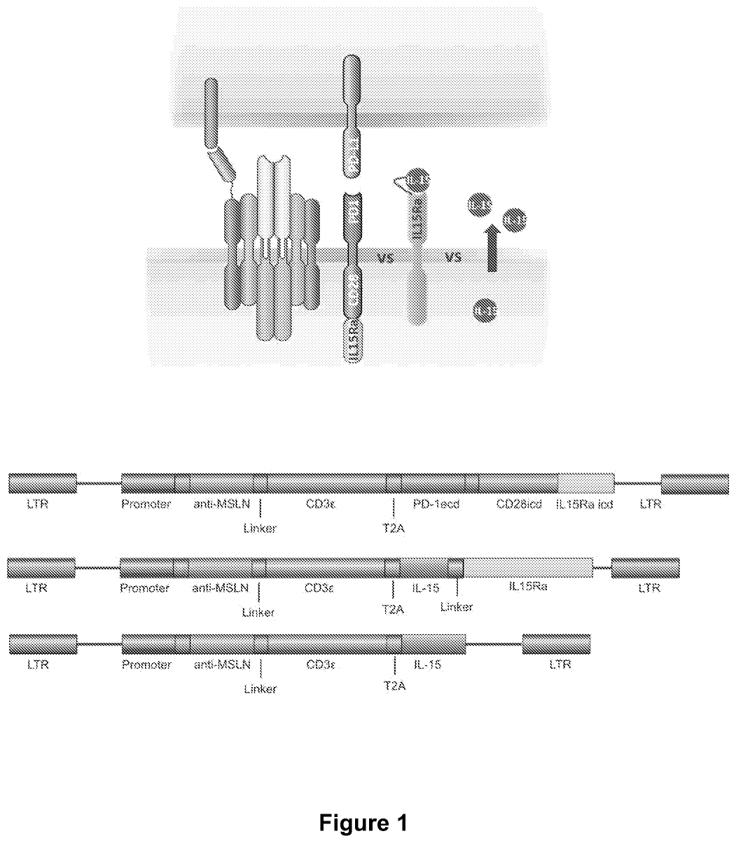

In some embodiments, TFP constructs are in a vector that further contains a sequence encoding an IL-15 peptide or an IL15-Rα peptide. The IL-15 may be encoded in the same open reading frame and separated by a self-cleaving peptide (e.g., a P2A or a T2A self-cleaving peptide). In some embodiments, the IL-15 peptide comprises a secreted IL-15. The secreted IL-15 can have the sequence of SEQ ID NO: 375. In some embodiments, the IL-15 peptide is an IL-15-IL15Rα fusion. In some embodiments, IL-15Rα comprises the sequence of SEQ ID NO: 383 or SEQ ID NO: 386. In some embodiments, the IL-15-IL15Rα fusion comprises a linker followed by a sushi domain linking IL-15 and IL-15Rα. In some embodiments, the IL-15-IL15Rα fusion comprises the sequence of SEQ ID NO: 389. In some embodiments, IL-15Rα peptide comprises the extracellular and transmembrane domain of PD-1. The extracellular and transmembrane domain of PD-1 can be fused to the intracellular domain of CD28. The IL-15Rα peptide can further comprise the intracellular domain of IL-15Rα fused to the C-terminus of CD28 (e.g., intracellular domain of CD28). In some embodiments, the PD-1-CD28-IL-15Rα fusion comprises the sequence of SEQ ID NO: 390. In some embodiments, the vector further contains a sequence encoding a PD-1-CD28 fusion protein. The fusion protein can have the transmembrane domain of PD-1. In some embodiment, the PD-1-CD28 fusion protein comprises the sequence of SEQ ID NO: 391. A schematic of the constructs used in this study is shown in FIG. 1.

IL-15-IL15Rα fusion:
(SEQ ID NO: 389)
GIHVFILGCFSAGLPKTEANWVNVISDLKKIEDLIQSMHIDATLYTESD
VHPSCKVTAMKCFLLELQVISLESGDASIHDTVENLIILANNSLSSNGN
VTESGCKECEELEEKNIKEFLQSFVHIVQMFINTSSGGGSGGGGSGGGG
SGGGGSGGGSLQITCPPPMSVEHADIWVKSYSLYSRERYICNSGFKRKA
GTSSLTECVLNKATNVAHWTTPSLKCIRDPALVHQRPAPPSTVTTAGVT
PQPESLSPSGKEPAASSPSSNNTAATTAAIVPGSQLMPSKSPSTGTTEI
SSHESSHGTPSQTTAKNWELTASASHQPPGVYPQGHSDTTVAISTSTVL
LCGLSAVSLLACYLKSRQTPPLASVEMEAMEALPVTWGTSSRDEDLENC
SHHL.
PD-1-CD28-IL-15Rα fusion:
(SEQ ID NO: 390)
PGWFLDSPDRPWNPPTFSPALLVVTEGDNATFTCSFSNTSESFVLNWYR
MSPSNQTDKLAAFPEDRSQPGQDCRFRVTQLPNGRDFHMSVVRARRNDS
GTYLCGAISLAPKAQIKESLRAELRVTERRAEVPTAHPSPSPRPAGQFQ
TLVVGVVGGLLGSLVLLVWVLAVIRSKRSRLLHSDYMNMTPRRPGPTRK
HYQPYAPPRDFAAYRSKSRQTPPLASVEMEAMEALPVTWGTSSRDEDLE
NCSHHL.
PD-1-CD28 fusion:
(SEQ ID NO: 391)
PGWFLDSPDRPWNPPTFSPALLVVTEGDNATFTCSFSNTSESFVLNWYR
MSPSNQTDKLAAFPEDRSQPGQDCRFRVTQLPNGRDFHMSVVRARRNDS
GTYLCGAISLAPKAQIKESLRAELRVTERRAEVPTAHPSPSPRPAGQFQ
TLVVGVVGGLLGSLVLLVWVLAVIRSKRSRLLHSDYMNMTPRRPGPTRK
HYQPYAPPRDFAAYRS.

### TFP Expression Vectors

Expression vectors are provided that include: a promoter (e.g., an EF1a promoter), a signal sequence to enable secretion, a polyadenylation signal and transcription terminator (Bovine Growth Hormone (BGH) gene), an element allowing episomal replication and replication in prokaryotes (e.g., SV40 origin and ColE1 or others known in the art) and elements to allow selection (ampicillin resistance gene and zeocin marker).

Preferably, the TFP-encoding nucleic acid construct with or without an IL-15/IL15Rα peptide and/or PD-1-CD28 fusion protein is cloned into a lentiviral expression vector and expression validated based on the quantity and quality of the effector T cell response of transduced T cells in response to MSLN+ target cells. Effector T cell responses include, but are not limited to, cellular expansion, proliferation, doubling, cytokine production and target cell lysis or cytolytic activity (e.g., degranulation).

### Example 2: Generation of T Cell Receptor Fusion Protein T Cells

T-Cell Activation, Transduction, and Expansion

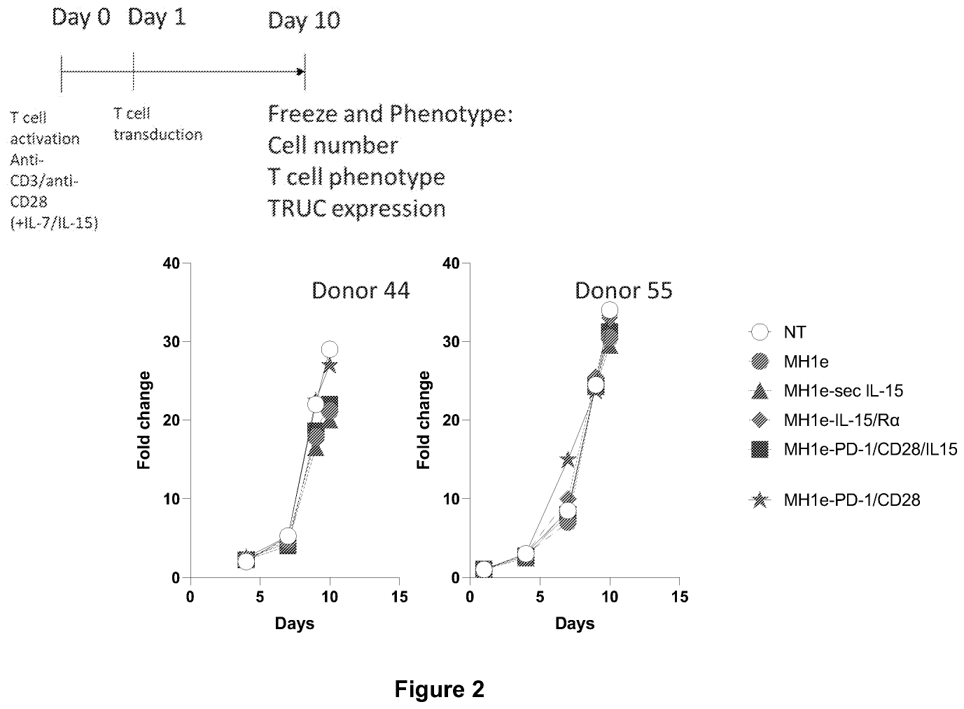

T cells were purified from healthy donor leukopak or PBMCs via positive selection of CD4+ and CD8+ T cells with CD4 and CD8 microbeads from Miltenyi Biotech. On day 0, T cells, freshly isolated or thawed from previously prepared frozen vials, were activated by MACS GMP T cell TransAct (Miltenyi Biotech), in the presence of human IL-7 and IL-15 (both from Miltenyi Biotech, premium grade). On day 1, activated T cells were transduced with lentivirus encoding the TFP. T cells were transduced with vectors expressing anti-MSLN TFP (referred to as MH1e, MH1e TFP, or TC-210), or the anti-MSLN TFP expressing secreted IL-15, an IL-15-IL15-Rα fusion, a PD-1-CD28-IL15-Rα fusion, or a PD-1-CD28 fusion described above. On day 4, the cells were washed, subcultured in fresh medium with cytokines and then expanded up to day 10 by supplementing fresh medium every 2 days. At each day of subculture, fresh medium with cytokines were added to maintain the cell suspension at 1×10⁶ cells/mL. The expansion of T cells, and non-transduced controls, is shown in FIG. 2 for two donors (Donor 44 and Donor 55). No dramatic difference in expansion was observed between TFP and non-transduced T cells.

Verification of TFP Expression and Phenotyping of TFP-Expressing Cells by Cell Staining

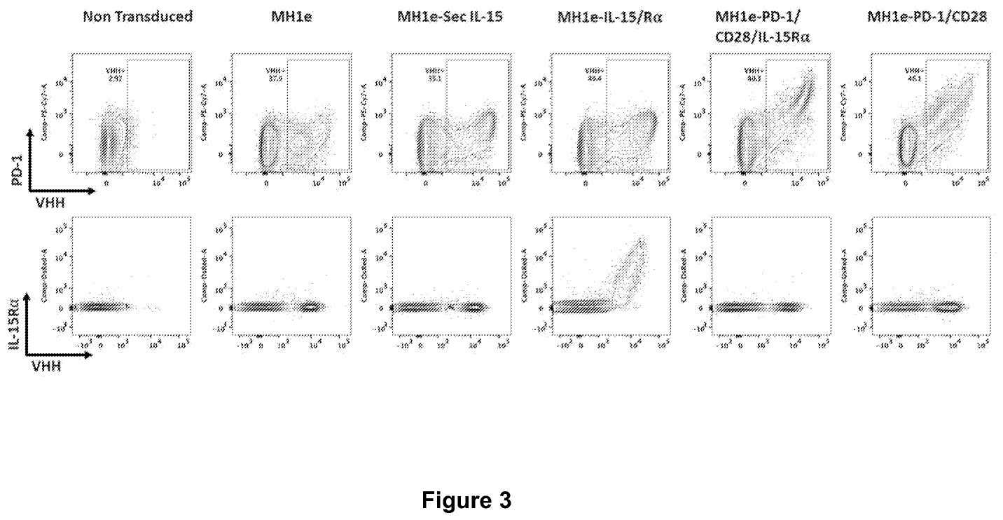

Following lentiviral transduction, expression of TFPs by transduced T cells was confirmed by flow cytometry, using an anti-VHH antibody, on day 10 of cell expansion. T cells were washed three times in PBS and then re-suspended in PBS at 1×10⁵ cells per well. For dead cell exclusion, cells were incubated with LIVE/DEAD® Fixable Aqua Dead Cell Stain (Invitrogen) for 30 minutes at 4° C. in the dark. Cells were then washed twice with PBS and re-suspended in 100 μL of staining buffer (PBS with 2% BSA). Cells were harvested, washed with PBS three times and blocked with human Fc block (BioLegend) for 10 minutes. Cells were then stained for 30 minutes at 4° C. in the dark. Cells were then washed twice with staining buffer (PBS with 2% BSA) and submitted to data acquisition on LSR Fortessa™-X20 (BD Biosciences) using FACS Diva software. The TFP expression was analyzed, with FlowJo® (BD Biosciences), for live T cells (CD3+ live cells). As is shown in FIG. 3, binding of the anti-VHH antibody was detected TFP-transduced T cells, but not in non-transduced control T cells from the same donors. The proportion of cells having cell surface staining for PD-1 and IL15-Rα was also measured. Cells expressing the TFP construct having the IL-15-IL15-Ra fusion showed cell surface staining of IL-15Rα, whereas cells expressing the TFP construct having the PD-1-CD28 fusion or the PD-1-CD28-IL15-Rα fusion showed cell surface staining for PD-1.

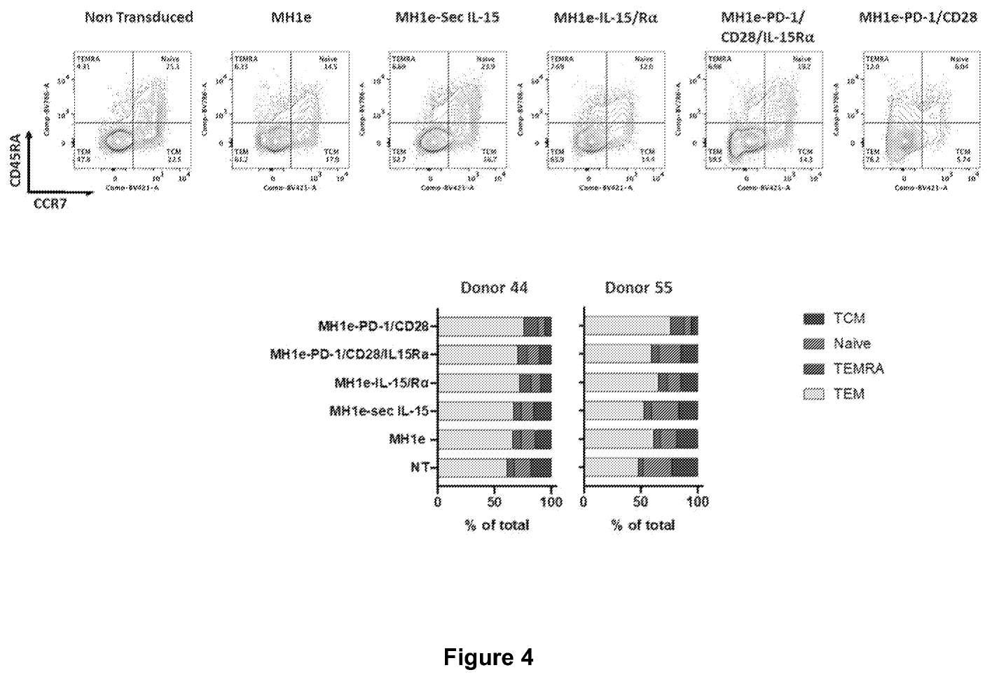

The memory status of the T cells was determined by flow cytometry to detect cell surface levels of CD45RA and CCR7 as is shown in FIG. 4. T cells expressing the MH1e TFP with secreted IL-15, the IL-15-IL15-Rα fusion or the PD-1-CD28-IL15-Rα fusion showed an increased naïve and central memory cells relative to cells expressing MH1e TFP with the PD-1-CD28 fusion.

### Example 3: In Vitro Persistence Assay of T Cell Receptor Fusion Protein T Cells

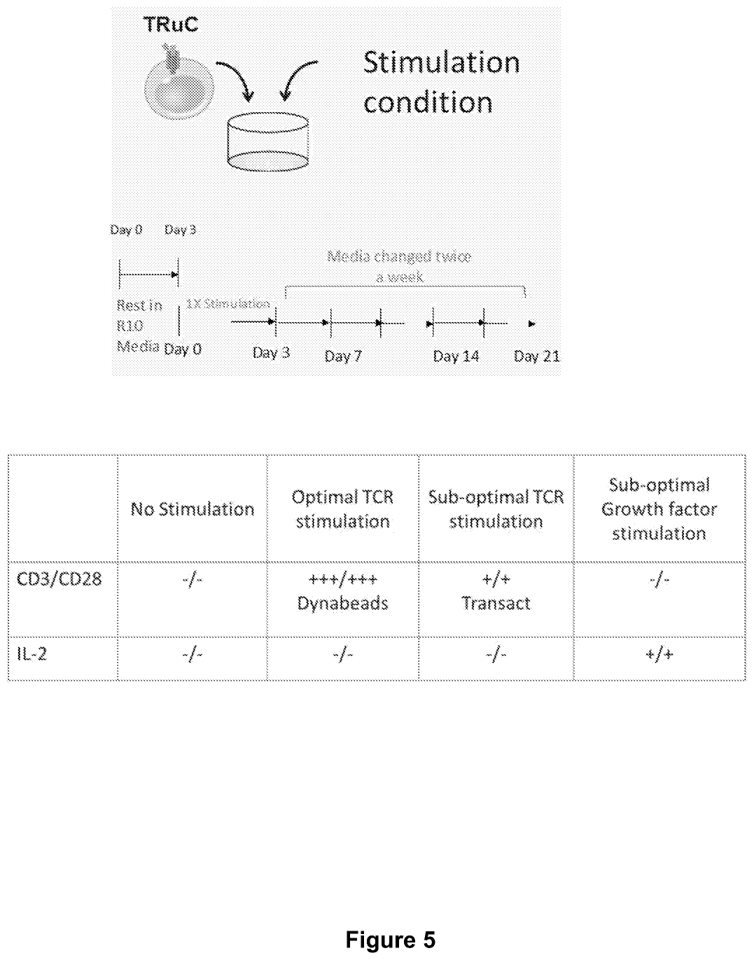

TFP-expressing T cells expanded to day 10, as described above, were rested for three days in R10 media and then exposed to one of four different stimulation conditions for three days (i) no stimulation; (ii) TCR stimulation with dynabeads (“optimal TCR stimulation”), which have high levels of anti-CD3 and anti-CD28 antibodies; (iii) TCR stimulation with transact, which has a lower levels of anti-CD3 and anti-CD28 antibodies (“sub-optimal TCR stimulation”); or (iv) stimulation with IL-2 (“sub-optimal growth factor stimulation”). Media was then changed to that without stimulating conditions, and was changed bi-weekly up to 21 days. T cell expansion and cell phenotype was assessed on days 7, 14 and 21. FIG. 5 is a diagram illustrating the design of the assay.

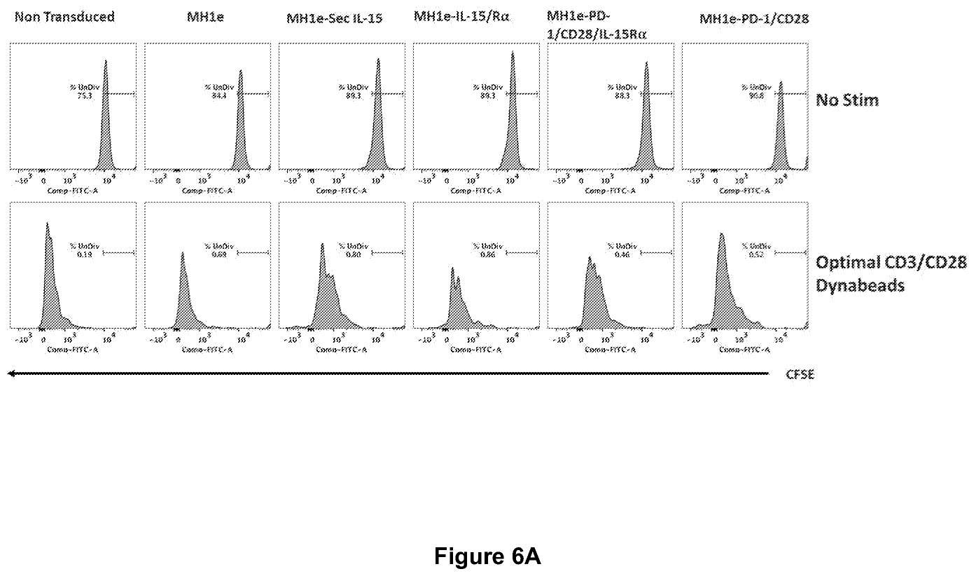

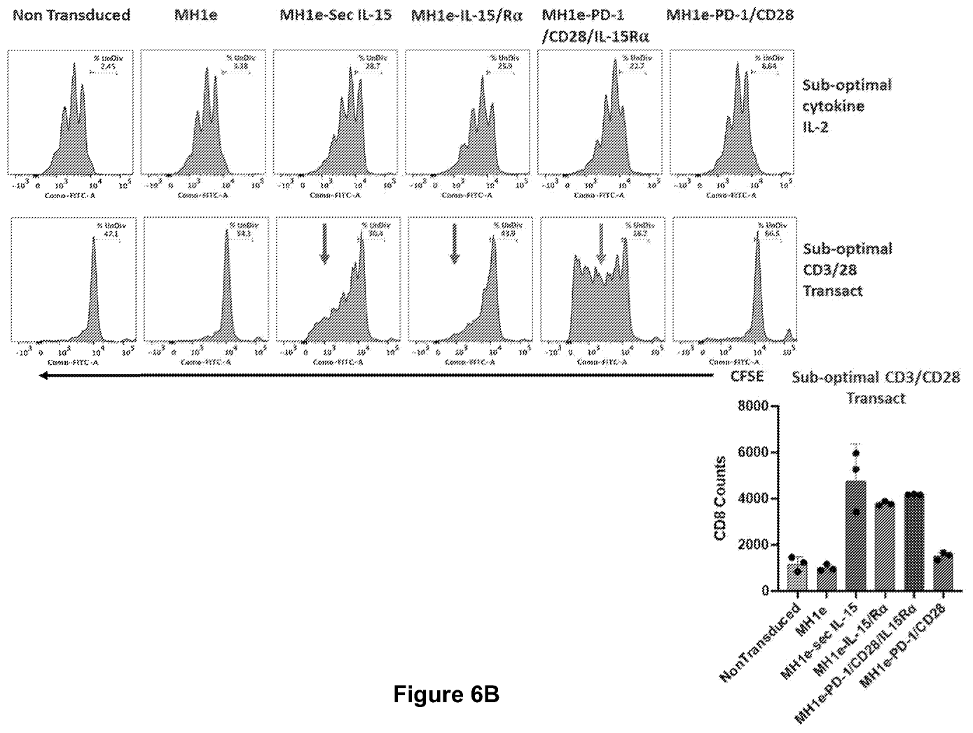

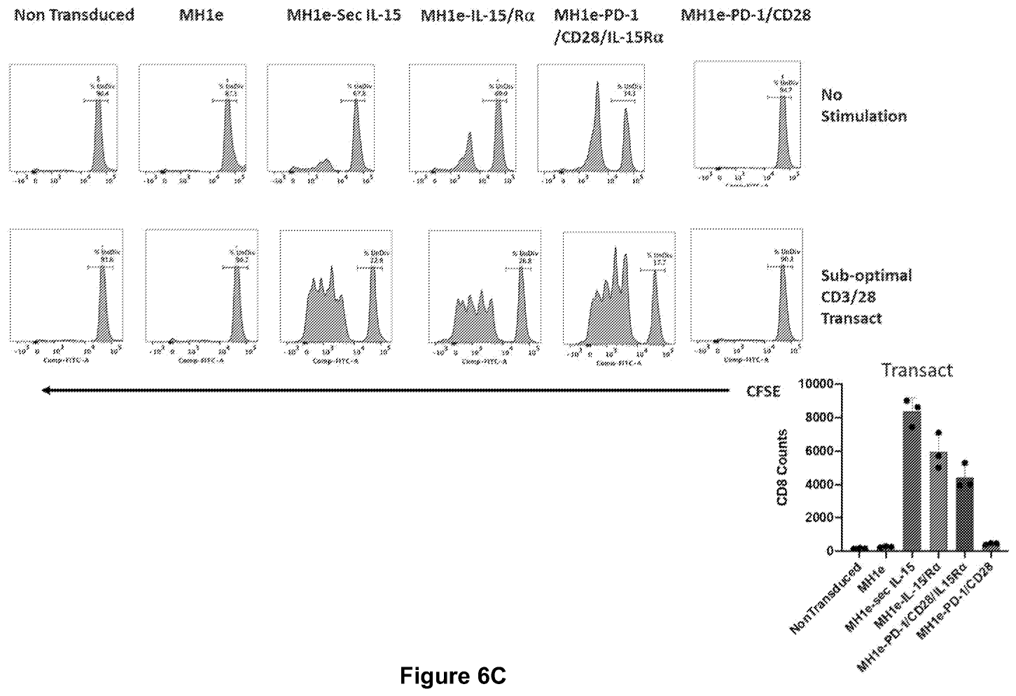

A CSFE dilution assay was performed to assess cell proliferation at days 7 (FIG. 6A and FIG. 6B) and 21 (FIG. 6C) after initiation of the stimulation conditions. VHH+ CD8+ cells were detected. In the CSFE assay, asymmetric cell division results in the accumulation of CSFE low cells, suggesting the cells are not dividing as quickly. As is shown in FIGS. 6A-6C, in all conditions, asymmetric cell division is seen in the TFP T cells expressing secreted IL-15, the IL-15-IL15-Rα fusion, and the PD-1-CD28-IL15-Rα fusion, suggesting these cells are not dividing as quickly as cells expressing TC-210 or the TFP on combination with the PD-1 CD28 switch.

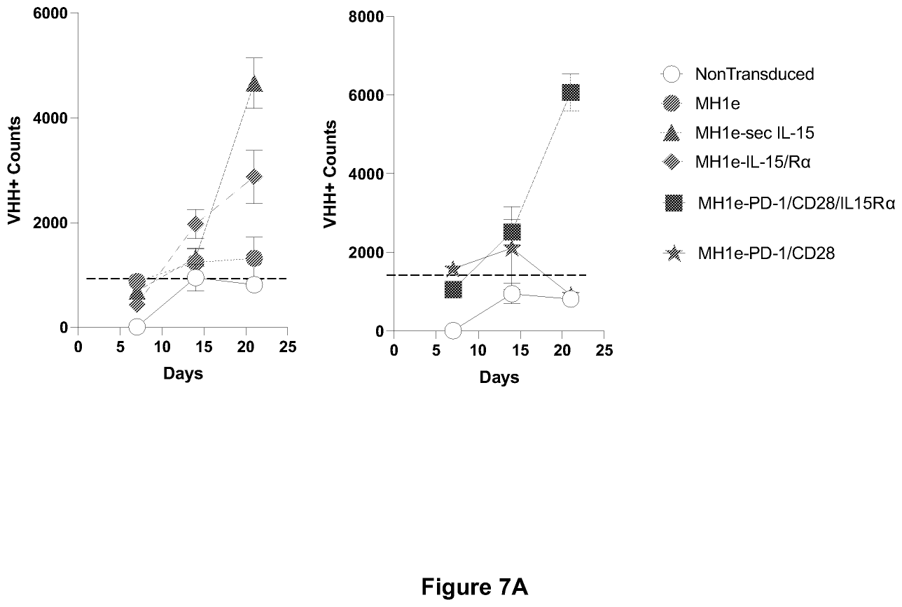

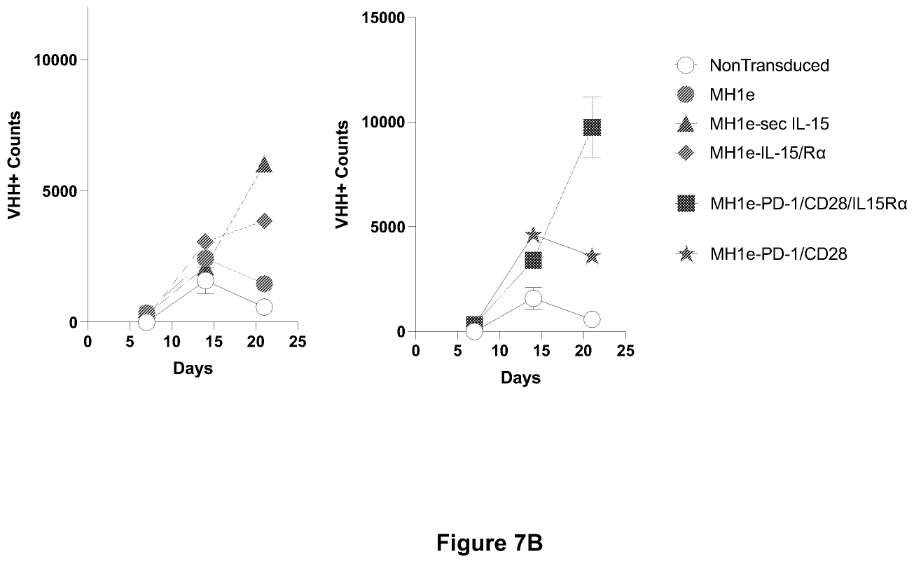

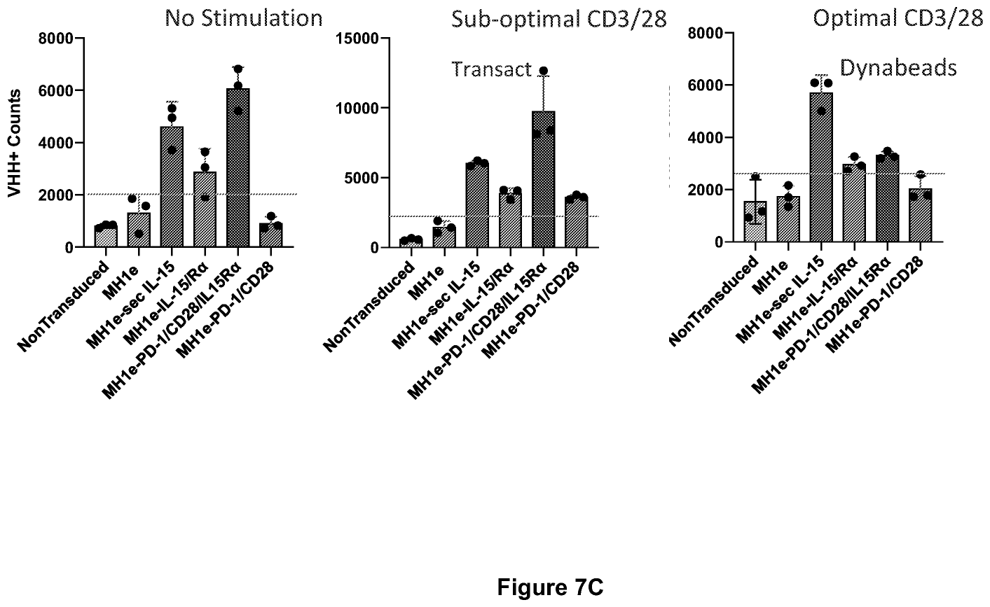

FIG. 7A and FIG. 7B show the expansion of unstimulated cells and cells stimulated in sub-optimal TCR stimulation conditions on days 7, 14, and 21. In both conditions, the TFP T cells expressing secreted IL-15 or the IL-15-IL15-Rα fusion showed expansion beyond that of TC-210 cells by day 21 and TFP T cells expressing the PD-1-CD28-IL15-Rα fusion showed expansion beyond that of TFP T cells expressing the PD-1-CD28 fusion by day 21. FIG. 7C shows the number of TFP+ cells at day 21 for unstimulated cells, and cells stimulated in the sub-optimal TCR stimulating and optimal TCR stimulating conditions. In all conditions, TFP T cells expressing secreted IL-15, the IL-15-IL15-Rα fusion, or the PD-1-CD28-IL15-Rα fusion showed greater expansion than TC-210 cells or TFP cells expressing the PD-1-CD28 fusion protein. These results demonstrate that TFP T cells expressing secreted IL-15, the IL-15-IL15-Rα fusion, or the PD-1-CD28-IL15-Rα fusion exhibit prolonged expansion, even in the absence of stimulating conditions.

### Example 4: Repeated Target Cell Stimulation Assay

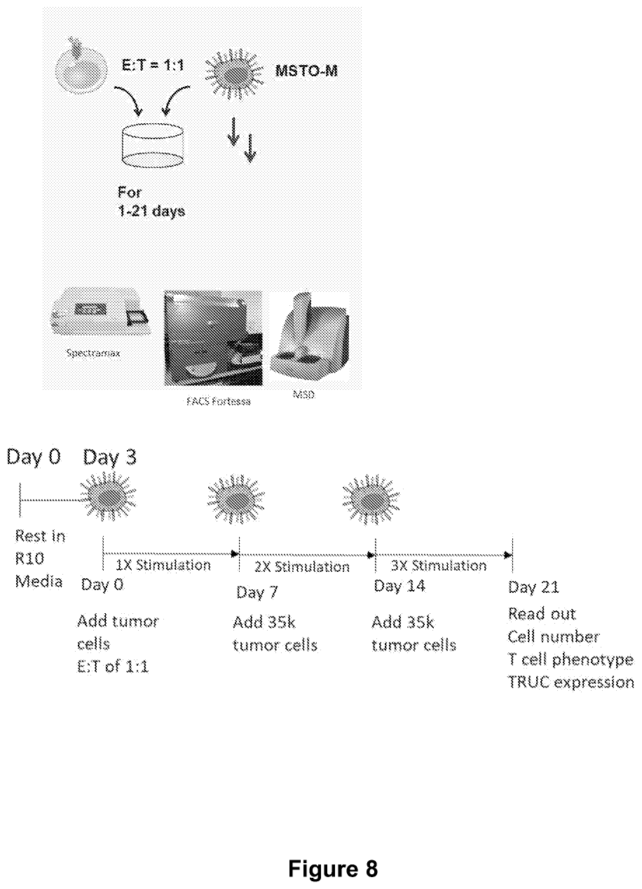

TFP-expressing T cells expanded to day 10, as described above, were rested for three days in R10 media and then contacted with MLSN-expressing MSTO-mlsn target cells at a 1:1 ratio in R10 media. All target cells used in this experiment were modified to overexpress firefly luciferase via transduction with firefly luciferase encoding lentivirus followed with antibiotic selection to generate stable cell line for the cytotoxicity assay described below. 7 and 14 days later, an additional 35 k target cells were added. A schematic illustrating this experiment is shown in FIG. 8.

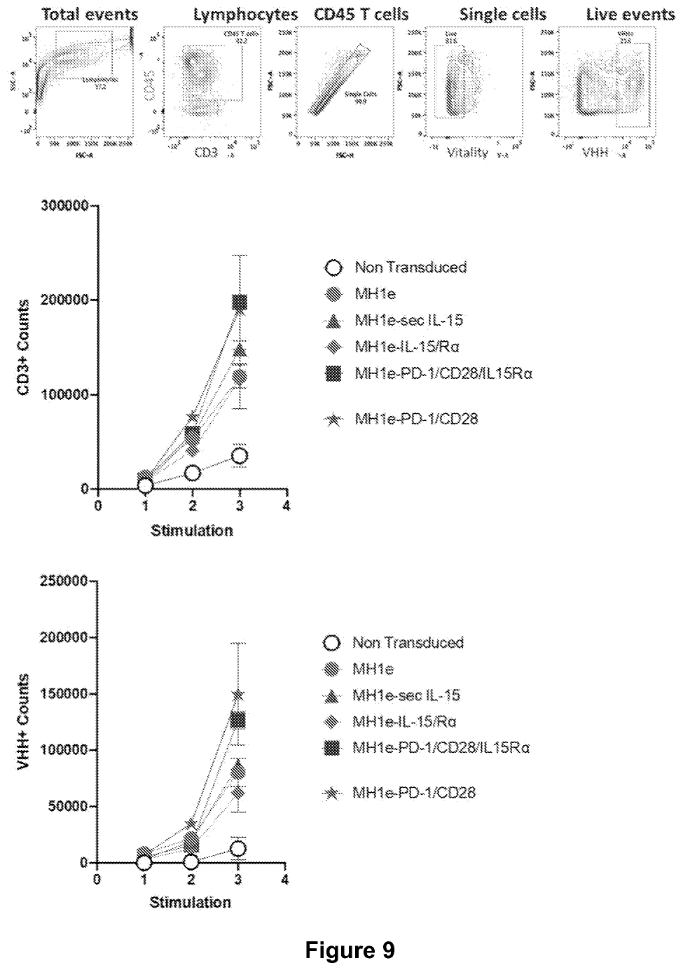

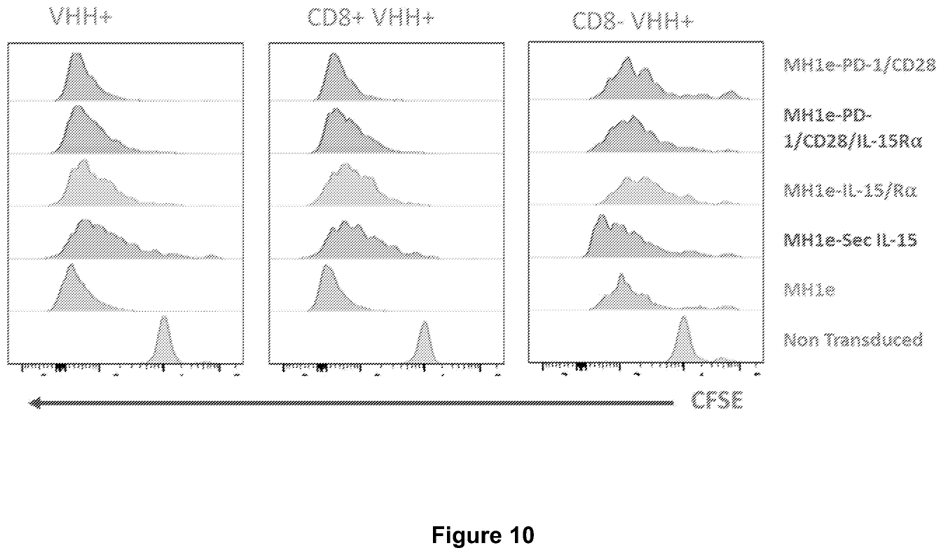

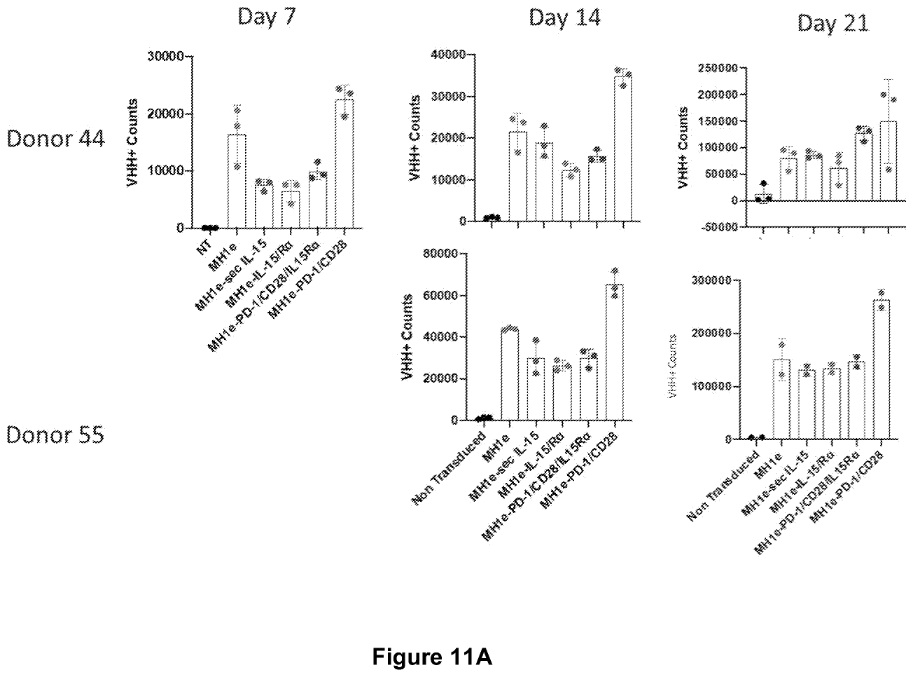

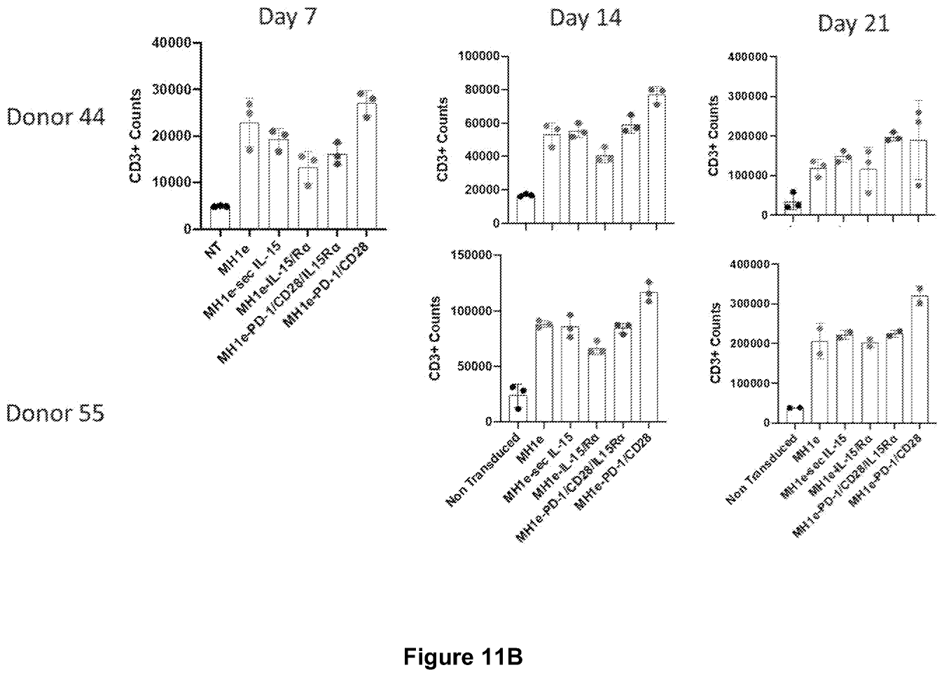

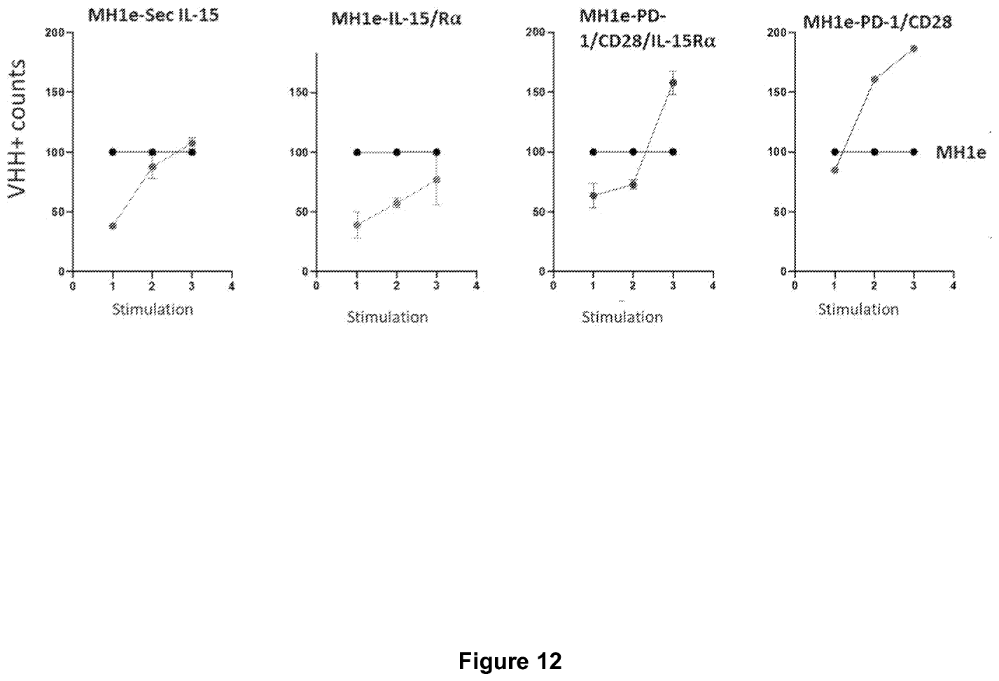

The expansion of VHH+ and CD3+ T cells after three rounds of stimulation for one donor (Donor 44) are shown in FIG. 9. The results of a CFSE assay done at day 7 are shown in FIG. 10. This shows asymmetric cell division in the TFP T cells expressing secreted IL-15, the IL-15-IL15-Rα fusion, and the PD-1-CD28-IL15-Rα fusion, suggesting these cells are not dividing as quickly as cells expressing TC-210 or the TFP in combination with the PD-1 CD28 switch. The level of VHH+ and CD3+ T cells at days 7, 14 and 21 post target cell addition is shown in FIG. 11A and FIG. 11B. At day 7, reduced expansion of TFP T cells expressing secreted IL-15, the IL-15-IL15-Rα fusion, and the PD-1-CD28-IL15-Rα fusion is seen. By days 14 and 21, the discrepancy between the level of VHH+ and CD3+ TC-210 cells and TFP T cells expressing secreted IL-15, the IL-15-IL15-Rα fusion, and the PD-1-CD28-IL15-Rα fusion is reduced, suggesting that cells expressing secreted IL-15, the IL-15-IL15-Rα fusion, and the PD-1-CD28-IL15-Rα fusion demonstrate prolonged expansion, or an increased expansion rate later in the experiment, relative to TC-210 cells. Indeed, FIG. 12 shows the level of VHH+ TFP T cells expressing secreted IL-15, the IL-15-IL15-Rα fusion, and the PD-1-CD28-IL15-Rα fusion normalized to the level of VHH+ TC-210 cells after each addition of target cells. The TC-210 cells were stimulated with MLSN-expressing MSTO-mlsn target cells for three rounds. As is shown, after the first round of stimulation, T cells expressing the fusion constructs initially have lower levels of VHH+ cells, and then the number of VHH+ T cells increases near or above that of TC-210 following subsequent rounds of stimulation, suggesting that cells with the fusion constructs are able to expand to a greater extent following the subsequent rounds of stimulation. The data shown is for Donor 44. Similar data was obtained for Donor 55.

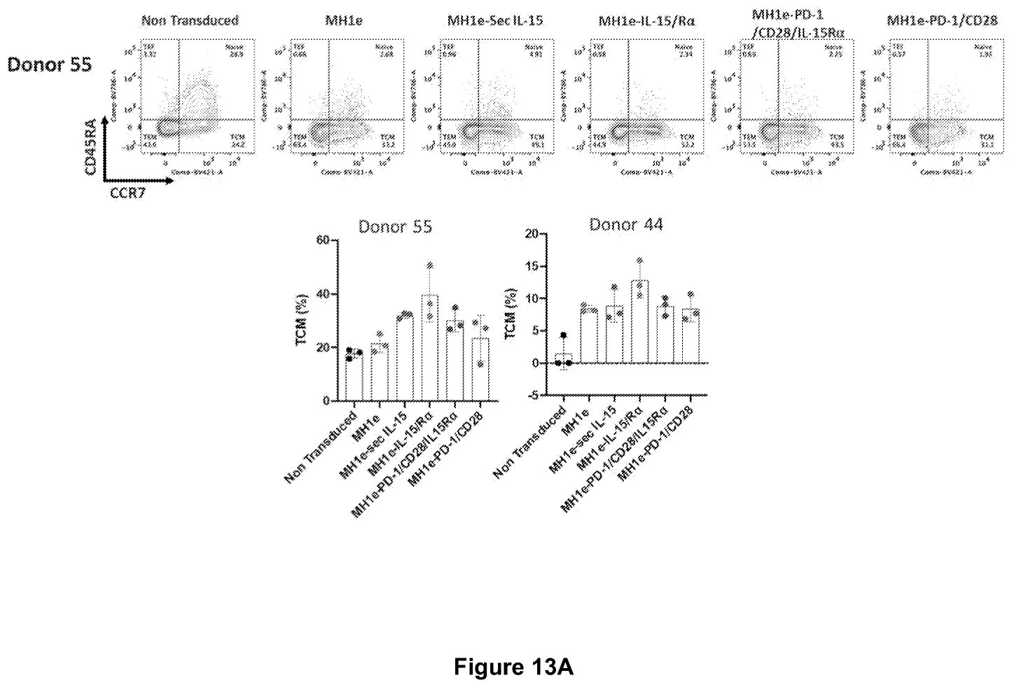

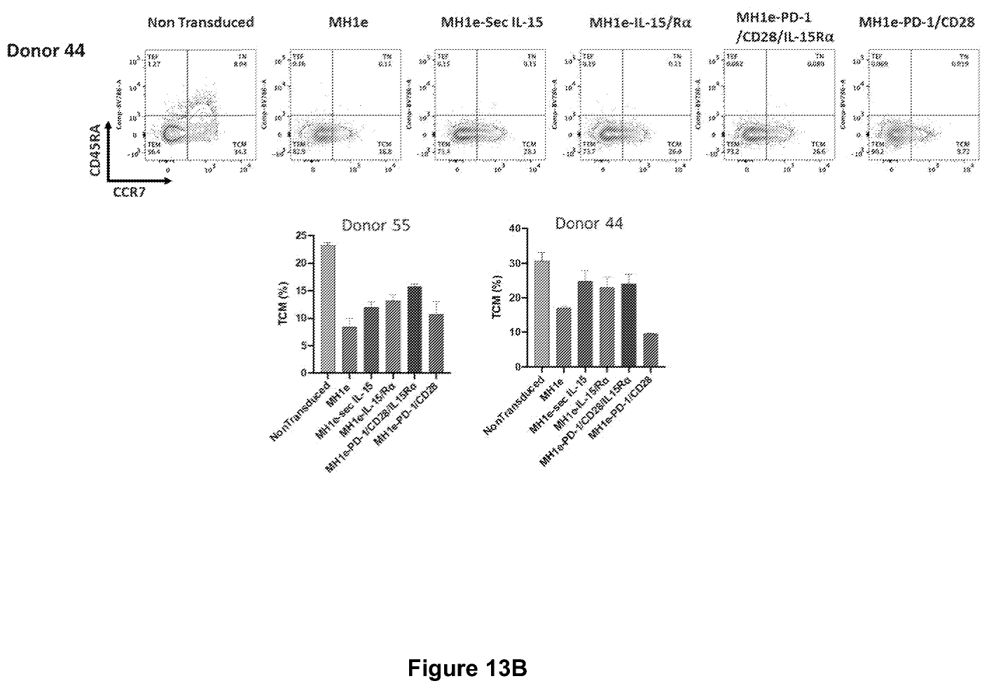

The memory status of CD4+ T cells was determined by flow cytometry to detect cell surface levels of CD45RA and CCR7 on days 7 and 21 after target cell addition as is shown in FIG. 13A and FIG. 13B. At day 7 (FIG. 13A), CD4+ T cells from donor 55 expressing the MH1e TFP with secreted IL-15, IL-15-IL15-Ra fusion or the PD-1-CD28-IL15-Rα fusion and CD4+ T cells from donor 44 expressing the MH1e TFP with the IL-15-IL15-Rα fusion showed an increased in the proportion of central memory cells relative to relative to cells expressing MH1e TFP with or without the PD-1-CD28 fusion. At day 21 (FIG. 13B), CD4+ T cells from both donors expressing the MH1e TFP with secreted IL-15, the IL-15-IL15-Rα fusion, or the PD-1-CD28-IL15-Rα fusion showed an increased in the proportion of central memory cells relative to relative to cells expressing MH1e TFP with or without the PD-1-CD28 fusion.

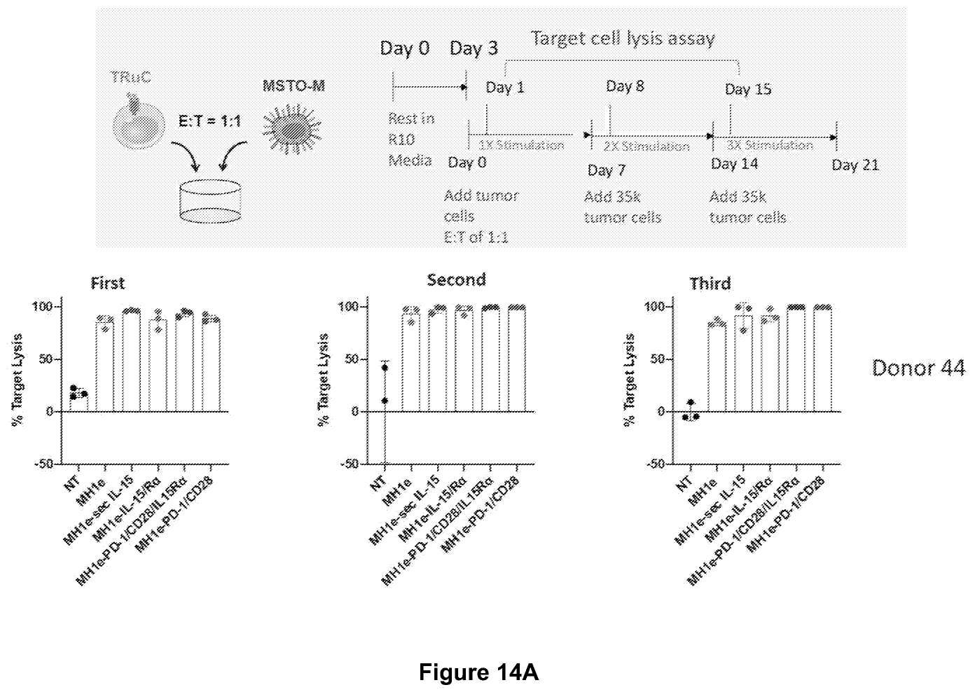

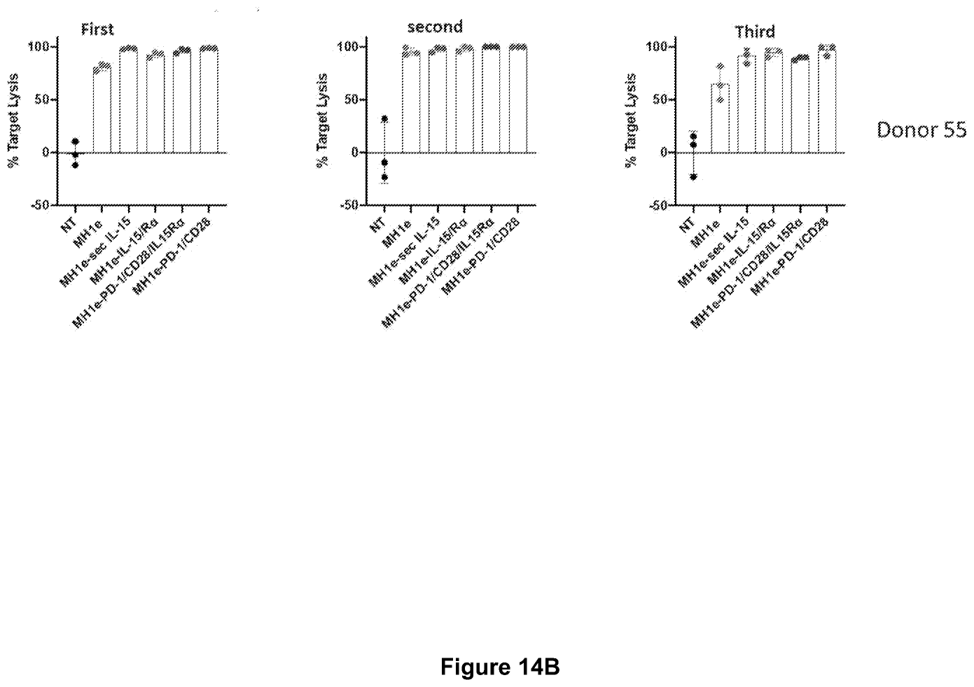

A target cell lysis assay was performed 24 hours after the addition of the 35 k target cells at days 7, 14, and 21. The luciferase-based cytotoxicity assay assesses the cytotoxicity of TFP T cells by indirectly measuring the luciferase enzymatic activity in the residual live target cells after co-culture. The luciferase enzymatic activity in the live target cells is measured by the Bright-Glo® Luciferase Assay System (Promega®, Catalogue number E2610). The cells are spun into a pellet and resuspended in medium containing the luciferase substrate. The percentage of tumor cell killing was then calculated with the following formula: % Cytotoxicity=100%×[1−RLU (Tumor cells+T cells)/RLU (Tumor cells)]. As is shown in FIG. 14A and FIG. 14B, for each of two donors, TC-210 cells showed a decrease in target cell lysis after the third round of stimulation relative to T cells expressing the MH1e TFP with secreted IL-15, the IL-15-IL15-Rα fusion the PD-1-CD28-IL15-Rα fusion, or the PD-1-CD28 fusion.

A measure of effector T-cell activation and proliferation associated with the recognition of cells bearing cognate antigen is the production of effector cytokines such as interferon-gamma (IFN-γ), interleukin 2 (IL-2), and GM-CSF.

Target-specific cytokine production including IFN-γ, IL-2, and GM-CSF by TFP T cells was measured from supernatants harvested after 3 days of co-culture of T cells with target cells using the U-PLEX® Biomarker Group I (hu) Assays (Meso Scale Diagnostics®, LLC, catalog number: K15067L-4).

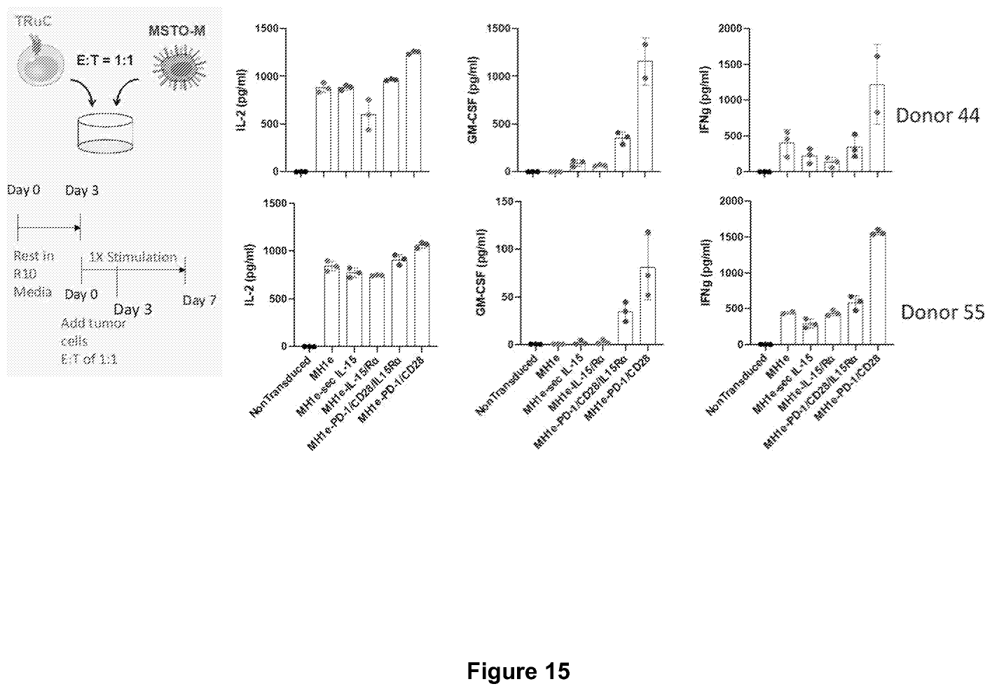

As is shown in FIG. 15, Decreased levels of IFN-γ, IL-2, TNF-α, and GM-CSF were observed in TC-210 cells and the MH1e TFP with secreted IL-15, the IL-15-IL15-Rα fusion, or the PD-1-CD28-IL15-Rα fusion relative to cells expressing the MH1e TFP with the PD-1-CD28 fusion.

### Example 5: Efficacy of Constructs In Vivo

NSG mice were used in a tumor xenograft model of tumor growth. T cells expressing the constructs described herein were tested in xenograft models of both MSLN and MSLN/PD-L1 expressing tumors.

For the MSLN model, NSG were injected SC with 10⁶ MSTO-MSLN-luc cells+Matrigel (1:1) in 200 ul. Approximately 28 days after tumor cell implant, when mice had tumors of 300-600 mm³, mice were injected IV with either TC-210, MH1e-IL15, MH1e-IL15-IL15Rα, MH1e-PD1/CD28/IL15Rα, or MH1e-PD1/CD28 (2×10⁶ transduced T cells), NT T cells or Vehicle. The day of T cell injection was Day 0 of the study.

For the MSLN-PD-L1 model, mice were injected SC with 10⁶ MSTO-MSLN-PDL1-luc cells+Matrigel (1:1) in 200 ul. Approximately 28 days after tumor cell implant, when mice had tumors of 300-600 mm³, mice were injected IV with either TC-210, MH1e-Sec IL15, MH1e-IL15/-IL15-Ra, MH1e-PD1/CD28/IL15Rα, or MH1e-PD1/CD28 (2×10⁶ transduced T cells), NT T cells or Vehicle. The day of T cell injection was Day 0 of the study.

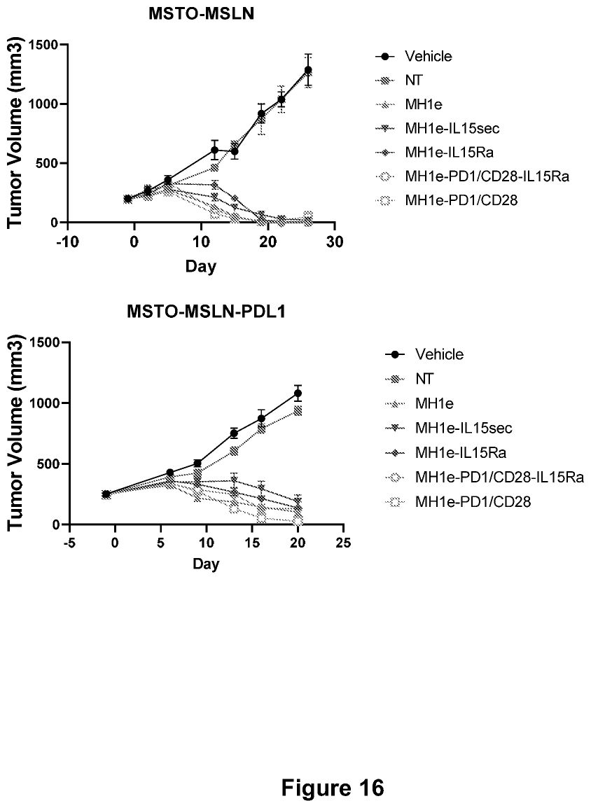

Tumor volume following T cell injection for both studies is shown in FIG. 16. All TFP-expressing T cells effectively reduced tumor volume.

### Example 6: Expression of an IL-15 Receptor Fusion Protein Enhances the Persistence of TRuC-T Cells

Adoptive cell therapies have shown great promise in hematological malignancies. To realize the potential of T cell therapies in solid tumors, T cell receptor fusion construct (TRuC®) T cells have been developed, which are equipped with an engineered T cell receptor that utilizes all TCR signaling subunits and recognizes tumor-associated antigens independent of HLA. In clinical trials, mesothelin-targeting TRuC-T cells (e.g., TC-210 or gavo-cel) have shown unprecedented results in patients suffering from advanced mesothelioma and ovarian cancer. To potentially increase the effector function and persistence of TRuC-T cells in the hostile tumor microenvironment, TC-210 T cells that express a membrane-tethered IL15Rα-IL15 fusion protein were generated. IL-15 is a common γ chain cytokine that promotes the differentiation, maintenance, and effector function of memory CD8+ T cell subsets and confers resistance to IL-2-mediated activation induced cell death (AICD). In vitro, the co-expression of the IL-15 fusion protein enhances T cell proliferation and persistence upon repeated stimulation with MSLN+ cancer cell lines, while exhaustion marker expression is decreased. Furthermore, IL-15 enhanced TC-210 T cells sustain a significantly higher TCF-1+ population. When tested in a mesothelioma xenograft mouse model, the presence of the IL-15 fusion protein increased tumor infiltration and persistence of TC-210 T cells. Altogether, the presented data support clinical studies that explore the impact of IL-15 enhancement on the persistence of TC-210 T cells and depth of response in patients with MSLN+ malignancies.

### Example 7: A Membrane-Tethered IL-15/IL-15 Receptor Fusion Protein Enhances the Persistence and Efficacy of Cd70-Targeted TRuC-T Cells

Adoptive cell therapies have shown great promise in hematological malignancies. To realize the potential of T cell therapies in solid tumors, T cell receptor fusion construct (TRuC®) T cells were developed, which are equipped with an engineered T cell receptor that utilizes all TCR signaling subunits and recognizes tumor-associated antigens independent of HLA. Previously, the discovery and preclinical efficacy of fratricide-resistant TRuC-T cells targeting CD70, a tumor antigen overexpressed in various solid and hematological malignancies, have been described. To enhance T cell effector function and persistence in the hostile tumor microenvironment, anti-CD70 TRuC-T cells were engineered to co-express a membrane-bound IL15Rα-IL15 fusion protein (IL-15fu). IL-15 is a common γ chain cytokine that promotes the differentiation, maintenance, and effector function of memory CD8+ T cell subsets and confers resistance to IL-2-mediated activation induced cell death (AICD).

T cells were activated by CD3/CD28 stimulation and lentivirally transduced with a T2A-containing bicistronic vector encoding the anti-CD70 CD3E-TRuC and the IL-15fu proteins; the cells were further expanded for 9 days in media containing IL-7/IL-15. Surface co-expression of the TRuC and IL-15fu proteins and the T cell memory phenotype was assessed by flow cytometry. In vitro persistence was tested in a repeated stimulation assay in which T cells were challenged by addition of fresh CD70+ target cells every four days with longitudinal assessment of T cell expansion, phenotype, cytokine production, and cytotoxicity. In vivo, the antitumor efficacy of the anti-CD70 TRuC/IL-15fu T cells was evaluated in MHC class I/II deficient NSG mice bearing human tumor xenografts.

The anti-CD70 TRuC and IL-15fu proteins showed high transduction efficiency and robust co-expression on the surface of T cells. The IL-15fu significantly increased the proportion of naïve cells within the TRuC-T cell product, most dramatically in the CD8⁺ subset. In vitro, TRuC-T cells bearing the IL-15fu showed greatly enhanced expansion and persistence upon repeated stimulation with CD70+ target cells. The IL-15 fu also enhanced T-cell survival and persistence under unstimulated, cytokine-free conditions. In vivo, the anti-tumor activity of Cd70-targeted TRuC-T cells was significantly improved by IL-15fu in multiple tumor models and was associated with enhanced intratumoral T cell accumulation and a preferential expansion of CD8⁺ T cells.

The addition of the IL-15fu improved the phenotype, persistence, and anti-tumor activity of Cd70-targeted TRuC-T cells, potentially increasing the likelihood of clinical benefit in patients with CD70 overexpressing solid and liquid cancers.

### Example 8: Characterization of Membrane-Tethered IL-15/IL-15 Receptor Fusion Proteins as Enhancement for Cd70-Targeted TRuC-T Cells

To further characterize the ability of co-expressed membrane-tethered IL-15/IL-15 receptor fusion proteins to enhance effector function and persistence of CD70 targeting TRuC-T cells, additional experiments were conducted. T-cells expressing a T-cell receptor fusion construct (TRuC®) having an anti-CD70 binding domain alone (CD70 TRuC) were compared to those additionally co-expressing a membrane-tethered IL-15/1L-15 receptor fusion protein (CD70 TRuC+mbIL-15fu; hereinafter referred to as mbIL-15fu) and non-treated and/or vehicle treated controls.

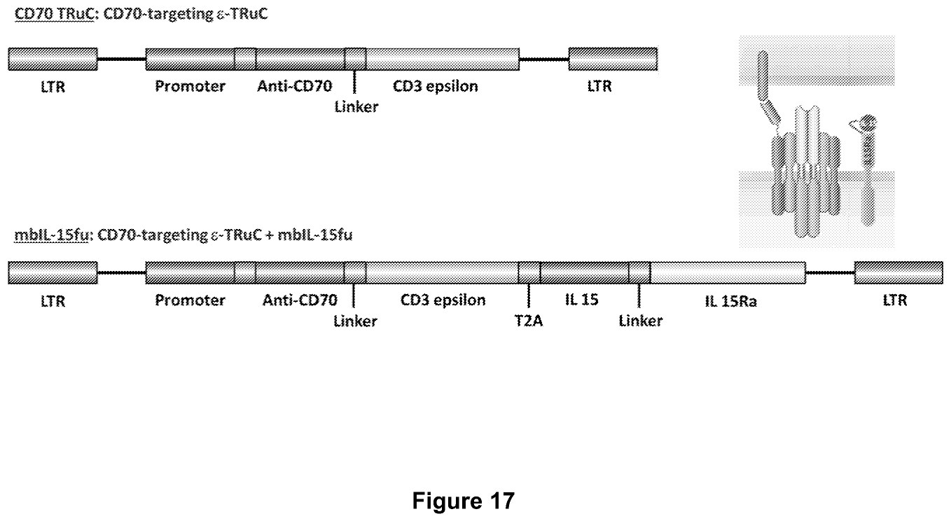

Sequences encoding the CD70 TRuC and the mbIL-15fu proteins were cloned into transgenes and packaged into lentiviral vectors for transduction of CD3/CD28 activated T-cells collected from healthy donor samples. The transgene prepared for the expression of the CD70 TRuC (SEQ ID NO: 400) included a left long terminal repeat (LTR), a promoter, an anti-CD70 binding sequence region comprising an antibody light chain (SEQ ID NO: 399) a linker (SEQ ID NO: 401) and an antibody heavy chain (SEQ ID NO: 395), a linker (SEQ ID NO: 387), a CD3-epsilon sequence (SEQ ID NO: 364), and a right LTR, when read 5′ to 3′. For co-expression of the membrane-tethered IL-15/IL-15 receptor fusion protein, a bicistronic transgene was generated (SEQ ID NO: 402), wherein sequence regions for a T2A linker (SEQ ID NO: 365), IL-15 (SEQ ID NO: 385), a linker (SEQ ID NO: 378), and IL-15Rα (SEQ ID NO: 403), were inserted 5′ to the CD3 epsilon region and 3′ to the right LTR of the CD70 TRuC construct. Schematics of the two transgenes are shown in FIG. 17.

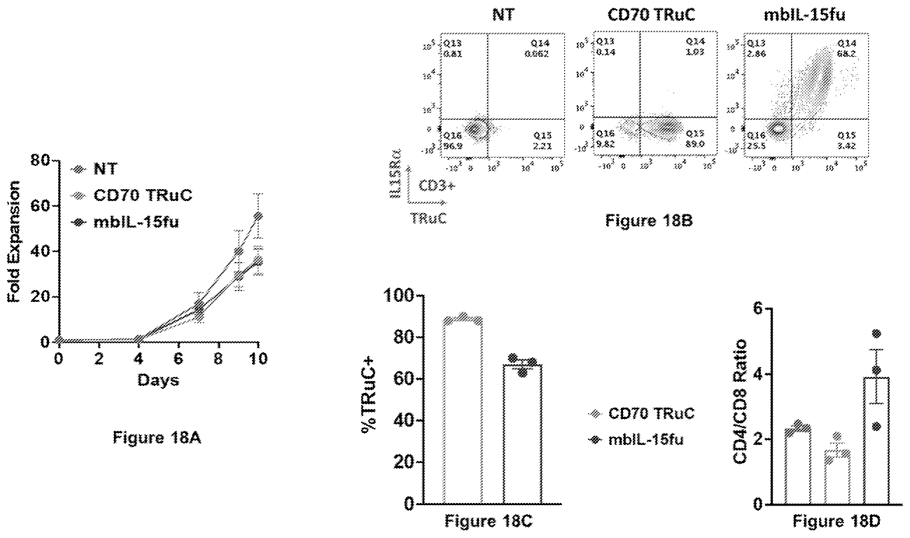

After transduction, T cells were further expanded for 7 days in media containing IL-7 and IL-15 then collected and measured by flow cytometry for surface expression and memory phenotype. As shown in FIG. 18A, T cells transduced with either CD70 TRuC or mbIL-15fu constructs demonstrated similar fold expansion over time and high transduction efficiency FIG. 18B and FIG. 18C. Co-expression of the membrane-tethered IL-15/IL-15 receptor fusion protein showed substantially no impact on CD70 TRuC transduction efficiency. Memory phenotype for TRuC expressing T-cells (TRuC-T cells) was determined by calculating the CD4+/CD8+ ratio with data shown in FIG. 18D. Data in FIG. 18A, FIG. 18B and FIG. 18D were collected across 3 healthy donors.

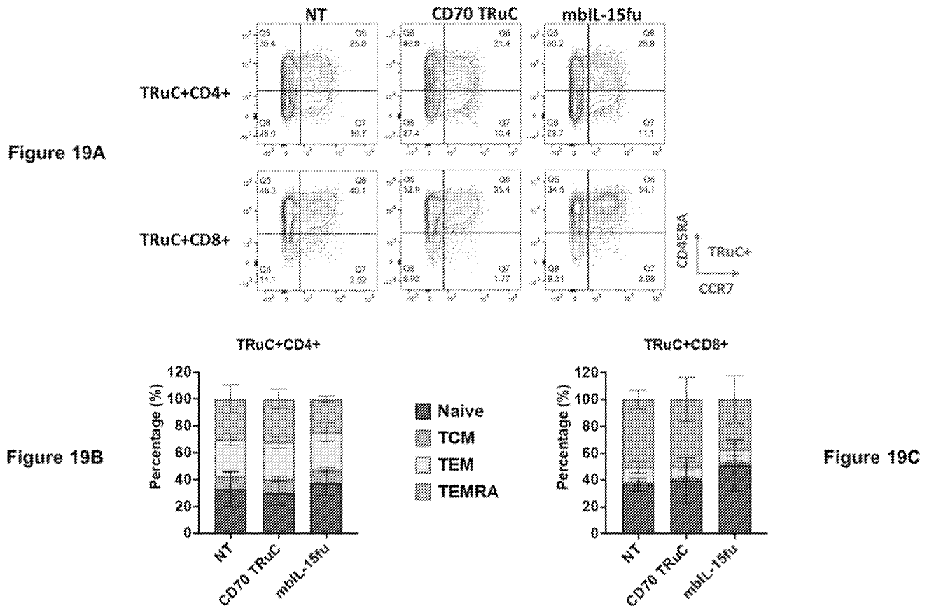

The differentiation state of CD4+ and CD8+ T cells transduced with CD70 TRuC or mbIL-15fu was evaluated by flow cytometry and the proportion for each phenotype (Naïve, TCM, TEM, TEMRA) quantified across 3 healthy donors. The data are shown in FIG. 19A, FIG. 19B, and FIG. 19C and demonstrated that co-expression of a membrane-tethered IL-15/IL-15 receptor fusion protein alongside a CD70 TRuC yielded a significantly increased proportion of naïve cells within the TRuC-T cell product.

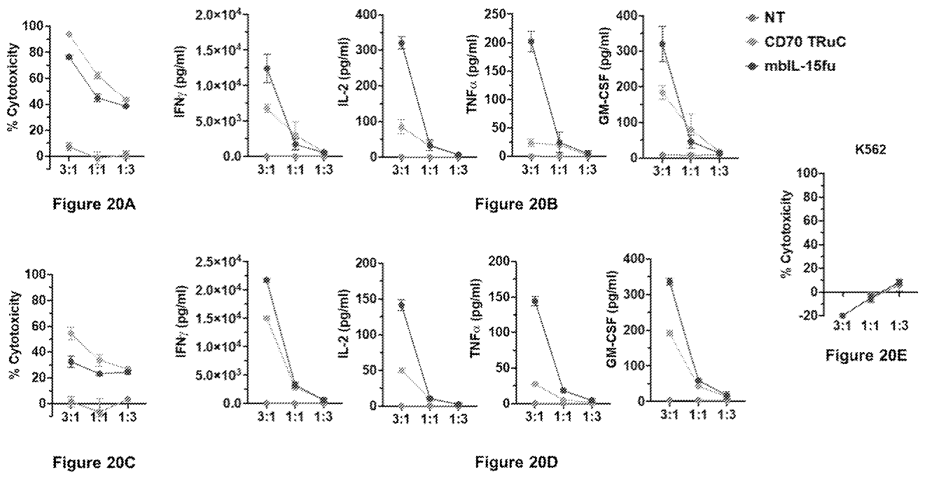

Cytotoxicity and cytokine production of CD70 TRuC or mbIL-15fu expressing T cells was evaluated in vitro by single round co-culture for 24 hours with CD70^{High} cell 786-O, CD70^{Moderate} cell ACHN or CD70^{Negative} cell K562. Data for percent cytotoxicity in CD70^{High} cell 786-O, CD70^{Moderate} cell ACHN and CD70^{Negative} cell K562 after 24 hours of co-culture with CD70 TRuC or mbIL-15fu expressing T cells are shown in FIG. 20A, FIG. 20C and FIG. 20E, respectively, while cytokine release data from CD70^{High} cell 786-O and CD70^{Moderate} cell ACHN are shown in FIG. 20B and FIG. 20D, respectively. After 24 hours of co-culture with K562 cells, no cytokine release was detected. Data are representative of 3 healthy donors. In these assays, T cells co-expressing CD70 TRuC and membrane-tethered IL-15/IL-15 receptor fusion protein (e.g., mbIL-15fu) showed robust tumor killing and cytokine production. When compared to T cells expressing CD70 TRuC alone, the mbIL-15fu T cells demonstrated similar cytotoxicity and enhanced cytokine production.

Persistence and expansion of CD70 TRuC or mbIL-15fu expressing T cells in vitro was tested in the absence of exogenous activating stimuli FIG. 21A or in a target cell stimulation assay. In the “one-hit” target cell stimulation assay, CD70 TRuC or mbIL-15fu expressing T cells were co-cultured with CD70^{High} target cell 786-O at a T cell to target ratio of 5:1 at initiation and then fold expansion measured after this single round of stimulation FIG. 21B. In the repeat target cell stimulation assay, CD70 TRuC or mbIL-15fu expressing T cells were co-cultured with CD70^{High} target cell 786-O at a T cell to target ratio of 1:1 at assay initiation. Additional target cells were introduced to the culture every 4 days and fold expansion calculated over time FIG. 21C. Data were collected across 3 healthy donors and fold expansion calculated as T cells in culture at a given timepoint against the number of T cells present at initiation of the assay. Co-expression of CD70 TRuC and membrane-tethered IL-15/IL-15 receptor fusion protein (mbIL-15fu condition) greatly enhanced persistence and expansion of T cells in vitro as compared to T-cells expressing only the CD70 TRuC or non-transduced controls and this was evident even under unstimulated, cytokine-free conditions.

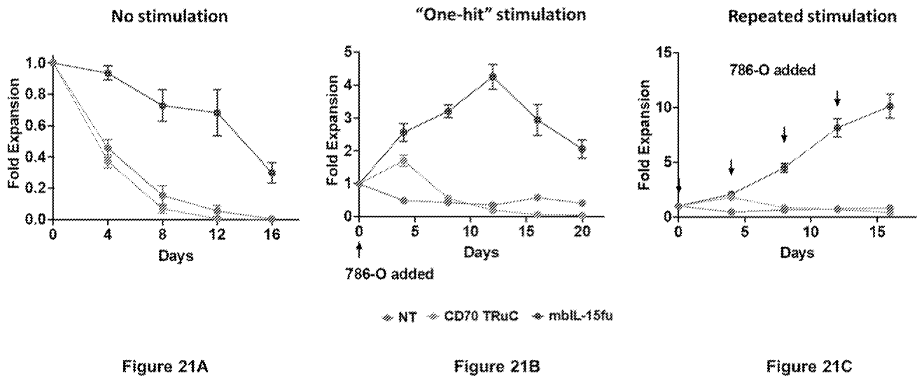

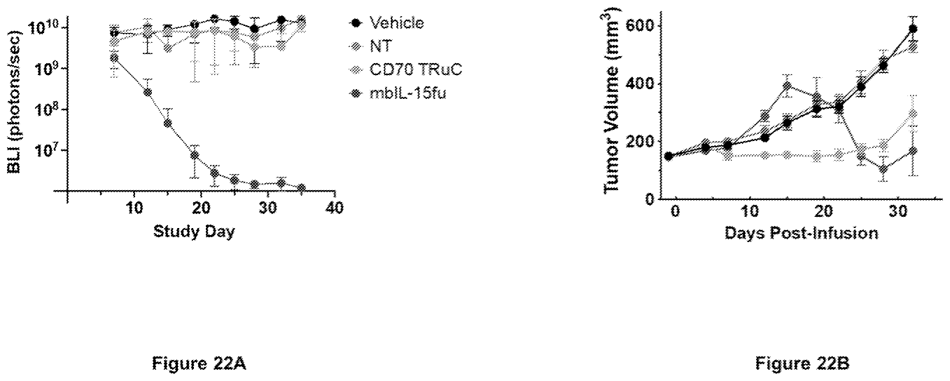

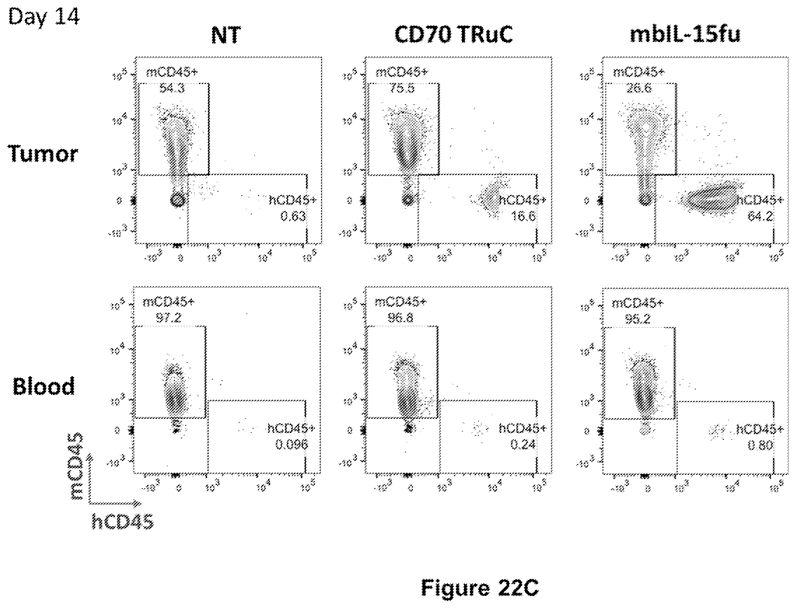

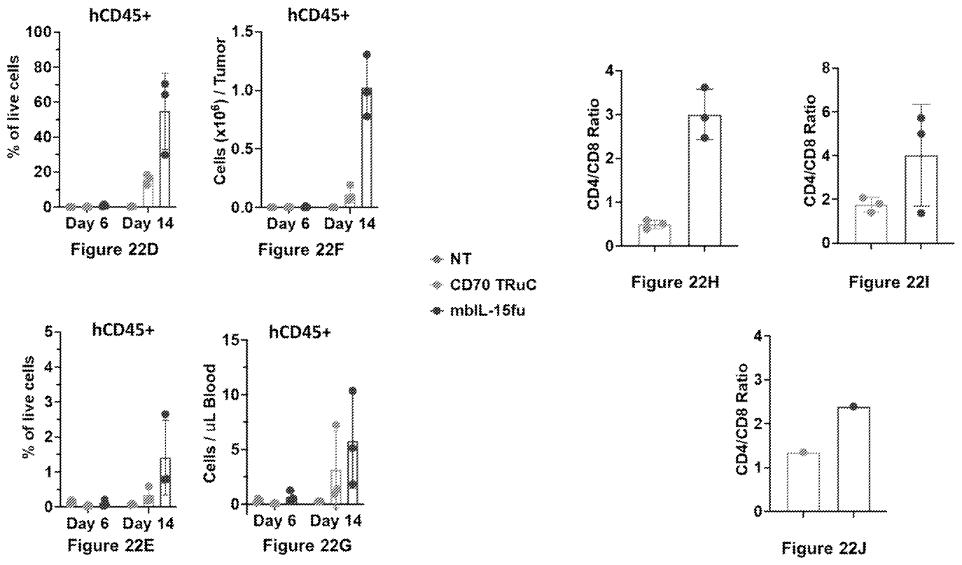

The anti-tumor efficacy and intratumoral accumulation of CD70 TruC or mbIL-15fu expressing T cells were evaluated in vivo in MHC class I/II deficient NSG mice having human tumor xenografts. First, in an ACHN s.c. RCC tumor model in NSG mice, 10⁶ CD70 TRuC or mbIL-15fu expressing T cells were intravenously injected on day 0 (n=5) and anti-tumor activity measured by bioluminescence imaging (BLI). Vehicle injected and non-treated control groups were also used. FIG. 22A shows the decrease in bioluminescent signal over time in mice provided mbIL-15fu expressing T cells as compared to CD70 TRuC only T cells, suggestive of enhanced anti-tumor efficacy of the mbIL-15fu co-expression. Tumor volume was also monitored by caliper measurement with those data shown in FIG. 22B. Human T cell accumulation in tumor and blood collected from a satellite group (n=3) on Day 14 were assessed by flow cytometry. Data from a representative mouse are shown in FIG. 22C. Quantifications of the percentage of human CD45+ cells in tumor and blood and the number of human CD45+ cells per tumor or μL of blood are shown in FIG. 22D, FIG. 22E, FIG. 22F, and FIG. 22G, respectively. Ratios of cD4:CD8 CD70 TRuC or mbIL-15fu expressing T cells in tumor, blood and in the originally infused T cells were also calculated (FIG. 22H, FIG. 22I, FIG. 22J, respectively). These experiments showed the improved in vivo intratumoral accumulation and anti-tumor efficacy of mbIL-15fu when compared to CD70 TRuC expression alone in an RCC tumor model.

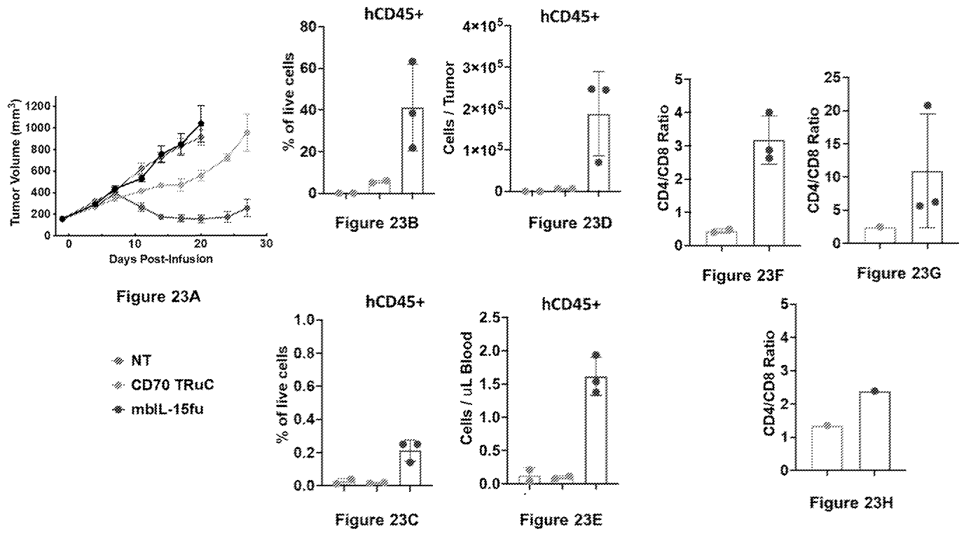

Similar experiments were also conducted in a second tumor model. On Day 0, 10⁶ CD70 TRuC or mbIL-15fu expressing T cells were intravenously injected into MHC class I/II deficient NSG mice having the H-1975 s.c. NSCLC tumor model (n=5). Tumor volume was measured by caliper and is reported in FIG. 23A. On Day 19, tumor and blood samples were collected and the percent or number of CD45+ cells were quantified (FIG. 23B, FIG. 23C, FIG. 23D, and FIG. 23E, respectively). Ratios of cD4:CD8 CD70 TRuC or mbIL-15fu expressing T cells in tumor, blood and in the originally infused T cells were also calculated (FIG. 23F, FIG. 23G, FIG. 23H, respectively). These experiments showed the enhanced in vivo anti-tumor efficacy and intratumoral accumulation of T cells co-expressing CD70 TRuC and membrane-tethered IL-15/IL-15 receptor fusion protein (mbIL-15fu) as compared to T cells expressing the CD70 TRuC alone or control T cells.

Overall, these studies suggested that T cells co-expressing membrane-tethered IL-15/IL-15 receptor fusion protein along with CD70-targeting TRuC (mbIL_15fu) had substantially comparable or improved anti-tumoral characteristics when compared to T cells expressing CD70-targeting TRuC alone. In these experiments, co-expression of membrane-tethered IL-15/IL-15 receptor fusion proteins was able to enhance effector function and persistence of CD70 targeting TRuC-T cells.

### Example 9: Characterization of TRuC T Cells Co-Expressing IL-15

The ability of co-expressed IL-15 variants (e.g., secreted or membrane bound) to improve the performance (e.g., persistence) of T cell receptor fusion construct (TRuC or TFP) expressing T cells was evaluated. IL-15 is known to be important in the maintenance of naïve and central memory CD8+ T cells, enhancing their survival by reducing activation induced cell death (see Dwyer et al 2019, Frontiers in Immunology; 10:263). Further, IL-15 may be considered T cell intrinsic, indicating that use as an enhancement in TRuC T cells may be effective for targeting all tumor types. The hypothesis underlying these studies was that co-expression of IL-15 in TRuC T cells would increase persistence and perhaps lead to enhanced duration of response in human patients. These studies showed that, as initially hypothesized, IL-15 expressing TRuC-T cells demonstrated increased persistence both in vitro and in vivo, as compared to TRuC T cells not co-expressing IL-15.

A series of constructs were designed for testing variations for co-expressing IL-15 in TRuC T cells. Constructs were designed using TC-210 as the base TFP, e.g., each construct included a mesothelin (MSLN) binding VHH antibody sequence region linked to a CD3-epsilon sequence region (SEQ ID NO: 195; TC-210; MH1e), with expression driven by a constitutive promoter. Construct variants were then built by adding a T2A self-cleaving peptide sequence linker and additional sequence component(s) to the 3′ or C-terminal end of the transgene sequence, such that, for example, for secreted IL-15, the construct comprised TC-210 followed by T2A and IL-15 sequences (SEQ ID NO: 380; TC-210 sIL15 or MH1e-sIL15). To test a membrane-bound variant for IL-15 co-expression, a T2A linker and an IL-15/IL-15Rα fusion protein sequence were added to TC-210 (SEQ ID NO: 381; TC-210 IL15Rα-IL15fu or MH1e-IL15Ra). As comparison, an alternate TRuC T cell enhancement strategy was also assessed in this study, wherein TC-210 was co-expressed with a PD1/CD28 fusion protein (TC-510 or MH1-PD1/CD28), as described in WO2018119298, the contents of which are herein incorporated by reference in their entirety. The last variant tested combined both these strategies into a TC-210 construct further comprising a T2A linker, a PD1 extra-cellular domain sequence, a PD1 transmembrane domain sequence, a CD28 intracellular domain sequence and an IL15Rα sequence (SEQ ID NO: 376; PD1:CD28-IL15Rα or TC-510 IL15Rα or MH1-PD1/CD28-IL15Ra). In some assays an additional construct was tested that further included an IL-15 sequence region (SEQ ID NO: 361). Schematics of some (not all) of these constructs and their individual components are exemplified in FIG. 1. Transgenes were cloned into plasmids and packaged into lentiviral vectors for delivery. In each assay, constructs were tested against non-transfected (NT) or vehicle control conditions.

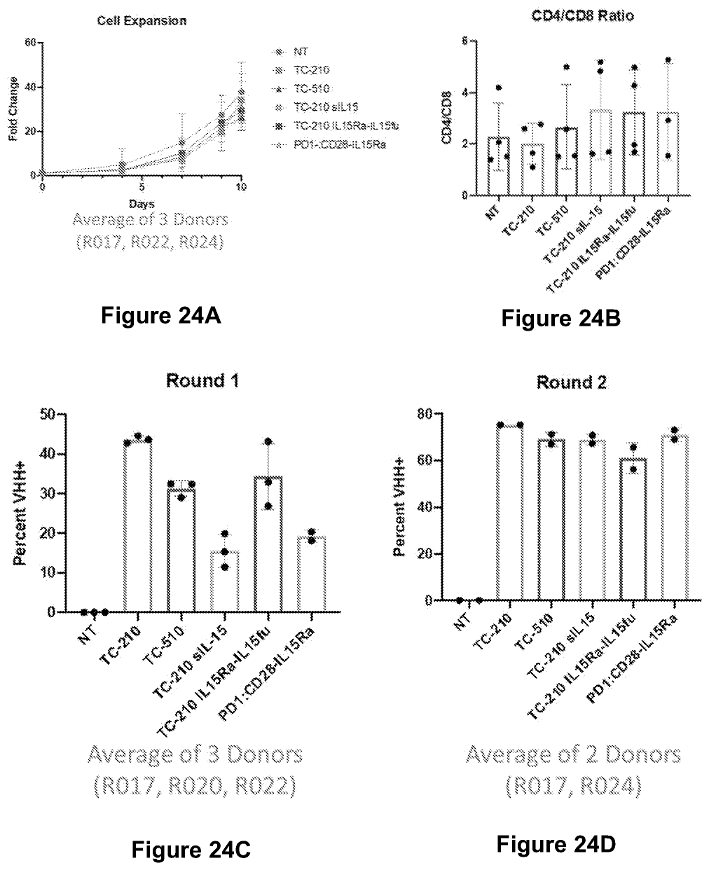

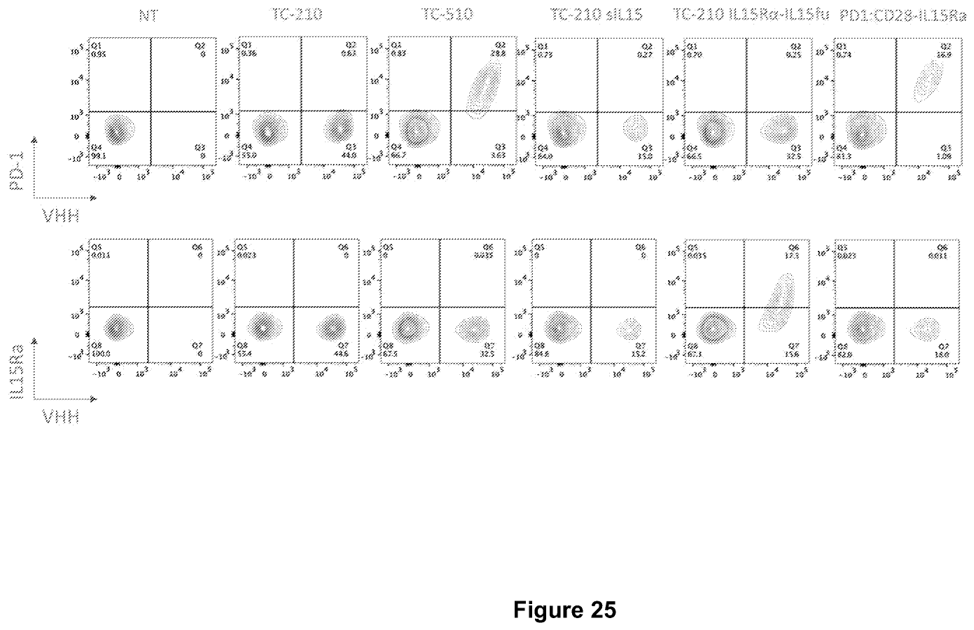

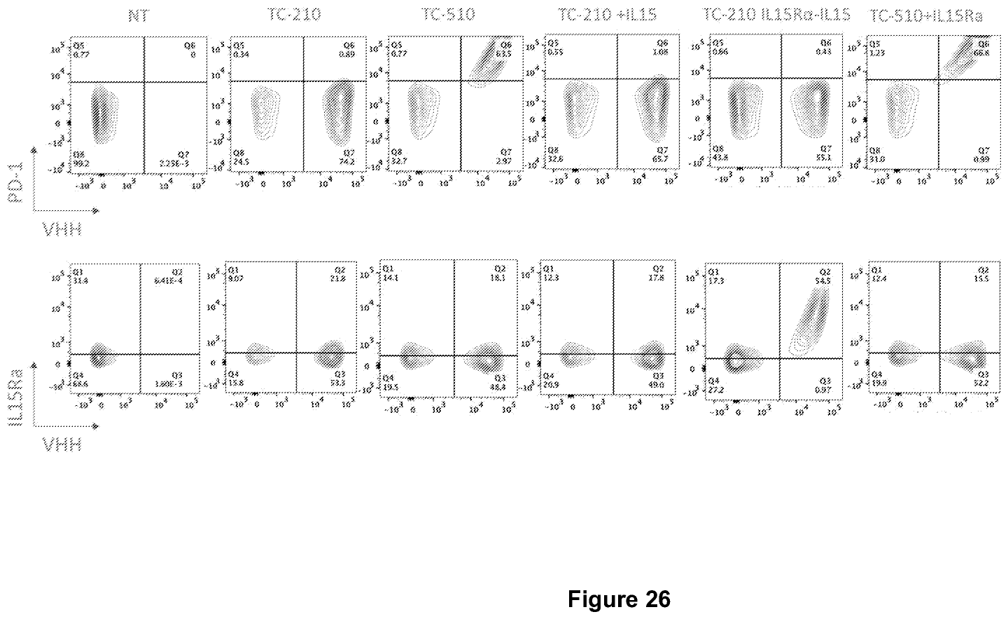

Five constructs (TC-210, TC-510, TC-210 sIL15, TC-210 I115Ra-IL15fu, PD1-CD28-IL15Ra) were transduced into activated T cells collected from healthy donors and evaluated in a T cell expansion assay, then collected and measured by flow cytometry to assess transduction efficiency and CD4/CD8 ratio, with data shown in FIGS. 24A-24D. T cell expansion quantified as the average across 3 donors over 10 days proceeded at a substantially similar rate regardless of the condition, including the non-transfected control. In a first round of assessing the transduction efficiency, the TC-210 sIL-15 and PD1:CD28-IL15Rα transductions appeared less effective than the other conditions, so a second round was assessed in 2 donors. In the second-round assessment, high transduction efficiency (˜60-80%) was evident across all conditions/for each construct. Determination of CD4/CD8 ratio was substantially similar across all conditions, including the non-transfected control. Flow cytometry data for round 1 and round 2 assessment of transduction efficiency based on surface expression of each construct are shown in FIG. 25 and FIG. 26, respectively.

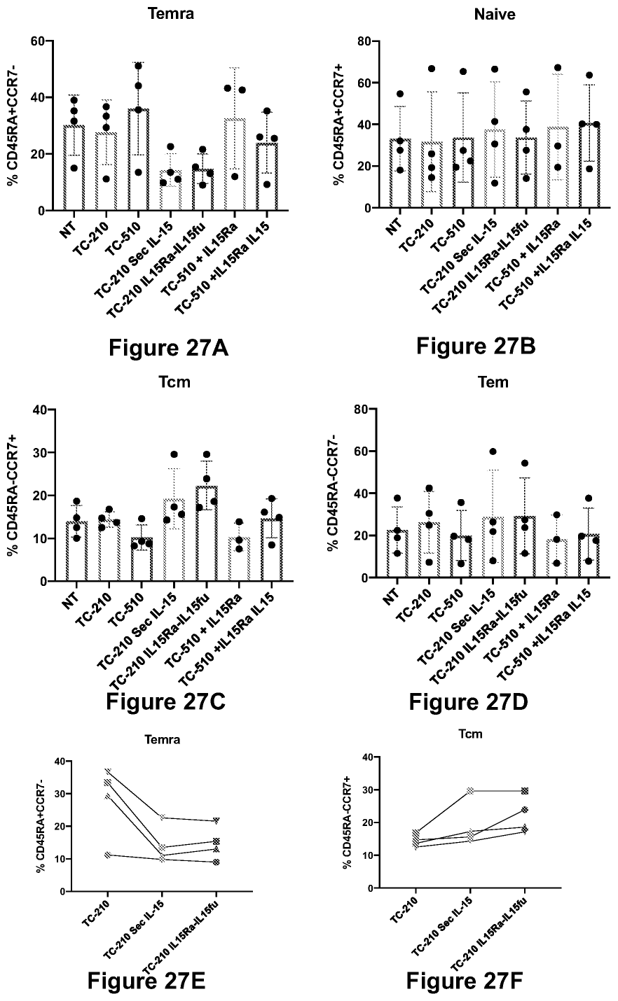

The differentiation state of CD4+ and CD8+ T cells transduced with TC-210, TC-510, TC-210 sIL15, TC-210 I115Ra-IL15fu, TC-510-IL15Ra or TC-510-IL15Ra-IL15 was evaluated by flow cytometry and the proportion for each phenotype (Naïve, Tcm, Tem, Temra) quantified. The data are shown in FIGS. 27A-27F. This quantification showed a slight decrease in Temra (CD45RA+/CCR7−) and slight increase in Tem (CD45RA−/CCR7+) phenotypes in TC-210 transduced T cells co-expressing IL-15 at day 10 post expansion.

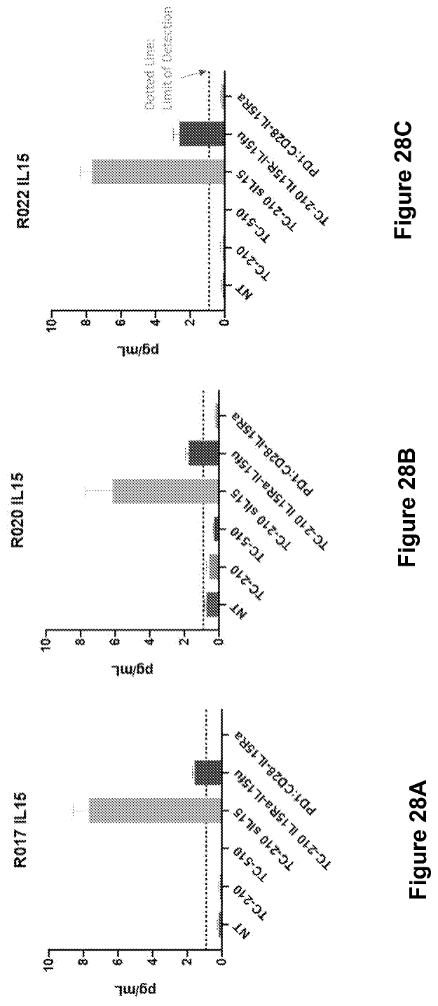

An IL-15 cytokine production assay was conducted using 1.8×10⁵ T cells (˜20% TRuC+) placed in 200 μL of media with TransACT for 24 hours. T cells from three healthy donors were used for this assay. The data are shown in FIGS. 28A-28C and show IL15 expression in T cells transduced with TC-210 sIL15 and TC-210 IL15Ra-IL15fu. T cells transduced with constructs lacking a co-expressed IL-15 did not show significant IL-15 cytokine production in this assay.

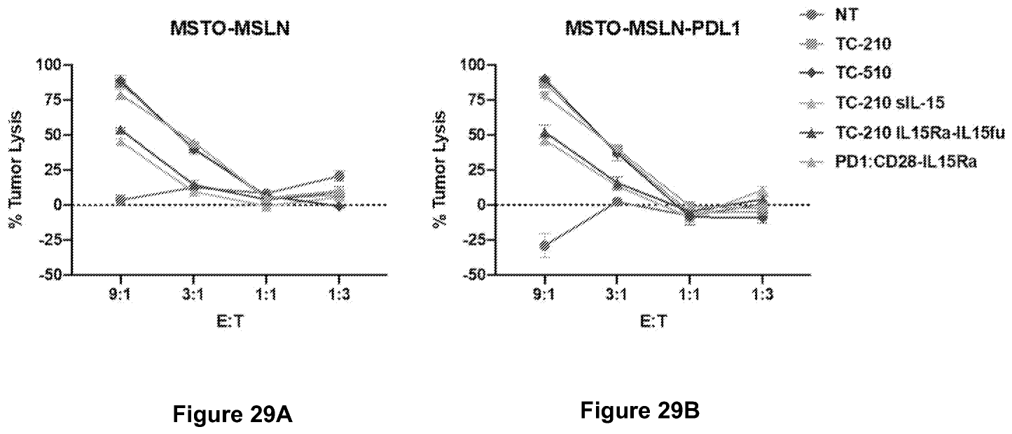

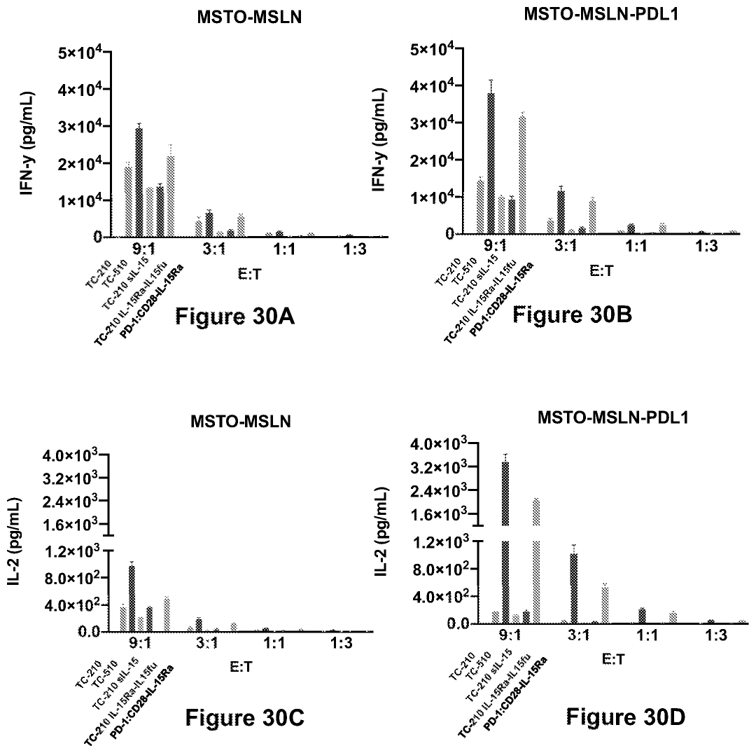

T cells transduced with TC-210, TC-510, TC-210 sIL15, TC-210 IL15Ra-IL15fu, or PD1-CD28-IL15Ra were tested in a cell killing (cytotoxicity) and cytokine response assay. 1×10⁴ tumor cells (C30, MSTO-MSLN, or MSTO-MSLN-PDL1) were plated in each well of a 96-well plate. Transduced T cells were prepared and added at varying effector cell:target cell (E:T) ratios and incubated for 24 hours. A luciferase readout was used to determine % tumor lysis or the cell killing ability for each population of transduced T cells and the supernatant from the assay collected for assessment of cytokine (IFN-γ and IL-2) response. FIGS. 29A and 29B show the data for the cytotoxicity assay in a representative donor and demonstrates that T cells transduced with each of the five constructs were able to successfully eliminate MSTO-MSLN or MSTO-MSLN-PDL1 cells. In this donor the IL15 expressing TRuCs TC-210 IL15 and TC-210 IL15R-IL15fu had slightly lower cytotoxicity compared to TC-210 and TC-510 and its variants. Similar results were observed for Donor R024. For Donor R022, no difference in killing was observed. Donor R022 had the greatest Tem population of the three donors tested. Likewise, FIGS. 30A-30D show the data of the cytokine assay where T cells transduced with each of the five constructs generated large amounts of cytokines when co-cultured with MSLN expressing cell lines. GM-CSF and TNFα showed similar trend to IFNγ. As shown in the FIGS. 30A-30D, for each E:T ratio, the bar graphs represent negative control (NT, not visible in figures), TC-210, TC-510, TC-210 sIL-15, TC-210 IL-15Ra-IL-15fu, and PD-1:CD28-IL-15Ra from left to right.

As shown in cytotoxicity and cytokine response assays, all TRuCs generated were cytotoxic and produced large amounts of cytokines upon co-culture with MSLN-expressing cell lines. Two of the three donors tested had decreased in vitro cytotoxicity against MSLN-expressing cell lines when the TRuCs also expressed IL15.

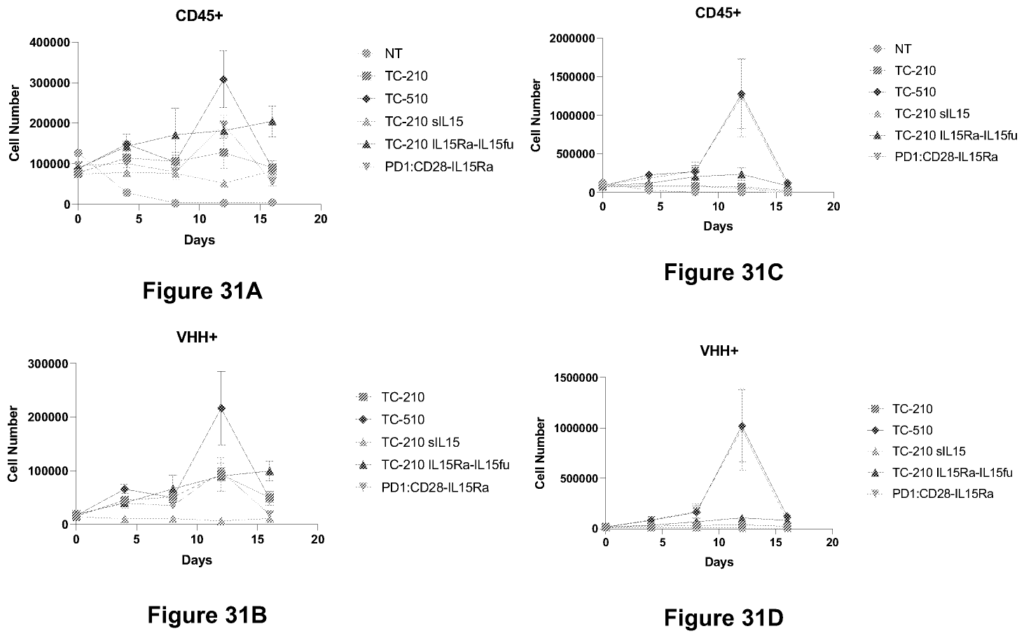

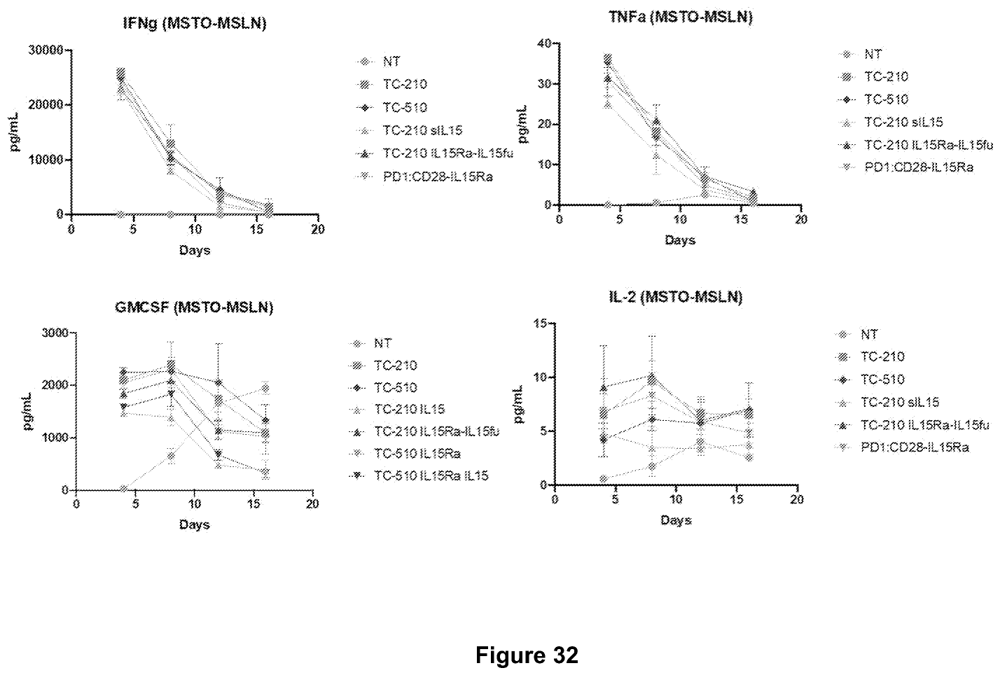

Next, T cells transduced with the aforementioned constructs were tested in a repeated stimulation assay. On Day 0, 3.5×10⁴ transduced T cells (negative controls, TC-210, TC-210+IL-15 sec, TC-210+IL-15Ra-IL-15fu, TC-510, TC-510+IL-15Ra, TC-510+IL-15Ra-IL-15 sec) were thawed, washed in R10 media, and normalized by % VHH+ and co-cultured with 3.5×10⁴ MSTO-MSLN or MSTO-MSLN-PDL1 tumor cells in 200 μL of R10 media in one well of a 96-well plate. On Days 4, 8, 12 and 16, supernatant (100 μL) was collected and frozen for cytokine release assessment and cells were collected for evaluation by FACs/flow cytometry. Alternatively, on Days 7, 14, 21, supernatant (100 μL) was collected and frozen for cytokine release assessment and cells were collected for evaluation by FACs/flow cytometry for 7-Day repeated stimulation assay. An additional 100 μL of new tumor cells were added to the co-culture at these time points. Quantification of cell number determined by CD45+ or VHH+ are shown in FIGS. 31A-31D and demonstrate that T cells transduced with TC-210 IL15Rα-IL15fu maintained cell expansion beyond the time point when T cells transduced with other constructs started to crash. Cytokine production data from the same assay conducted on in MSTO-MSLN cells are shown in FIG. 32 and demonstrate that cytokine release, as measured by IFNγ, TNFα, GMCSF and IL-2, decreased over time for each of the constructs tested.

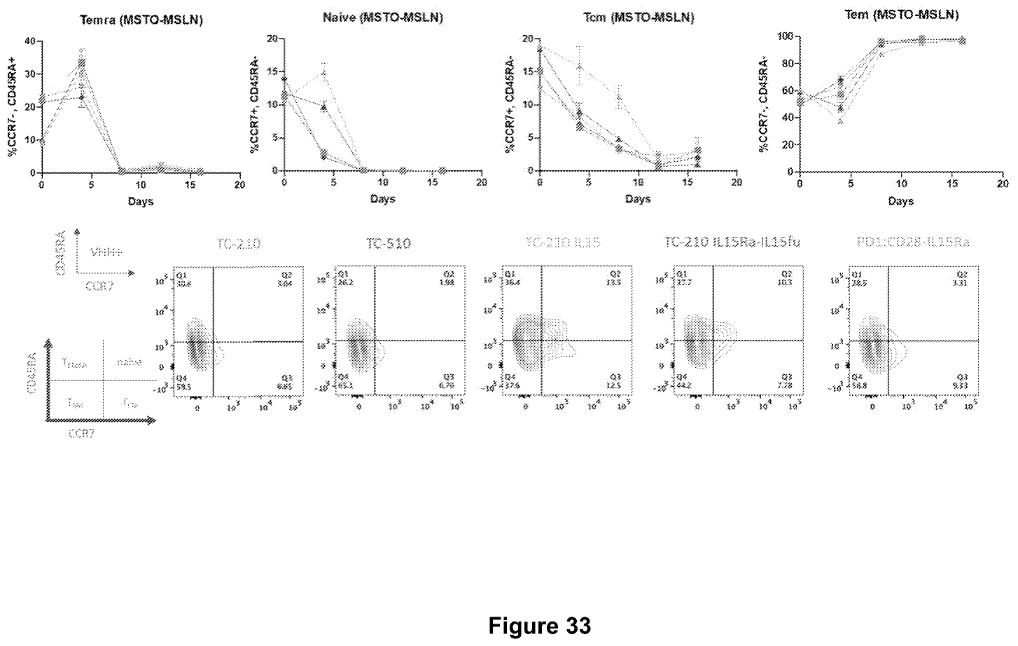

The differentiation state (Naïve, Temra, Tcm, and Tem) of transduced T cells from the repeated stimulation assay was determined based on CCR7 and/or CD45Ra identification and plotted as shown in FIG. 33. T cells co-expressing IL15 maintained a naïve phenotype for longer in culture while those specifically expressing a secreted IL15 maintained a Tem phenotype for a longer duration. The anti-MLSN TRuC-T cells expressing the IL15Rα-IL15 fusion protein continued to expand as the TC-210 and TC-510 constructs crashed later in culture.

The repeated stimulation assay showed that IL15 promoted the maintenance of Naïve and Tem cells early in culture, but repeated stimulation led to an effector memory phenotype. IL15 did not have a major effect on the decreasing cytokine production by TRuCs upon repeated stimulations.

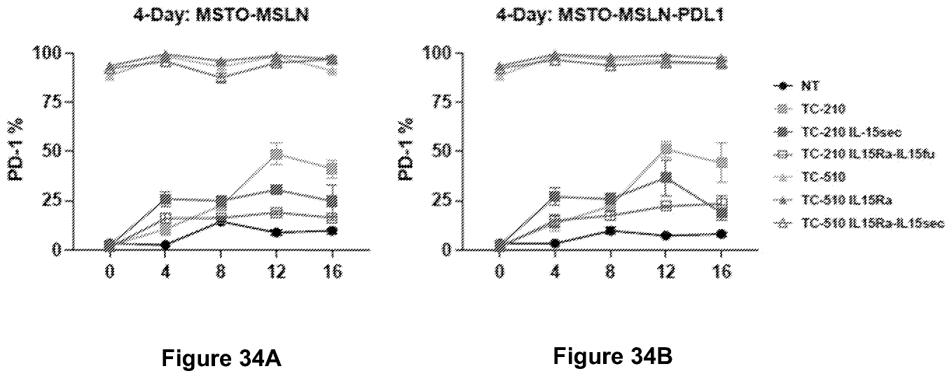

PD1 expression was also assessed in the repeated stimulation assays, with data shown in FIG. 34A and FIG. 34B. The assessment was gated on CD3+VHH+ cells and showed high PD1 expression in conditions wherein PD1 was co-expressed (e.g., TC-510, TC-510 IL15Ra, and TC-510 IL15Ra-IL15fu). The data were gated on CD3+VHH+ cells. NT is the total CD3+ population.

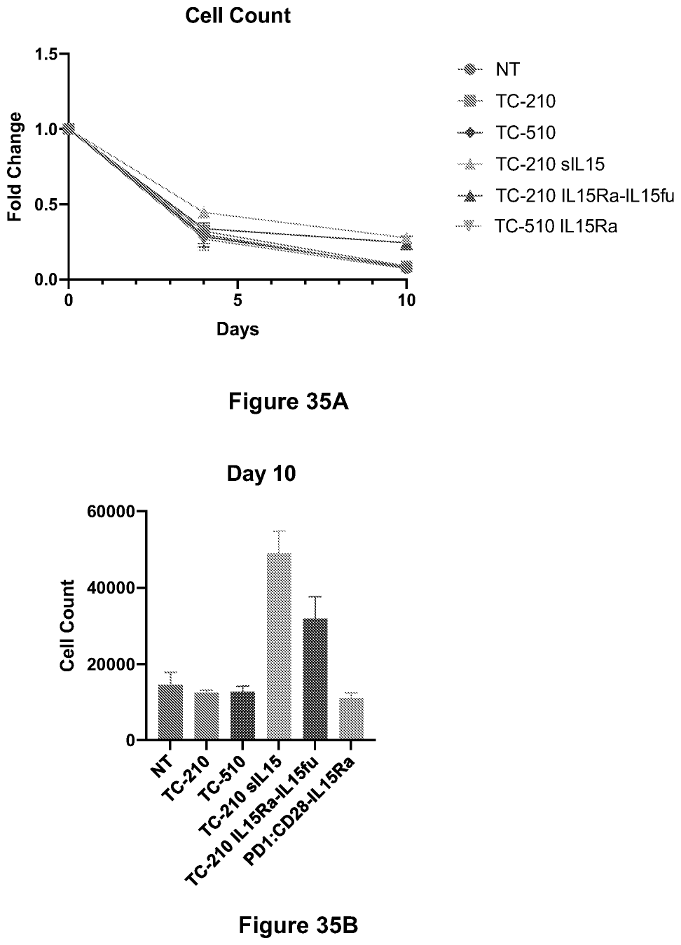

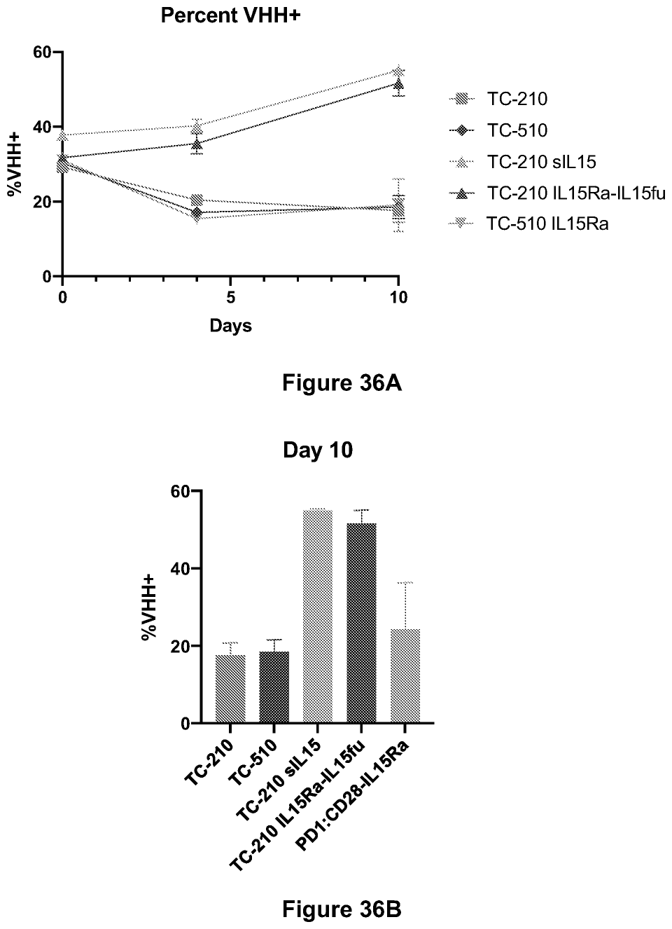

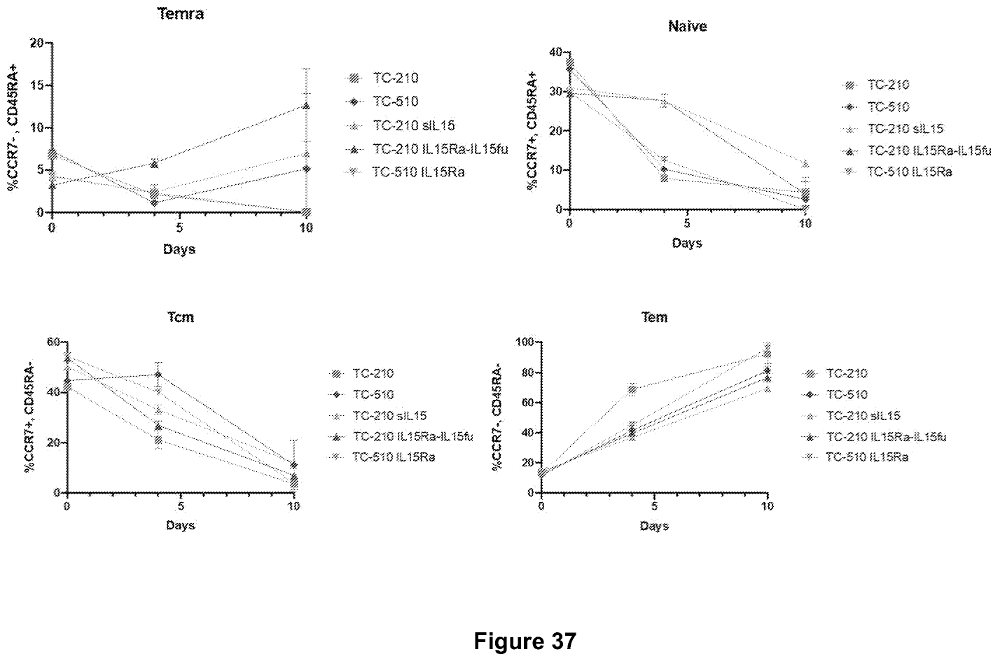

The persistence of T cells transduced with each of the constructs was tested using an antigen independent expansion assay. On Day 0, transduced T cells (TC-210, TC-510, TC-210 IL15, TC-210 IL15-IL15Ra, TC-510 IL15Ra, TC-510 IL15Ra-IL15) and non-transduced controls were plated into 3×96 well plates at 0.5×10⁶ cells per 1 mL of media (+/−IL-2 or IL-15). In the cases where a PD1-CD28 component was included in the construct, the plates were coated with PDL1-Fc (5 μg/mL). At day 4, cells were collected for determination of transduction efficiency by gDNA assay and for FACs analysis for cell count, IL-15 and VHH expression, CD4/CD8 ratio and memory phenotype. At Day 6, 50% of the media and cells were replaced and at Day 8 this was repeated. Cells were harvested at Day 10. Without exogenous stimuli, transduced T cell numbers decreased over time, but those co-expressing IL15 showed a tendency to persist better over time. These data are summarized in FIGS. 35A and 35B. Evaluation of percent VHH+ in these cultures showed a higher percentage of VHH+ cells in transduced T cells co-expressing IL-15. Additionally, the percentage of VHH+ cells increased overtime, further supporting the finding that co-expression of IL-15 improved persistence of transduced T cells. These data are shown in FIGS. 36A and 36B. Analysis of the memory phenotype as shown in FIG. 37 showed that although all cells in this assay progressed toward an effector memory phenotype (Tem) over time, those co-expressing IL-15 maintained a naïve phenotype for longer. These data were collected without the addition of exogenous cytokines and were gated on VHH+.

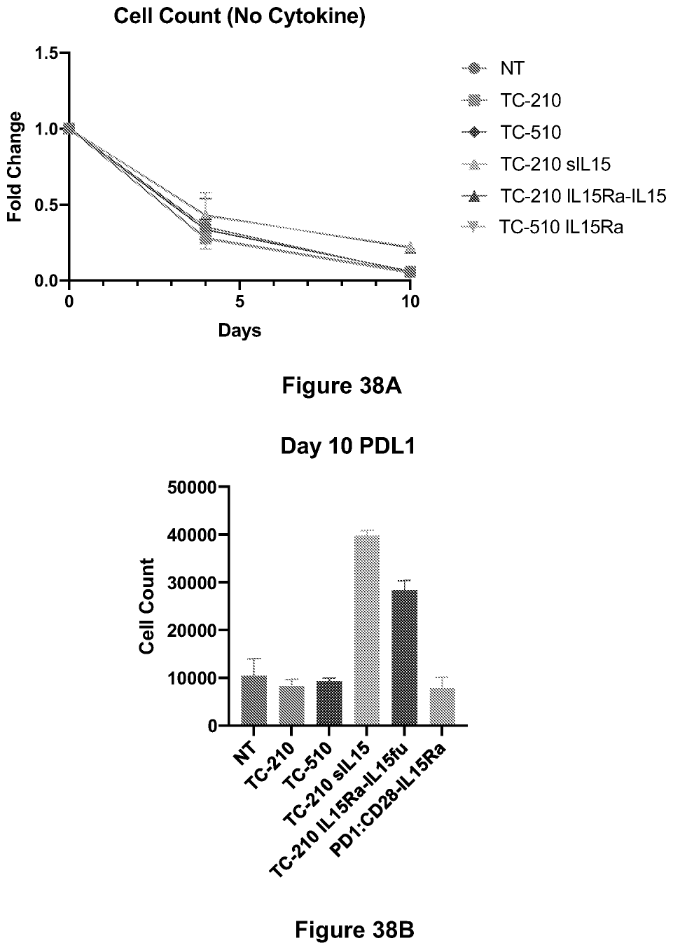

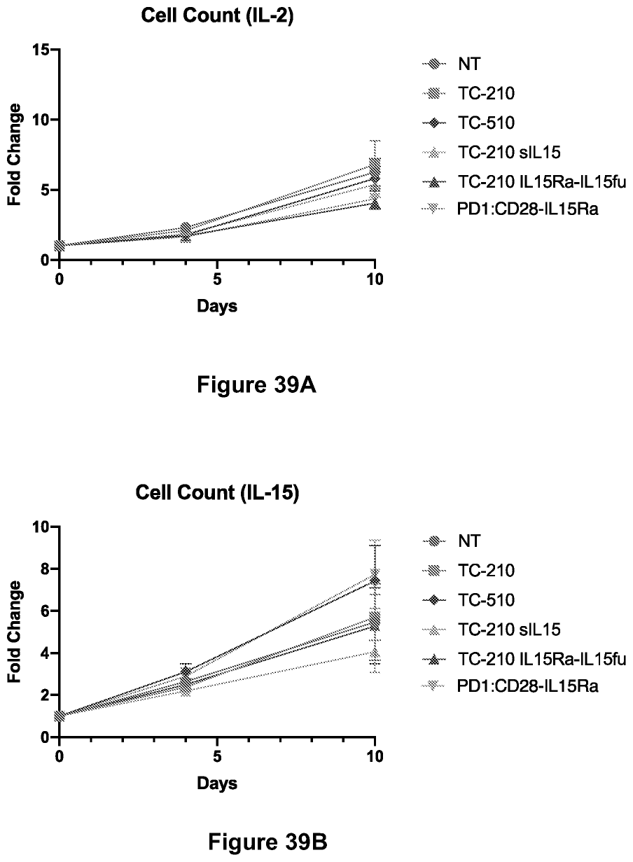

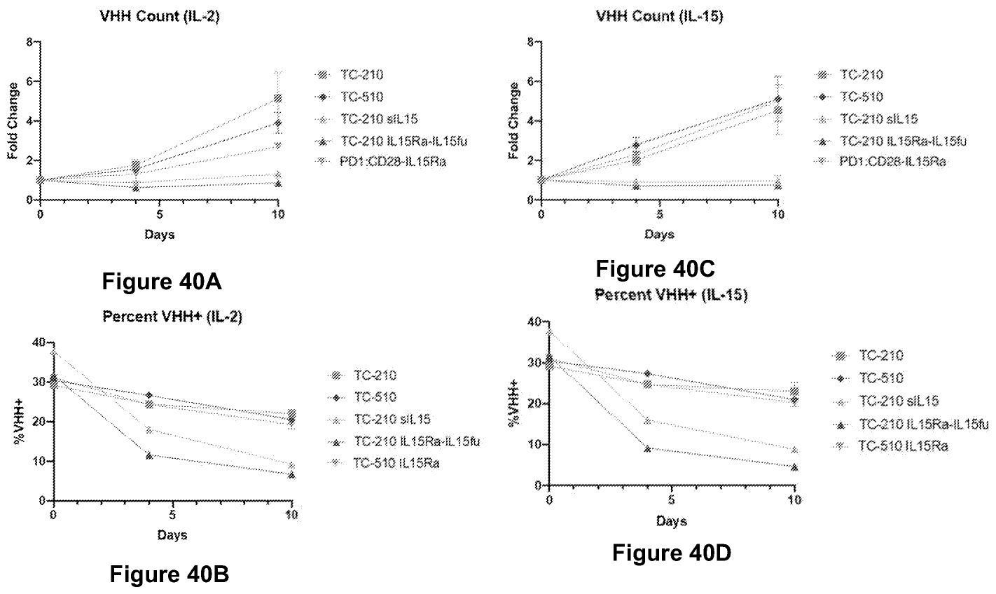

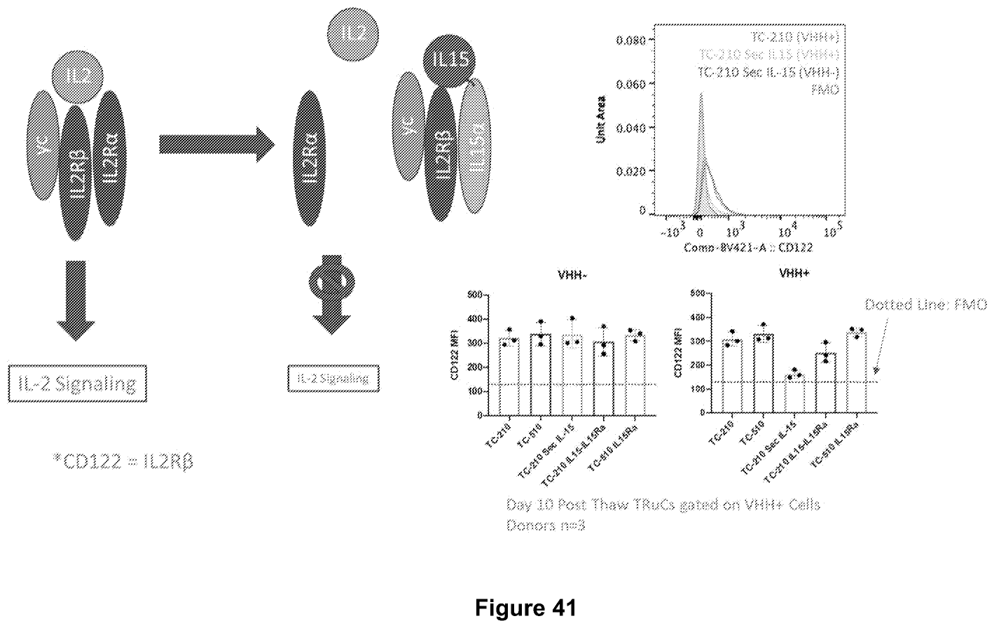

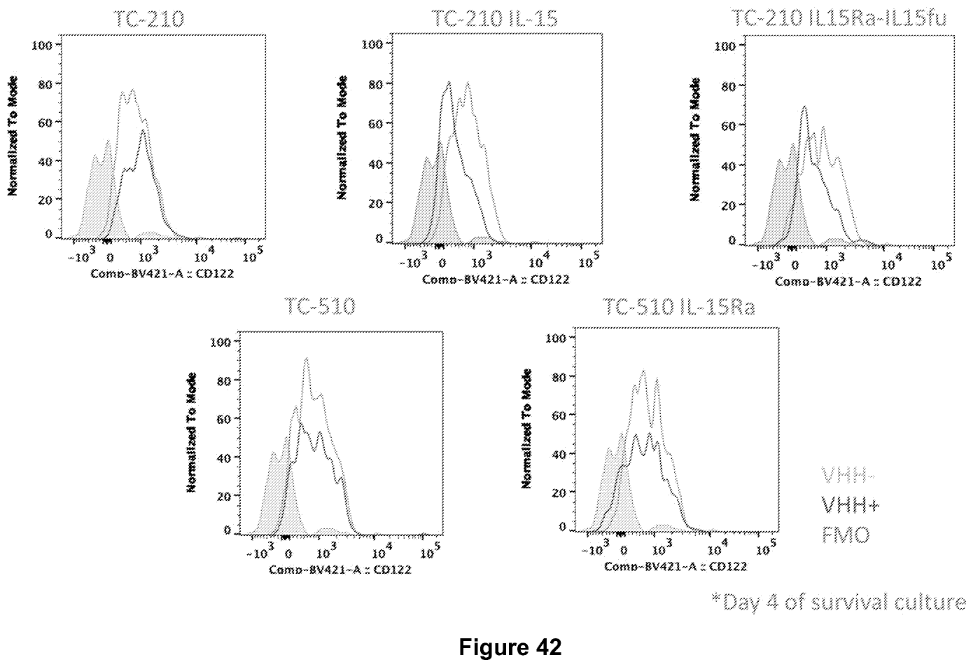

In this antigen independent expansion assay, it was also tested whether PDL1 exposure would induce a TC-510 IL15Ra transduced T cell to mimic the behavior of TC-210 transduced T cells co-expressing I1-15. Coating the bottom of the plate with PDL1-Fc did not induce this response, and these data are shown in FIGS. 38A and 38B. When IL-2 or IL-15 were exogenously added in this assay, transduced T cells expanded over time, but less of an increase in cell number was evident in transduced T cells co-expressing IL-15 as shown in FIGS. 39A and 39B. Fold change based on VHH count and quantification of percent VHH+ over time demonstrated that transduced T cells co-expressing IL-15 did not expand well in the presence of exogenous IL-2 or IL-15 as shown in FIGS. 40A-40D. FIG. 41 and FIG. 42 propose a schematic and related data for why transduced T cells co-expressing IL-15 did not respond as well in the presence of exogenous stimuli in this assay. Whilst not wishing to be bound by theory, it was proposed that the presence of co-expressed IL-15 may have competed with IL-2 for CD122 (IL2Rβ) as shown by the schematic in FIG. 41. Quantification of CD122 showed that transduced T cells co-expressing IL-15 had less CD122 available at their surface. Flow cytometry plots shown in FIG. 42 are representative images from Day 4 in culture.

The persistence assay demonstrated that IL15 expressed by TRuCs helped maintain T cell numbers in culture without external stimuli. IL15 helped maintain Naïve cells longer in the persistence culture compared to TC-210 alone or TC-510. Plate bound PDL1 alone did not provide a survival advantage to TC-510 TRuCs. TRuCs expanded when cultured with exogenous IL-2 or IL15, but IL15 expressing TRuCs did not respond as well to these exogenous stimuli, which may be due to a decrease in available CD122 on the surface of IL15 TRuCs.

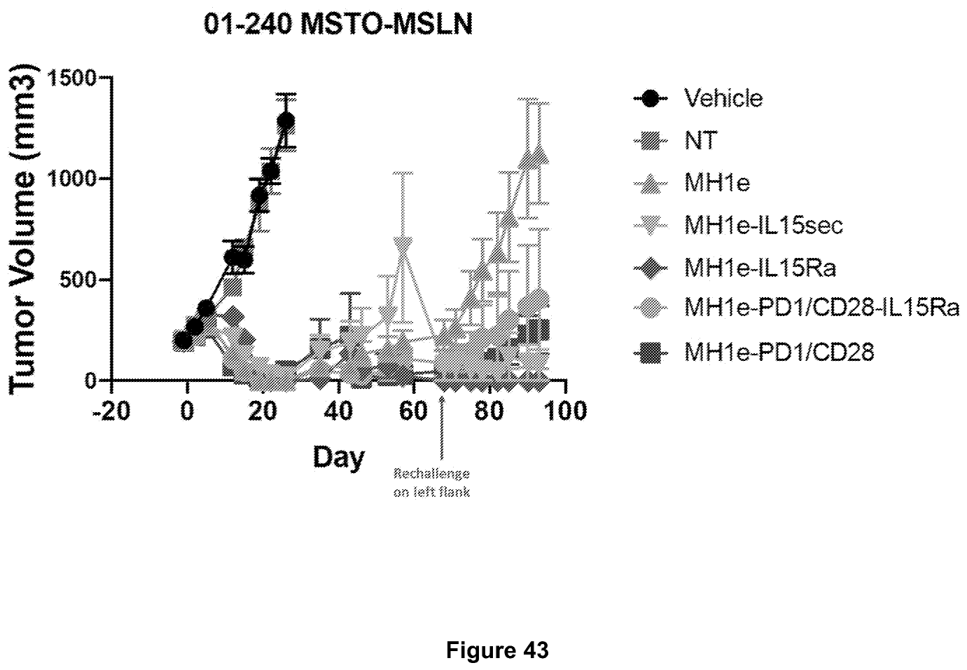

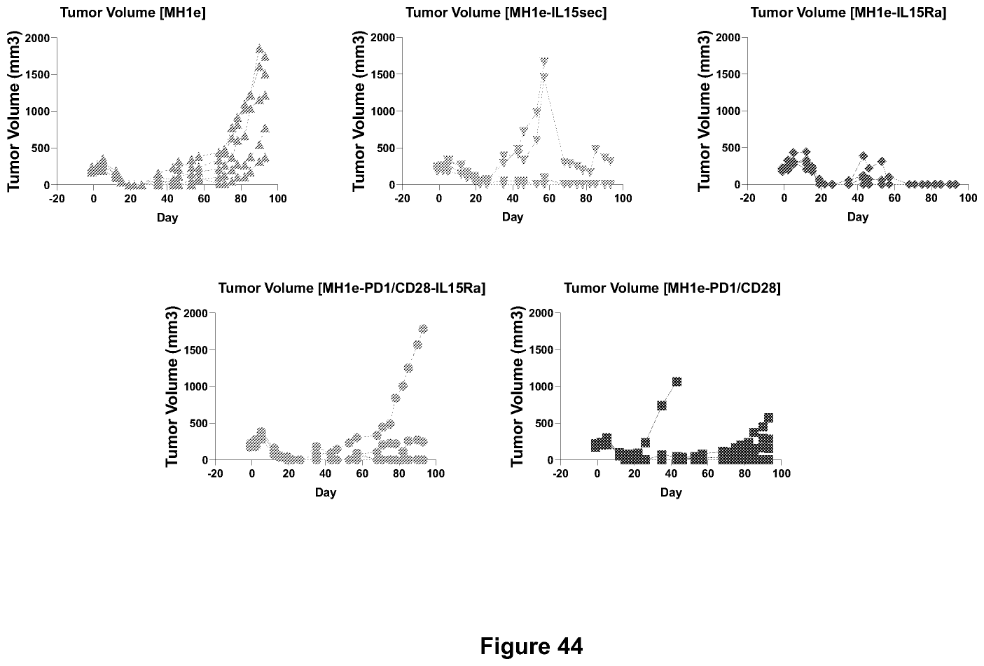

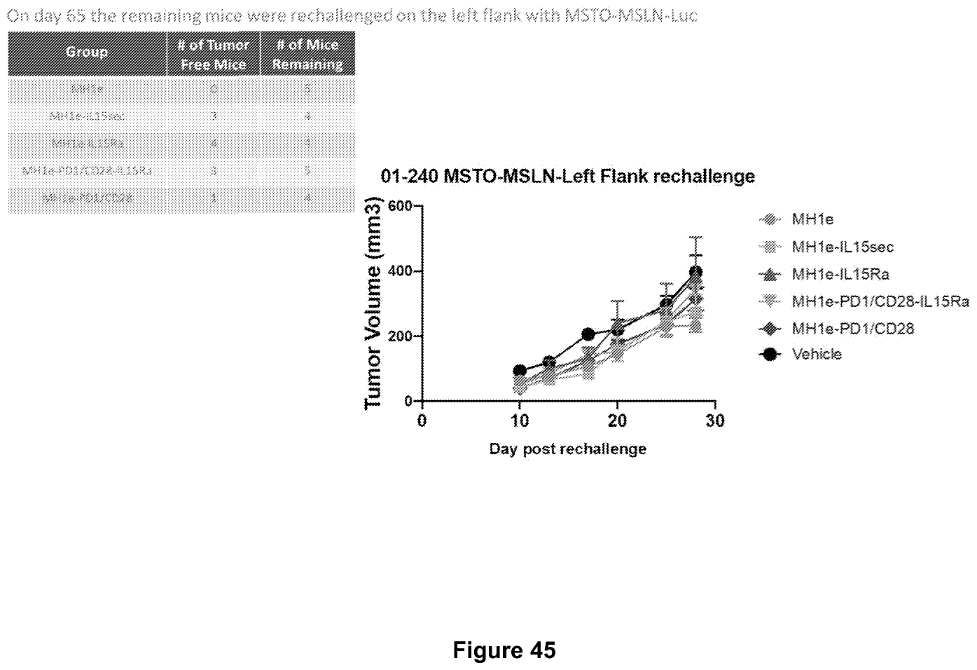

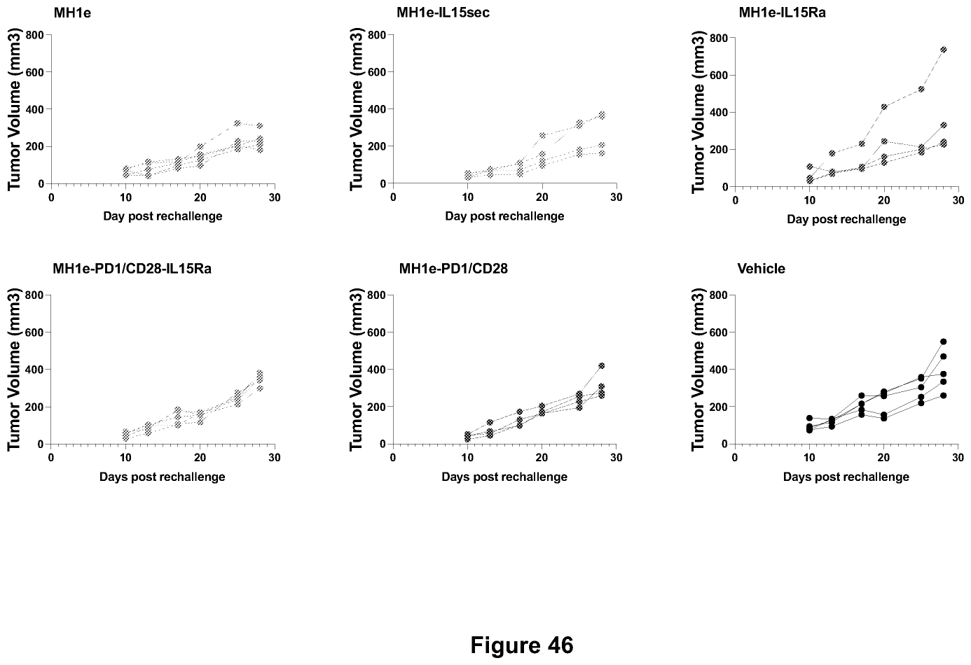

Efficacy of T cells transduced with TC-210, TC-510, TC-210 IL15, TC-210 IL15-IL15Ra, TC-510 IL15Ra was tested in vivo in MSTO-MSLN bearing animals. These in vivo studies can be used determine efficacy of different IL15 constructs compared to TC210 or TC510 in MSTO-MSLN bearing animals. MHC I/II deficient mice received implants of 10⁶ MSTO-MSLN-Luc and three weeks later (Day 0) received intravenous injection of 2×10⁶ transduced T cells normalized to 40% transduction efficiency. At this time point, tumors were typically 300-600 mm³. Tumor volume and weight measurements were taken regularly and at Day 65, any remaining mice received a tumor re-challenge in the left flank. FIG. 43 shows tumor volume measurement over time across all groups while FIG. 44 shows the same data split by the treatment (e.g., which transduced T cells were provided on Day 0). FIG. 45 shows data regarding the number of tumor free mice and the number of mice remaining on Day 65 of the study and further shows tumor volume over time subsequent to the re-challenge. FIG. 46 shows tumor volume measurements over time subsequent to the re-challenge on Day 65, split by treatment. Body weight measurements stayed substantially similar across groups and over time in this study, with no major weight loss evident. Each of the treatments tested in this study were able to successfully control the primary tumor.

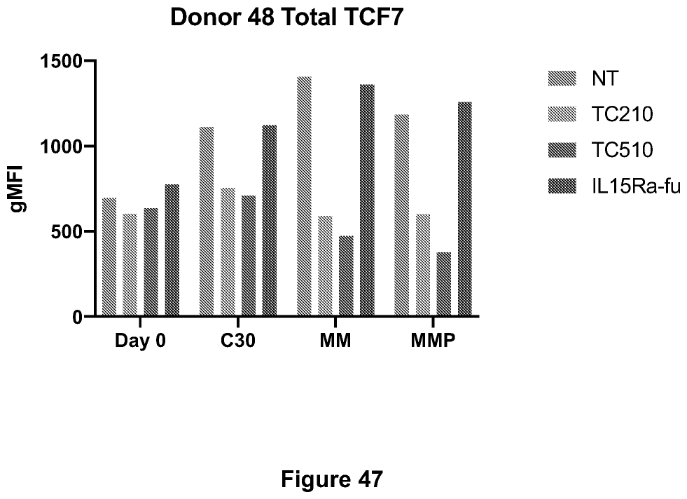

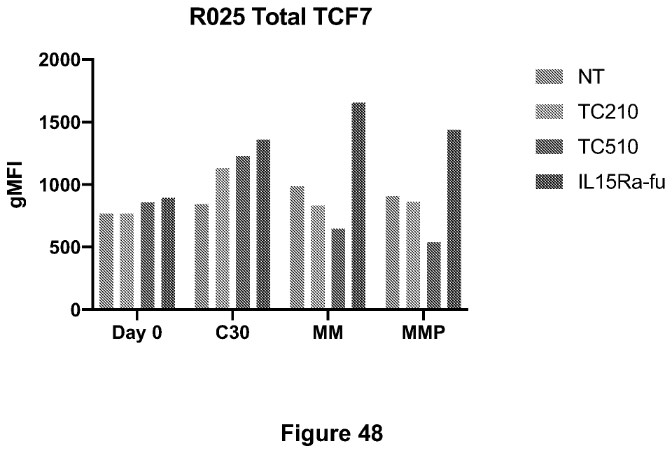

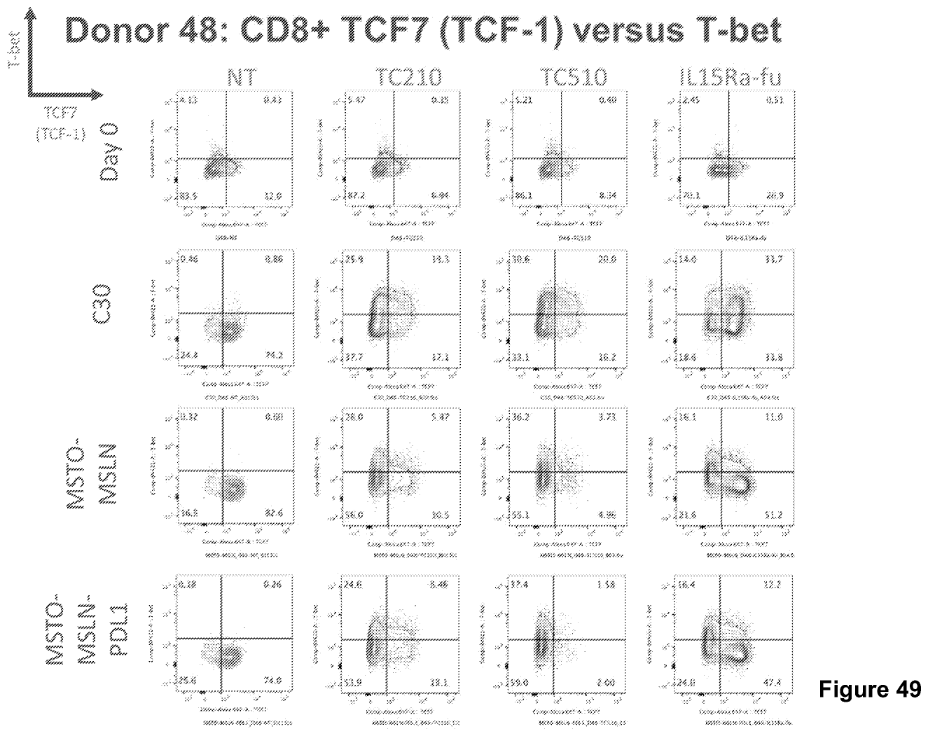

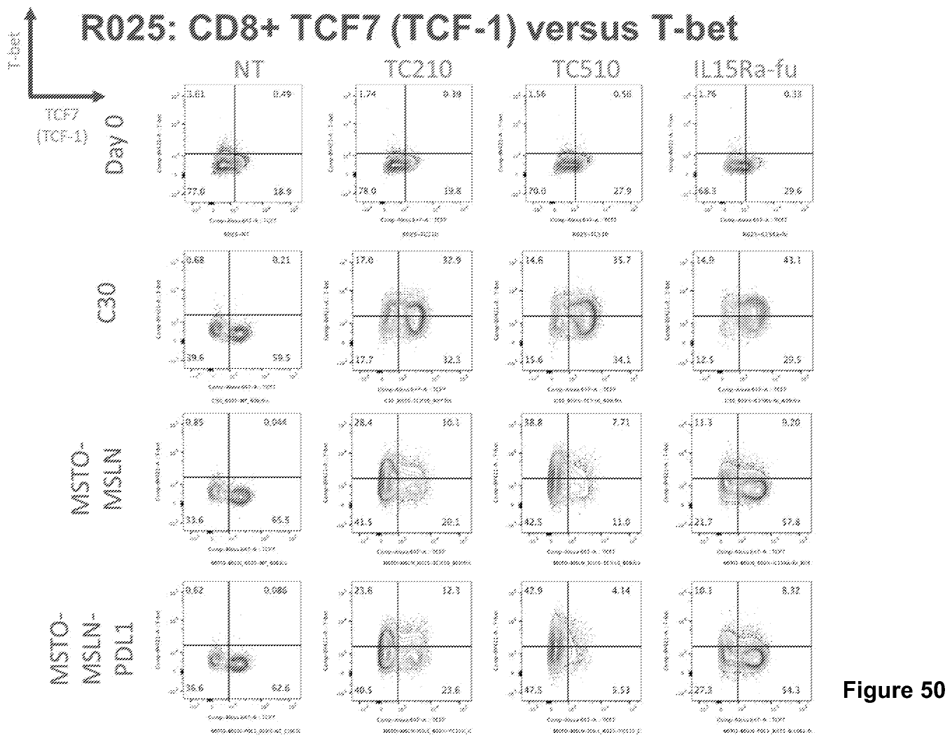

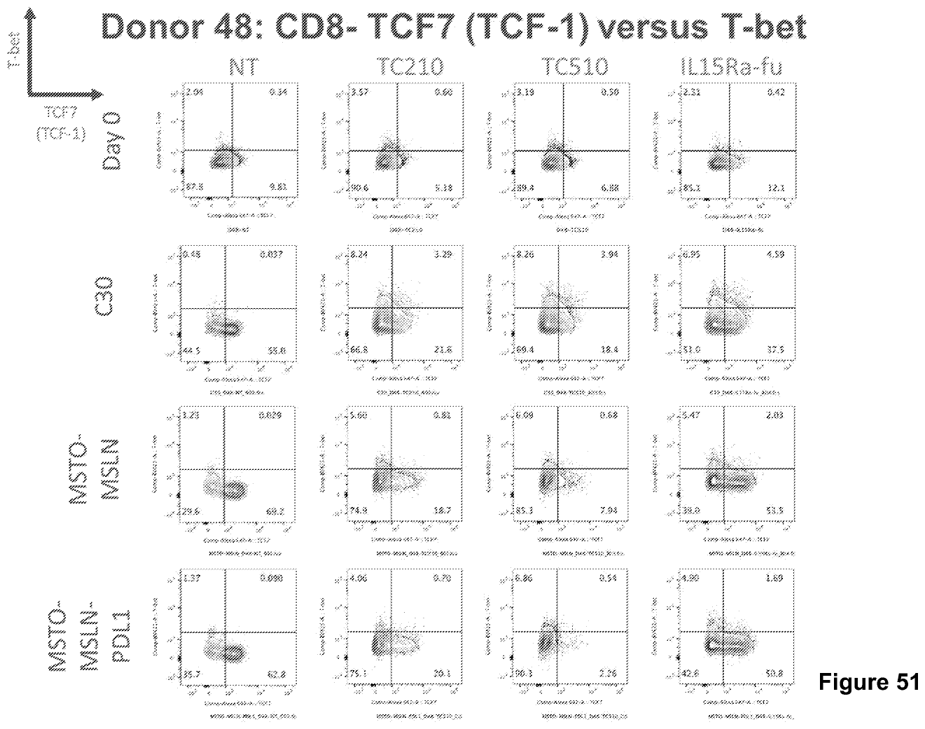

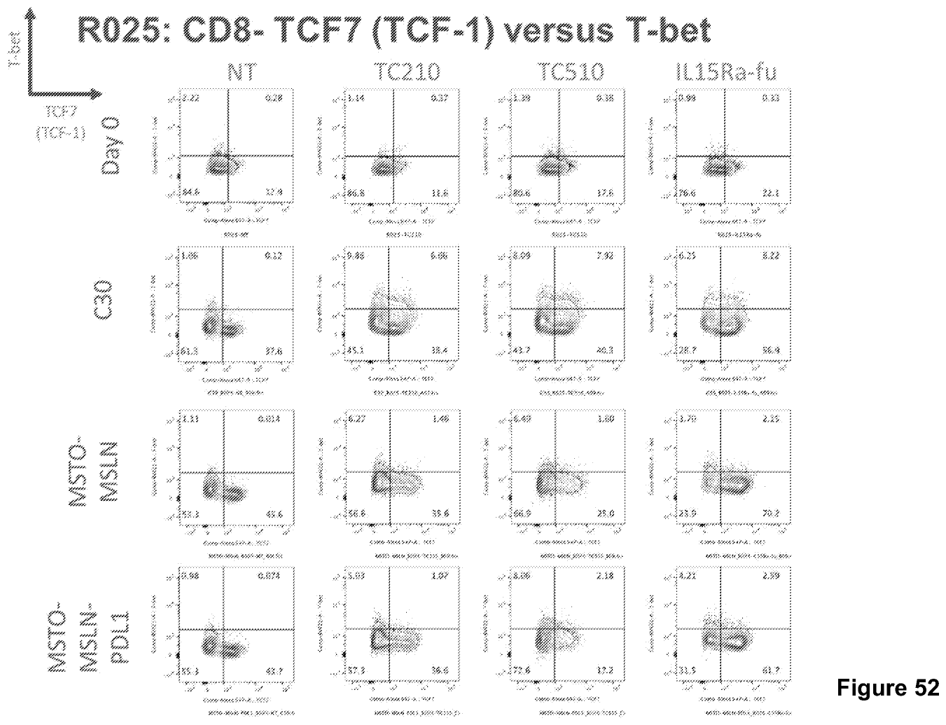

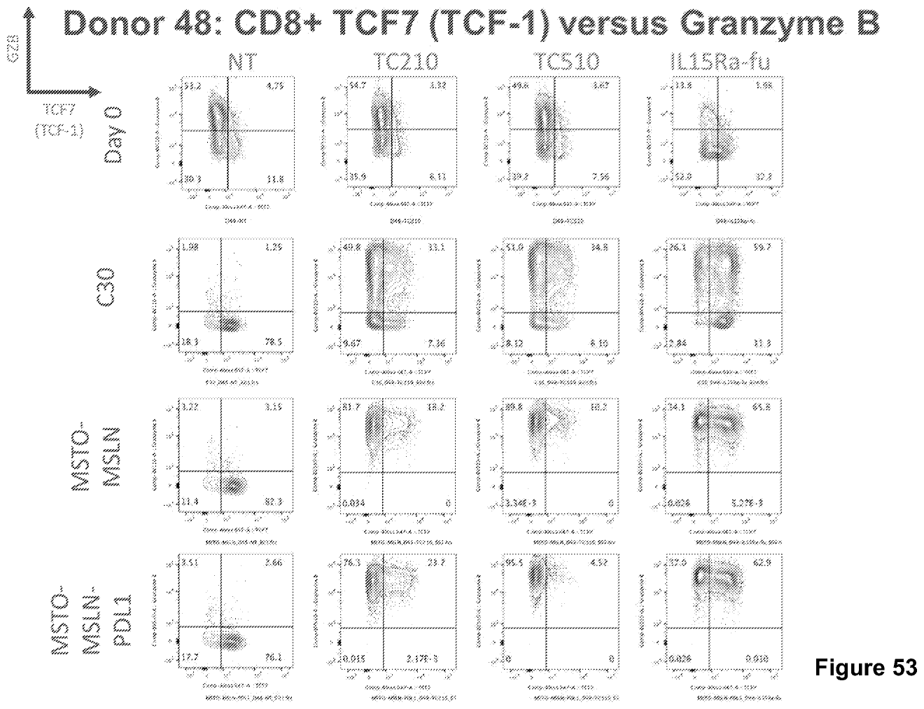

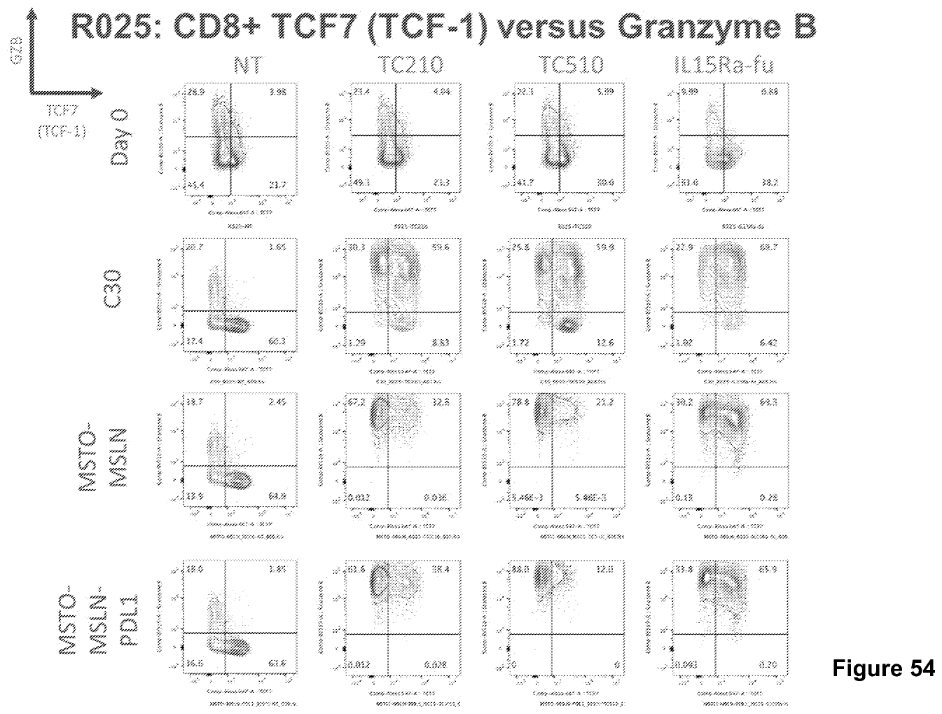

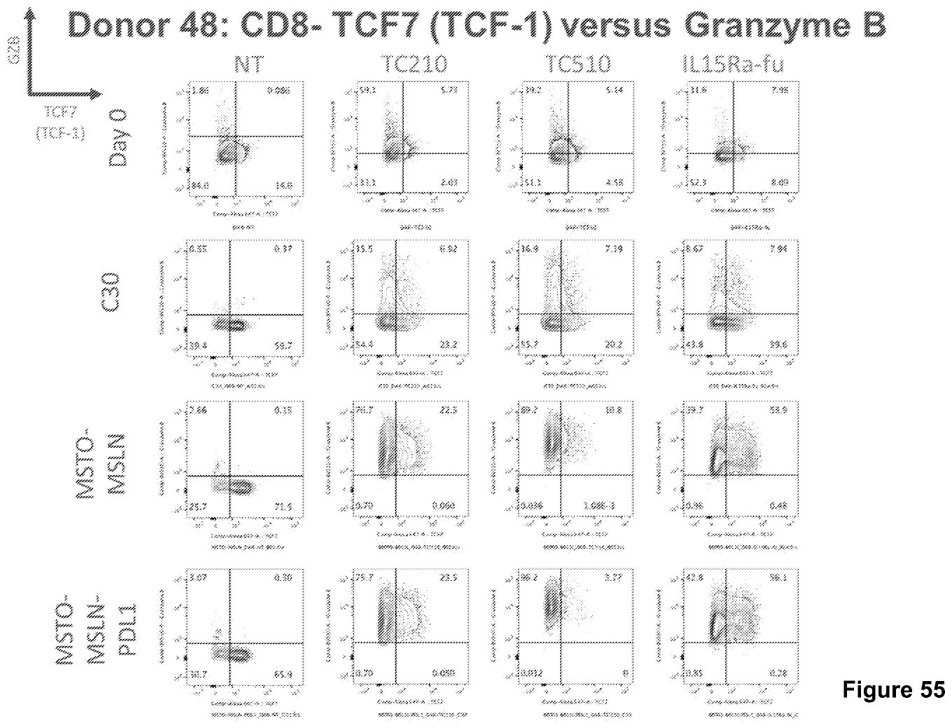

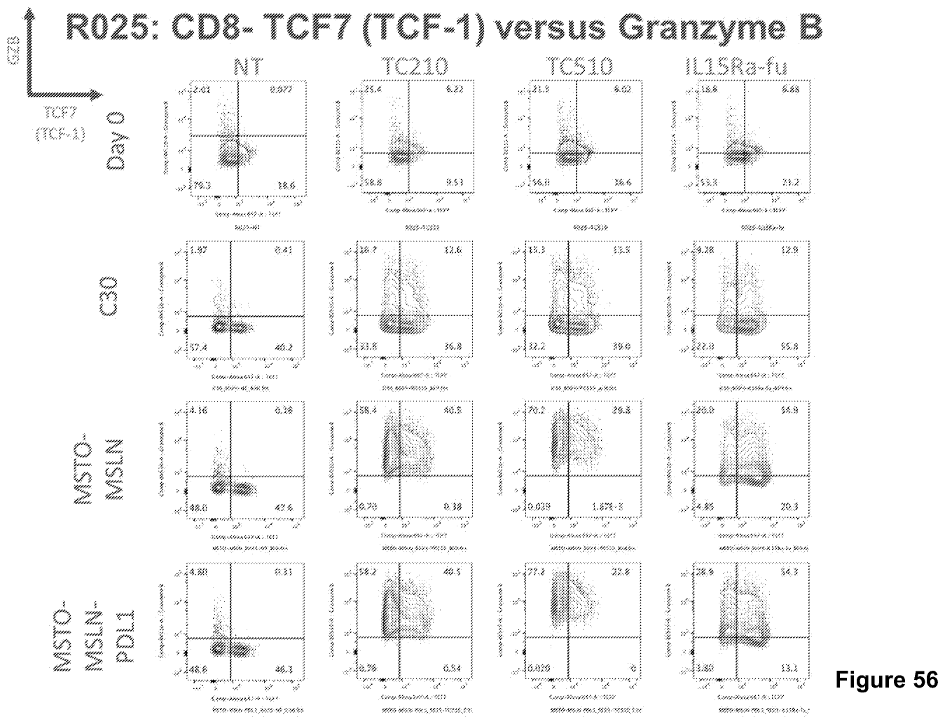

A TCF-7 (also known as TCF-1) expression assay was conducted with T cells transduced with a subset of the constructs of interest, specifically TC-210, TC-510 and TC-210 IL15Rα-IL15fu. On Day 0, C30, MSTO-MSLN (MM), or MSTO-MSLN-PDL1 (MMP) tumor cells were plated at a 1:1 ET ratio with transduced T cells. At 72 hours, the supernatant was harvested for cytokine assessment and at 96 hours the cells were collected for flow cytometric analysis. Quantification of TCF-7 from two separate donors is shown in FIG. 47 and FIG. 48, respectively. Flow cytometry plots showing surface expression of TCF-7 and T-bet in CD8+ cells for each of the two donors and across experimental conditions are shown in FIG. 49 and FIG. 50, respectively. Flow cytometry plots showing surface expression of TCF-7 and T-bet in CD8−cells for each of the two donors and across experimental conditions are shown in FIG. 51 and FIG. 52, respectively. Flow cytometry plots showing surface expression of TCF-7 and granzyme B in CD8+ cells for each of the two donors and across experimental conditions are shown in FIG. 53 and FIG. 54, respectively. Flow cytometry plots showing surface expression of TCF-7 and granzyme B in CD8−cells for each of the two donors and across experimental conditions are shown in FIG. 55 and FIG. 56, respectively.

In a second in vivo efficacy experiment testing transduced T cells co-expressing IL-15, MHC I/II deficient NSG mice were implanted at 6-7 weeks with 10⁶ MSTO-MSLN-luc cells and Matrigel (1:1). Fourteen days after tumor implant (tumors measuring approximately 80-200 mm³), mice received an intravenous infusion of 2×10⁶ transduced T cells (TC-210, TC-210 sIL15, TC-210 IL15Ra-IL15fu, TC-510). Fourteen days later, tissues (blood, spleen, liver, tumor) were harvested from three mice per group.

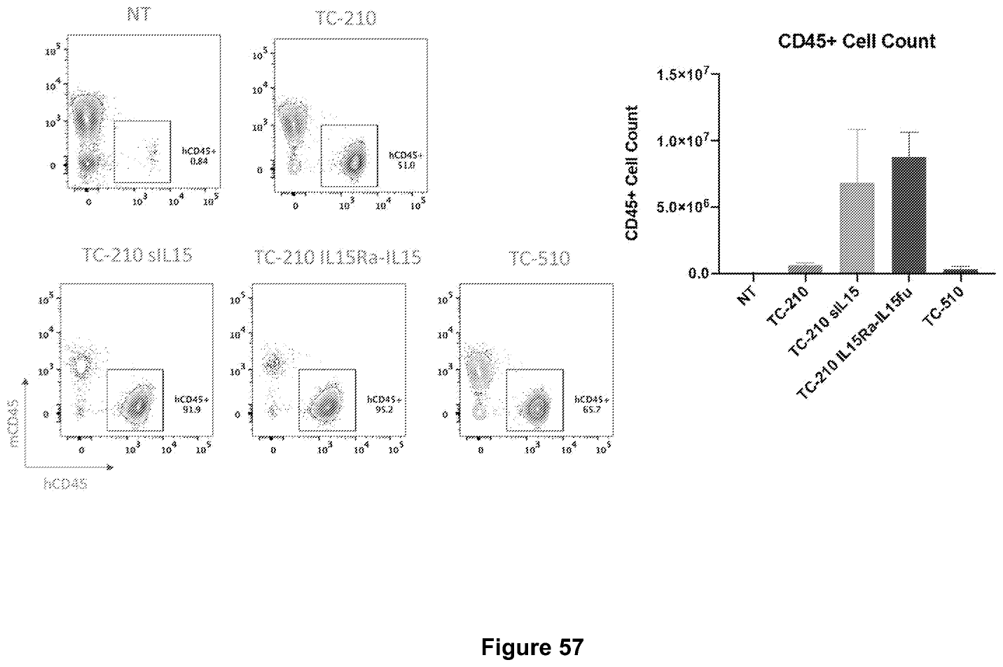

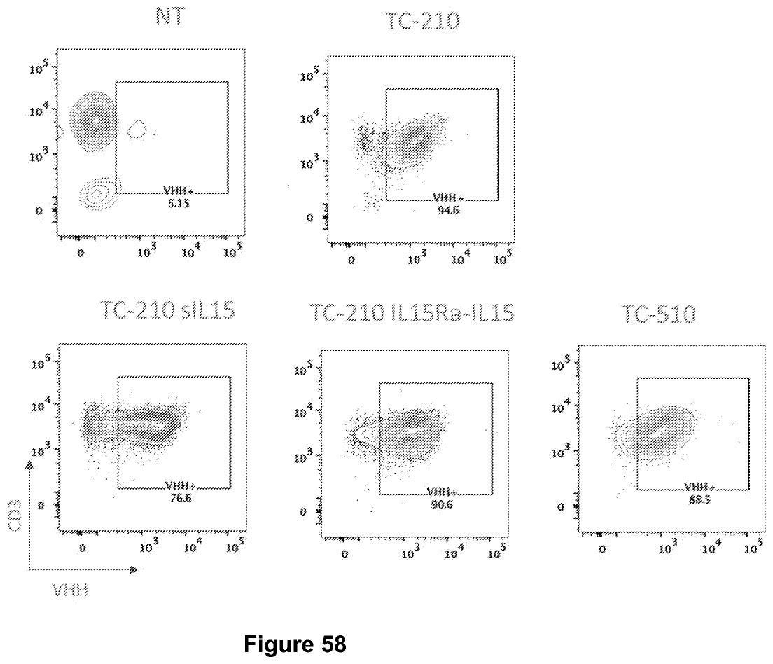

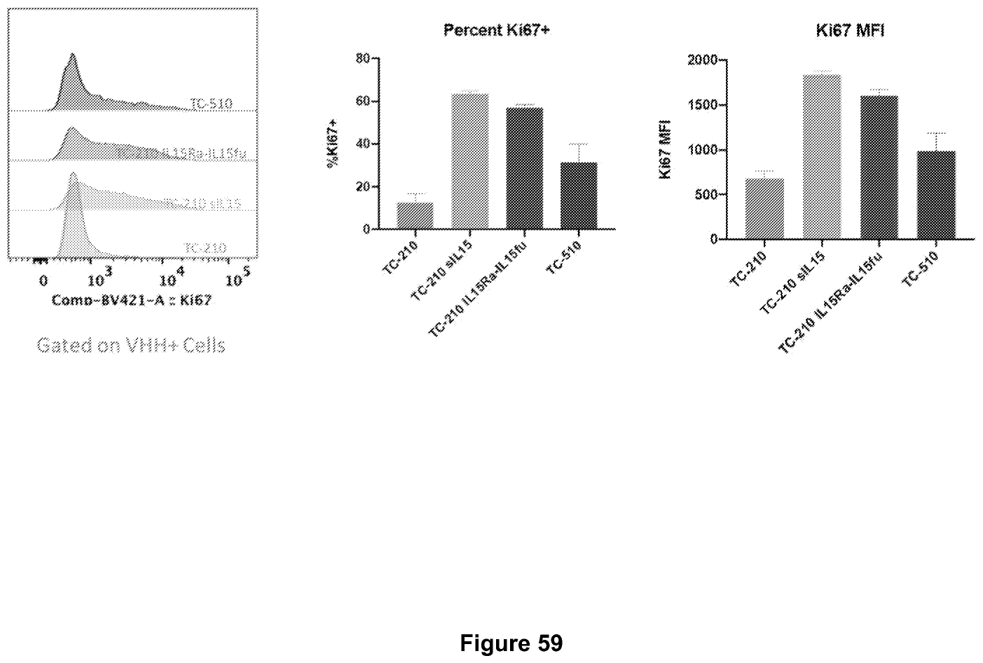

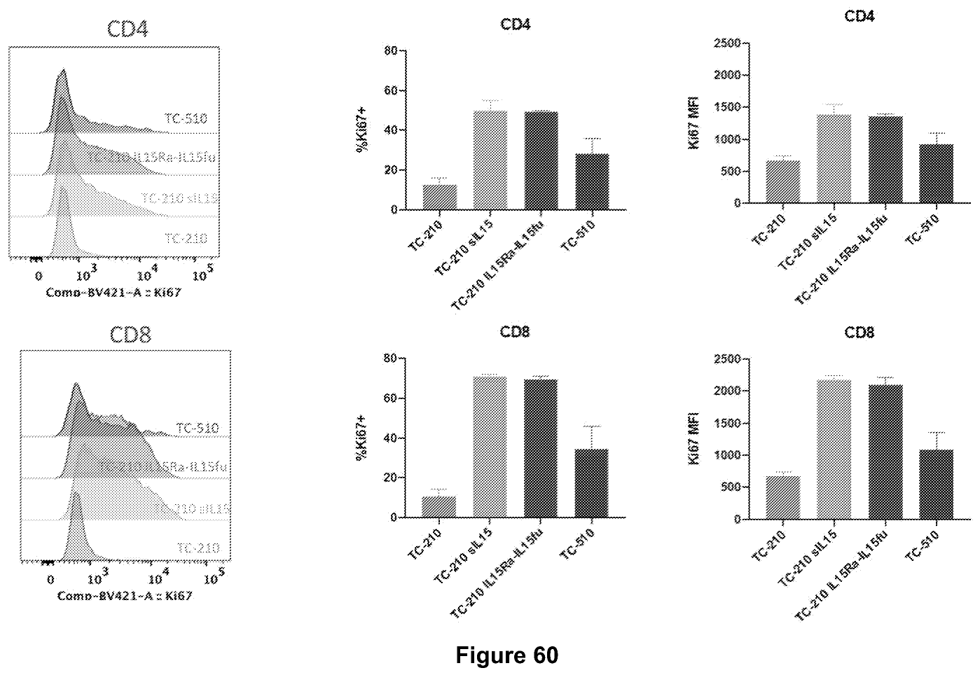

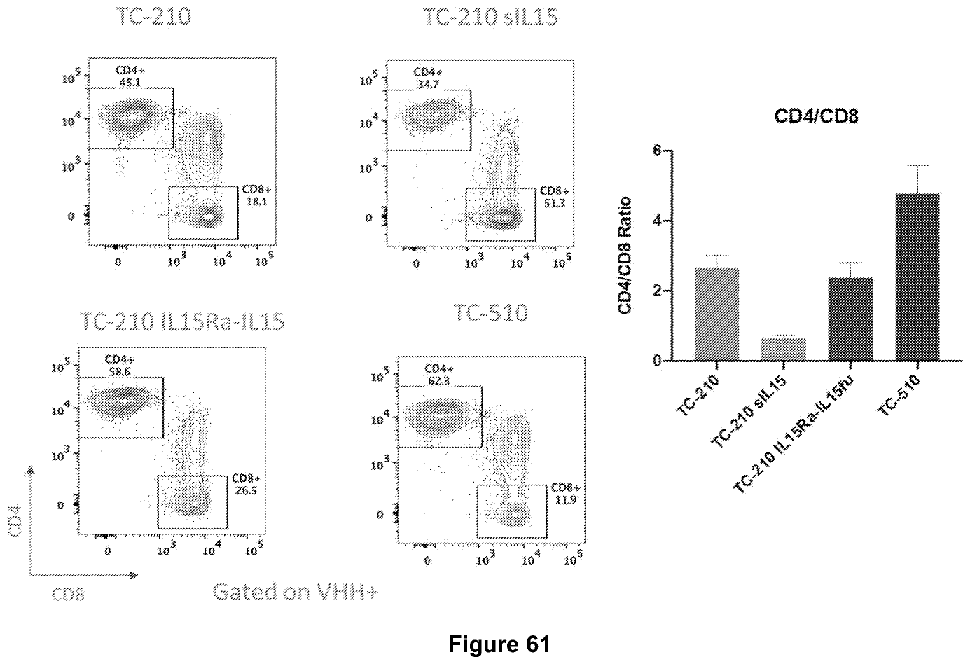

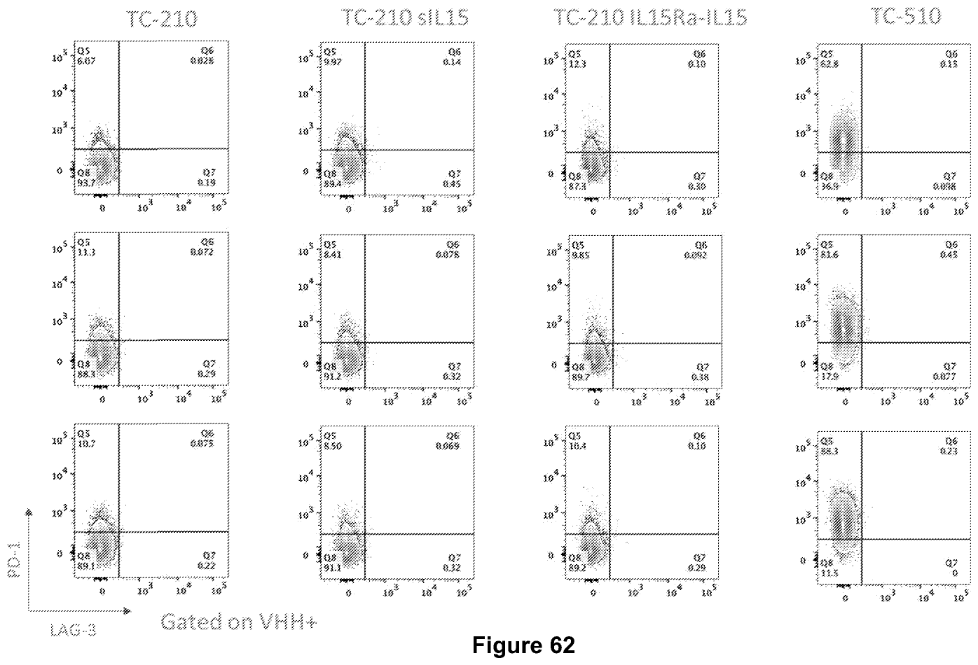

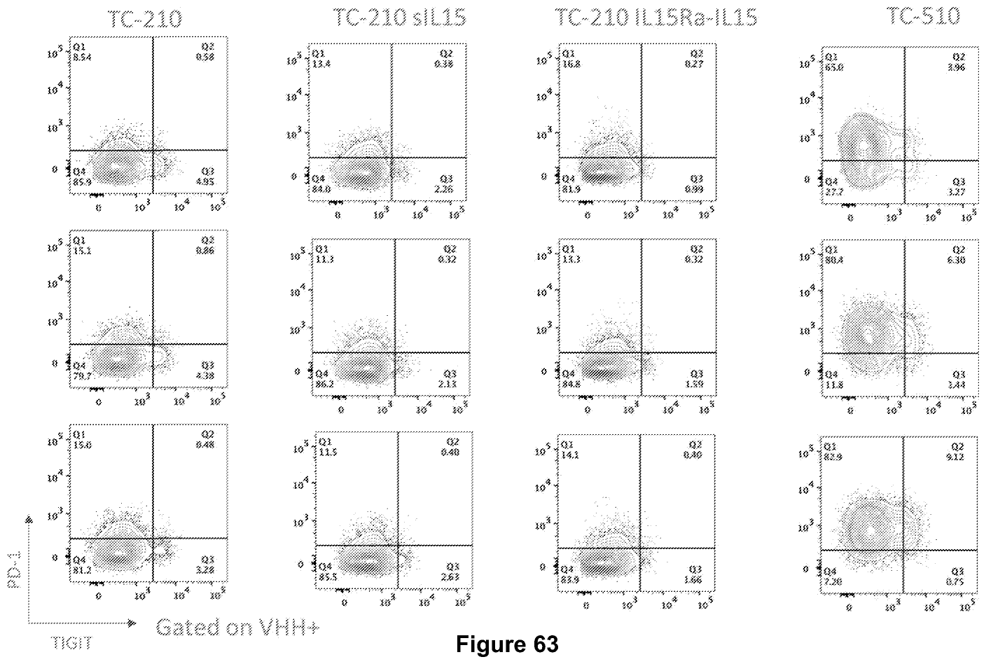

The tumor tissue collected from mice treated with transduced T cells was assessed for several markers. First, hCD45 surface expression was measured by flow cytometry and quantified as a CD45+ cell count. A histogram showing the cell count quantification and representative flow cytometry plots for each treatment group are shown in FIG. 57. Tumors of mice treated with transduced T cells co-expressing IL-15 showed increased levels of CD45 surface expression and increased CD45+ cell counts. VHH assessment by flow cytometry is shown in FIG. 58 and demonstrated that TC-210 sIL15 and TC-510 yielded a lower percentage of VHH in tumor tissues than the other tested treatments in this study. As a measure of proliferation, the presence of nuclear protein Ki67 was evaluated and the data are shown in FIG. 59. Transduced T cells co-expressing IL-15 showed higher levels of percent Ki67+ cell counts (gated on VHH+ cells) and higher levels of Ki67 mean fluorescence, suggesting increased in vivo proliferation of transduced T cells co-expressing IL-15. When these data were split by CD4+ versus CD8+ cell type as shown in FIG. 60, the Ki67 expression patterns remained the same, but were increased in CD8+ cells, indicating higher levels of proliferation in CD8+ transduced T cells co-expressing IL-15 in vivo. Evaluation of CD4+ versus CD8+ T cell populations (gated on VHH+) as shown in FIG. 61 demonstrated an enrichment of CD8+ cells in vivo in mice treated with TC-210 sIL15. Flow cytometric assessment of PD-1 and LAG-3 expression as shown in FIG. 62 showed minimal changes in inhibitory marker expression in tumors collected from mice treated with transduced T cells co-expressing IL-15. Assessment of PD-1 and TIGIT expression by flow cytometry showed similarly minimal changes in these inhibitory markers in tumors collected from mice treated with transduced T cells co-expressing IL-15 as shown in FIG. 63.

### Example 10: Expression of a Membrane-Tethered IL-15/IL-15 Receptor Fusion Protein Enhances Persistence of MSLN-Targeted TRuC T Cells

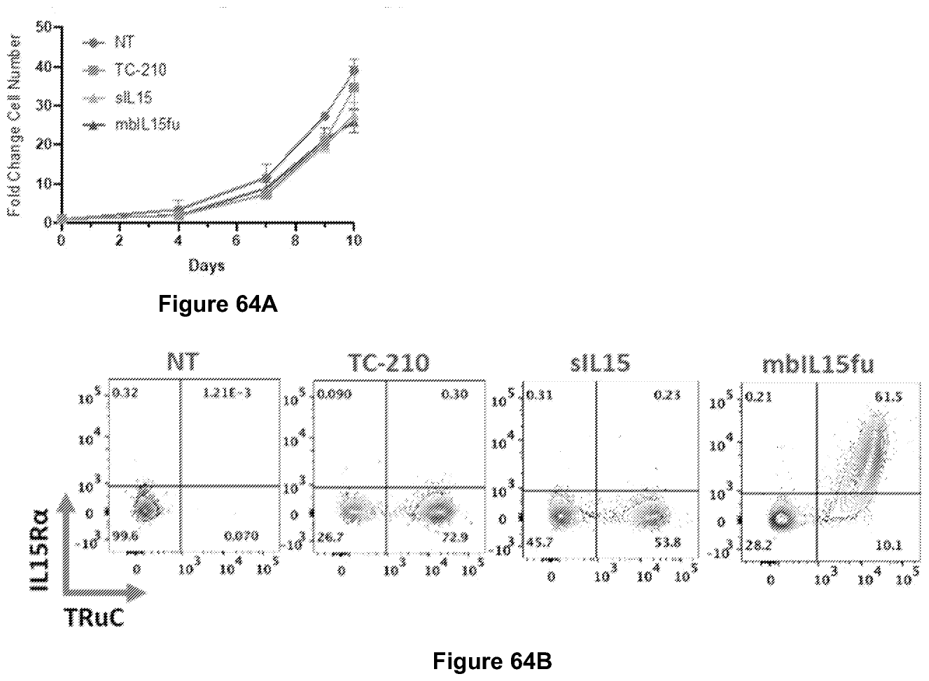

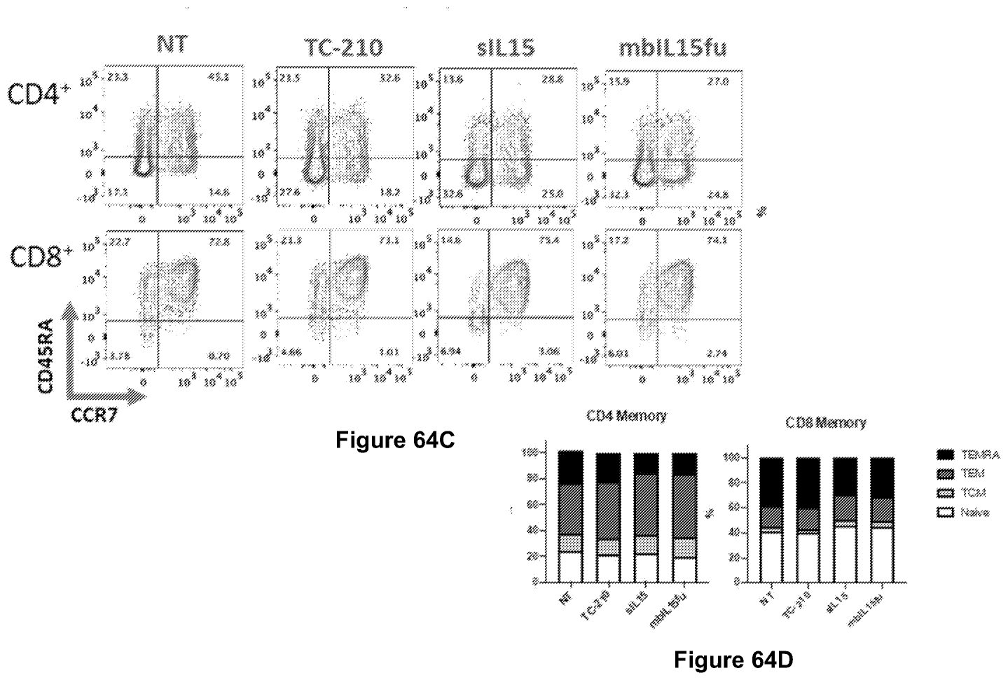

The potential of IL-15 co-expression as an enhancement to TRuC T cell phenotype, persistence, and function against MSLN+ targets was further evaluated with a series of studies. Naïve donor T cells activated by CD3/CD28 stimulation for 24 hour were transduced with lentiviral vectors packaging a sequence encoding (i) TC-210 (SEQ ID NO: 195), (ii) TC-210 sIL15 (SEQ ID NO: 380), or (iii) TC-210 mbIL15fu (SEQ ID NO: 381). Non-transfected (NT) T cells were used as a control group. T cells were further expanded for 9 days in media containing IL-7/IL-15. T cells transduced with TC-210, TC-210 sIL15 or TC-210 mbIL15fu all showed fold change expansion substantially similar to that seen in non-transfected control cells over this time period (FIG. 64A).

Cells were then harvested and subjected to flow cytometry to assess surface expression of TC-210 (VHH+) and IL15Ra. These data are shown in FIGS. 64B, which showed high co-expression of TC-210 and the membrane bound IL-15/IL-15Ra fusion construct (mbIL15fu).

The memory phenotype of transduced T cells was also assessed by flow cytometric analysis of CD45Ra and CCR7 presence in CD4+ or CD8+ cells, with data shown in FIG. 64C. Memory subsets (naïve, Temra, Tem and Tem) were quantified and plotted as percent of CD4+ and CD8+ transduced T cells across 7 donors as shown in FIG. 64D. Transduced T cells co-expressing IL-15 showed an increased proportion of CD8+ naïve/Tcm cells.

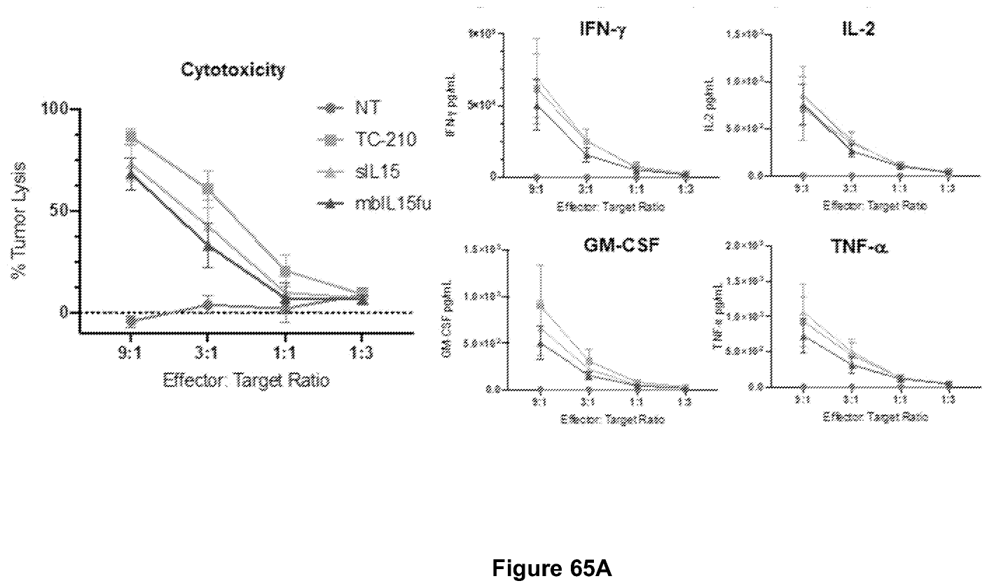

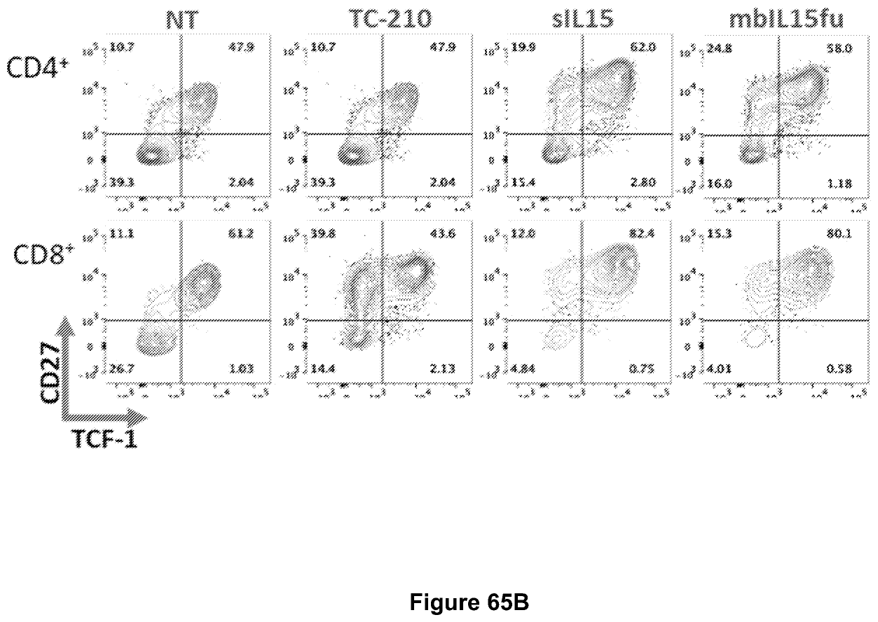

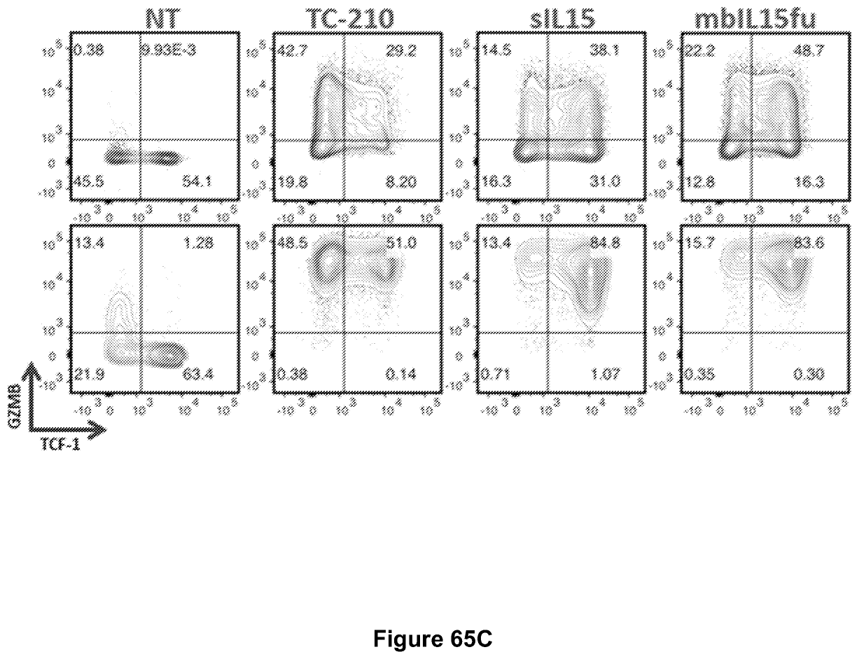

In vitro cytotoxicity and cytokine production were evaluated in single round 24 hr co-culture of transduced T cells with MSTO-MSLN cells. Data are shown in FIG. 65A. The data demonstrated that transduced T cells were able to successfully lyse tumor cells and produce a cytokine response in culture, as measured by IFNγ, IL-2, GM-CSF and TNFα.

The activation phenotype of transduced T cells was then evaluated using a 96 hr activation assay wherein 1.0e5 transduced (or NT) T cells were co-cultured with MSTO-MSLN cells at a 1:1 ratio, then after 96 hr, analyzed by flow cytometry for TRuC, TCF-1, CD27 and Granzyme B expression in CD4+ or CD8+ cells (FIG. 65B and FIG. 65C). These data suggested that transduced T cells co-expressing IL-15 show upregulated markers of stemness following activation.

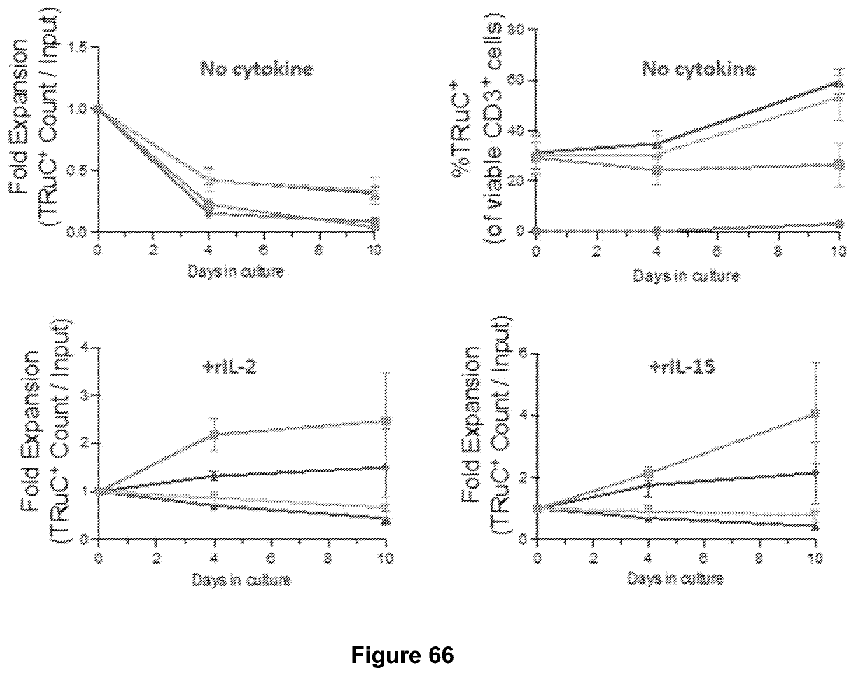

Transduced T cells were cultured for 10 days in R10 media alone, without cytokine supplement or in R10 media supplemented with IL-2 or IL-15. At each time point indicated, cells were harvested and stained for TRuC expression. Cell numbers were quantified and plotted as fold expansion relative to the number of cells cultured on Day 0. Transduced T cells co-expressing IL-15 showed an increased persistence under cytokine free culture conditions over 10 days. Transduced T cells grown in R10 media supplemented with cytokines were refractory to expansion. These data are shown in FIG. 66.

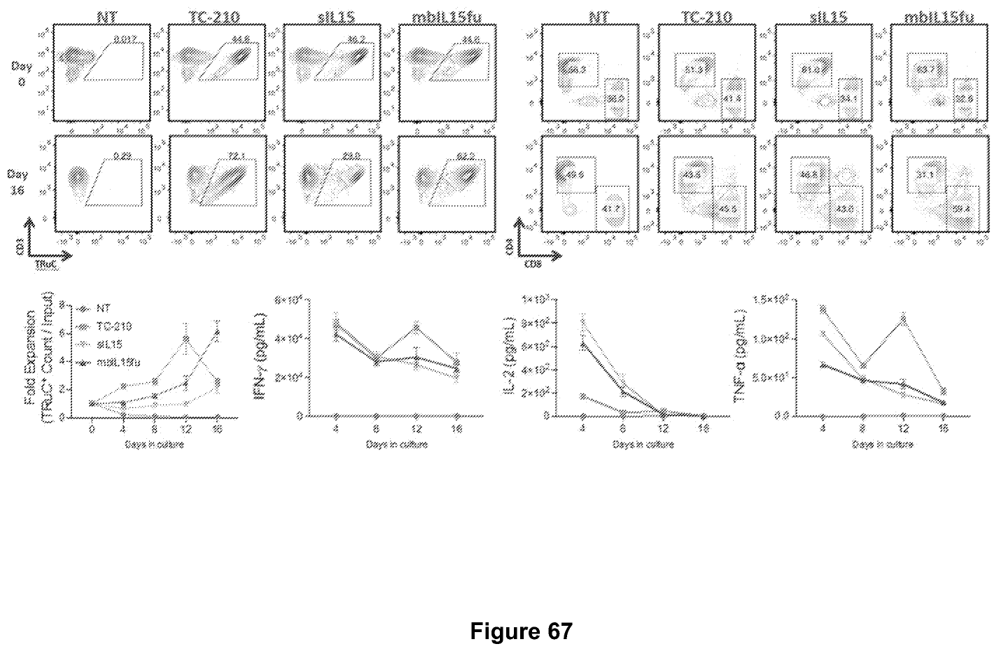

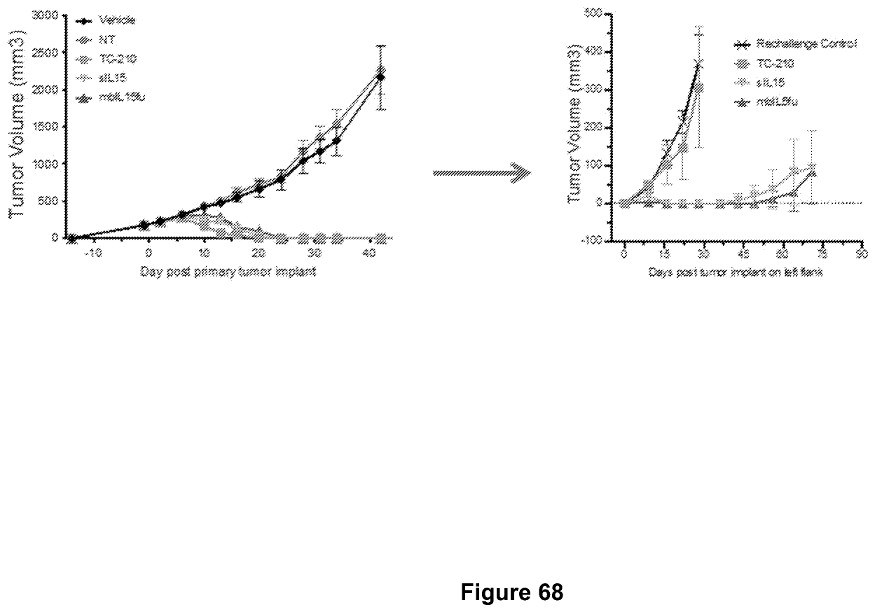

A repetitive stimulation assay was conducted to further test the transduced T cells and co-expression of an IL-15 enhancement strategy. T cells were cultured at 1:1 ratio with 3.0e4 MSTO-MSLN cells and re-stimulated every 4 days with additional tumor cells. Supernatants were collected for evaluation of cytokine release and the number of TRuC positive cells was determined by flow cytometry. FIG. 67 shows data from these studies, including flow cytometry plots of the frequency of TRuC+ (left) or CD4+/CD8+ (right) cells at Day 0 and Day 16. T cells transduced with TC-210 mbIL15fu maintained responsiveness and showed an expanded CD8+ population during repeated challenge with tumor cells.

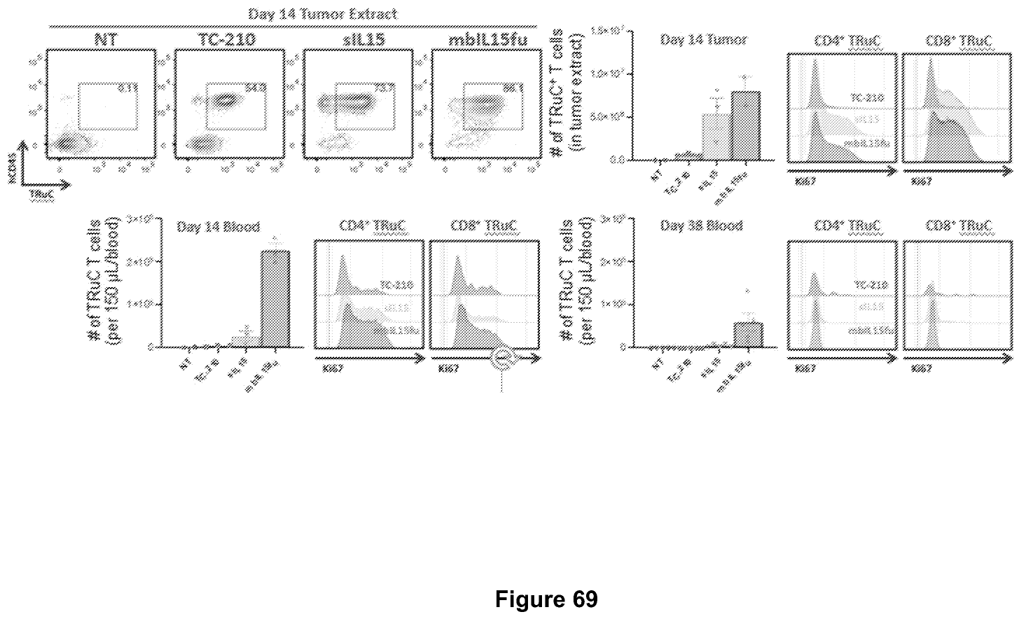

The anti-tumor efficacy of transduced T cells co-expressing an IL-15 enhancement was tested in vivo in NSG MHC class I/II deficient mice with implanted MSTO-MSLN tumor cells. Fourteen days after tumor implant, TC-210, TC-210 sIL or TC-210 mbIL15fu transduced T cells were provided. At 44 days after T cell infusion, cured mice were re-challenged with MSTO-MSLN tumors on the opposing flank. Results are shown in FIG. 68 and indicated that transduced T cells co-expressing IL-15 had an increased persistence that protected cured mice from tumor re-challenge. Fourteen days after T cell infusion, tumor and blood samples were collected and subjected to flow cytometric analysis for TRuC receptor and Ki67 expression. On Day 38 after T cell infusion, a second sample of blood was collected in order to evaluate TRuC T cells and TRuC+ cells were enumerated using counting beads. These data are summarized in FIG. 69. Higher tumor infiltration by transduced T cells co-expressing IL-15 was associated with increased T cell proliferation, expansion, and persistence. Taken together, transduced T cells co-expressing an IL-15 enhancement showed improved anti-tumoral efficacy in an in vivo tumor rechallenge xenogeneic model of mesothelioma.

TABLE 5
Antigen binding domain sequences.

| SEQ | | |
|---|---|---|
| ID | | |
| NO: | Name | Sequence |
| 24 | Anti-MSLN Light | DVVMTQTPLSLPVSLGDQASISCRSSQSLVHSNGNTYLHWYLQKP |
| | Chain amino acid | GQSPKLLIYKVSNRFSGVPDRFSGSGSGTDFTLKITRVEAEDLGVFF |
| | (MHC1445LC.1) | CSQSTHVPFTFGSGTKLEIK |
| 25 | Anti-MSLN Light | gatgttgtgatgacccaaactccactctccctgcctgtcagtcttggagatcaagcctccatctcttgcagat |
| | Chain | ctagtcagagccttgtacacagtaatggaaacacctatttacattggtacctgcagaagccaggccagtctc |
| | DNA (MHC1445LC.1) | caaagctcctgatctacaaagtttccaaccgattttctggggtcccagacaggttcagtggcagtggatcag |
| | | ggactgatttcacactcaagatcaccagagtggaggctgaggatctgggagtttttttctgctctcaaagtac |
| | | acatgttccattcacgttcggctcggggacaaagttggaaataaaa |
| 26 | Anti-MSLN Heavy | QVQLQQSGAELVRPGASVTLSCKASGYTFFDYEMHWVKQTPVHG |
| | Chain amino acid | LEWIGAIDPEIDGTAYNQKFKGKAILTADKSSSTAYMELRSLTSEDS |
| | (MHC1445HC.1) | AVYYCTDYYGSSYWYFDVWGTGTTVTVSS |
| 27 | Anti-MSLN Heavy | caggttcaactgcagcagtctggggctgagctggtgaggcctggggcttcagtgacgctgtcctgcaag |
| | Chain | gcttcgggctacacattttttgactatgaaatgcactgggtgaagcagacacctgtgcatggcctggaatgg |
| | DNA (MHC1445HC.1) | attggagctattgatcctgaaattgatggtactgcctacaatcagaagttcaagggcaaggccatactgact |
| | | gcagacaaatcctccagcacagcctacatggagctccgcagcctgacatctgaggactctgccgtctatta |
| | | ctgtacagattactacggtagtagctactggtacttcgatgtctggggcacagggaccacggtcaccgtct |
| | | cctc |
| 28 | Anti-MSLN Light | DVMMTQTPLSLPVSLGDQASISCRSSQSLVHSNGNTYLHWFLQKP |
| | Chain amino acid | GQSPKLLIYKVSNRFSGVPDRFSGSGSGTDFTLKISRVEAEDLGVYF |
| | (MHC1446LC.1) | CSQTTHVPLTFGAGTKLELK |
| 29 | Anti-MSLN Light | gatgttatgatgacccaaactccactctccctgcctgtcagtcttggagatcaagcctccatctcttgcagat |
| | Chain | ctagtcagagccttgtacacagtaatggaaacacctatttacattggttcctgcagaagccaggccagtctc |
| | DNA (MHC1446LC.1) | caaagctcctgatctacaaagtttccaaccgattttctggggtcccagacaggttcagtggcagtggatcag |
| | | ggacagatttcacactcaagatcagcagagtggaggctgaggatctgggagtttatttctgctctcaaacta |
| | | cacatgttccgctcacgttcggtgctgggaccaagctggagctgaaa |
| 30 | Anti-MSLN Heavy | QVQLQQSGAELVRPGASVTLSCKASGYTFTDYEMHWVKQTPVHG |
| | Chain amino acid | LEWIGAIDPEIAGTAYNQKFKGKAILTADKSSSTAYMELRSLTSEDS |
| | (MHC1446HC.3) | AVYYCSRYGGNYLYYFDYWGQGTTLTVSS |
| 31 | Anti-MSLN Heavy | caggttcaactgcagcagtctggggctgagctggtgaggcctggggcttcagtgacgctgtcctgcaag |
| | Chain | gcttcgggctacacttttactgactatgaaatgcactgggtgaagcagacacctgtccatggcctggaatg |
| | DNA (MHC1446HC.3) | gattggagctattgatcctgaaattgctggtactgcctacaatcagaagttcaagggcaaggccatactgac |
| | | tgcagacaaatcctccagcacagcctacatggagctccgcagcctgacatctgaggactctgccgtctatt |
| | | actgttcaagatacggtggtaactacctttactactttgactactggggccaaggcaccactctcacagtctc |
| | | ctca |
| 32 | Anti-MSLN Light | DVLMTQIPLSLPVSLGDQASISCRSSQNIVYSNGNTYLEWYLQKPG |
| | Chain amino acid | QSPKLLIYKVSNRFSGVPDRFSGSGSGTDFTLKISRVEAEDLGVYYC |
| | (MHC1447LC.5) | FQGSHVPFTFGSGTKLEIK |
| 33 | Anti-MSLN Light | gatgttttgatgacccaaattccactctccctgcctgtcagtcttggagatcaagcctccatctcttgcagatct |
| | Chain | agtcagaacattgtgtatagtaatggaaacacctatttagagtggtacctgcagaaaccaggccagtctcca |
| | DNA (MHC1447LC.5) | aagctcctgatctacaaagtttccaaccgattttctggggtcccagacaggttcagtggcagtggatcagg |
| | | gacagatttcacactcaagatcagcagagtggaggctgaggatctgggagtttattactgctttcaaggttc |
| | | acatgttccattcacgttcggctcggggacaaagttggaaataaaa |
| 34 | Anti-MSLN Heavy | QVQLQQSGAELVRPGASVTLSCKASGYTFTDYEMHWVKQTPVHG |
| | Chain amino acid | LEWIGAIDPEIGGSAYNQKFKGRAILTADKSSSTAYMELRSLTSEDS |
| | (MHC1447HC.5) | AVYYCTGYDGYFWFAYWGQGTLVTVSS |
| 35 | Anti-MSLN Heavy | caggttcaactgcagcagtccggggctgagctggtgaggcctggggcttcagtgacgctgtcctgcaag |
| | Chain | gcttcgggctacacatttactgactatgaaatgcactgggtgaagcagacacctgtgcatggcctggaatg |
| | DNA (MHC1447HC.5) | gattggagctattgatcctgaaattggtggttctgcctacaatcagaagttcaagggcagggccatattgact |
| | | gcagacaaatcctccagcacagcctacatggagctccgcagcctgacatctgaggactctgccgtctatta |
| | | ttgtacgggctatgatggttacttttggtttgcttactggggccaagggactctggtcactgtctcttca |
| 36 | Anti-MSLN Light | ENVLTQSPAIMSASPGEKVTMTCSASSSVSYMHWYQQKSSTSPKL |
| | Chain amino acid | WIYDTSKLASGVPGRFSGSGSGNSYSLTISSMEAEDVATYYCFQGS |
| | (MHC1448LC.4) | GYPLTFGSGTKLEIK |
| 37 | Anti-MSLN Light | gaaaatgttctcacccagtctccagcaatcatgtccgcatctccaggggaaaaggtcaccatgacctgcag |
| | Chain | tgctagctcaagtgtaagttacatgcactggtaccagcagaagtcaagcacctcccccaaactctggattta |
| | DNA (MHC1448LC.4) | tgacacatccaaactggcttctggagtcccaggtcgcttcagtggcagtgggtctggaaactcttactctct |
| | | cacgatcagcagcatggaggctgaagatgttgccacttattactgttttcaggggagtgggtacccactca |
| | | cgttcggctcggggacaaagttggaaataaaa |
| 38 | Anti-MSLN Heavy | QVQLQQSGAELVRPGASVTLSCKASGYTFTDYEMHWVKQTPVHG |
| | Chain amino acid | LEWIGGIDPETGGTAYNQKFKGKAILTADKSSSTAYMELRSLTSED |
| | (MHC1448HC.3) | SAVYYCTSYYGSRVFWGTGTTVTVSS |
| 39 | Anti-MSLN Heavy | caggttcaactgcagcagtctggggctgagctggtgaggcctggggcttcagtgacgctgtcctgcaag |
| | Chain | gcttcgggctacacatttactgactatgaaatgcactgggtgaaacagacacctgtgcatggcctggaatg |
| | DNA (MHC1448HC.3) | gattggaggtattgatcctgaaactggtggtactgcctacaatcagaagttcaagggtaaggccatactga |
| | | ctgcagacaaatcctccagcacagcctacatggagctccgcagcctgacatctgaggactctgccgtctat |
| | | tactgtacaagttactatggtagtagagtcttctggggcacagggaccacggtcaccgtctcctca |
| 40 | Anti-MSLN Light | QIVLSQSPAILSAFPGEKVTMTCRASSSVSYMHWYQQKPGSSPKPW |
| | Chain amino acid | IYATSNLASGVPARFSGSGSGTSYSLTISSVEAEDAATYYCQQWSS |
| | (MHC1449LC.3) | NPPTLTFGAGTKLELK |
| 41 | Anti-MSLN Light | caaattgttctctcccagtctccagcaatcctgtctgcatttccaggggagaaggtcactatgacttgcaggg |
| | Chain | ccagctcaagtgtaagttacatgcactggtaccagcagaagccaggatcctcccccaaaccctggatttat |
| | DNA (MHC1449LC.3) | gccacatccaacctggcttctggagtccctgctcgcttcagtggcagtgggtctgggacctcttactctctc |
| | | acaatcagcagtgtggaggctgaagatgctgccacttattactgccagcagtggagtagtaacccaccca |
| | | cgctcacgttcggtgctgggaccaagctggagctgaaa |
| 42 | Anti-MSLN Heavy | QVQLQQSGAELARPGASVKLSCKASGYTFTSYGISWVKQRTGQGL |
| | Chain amino acid | EWIGEIYPRSGNTYYNESFKGKVTLTADKSSGTAYMELRSLTSEDS |
| | (MHC1449HC.3) | AVYFCARWGSYGSPPFYYGMDYWGQGTSVTVSS |
| 43 | Anti-MSLN Heavy | caggttcagctgcagcagtctggagctgagctggcgaggcctggggcttcagtgaagctgtcctgcaag |
| | Chain | gcttctggctacaccttcacaagctatggtataagctgggtgaagcagaggactggacagggccttgagt |
| | DNA (MHC1449HC.3) | ggattggagagatttatcctagaagtggtaatacttactacaatgagagcttcaagggcaaggtcacactga |
| | | ccgcagacaaatcttccggcacagcgtacatggagctccgcagcctgacatctgaggactctgcggtcta |
| | | tttctgtgcaagatggggctcctacggtagtccccccttttactatggtatggactactggggtcaaggaacc |
| | | tcagtcaccgtctcctca |
| 44 | Anti-MSLN Light | DVLMTQTPLSLPVSLGNQASISCRSSQSIVHSSGSTYLEWYLQKPG |
| | Chain amino acid | QSPKLLIYKVSNRFSGVPDRFSGSGSGTDFTLKISRVEAEDLGVYYC |
| | (MHC1450LC.3) | FQGSHVPYTFGGGTKLEIK |
| 45 | Anti-MSLN Light | gatgttttgatgacccaaactccactctccctgcctgtcagtcttggaaatcaagcctccatctcttgcagatc |
| | Chain | tagtcagagcattgtacatagtagtggaagcacctatttagaatggtacctgcagaaaccaggccagtctc |
| | DNA (MHC1450LC.3) | caaagctcctgatctacaaagtttccaaccgattttctggggtcccagacaggttcagtggcagtggatcag |
| | | ggacagatttcacactcaagatcagcagagtggaggctgaggatctgggagtttattactgctttcaaggct |
| | | cacatgttccatacacgttcggaggggggaccaagctggaaataaaa |
| 46 | Anti-MSLN Heavy | QVQLQQSGAELARPGTSVKVSCKASGYTFTSYGISWVKQRIGQGL |
| | Chain amino acid | EWIGEIHPRSGNSYYNEKIRGKATLTADKSSSTAYMELRSLISEDSA |
| | (MHC1450HC.5) | VYFCARLITTVVANYYAMDYWGQGTSVTVSS |
| 47 | Anti-MSLN Heavy | caggttcagctgcagcagtctggagctgagctggcgaggcctgggacttcagtgaaggtgtcctgcaag |
| | Chain | gcttctggctataccttcacaagttatggtataagctgggtgaagcagagaattggacagggccttgagtg |
| | DNA (MHC1450HC.5) | gattggagagattcatcctagaagtggtaatagttactataatgagaagatcaggggcaaggccacactga |
| | | ctgcagacaaatcctccagcacagcgtacatggagctccgcagcctgatatctgaggactctgcggtctat |
| | | ttctgtgcaaggctgattactacggtagttgctaattactatgctatggactactggggtcaaggaacctcag |
| | | tcaccgtctcctca |
| 48 | Anti-MSLN Light | DIVMSQSPSSLAVSAGEKVTMSCKSSQSLLNSRTRKNYLAWYQQK |
| | Chain amino acid | PGQSPKLLIYWASTRESGVPDRFTGSGSGTDFTLTISSVQAEDLAVY |
| | (MHC1451LC.1) | YCKQSYNLVTFGAGTKLELK |
| 49 | Anti-MSLN Light | gacattgtgatgtcacagtctccatcctccctggctgtgtcagcaggagagaaggtcactatgagctgcaa |
| | Chain | atccagtcagagtctgctcaacagtagaacccgaaagaactacttggcttggtaccagcagaaaccaggg |
| | DNA (MHC1451LC.1) | cagtctcctaaactgctgatctactgggcatccactagggaatctggggtccctgatcgcttcacaggcagt |
| | | ggatctgggacagatttcactctcaccatcagcagtgtgcaggctgaagacctggcagtttattactgcaaa |
| | | caatcttataatctggtcacgttcggtgctgggaccaagctggagctgaaa |
| 50 | Anti-MSLN Heavy | QVQLQQSGAELVRPGASVTLSCKASGYTFFDYEMHWVKQTPVHG |
| | Chain amino acid | LEWIGAIDPEIDGTAYNQKFKGKAILTADKSSSTAYMELRSLTSEDS |
| | (MHC1451HC.2) | AVYYCTDYYGSSYWYFDVWGTGTTVTVSS |
| 51 | Anti-MSLN Heavy | caggttcaactgcagcagtctggggctgagctggtgaggcctggggcttcagtgacgctgtcctgcaag |
| | Chain | gcttcgggctacacattttttgactatgaaatgcactgggtgaagcagacacctgtgcatggcctggaatgg |
| | DNA (MHC1451HC.2) | attggagctattgatcctgaaattgatggtactgcctacaatcagaagttcaagggcaaggccatactgact |
| | | gcagacaaatcctccagcacagcctacatggagctccgcagcctgacatctgaggactctgccgtctatta |
| | | ctgtacagattactacggtagtagctactggtacttcgatgtctggggcacagggaccacggtcaccgtct |
| | | cctc |
| 52 | Anti-MSLN Light | QIVLTQSPAIMSASPGEKVTISCSASSSVSYMYWYQQKPGSSPKPWI |
| | Chain amino acid | YRTSNLASGVPARFSGSGSGTSYSLTISSMEAEDAATYYCQQYHSY |
| | (MHC1452LC.1) | PLTFGAGTKLELK |
| 53 | Anti-MSLN Light | caaattgttctcacccagtctccagcaatcatgtctgcatctccaggggagaaggtcaccatatcctgcagt |
| | Chain | gccagctcaagtgtaagttacatgtactggtaccagcagaagccaggatcctcccccaaaccctggattta |
| | DNA (MHC1452LC.1) | tcgcacatccaacctggcttctggagtccctgctcgcttcagtggcagtgggtctgggacctcttactctctc |
| | | acaatcagcagcatggaggctgaagatgctgccacttattactgccagcagtatcatagttacccactcac |
| | | gttcggtgctgggaccaagctggagctgaaa |
| 54 | Anti-MSLN Light | QIVLTQSPAIMSASPGERVTMTCSASSSVSSSYLYWYQQKSGSSPK |
| | Chain amino acid | LWIYSISNLASGVPARFSGSGSGTSYSLTINSMEAEDAATYYCQQW |
| | (MHC1452LC.6) | SSNPQLTFGAGTKLELK |
| 55 | Anti-MSLN Light | caaattgttctcacccagtctccagcaatcatgtctgcatctcctggggaacgggtcaccatgacctgcagt |
| | Chain | gccagctcaagtgtaagttccagctacttgtactggtaccagcagaagtcaggatcctccccaaaactctg |
| | DNA (MHC1452LC.6) | gatttatagcatatccaacctggcttctggagtcccagctcgcttcagtggcagtgggtctgggacctcttac |
| | | tctctcacaatcaacagcatggaggctgaagatgctgccacttattactgccagcagtggagtagtaaccc |
| | | acagctcacgttcggtgctgggaccaagctggagctgaaa |
| 56 | Anti-MSLN Heavy | QVQLKQSGAELVKPGASVKISCKASGYTFTDYYINWVKQRPGQGL |
| | Chain amino acid | EWIGKIGPGSGSTYYNEKFKGKATLTADKSSSTAYMQLSSLTSEDS |
| | (MHC1452HC.2) | AVYFCARTGYYVGYYAMDYWGQGTSVTVSS |
| 57 | Anti-MSLN Heavy | caggtccagctgaagcagtctggagctgagctggtgaagcctggggcttcagtgaagatatcctgcaag |
| | Chain | gcttctggctacaccttcactgactactatataaactgggtgaagcagaggcctggacagggccttgagtg |
| | DNA (MHC1452HC.2) | gattggaaagattggtcctggaagtggtagtacttactacaatgagaagttcaagggcaaggccacactga |
| | | ctgcagacaaatcctccagcacagcctacatgcagctcagcagcctgacatctgaggactctgcagtctat |
| | | ttctgtgcaagaactggttactacgttggttactatgctatggactactggggtcaaggaacctcagtcaccg |
| | | tctcctca |
| 58 | Anti-MSLN Heavy | QVQLQQSGAELARPGASVKLSCKASGYTFTIYGISWVKQRTGQGL |
| | Chain amino acid | EWIGEIYPRSDNTYYNEKFKGKATLTADKSSSTAYMELRSLTSEDS |
| | (MHC1452HC.4) | AVYFCARWYSFYAMDYWGQGTSVTVSS |
| 59 | Anti-MSLN Heavy | caggttcagctgcagcagtctggagctgagctggcgaggcctggggcttcagtgaagctgtcctgcaag |
| | Chain | gcttctggctacaccttcacaatctatggtataagctgggtgaaacagagaactggacagggccttgagtg |
| | DNA (MHC1452HC.4) | gattggagagatttatcctagaagtgataatacttactacaatgagaagttcaagggcaaggccacactga |
| | | ctgcagacaaatcctccagcacagcgtacatggagctccgcagcctgacatctgaggactctgcggtcta |
| | | tttctgtgcaagatggtactcgttctatgctatggactactggggtcaaggaacctcagtcaccgtctcctca |
| 60 | anti-MSLN (SD1) | GGDWSANFMY |
| | CDR1 | |
| 61 | anti-MSLN (SD1) | RISGRGVVDYVESVKGRFT |
| | CDR2 | |
| 62 | anti-MSLN (SD1) | ASY |
| | CDR3 | |
| 63 | anti-MSNL (SD4) | GSTSSINTMY |
| | CDR1 | |
| 64 | anti-MSNL (SD4) | FISSGGSTNVRDSVKGRFT |
| | CDR2 | |
| 65 | anti-MSNL (SD4) | YIPYGGTLHDF |
| | CDR3 | |
| 66 | anti-MSNL (SD6) | GSTFSIRAMR |
| | CDR1 | |
| 67 | anti-MSNL (SD6) | VIYGSSTYYADAVKGRFT |
| | CDR2 | |
| 68 | anti-MSNL (SD6) | DTIGTARDY |
| | CDR3 | |
| 69 | Single domain | EVQLVESGGGLVQPGGSLRLSCAASGGDWSANFMYWYRQAPGK |
| | anti- | QRELVARISGRGVVDYVESVKGRFTISRDNSKNTLYLQMNSLRAE |
| | MSLN binder 1 | DTAVYYCAVASYWGQGTLVTVSS |
| | (SD1) | |
| 70 | Single domain | EVQLVESGGGLVQPGGSLRLSCAASGSTSSINTMYWYRQAPGKER |
| | anti- | ELVAFISSGGSTNVRDSVKGRFTISRDNSKNTLYLQMNSLRAEDTA |
| | MSLN binder 4 | VYYCNTYIPYGGTLHDFWGQGTLVTVSS |
| | (SD4) | |
| 71 | Single domain | QVQLVESGGGVVQAGGSLRLSCAASGSTFSIRAMRWYRQAPGTER |
| | anti- | DLVAVIYGSSTYYADAVKGRFTISRDNSKNTLYLQMNSLRAEDTA |
| | MSLN binder 6 | VYYCNADTIGTARDYWGQGTLVTVSS |
| | (SD6) | |
| 72 | Anti-CD19 light | AGGGCAAGTCAGGACATTAGTAAA |
| | chain | |
| | CDR1 coding | |
| | sequence | |
| 73 | Anti-CD19 light | RASQDISK |
| | chain | |
| | CDR1 | |
| 74 | Anti-CD19 light | ATCTACCATACATCAAGATTA |
| | chain | |
| | CDR2 coding | |
| | sequence | |
| 75 | Anti-CD19 light | IYHTSRL |
| | chain CDR2 | |
| 76 | Anti-CD19 light | CAACAGGGTAATACGCTTCCGTACACG |
| | chain | |
| | CDR3 coding | |
| | sequence | |
| 77 | Anti-CD19 light | QQGNTLPYT |
| | chain CDR3 | |
| 78 | Anti-CD19 heavy | GGGGTCTCATTACCCGACTATGGTGTAAGC |
| | chain | |
| | CDR1 coding | |
| | sequence | |
| 79 | Anti-CD19 heavy | GVSLPDYGVS |
| | chain CDR1 | |
| 80 | Anti-CD19 heavy | GTAATATGGGGTAGTGAAACCACATACTATAATTCAGCTCTC |
| | chain | |
| | CDR2 coding | |
| | sequence | |
| 81 | Anti-CD19 heavy | VIWGSETTYYNSAL |
| | chain CDR2 | |
| 82 | Anti-CD19 heavy | CATTATTACTACGGTGGTAGCTATGCTATGGACTAC |
| | chain | |
| | CDR3 coding | |
| | sequence | |
| 83 | Anti-CD19 heavy | HYYYGGSYAMDY |
| | chain CDR3 | |
| 84 | Anti-CD19 light | GACATCCAGATGACACAGACTACATCCTCCCTGTCTGCCTCTCT |
| | chain | GGGAGACAGAGTCACCATCAGTTGCAGGGCAAGTCAGGACATT |
| | region coding | AGTAAATATTTAAATTGGTATCAGCAGAAACCAGATGGAACTG |
| | sequence | TTAAACTCCTGATCTACCATACATCAAGATTACACTCAGGAGTC |
| | | CCATCAAGGTTCAGTGGCAGTGGGTCTGGAACAGATTATTCTCT |
| | | CACCATTAGCAACCTGGAGCAAGAAGATATTGCCACTTACTTTT |
| | | GCCAACAGGGTAATACGCTTCCGTACACGTTCGGAGGGGGGAC |
| | | TAAGTTGGAAATAACA |
| 85 | Anti-CD19 light | DIQMTQTTSSLSASLGDRVTISCRASQDISKYLNWYQQKPDGTVKL |
| | chain | LIYHTSRLHSGVPSRFSGSGSGTDYSLTISNLEQEDIATYFCQQGNT |
| | variable region | LPYTFGGGTKLEIT |
| 86 | Anti-CD19 heavy | GAGGTGAAACTGCAGGAGTCAGGACCTGGCCTGGTGGCGCCCT |
| | chain variable | CACAGAGCCTGTCCGTCACATGCACTGTCTCAGGGGTCTCATTA |
| | region coding | CCCGACTATGGTGTAAGCTGGATTCGCCAGCCTCCACGAAAGG |
| | sequence | GTCTGGAGTGGCTGGGAGTAATATGGGGTAGTGAAACCACATA |
| | | CTATAATTCAGCTCTCAAATCCAGACTGACCATCATCAAGGACA |
| | | ACTCCAAGAGCCAAGTTTTCTTAAAAATGAACAGTCTGCAAACT |
| | | GATGACACAGCCATTTACTACTGTGCCAAACATTATTACTACGG |
| | | TGGTAGCTATGCTATGGACTACTGGGGTCAAGGAACCTCAGTCA |
| | | CCGTCTCCTCA |
| 87 | Anti-CD19 heavy | EVKLQESGPGLVAPSQSLSVTCTVSGVSLPDYGVSWIRQPPRKGLE |
| | chain | WLGVIWGSETTYYNSALKSRLTIIKDNSKSQVFLKMNSLQTDDTAI |
| | variable region | YYCAKHYYYGGSYAMDYWGQGTSVTVSS |
| 88 | CD70 CDR1 | SIFDIVRGS |
| 89 | CD70 CDR2 | AIITSGGATNYA |
| 90 | CD70 CDR3 | CNMESVRYRNYW |
| 91 | CD70 variable | QVQLQESGGGLVQTGGSLRLSCTASGSIFDIVRGSWYRQAPGNQR |
| | domain | ELVAIITSGGATNYADSVAGRFTISRDSAWKALYLQMNSLKPEDTA |
| | | VYFCNMESVRYRNYWGQGTQVTVSS |
| 92 | CD70 CDR1 | FTLEHYSIG |
| 93 | CD70 CDR2 | SCITSSGGIPKYA |
| 94 | CD70 CDR3 | CGAATPDDDCSVPGHYGLNYW |
| 95 | CD70 variable | QVQLQESGGGLVQPGGSLRLSCAASGFTLEHYSIGWFRQAPGKDL |
| | domain | EGVSCITSSGGIPKYADSVKGRFIISRDNAKNTGYLQMNSLKPEDT |
| | | AVYYCGAATPDDDCSVPGHYGLNYWGKGTQVTVSS |
| 96 | CD70 CDR1 | FTFDAYAIG |
| 97 | CD70 CDR2 | ICLSPSDGSTYYA |
| 98 | CD70 CDR3 | CATPSWCSLKADFGSW |
| 99 | CD70 variable | QVQLQESGGGLVQAGGSLRLSCAAPGFTFDAYAIGWFRQAPGKER |
| | domain | EGVICLSPSDGSTYYADSVKGRFTISSDNAKNTVYLQMNSLKPEDT |
| | | AVYYCATPSWCSLKADFGSWGQGTQVTVSS |
| 100 | CD70 CDR1 | SIFSATRME |
| 101 | CD70 CDR2 | AIVTSGGRTNYA |
| 102 | CD70 CDR3 | CKFERYDYVNYW |
| 103 | CD70 variable | QVQLQESGGGLVQPGGSLRLSCTASGSIFSATRMEWYRQAPGKQR |
| | domain | ELVAIVTSGGRTNYADSVNGRFTISRDNAKNTLYLQMNNLKPEDT |
| | | AVYYCKFERYDYVNYWGRGTQVTVSS |
| 104 | CD70 CDR1 | SIFSIARMN |
| 105 | CD70 CDR2 | AILNRAGRTDYA |
| 106 | CD70 CDR3 | CNLQTISYHDFW |
| 107 | CD70 variable | QVQLQESGGGLVQPGGSLRLSCVASGSIFSIARMNWYRQAPGKQR |
| | domain | ELVAILNRAGRTDYADSVKGRFTISSDNAKTTVYLQMNSLKPEDT |
| | | ALYYCNLQTISYHDFWGQGTQVTVSS |
| 108 | CD70 CDR1 | SIFDIARGN |
| 109 | CD70 CDR2 | AIITSGGATNYA |
| 110 | CD70 CDR3 | CNMESLSYRHYW |
| 111 | CD70 variable | QVQLQESGGGLVQTGGSLRLSCTASGSIFDIARGNWYRQAPGKQR |
| | domain | ELVAIITSGGATNYADSVAGRFTISRDDAKNTVYLQMNGLKPEDT |
| | | AVYFCNMESLSYRHYWGQGTQVTVSS |
| 112 | CD70 CDR1 | SIIRDNVMA |
| 113 | CD70 CDR2 | AIINTGGSANVD |
| 114 | CD70 CDR3 | CNVYYRDLW |
| 115 | CD70 variable | QVQLQESGGGLVQAGGSLRLSCAASKSIIRDNVMAWHRQAPGKQ |
| | domain | RELVAIINTGGSANVDDSVKGRFTISRDNAKNMVYLQMNNLKPED |
| | | TAVYYCNVYYRDLWGQGTQVTVSS |
| 116 | CD70 CDR1 | FTLDRYAVG |
| 117 | CD70 CDR2 | SCISSSGDIIKYA |
| 118 | CD70 CDR3 | CTAADPKDDCSVPGYYGLNYW |
| 119 | CD70 variable | QVQLQESGGGLVQPGGSLRLSCVASGFTLDRYAVGWFRQAPGKE |
| | domain | LEGVSCISSSGDIIKYADSAKGRFTIARDNAKNTAYLQMNSLKPED |
| | | TAVYYCTAADPKDDCSVPGYYGLNYWGKGTQVTVSS |
| 120 | CD70 CDR1 | FTLDKYAIG |
| 121 | CD70 CDR2 | SCITSSSGVVKYA |
| 122 | CD70 CDR3 | CAAAGPPDDCSVPGYYGLNYW |
| 123 | CD70 variable | QVQLQESGGGLVQPGGSLRLSCVASGFTLDKYAIGWFRQAPGKEL |
| | domain | EGVSCITSSSGVVKYADSVKGRFIISRDNTNNRAFLQMSSLKPEDT |
| | | AVYYCAAAGPPDDCSVPGYYGLNYWGKGTQVTVSS |
| 392 | CD70 CDR1 | GYSFTSYWIG |
| 393 | CD70 CDR2 | GIIYPGDSDTRYSPS |
| 394 | CD70 CDR3 | AISRTESYVMDV |
| 395 | CD70 heavy chain | EVQLVQSGAEVKKPGESLKISCKGSGYSFTSYWIGWVRQMPGKGL |
| | variable domain | EWMGIIYPGDSDTRYSPSFQGQVTISADKSISTAYLQWSSLKASDT |
| | | AMYYCAISRTESYVMDVWGQGTTVTVSS |
| 396 | CD70 CDR1 | TGTSSDVGGYNYVS |
| 397 | CD70 CDR2 | YDVTNRPS |
| 398 | CD70 CDR3 | SSYAGSHEL |
| 399 | CD70 light chain | QSALTQPRSVSGSPGQSVTISCTGTSSDVGGYNYVSWYQQHPGKA |
| | variable | PKLMIYDVTNRPSGVPDRFSASKSDNTATLTVSGVQAEDEADYYC |
| | domain | SSYAGSHELFGGGTKLTVL |

TABLE 6
TRA and TRB sequences

| SEQ | | |
|---|---|---|
| ID | | |
| NO: | Component | Sequence |
| 17 | [mm]TRAC | ATYPSSDVPCDATLTEKSFETDMNLNFQNLSVMGLRILLLKVAGFNLLMT |
| | (82-137) | LRLWSS |
| 18 | [mm]TRBC1 | GRADCGITSASYQQGVLSATILYEILLGKATLYAVLVSTLVVMAMVKRK |
| | (123-173) | NS |
| 207 | [mm]TRAC | IQNPEPAVYQLKDPRSQDSTLCLFTDFDSQINVPKTMESGTFITDKTVLDM |
| | (2-137) | KAMDSKSNGAIAWSNQTSFTCQDIFKETNATYPSSDVPCDATLTEKSFET |
| | | DMNLNFQNLSVMGLRILLLKVAGFNLLMTLRLWSS |
| 209 | [mm]TRBC1 | DLRNVTPPKVSLFEPSKAEIANKQKATLVCLARGFFPDHVELSWWVNGK |
| | (2-173) | EVHSGVSTDPQAYKESNYSYCLSSRLRVSATFWHNPRNHFRCQVQFHGL |
| | | SEEDKWPEGSPKPVTQNISAEAWGRADCGITSASYQQGVLSATILYEILLG |
| | | KATLYAVLVSTLVVMAMVKRKNS |
| 243 | [hs]TRDC(1-153) | SQPHTKPSVFVMKNGTNVACLVKEFYPKDIRINLVSSKKITEFDPAIVISPS |
| | | GKYNAVKLGKYEDSNSVTCSVQHDNKTVHSTDFEVKTDSTDHVKPKETE |
| | | NTKQPSKSCHKPKAIVHTEKVNMMSLTVLGLRMLFAKTVAVNFLLTAKL |
| | | FFL |
| 21 | [hs]TRGC(1-173) | DKQLDADVSPKPTIFLPSIAETKLQKAGTYLCLLEKFFPDVIKIHWQEKKS |
| | | NTILGSQEGNTMKTNDTYMKFSWLTVPEKSLDKEHRCIVRHENNKNGVD |
| | | QEIIFPPIKTDVITMDPKDNCSKDANDTLLLQLTNTSAYYMYLLLLLKSVV |
| | | YFAIITCCLLRRTAFCCNGEKS |
| 255 | TCRy9G115 | AGHLEQPQISSTKTLSKTARLECVVSGITISATSVYWYRERPGEVIQFLVSI |
| | | SYDGTVRKESGIPSGKFEVDRIPETSTSTLTIHNVEKQDIATYYCALWEAQ |
| | | QELGKKIKVFGPGTKLIITDKQLDADVSPKPTIFLPSIAETKLQKAGTYLCL |
| | | LEKFFPDVIKIHWEEKKSNTILGSQEGNTMKTNDTYMKFSWLTVPEKSLD |
| | | KEHRCIVRHENNKNGVDQEIIFPPIKTDVITMDPKDNCSKDANDTLLLQLT |
| | | NTSAYYMYLLLLLKSVVYFAIITCCLLRRTAFCCNGEKS |
| 256 | TCR82c15 | MQRISSLIHLSLFWAGVMSAIELVPEHQTVPVSIGVPATLRCSMKGEAIGN |
| | | YYINWYRKTQGNTMTFIYREKDIYGPGFKDNFQGDIDIAKNLAVLKILAP |
| | | SERDEGSYYCACDALKRTDTDKLIFGKGTRVTVEPRSQPHTKPSVFVMKN |
| | | GTNVACLVKEFYPKDIRINLVSSKKITEFDPAIVISPSGKYNAVKLGKYEDS |
| | | NSVTCSVQHDNKTVHSTDFEVKTDSTDHVKPKETENTKQPSKSCHKPKAI |
| | | VHTEKVNMMSLTVLGLRMLFAKTVAVNFLLTAKLFFL |
| 265 | [hs]TRDC(1-129) | SQPHTKPSVFVMKNGTNVACLVKEFYPKDIRINLVSSKKITEFDPAIVISPS |
| | | GKYNAVKLGKYEDSNSVTCSVQHDNKTVHSTDFEVKTDSTDHVKPKETE |
| | | NTKQPSKSCHKPKAIVHTEKVNMMSLTV |
| 267 | [hs]TRGC(1-106) | DKQLDADVSPKPTIFLPSIAETKLQKAGTYLCLLEKFFPDVIKIHWQEKKS |
| | | NTILGSQEGNTMKTNDTYMKFSWLTVPEKSLDKEHRCIVRHENNKNGVD |
| | | QEIIFP |

Anti-MSLN-CD3 epsilon
(SEQ ID NO: 195)
MLLLVTSLLLCELPHPAFLLIPEVQLVESGGGLVQPGGSLRLSCAASGG
DWSANFMYWYRQAPGKQRELVARISGRGVVDYVESVKGRFTISRDNSKN
TLYLQMNSLRAEDTAVYYCAVASYWGQGTLVTVSSAAAGGGGSGGGGSG
GGGSLEDGNEEMGGITQTPYKVSISGTTVILTCPQYPGSEILWQHNDKN
IGGDEDDKNIGSDEDHLSLKEFSELEQSGYYVCYPRGSKPEDANFYLYL
RARVCENCMEMDVMSVATIVIVDICITGGLLLLVYYWSKNRKAKAKPVT
RGAGAGGRQRGQNKERPPPVPNPDYEPIRKGQRDLYSGLNQRRI
Anti-CD19-CD3 epsilon
(SEQ ID NO: 196)
MLLLVTSLLLCELPHPAFLLIPDIQMTQTTSSLSASLGDRVTISCRASQ
DISKYLNWYQQKPDGTVKLLIYHTSRLHSGVPSRFSGSGSGTDYSLTIS
NLEQEDIATYFCQQGNTLPYTFGGGTKLEITGGGGSGGGGSGGGGSEVK
LQESGPGLVAPSQSLSVTCTVSGVSLPDYGVSWIRQPPRKGLEWLGVIW
GSETTYYNSALKSRLTIIKDNSKSQVFLKMNSLQTDDTAIYYCAKHYYY
GGSYAMDYWGQGTSVTVSSAAAGGGGSGGGGSGGGGSLEDGNEEMGGIT
QTPYKVSISGTTVILTCPQYPGSEILWQHNDKNIGGDEDDKNIGSDEDH
LSLKEFSELEQSGYYVCYPRGSKPEDANFYLYLRARVCENCMEMDVMSV
ATIVIVDICITGGLLLLVYYWSKNRKAKAKPVTRGAGAGGRQRGQNKER
PPPVPNPDYEPIRKGQRDLYSGLNQRRI

### OTHER EMBODIMENTS

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in this application, in applications claiming priority from this application, or in related applications. Such claims, whether directed to a different invention or to the same invention, and whether broader, narrower, equal, or different in scope in comparison to the original claims, also are regarded as included within the subject matter of the inventions of the present disclosure.

SEQUENCES

| | SEQ ID | | |
|---|---|---|---|
| Construct | NO: | Component | Sequence |
| VIN70080 | 361 | Full | MLLLVTSLLLCELPHPAFLLIPEVQLVESGGGLVQPGGS |
| pLRPC | | Sequence | LRLSCAASGGDWSANFMYWYRQAPGKQRELVARISGR |
| MH1ε T2A | | | GVVDYVESVKGRFTISRDNSKNTLYLQMNSLRAEDTA |
| PD-1(EC- | | | VYYCAVASYWGQGTLVTVSSAAAGGGGSGGGGSGGG |
| TM)CD28 | | | GSLEDGNEEMGGITQTPYKVSISGTTVILTCPQYPGSEIL |
| (IC)IL- | | | WQHNDKNIGGDEDDKNIGSDEDHLSLKEFSELEQSGYY |
| 15Ra(IC) | | | VCYPRGSKPEDANFYLYLRARVCENCMEMDVMSVATI |
| P2A IL-15 | | | VIVDICITGGLLLLVYYWSKNRKAKAKPVTRGAGAGG |
| | | | RQRGQNKERPPPVPNPDYEPIRKGQRDLYSGLNQRRIG |
| | | | SGEGRGSLLTCGDVEENPGPGMQIPQAPWPVVWAVLQ |
| | | | LGWRPGWFLDSPDRPWNPPTFSPALLVVTEGDNATFTC |
| | | | SFSNTSESFVLNWYRMSPSNQTDKLAAFPEDRSQPGQD |
| | | | CRFRVTQLPNGRDFHMSVVRARRNDSGTYLCGAISLAP |
| | | | KAQIKESLRAELRVTERRAEVPTAHPSPSPRPAGQFQTL |
| | | | VVGVVGGLLGSLVLLVWVLAVIRSKRSRLLHSDYMNM |
| | | | TPRRPGPTRKHYQPYAPPRDFAAYRSKSRQTPPLASVE |
| | | | MEAMEALPVTWGTSSRDEDLENCSHHLGSGATNFSLL |
| | | | KQAGDVEENPGPMRISKPHLRSISIQCYLCLLLNSHFLT |
| | | | EAGIHVFILGCFSAGLPKTEANWVNVISDLKKIEDLIQS |
| | | | MHIDATLYTESDVHPSCKVTAMKCFLLELQVISLESGD |
| | | | ASIHDTVENLIILANNSLSSNGNVTESGCKECEELEEKNI |
| | | | KEFLQSFVHIVQMFINT |
| | 362 | CSF2RA | MLLLVTSLLLCELPHPAFLLIP |
| | | Signal | |
| | | Peptide | |
| | 363 | MH1 | EVQLVESGGGLVQPGGSLRLSCAASGGDWSANFMYW |
| | | antiMSLN | YRQAPGKQRELVARISGRGVVDYVESVKGRFTISRDNS |
| | | sdAb | KNTLYLQMNSLRAEDTAVYYCAVASYWGQGTLVTVS |
| | | | SAAAGGGGSGGGGSGGGGSLE |
| | 364 | CD3ε | DGNEEMGGITQTPYKVSISGTTVILTCPQYPGSEILWQH |
| | | | NDKNIGGDEDDKNIGSDEDHLSLKEFSELEQSGYYVCY |
| | | | PRGSKPEDANFYLYLRARVCENCMEMDVMSVATIVIV |
| | | | DICITGGLLLLVYYWSKNRKAKAKPVTRGAGAGGRQR |
| | | | GQNKERPPPVPNPDYEPIRKGQRDLYSGLNQRRI |
| | 365 | T2A | GSGEGRGSLLTCGDVEENPGP |
| | (203) | | |
| | 366 | PD-1 Signal | MQIPQAPWPVVWAVLQLGWR |
| | | Peptide | |
| | 367 | PD-1 N- | PGWFLDSPDRPWNP |
| | | Loop | |
| | 368 | PD-1 IgV | PTFSPALLVVTEGDNATFTCSFSNTSESFVLNWYRMSPS |
| | | | NQTDKLAAFPEDRSQPGQDCRFRVTQLPNGRDFHMSV |
| | | | VRARRNDSGTYLCGAISLAPKAQIKESLRAELRVT |
| | 369 | PD-1 Stalk | ERRAEVPTAHPSPSPRPAGQFQTLV |
| | 370 | PD-1 TMD | VGVVGGLLGSLVLLVWVLAVI |
| | 371 | CD28 IC | RSKRSRLLHSDYMNMTPRRPGPTRKHYQPYAPPRDFA |
| | | | AYRS |
| | 372 | IL-15Rα | KSRQTPPLASVEMEAMEALPVTWGTSSRDEDLENCSH |
| | | | HL |
| | 373 | P2A | GSGATNFSLLKQAGDVEENPGP |
| | 374 | IL-15 Signal | MRISKPHLRSISIQCYLCLLLNSHFLTEA |
| | | Peptide | |
| | 375 | IL-15 | GIHVFILGCFSAGLPKTEANWVNVISDLKKIEDLIQSMHI |
| | | | DATLYTESDVHPSCKVTAMKCFLLELQVISLESGDASIH |
| | | | DTVENLIILANNSLSSNGNVTESGCKECEELEEKNIKEFL |
| | | | QSFVHIVQMFINTS |
| VIN10299 | 376 | Full | MLLLVTSLLLCELPHPAFLLIPEVQLVESGGGLVQPGGS |
| pLRPO | | Sequence | LRLSCAASGGDWSANFMYWYRQAPGKQRELVARISGR |
| MH1ε | | | GVVDYVESVKGRFTISRDNSKNTLYLQMNSLRAEDTA |
| T2AW PD- | | | VYYCAVASYWGQGTLVTVSSAAAGGGGSGGGGSGGG |
| 1(EC- | | | GSLEDGNEEMGGITQTPYKVSISGTTVILTCPQYPGSEIL |
| TM)CD28 | | | WQHNDKNIGGDEDDKNIGSDEDHLSLKEFSELEQSGYY |
| (IC)IL- | | | VCYPRGSKPEDANFYLYLRARVCENCMEMDVMSVATI |
| 15Ra(IC) | | | VIVDICITGGLLLLVYYWSKNRKAKAKPVTRGAGAGG |
| | | | RQRGQNKERPPPVPNPDYEPIRKGQRDLYSGLNQRRIG |
| | | | SGEGRGSLLTCGDVEENPGPGMQIPQAPWPVVWAVLQ |
| | | | LGWRPGWFLDSPDRPWNPPTFSPALLVVTEGDNATFTC |
| | | | SFSNTSESFVLNWYRMSPSNQTDKLAAFPEDRSQPGQD |
| | | | CRFRVTQLPNGRDFHMSVVRARRNDSGTYLCGAISLAP |
| | | | KAQIKESLRAELRVTERRAEVPTAHPSPSPRPAGQFQTL |
| | | | VVGVVGGLLGSLVLLVWVLAVIRSKRSRLLHSDYMNM |
| | | | TPRRPGPTRKHYQPYAPPRDFAAYRSKSRQTPPLASVE |
| | | | MEAMEALPVTWGTSSRDEDLENCSHHL |
| | 362 | CSF2RA | MLLLVTSLLLCELPHPAFLLIP |
| | | Signal | |
| | | Peptide | |
| | 363 | MH1 | EVQLVESGGGLVQPGGSLRLSCAASGGDWSANFMYW |
| | | antiMSLN | YRQAPGKQRELVARISGRGVVDYVESVKGRFTISRDNS |
| | | sdAb | KNTLYLQMNSLRAEDTAVYYCAVASYWGQGTLVTVS |
| | | | SAAAGGGGSGGGGSGGGGSLE |
| | 364 | CD3ε | DGNEEMGGITQTPYKVSISGTTVILTCPQYPGSEILWQH |
| | | | NDKNIGGDEDDKNIGSDEDHLSLKEFSELEQSGYYVCY |
| | | | PRGSKPEDANFYLYLRARVCENCMEMDVMSVATIVIV |
| | | | DICITGGLLLLVYYWSKNRKAKAKPVTRGAGAGGRQR |
| | | | GQNKERPPPVPNPDYEPIRKGQRDLYSGLNQRRI |
| | 365 | T2A | GSGEGRGSLLTCGDVEENPGP |
| | 366 | PD-1 Signal | MQIPQAPWPVVWAVLQLGWR |
| | | Peptide | |
| | 367 | PD-1 N- | PGWFLDSPDRPWNP |
| | | Loop | |
| | 368 | PD-1 IgV | PTFSPALLVVTEGDNATFTCSFSNTSESFVLNWYRMSPS |
| | | | NQTDKLAAFPEDRSQPGQDCRFRVTQLPNGRDFHMSV |
| | | | VRARRNDSGTYLCGAISLAPKAQIKESLRAELRVT |
| | 369 | PD-1 Stalk | ERRAEVPTAHPSPSPRPAGQFQTLV |
| | 370 | PD-1 TMD | VGVVGGLLGSLVLLVWVLAVI |
| | 371 | CD28 IC | RSKRSRLLHSDYMNMTPRRPGPTRKHYQPYAPPRDFA |
| | | | AYRS |
| | 372 | IL-15Rα | KSRQTPPLASVEMEAMEALPVTWGTSSRDEDLENCSH |
| | | | HL |
| VIN11023_ | 377 | Full | MLLLVTSLLLCELPHPAFLLIPEVQLVESGGGLVQPGGS |
| pLKaUS | | Sequence | LRLSCAASGGDWSANFMYWYRQAPGKQRELVARISGR |
| MH1ε | | | GVVDYVESVKGRFTISRDNSKNTLYLQMNSLRAEDTA |
| T2AW | | | VYYCAVASYWGQGTLVTVSSAAAGGGGSGGGGSGGG |
| solubleIL- | | | GSLEDGNEEMGGITQTPYKVSISGTTVILTCPQYPGSEIL |
| 15Rα | | | WQHNDKNIGGDEDDKNIGSDEDHLSLKEFSELEQSGYY |
| | | | VCYPRGSKPEDANFYLYLRARVCENCMEMDVMSVATI |
| | | | VIVDICITGGLLLLVYYWSKNRKAKAKPVTRGAGAGG |
| | | | RQRGQNKERPPPVPNPDYEPIRKGQRDLYSGLNQRRIG |
| | | | SGEGRGSLLTCGDVEENPGPMRISKPHLRSISIQCYLCLL |
| | | | LNSHFLTEAGIHVFILGCFSAGLPKTEANWVNVISDLKK |
| | | | IEDLIQSMHIDATLYTESDVHPSCKVTAMKCFLLELQVI |
| | | | SLESGDASIHDTVENLIILANNSLSSNGNVTESGCKECEE |
| | | | LEEKNIKEFLQSFVHIVQMFINTSSGGGSGGGGSGGGGS |
| | | | GGGGSGGGSLQITCPPPMSVEHADIWVKSYSLYSRERYI |
| | | | CNSGFKRKAGTSSLTECVLNKATNVAHWTTPSLKCIRD |
| | | | PALVHQRPAPPSTVTTAGVTPQPESLSPSGKEPAASSPSS |
| | | | NNTAATTAAIVPGSQLMPSKSPSTGTTEISSHESSHGTPS |
| | | | QTTAKNWELTASASHQPPGVYPQGHSDTT |
| | 362 | CSF2RA | MLLLVTSLLLCELPHPAFLLIP |
| | | Signal | |
| | | Peptide | |
| | 363 | MH1 | EVQLVESGGGLVQPGGSLRLSCAASGGDWSANFMYW |
| | | antiMSLN | YRQAPGKQRELVARISGRGVVDYVESVKGRFTISRDNS |
| | | sdAb | KNTLYLQMNSLRAEDTAVYYCAVASYWGQGTLVTVS |
| | | | SAAAGGGGSGGGGSGGGGSLE |
| | 364 | CD3ε | DGNEEMGGITQTPYKVSISGTTVILTCPQYPGSEILWQH |
| | | | NDKNIGGDEDDKNIGSDEDHLSLKEFSELEQSGYYVCY |
| | | | PRGSKPEDANFYLYLRARVCENCMEMDVMSVATIVIV |
| | | | DICITGGLLLLVYYWSKNRKAKAKPVTRGAGAGGRQR |
| | | | GQNKERPPPVPNPDYEPIRKGQRDLYSGLNQRRI |
| | 365 | T2A | GSGEGRGSLLTCGDVEENPGP |
| | 374 | IL-15 Signal | MRISKPHLRSISIQCYLCLLLNSHFLTEA |
| | | Peptide | |
| | 375 | IL-15 | GIHVFILGCFSAGLPKTEANWVNVISDLKKIEDLIQSMHI |
| | | | DATLYTESDVHPSCKVTAMKCFLLELQVISLESGDASIH |
| | | | DTVENLIILANNSLSSNGNVTESGCKECEELEEKNIKEFL |
| | | | QSFVHIVQMFINTS |
| | 378 | SG3(SG4)3 | SGGGSGGGGSGGGGSGGGGSGGGSLQ |
| | | SG3SLQ | |
| | | Linker | |
| | 379 | Soluble IL- | ITCPPPMSVEHADIWVKSYSLYSRERYICNSGFKRKAGT |
| | | 15Rα (sIL- | SSLTECVLNKATNVAHWTTPSLKCIRDPALVHQRPAPP |
| | | 15Rα) | STVTTAGVTPQPESLSPSGKEPAASSPSSNNTAATTAAI |
| | | | VPGSQLMPSKSPSTGTTEISSHESSHGTPSQTTAKNWEL |
| | | | TASASHQPPGVYPQGHSDTT |
| MH1e | 380 | Full | MLLLVTSLLLCELPHPAFLLIPEVQLVESGGGLVQPGGS |
| T2Aw IL- | | Sequence | LRLSCAASGGDWSANFMYWYRQAPGKQRELVARISGR |
| 15 | | | GVVDYVESVKGRFTISRDNSKNTLYLQMNSLRAEDTA |
| | | | VYYCAVASYWGQGTLVTVSSAAAGGGGSGGGGSGGG |
| | | | GSLEDGNEEMGGITQTPYKVSISGTTVILTCPQYPGSEIL |
| | | | WQHNDKNIGGDEDDKNIGSDEDHLSLKEFSELEQSGYY |
| | | | VCYPRGSKPEDANFYLYLRARVCENCMEMDVMSVATI |
| | | | VIVDICITGGLLLLVYYWSKNRKAKAKPVTRGAGAGG |
| | | | RQRGQNKERPPPVPNPDYEPIRKGQRDLYSGLNQRRIG |
| | | | SGEGRGSLLTCGDVEENPGPMRISKPHLRSISIQCYLCLL |
| | | | LNSHFLTEAGIHVFILGCFSAGLPKTEANWVNVISDLKK |
| | | | IEDLIQSMHIDATLYTESDVHPSCKVTAMKCFLLELQVI |
| | | | SLESGDASIHDTVENLIILANNSLSSNGNVTESGCKECEE |
| | | | LEEKNIKEFLQSFVHIVQMFINTS |
| | 362 | CSF2RA | MLLLVTSLLLCELPHPAFLLIP |
| | | Signal | |
| | | Peptide | |
| | 363 | MH1 | EVQLVESGGGLVQPGGSLRLSCAASGGDWSANFMYW |
| | | antiMSLN | YRQAPGKQRELVARISGRGVVDYVESVKGRFTISRDNS |
| | | sdAb | KNTLYLQMNSLRAEDTAVYYCAVASYWGQGTLVTVS |
| | | | SAAAGGGGSGGGGSGGGGSLE |
| | 364 | CD3ε | DGNEEMGGITQTPYKVSISGTTVILTCPQYPGSEILWQH |
| | | | NDKNIGGDEDDKNIGSDEDHLSLKEFSELEQSGYYVCY |
| | | | PRGSKPEDANFYLYLRARVCENCMEMDVMSVATIVIV |
| | | | DICITGGLLLLVYYWSKNRKAKAKPVTRGAGAGGRQR |
| | | | GQNKERPPPVPNPDYEPIRKGQRDLYSGLNQRRI |
| | 365 | T2A | GSGEGRGSLLTCGDVEENPGP |
| | 374 | IL-15 Signal | MRISKPHLRSISIQCYLCLLLNSHFLTEA |
| | | Peptide | |
| | 375 | IL-15 | GIHVFILGCFSAGLPKTEANWVNVISDLKKIEDLIQSMHI |
| | | | DATLYTESDVHPSCKVTAMKCFLLELQVISLESGDASIH |
| | | | DTVENLIILANNSLSSNGNVTESGCKECEELEEKNIKEFL |
| | | | QSFVHIVQMFINTS |
| MH1ε T2A | 381 | Full | MLLLVTSLLLCELPHPAFLLIPEVQLVESGGGLVQPGGS |
| mbIL- | | Sequence | LRLSCAASGGDWSANFMYWYRQAPGKQRELVARISGR |
| 15Rα (IL- | | | GVVDYVESVKGRFTISRDNSKNTLYLQMNSLRAEDTA |
| 15-IL- | | | VYYCAVASYWGQGTLVTVSSAAAGGGGSGGGGSGGG |
| 15RA | | | GSLEDGNEEMGGITQTPYKVSISGTTVILTCPQYPGSEIL |
| fusion) | | | WQHNDKNIGGDEDDKNIGSDEDHLSLKEFSELEQSGYY |
| | | | VCYPRGSKPEDANFYLYLRARVCENCMEMDVMSVATI |
| | | | VIVDICITGGLLLLVYYWSKNRKAKAKPVTRGAGAGG |
| | | | RQRGQNKERPPPVPNPDYEPIRKGQRDLYSGLNQRRIG |
| | | | SGEGRGSLLTCGDVEENPGPMRISKPHLRSISIQCYLCLL |
| | | | LNSHFLTEAGIHVFILGCFSAGLPKTEANWVNVISDLKK |
| | | | IEDLIQSMHIDATLYTESDVHPSCKVTAMKCFLLELQVI |
| | | | SLESGDASIHDTVENLIILANNSLSSNGNVTESGCKECEE |
| | | | LEEKNIKEFLQSFVHIVQMFINTSSGGGSGGGGSGGGGS |
| | | | GGGGSGGGSLQITCPPPMSVEHADIWVKSYSLYSRERYI |
| | | | CNSGFKRKAGTSSLTECVLNKATNVAHWTTPSLKCIRD |
| | | | PALVHQRPAPPSTVTTAGVTPQPESLSPSGKEPAASSPSS |
| | | | NNTAATTAAIVPGSQLMPSKSPSTGTTEISSHESSHGTPS |
| | | | QTTAKNWELTASASHQPPGVYPQGHSDTTVAISTSTVL |
| | | | LCGLSAVSLLACYLKSRQTPPLASVEMEAMEALPVTW |
| | | | GTSSRDEDLENCSHHLDYKDDDDKDYKDDDDKDYKD |
| | | | DDDK |
| | 362 | CSF2RA | MLLLVTSLLLCELPHPAFLLIP |
| | | Signal | |
| | | Peptide | |
| | 363 | MH1 | EVQLVESGGGLVQPGGSLRLSCAASGGDWSANFMYW |
| | | antiMSLN | YRQAPGKQRELVARISGRGVVDYVESVKGRFTISRDNS |
| | | sdAb | KNTLYLQMNSLRAEDTAVYYCAVASYWGQGTLVTVS |
| | | | SAAAGGGGSGGGGSGGGGSLE |
| | 364 | CD3ε | DGNEEMGGITQTPYKVSISGTTVILTCPQYPGSEILWQH |
| | | | NDKNIGGDEDDKNIGSDEDHLSLKEFSELEQSGYYVCY |
| | | | PRGSKPEDANFYLYLRARVCENCMEMDVMSVATIVIV |
| | | | DICITGGLLLLVYYWSKNRKAKAKPVTRGAGAGGRQR |
| | | | GQNKERPPPVPNPDYEPIRKGQRDLYSGLNQRRI |
| | 365 | T2A | GSGEGRGSLLTCGDVEENPGP |
| | 374 | IL-15 Signal | MRISKPHLRSISIQCYLCLLLNSHFLTEA |
| | | Peptide | |
| | 375 | IL-15 | GIHVFILGCFSAGLPKTEANWVNVISDLKKIEDLIQSMHI |
| | | | DATLYTESDVHPSCKVTAMKCFLLELQVISLESGDASIH |
| | | | DTVENLIILANNSLSSNGNVTESGCKECEELEEKNIKEFL |
| | | | QSFVHIVQMFINTS |
| | 378 | SG3(SG4)3 | SGGGSGGGGSGGGGSGGGGSGGGSLQ |
| | | SG3SLQ | |
| | | Linker | |
| | 382 | Sushi | ITCPPPMSVEHADIWVKSYSLYSRERYICNSGFKRKAGT |
| | | | SSLTECVLNKATNVAHWTTPSLKCIR |
| | 383 | hIL-15Rα | DPALVHQRPAPPSTVTTAGVTPQPESLSPSGKEPAASSP |
| | | | SSNNTAATTAAIVPGSQLMPSKSPSTGTTEISSHESSHGT |
| | | | PSQTTAKNWELTASASHQPPGVYPQGHSDTTVAISTST |
| | | | VLLCGLSAVSLLACYLKSRQTPPLASVEMEAMEALPVT |
| | | | WGTSSRDEDLENCSHHL |
| | 384 | Flag x3 | DYKDDDDKDYKDDDDKDYKDDDDK |
| CD70 | 400 | Full | MLLLVTSLLLCELPHPAFLLIPQSALTQPRSVSGSPGQSV |
| CD3ε | | Sequence | TISCTGTSSDVGGYNYVSWYQQHPGKAPKLMIYDVTN |
| | | | RPSGVPDRFSASKSDNTATLTVSGVQAEDEADYYCSSY |
| | | | AGSHELFGGGTKLTVLGSTSGSGKPGSGEGSTKGEVQL |
| | | | VQSGAEVKKPGESLKISCKGSGYSFTSYWIGWVRQMP |
| | | | GKGLEWMGIIYPGDSDTRYSPSFQGQVTISADKSISTAY |
| | | | LQWSSLKASDTAMYYCAISRTESYVMDVWGQGTTVT |
| | | | VSSAAAGGGGSGGGGSGGGGSLEDGNEEMGGITQTPY |
| | | | KVSISGTTVILTCPQYPGSEILWQHNDKNIGGDEDDKNI |
| | | | GSDEDHLSLKEFSELEQSGYYVCYPRGSKPEDANFYLY |
| | | | LRARVCENCMEMDVMSVATIVIVDICITGGLLLLVYYW |
| | | | SKNRKAKAKPVTRGAGAGGRQRGQNKERPPPVPNPDY |
| | | | EPIRKGQRDLYSGLNQRRI |
| | 362 | CSF2RA | MLLLVTSLLLCELPHPAFLLIP |
| | | signal | |
| | | peptide | |
| | 399 | C10 vL | QSALTQPRSVSGSPGQSVTISCTGTSSDVGGYNYVSWY |
| | | | QQHPGKAPKLMIYDVTNRPSGVPDRFSASKSDNTATLT |
| | | | VSGVQAEDEADYYCSSYAGSHELFGGGTKLTVL |
| | 401 | Whitlow | GSTSGSGKPGSGEGSTKG |
| | | Linker | |
| | 395 | C10 vH | EVQLVQSGAEVKKPGESLKISCKGSGYSFTSYWIGWVR |
| | | | QMPGKGLEWMGIIYPGDSDTRYSPSFQGQVTISADKSIS |
| | | | TAYLQWSSLKASDTAMYYCAISRTESYVMDVWGQGT |
| | | | TVTVSS |
| | 387 | A3(G4S)3L | AAAGGGGSGGGGSGGGGSLE |
| | | E Linker | |
| | 364 | CD3ε | DGNEEMGGITQTPYKVSISGTTVILTCPQYPGSEILWQH |
| | | | NDKNIGGDEDDKNIGSDEDHLSLKEFSELEQSGYYVCY |
| | | | PRGSKPEDANFYLYLRARVCENCMEMDVMSVATIVIV |
| | | | DICITGGLLLLVYYWSKNRKAKAKPVTRGAGAGGRQR |
| | | | GQNKERPPPVPNPDYEPIRKGQRDLYSGLNQRRI |
| CD70 | 402 | Full | MLLLVTSLLLCELPHPAFLLIPQSALTQPRSVSGSPGQSV |
| CD3ε + | | Sequence | TISCTGTSSDVGGYNYVSWYQQHPGKAPKLMIYDVTN |
| mbIL-15fu | | | RPSGVPDRFSASKSDNTATLTVSGVQAEDEADYYCSSY |
| | | | AGSHELFGGGTKLTVLGSTSGSGKPGSGEGSTKGEVQL |
| | | | VQSGAEVKKPGESLKISCKGSGYSFTSYWIGWVRQMP |
| | | | GKGLEWMGIIYPGDSDTRYSPSFQGQVTISADKSISTAY |
| | | | LQWSSLKASDTAMYYCAISRTESYVMDVWGQGTTVT |
| | | | VSSAAAGGGGSGGGGSGGGGSLEDGNEEMGGITQTPY |
| | | | KVSISGTTVILTCPQYPGSEILWQHNDKNIGGDEDDKNI |
| | | | GSDEDHLSLKEFSELEQSGYYVCYPRGSKPEDANFYLY |
| | | | LRARVCENCMEMDVMSVATIVIVDICITGGLLLLVYYW |
| | | | SKNRKAKAKPVTRGAGAGGRQRGQNKERPPPVPNPDY |
| | | | EPIRKGQRDLYSGLNQRRIGSGEGRGSLLTCGDVEENP |
| | | | GPMRISKPHLRSISIQCYLCLLLNSHFLTEAGIHVFILGCF |
| | | | SAGLPKTEANWVNVISDLKKIEDLIQSMHIDATLYTESD |
| | | | VHPSCKVTAMKCFLLELQVISLESGDASIHDTVENLIILA |
| | | | NNSLSSNGNVTESGCKECEELEEKNIKEFLQSFVHIVQM |
| | | | FINTSSGGGSGGGGSGGGGSGGGGSGGGSLQITCPPPMS |
| | | | VEHADIWVKSYSLYSRERYICNSGFKRKAGTSSLTECV |
| | | | LNKATNVAHWTTPSLKCIRDPALVHQRPAPPSTVTTAG |
| | | | VTPQPESLSPSGKEPAASSPSSNNTAATTAAIVPGSQLM |
| | | | PSKSPSTGTTEISSHESSHGTPSQTTAKNWELTASASHQP |
| | | | PGVYPQGHSDTTVAISTSTVLLCGLSAVSLLACYLKSRQ |
| | | | TPPLASVEMEAMEALPVTWGTSSRDEDLENCSHHL |
| | 362 | CSF2RA | MLLLVTSLLLCELPHPAFLLIP |
| | | signal | |
| | | peptide | |
| | 399 | C10 vL | QSALTQPRSVSGSPGQSVTISCTGTSSDVGGYNYVSWY |
| | | | QQHPGKAPKLMIYDVTNRPSGVPDRFSASKSDNTATLT |
| | | | VSGVQAEDEADYYCSSYAGSHELFGGGTKLTVL |
| | 401 | Whitlow | GSTSGSGKPGSGEGSTKG |
| | | Linker | |
| | 395 | C10 vH | EVQLVQSGAEVKKPGESLKISCKGSGYSFTSYWIGWVR |
| | | | QMPGKGLEWMGIIYPGDSDTRYSPSFQGQVTISADKSIS |
| | | | TAYLQWSSLKASDTAMYYCAISRTESYVMDVWGQGT |
| | | | TVTVSS |
| | 387 | A3(G4S)3L | AAAGGGGSGGGGSGGGGSLE |
| | | E Linker | |
| | 364 | CD3ε | DGNEEMGGITQTPYKVSISGTTVILTCPQYPGSEILWQH |
| | | | NDKNIGGDEDDKNIGSDEDHLSLKEFSELEQSGYYVCY |
| | | | PRGSKPEDANFYLYLRARVCENCMEMDVMSVATIVIV |
| | | | DICITGGLLLLVYYWSKNRKAKAKPVTRGAGAGGRQR |
| | | | GQNKERPPPVPNPDYEPIRKGQRDLYSGLNQRRI |
| | 365 | T2A linker | GSGEGRGSLLTCGDVEENPGP |
| | 385 | IL-15 | MRISKPHLRSISIQCYLCLLLNSHFLTEAGIHVFILGCFSA |
| | | | GLPKTEANWVNVISDLKKIEDLIQSMHIDATLYTESDV |
| | | | HPSCKVTAMKCFLLELQVISLESGDASIHDTVENLIILAN |
| | | | NSLSSNGNVTESGCKECEELEEKNIKEFLQSFVHIVQMF |
| | | | INTS |
| | 378 | Linker | SGGGSGGGGSGGGGSGGGGSGGGSLQ |
| | 403 | hIL-15Ra | ITCPPPMSVEHADIWVKSYSLYSRERYICNSGFKRKAGT |
| | | | SSLTECVLNKATNVAHWTTPSLKCIRDPALVHQRPAPP |
| | | | STVTTAGVTPQPESLSPSGKEPAASSPSSNNTAATTAAI |
| | | | VPGSQLMPSKSPSTGTTEISSHESSHGTPSQTTAKNWEL |
| | | | TASASHQPPGVYPQGHSDTTVAISTSTVLLCGLSAVSLL |
| | | | ACYLKSRQTPPLASVEMEAMEALPVTWGTSSRDEDLE |
| | | | NCSHHL |
| CD70 | 404 | Full | MLLLVTSLLLCELPHPAFLLIPQSALTQPRSVSGSPGQSV |
| CD3ε + | | Sequence | TISCTGTSSDVGGYNYVSWYQQHPGKAPKLMIYDVTN |
| mbIL-15fu | | | RPSGVPDRFSASKSDNTATLTVSGVQAEDEADYYCSSY |
| variant | | | AGSHELFGGGTKLTVLGSTSGSGKPGSGEGSTKGEVQL |
| | | | VQSGAEVKKPGESLKISCKGSGYSFTSYWIGWVRQMP |
| | | | GKGLEWMGIIYPGDSDTRYSPSFQGQVTISADKSISTAY |
| | | | LQWSSLKASDTAMYYCAISRTESYVMDVWGQGTTVT |
| | | | VSSAAAGGGGSGGGGSGGGGSLEDGNEEMGGITQTPY |
| | | | KVSISGTTVILTCPQYPGSEILWQHNDKNIGGDEDDKNI |
| | | | GSDEDHLSLKEFSELEQSGYYVCYPRGSKPEDANFYLY |
| | | | LRARVCENCMEMDVMSVATIVIVDICITGGLLLLVYYW |
| | | | SKNRKAKAKPVTRGAGAGGRQRGQNKERPPPVPNPDY |
| | | | EPIRKGQRDLYSGLNQRRIGSGEGRGSLLTCGDVEENP |
| | | | GPMRISKPHLRSISIQCYLCLLLNSHFLTEAGIHVFILGCF |
| | | | SAGLPKTEANWVNVISDLKKIEDLIQSMHIDATLYTESD |
| | | | VHPSCKVTAMKCFLLELQVISLESGDASIHDTVENLIILA |
| | | | NNSLSSNGNVTESGCKECEELEEKNIKEFLQSFVHIVQM |
| | | | FINTSSGGGSGGGGSGGGGSGGGGSGGGSLEITCPPPMS |
| | | | VEHADIWVKSYSLYSRERYICNSGFKRKAGTSSLTECV |
| | | | LNKATNVAHWTTPSLKCIRDPALVHQRPAPPSTVTTAG |
| | | | VTPQPESLSPSGKEPAASSPSSNNTAATTAAIVPGSQLM |
| | | | PSKSPSTGTTEISSHESSHGTPSQTTAKNWELTASASHQP |
| | | | PGVYPQGHSDTTVAISTSTVLLCGLSAVSLLACYLKSRQ |
| | | | TPPLASVEMEAMEALPVTWGTSSRDEDLENCSHHL |
| | 362 | CSF2RA | MLLLVTSLLLCELPHPAFLLIP |
| | | signal | |
| | | peptide | |
| | 399 | C10 vL | QSALTQPRSVSGSPGQSVTISCTGTSSDVGGYNYVSWY |
| | | | QQHPGKAPKLMIYDVTNRPSGVPDRFSASKSDNTATLT |
| | | | VSGVQAEDEADYYCSSYAGSHELFGGGTKLTVL |
| | 401 | Whitlow | GSTSGSGKPGSGEGSTKG |
| | | Linker | |
| | 395 | C10 vH | EVQLVQSGAEVKKPGESLKISCKGSGYSFTSYWIGWVR |
| | | | QMPGKGLEWMGIIYPGDSDTRYSPSFQGQVTISADKSIS |
| | | | TAYLQWSSLKASDTAMYYCAISRTESYVMDVWGQGT |
| | | | TVTVSS |
| | 387 | A3(G4S)3L | AAAGGGGSGGGGSGGGGSLE |
| | | E Linker | |
| | 364 | CD3ε | DGNEEMGGITQTPYKVSISGTTVILTCPQYPGSEILWQH |
| | | | NDKNIGGDEDDKNIGSDEDHLSLKEFSELEQSGYYVCY |
| | | | PRGSKPEDANFYLYLRARVCENCMEMDVMSVATIVIV |
| | | | DICITGGLLLLVYYWSKNRKAKAKPVTRGAGAGGRQR |
| | | | GQNKERPPPVPNPDYEPIRKGQRDLYSGLNQRRI |
| | 365 | T2A linker | GSGEGRGSLLTCGDVEENPGP |
| | 385 | IL-15 | MRISKPHLRSISIQCYLCLLLNSHFLTEAGIHVFILGCFSA |
| | | | GLPKTEANWVNVISDLKKIEDLIQSMHIDATLYTESDV |
| | | | HPSCKVTAMKCFLLELQVISLESGDASIHDTVENLIILAN |
| | | | NSLSSNGNVTESGCKECEELEEKNIKEFLQSFVHIVQMF |
| | | | INTS |
| | 405 | Linker | SGGGSGGGGSGGGGSGGGGSGGGSLE |
| | 403 | hIL-15Ra | ITCPPPMSVEHADIWVKSYSLYSRERYICNSGFKRKAGT |
| | | | SSLTECVLNKATNVAHWTTPSLKCIRDPALVHQRPAPP |
| | | | STVTTAGVTPQPESLSPSGKEPAASSPSSNNTAATTAAI |
| | | | VPGSQLMPSKSPSTGTTEISSHESSHGTPSQTTAKNWEL |
| | | | TASASHQPPGVYPQGHSDTTVAISTSTVLLCGLSAVSLL |
| | | | ACYLKSRQTPPLASVEMEAMEALPVTWGTSSRDEDLE |
| | | | NCSHHL |

What is claimed is:

1.-218. (canceled)

219. A recombinant nucleic acid molecule comprising:
a first nucleic acid sequence encoding a T cell receptor (TCR) fusion protein (TFP) wherein the TFP comprises:
(a) a TCR subunit comprising:
(i) at least a portion of a TCR extracellular domain, and
(ii) a TCR transmembrane domain, and
(b) an antigen binding domain; and
wherein the TCR subunit and the antigen binding domain are operatively linked, and a second nucleic acid sequence encoding an interleukin-15 (IL-15) polypeptide or a fragment thereof and/or an IL-15 (IL-15R) polypeptide or a fragment thereof.

220. The recombinant nucleic acid molecule of claim 219, wherein the TFP further comprises a TCR intracellular signaling domain.

221. The recombinant nucleic acid molecule of claim 219, wherein the first nucleic acid sequence and the second nucleic acid sequence are operatively linked by a first linker comprising a protease cleavage site.

222. The recombinant nucleic acid molecule of claim 219, wherein the IL-15 polypeptide is secreted when expressed in a T cell.

223. The recombinant nucleic acid molecule of claim 219, wherein the second nucleic acid sequence encodes an IL-15 polypeptide and an IL-15R alpha (IL-15Rα) polypeptide.

224. The recombinant nucleic acid molecule of claim 223, wherein the IL-15 polypeptide comprises a sequence of SEQ ID NO: 375 and the IL-15Rα polypeptide comprises a sequence of SEQ ID NO: 403.

225. The recombinant nucleic acid molecule of claim 223, wherein the IL-15 polypeptide and the IL-15Rα polypeptide are operatively linked by a second linker.

226. The recombinant nucleic acid molecule of claim 225, wherein the second linker comprises a sequence of SEQ ID NO: 378 or 405.

227. The recombinant nucleic acid molecule of claim 223, wherein the second nucleic acid sequence encodes a fusion protein comprising the IL-15 polypeptide linked to the N-terminus of the TL-15Rα polypeptide.

228. The recombinant nucleic acid molecule of claim 227, wherein the fusion protein comprises a sequence having at least 95% identity to SEQ ID NO: 389.

229. The recombinant nucleic acid molecule of claim 227, wherein the fusion protein is expressed on cell surface when expressed in a T cell.

230. The recombinant nucleic acid molecule of claim 219, wherein the recombinant nucleic acid molecule encodes an amino acid sequence of SEQ ID NO: 377, SEQ ID NO: 380, SEQ ID NO: 381, SEQ ID NO: 402, or SEQ ID NO: 404.

231. The recombinant nucleic acid molecule of claim 220, wherein the TCR intracellular signaling domain comprises a stimulatory domain from an intracellular signaling domain of CD3 gamma, CD3 delta, or CD3 epsilon.

232. The recombinant nucleic acid molecule of claim 219, wherein the antigen binding domain is connected to the TCR extracellular domain by a third linker sequence.

233. The recombinant nucleic acid molecule of claim 220, wherein at least two of the TCR extracellular domain, the TCR transmembrane domain, and the TCR intracellular signaling domain are from the same TCR subunit, and wherein the same TCR subunit is selected from the group consisting of CD3 epsilon, CD3 delta, and CD3 gamma.

234. The recombinant nucleic acid molecule of claim 219, wherein the antigen binding domain is a camelid antibody or binding fragment thereof; a murine antibody or binding fragment thereof; or a human or humanized antibody or binding fragment thereof.

235. The recombinant nucleic acid molecule of claim 219, wherein the antigen binding domain is a single-chain variable fragment (scFv) or a single domain antibody (sdAb) domain.

236. The recombinant nucleic acid molecule of claim 219, wherein the antigen binding domain is selected from the group consisting of an anti-CD19 binding domain, an anti-B-cell maturation antigen (BCMA) binding domain, and an anti-mesothelin (MSLN) binding domain, an anti-CD20 binding domain, an anti-CD70 binding domain, anti-MUC16 binding domain, an anti-Nectin-4 binding domain, an anti-GPC3 binding domain, and an anti-TROP-2 binding domain.

237. A polypeptide encoded by the recombinant nucleic acid molecule of claim 219.

238. A vector comprising the recombinant nucleic acid molecule of claim 219.

239. A cell comprising the recombinant nucleic acid molecule of claim 219.

240. The cell of claim 239, wherein the cell is a T cell.

241. A cell comprising:
a first nucleic acid sequence encoding a T cell receptor (TCR) fusion protein (TFP) wherein the TFP comprises:
(a) a TCR subunit comprising:
(i) at least a portion of a TCR extracellular domain, and
(ii) a TCR transmembrane domain, and
(b) an antigen binding domain; and
wherein the TCR subunit and the antigen binding domain are operatively linked, and a second nucleic acid sequence encoding a fusion protein comprising an IL-15 polypeptide or a fragment thereof and an IL-15Rα polypeptide or a fragment thereof.

242. The cell of claim 241, wherein the first nucleic acid sequence and the second nucleic acid sequence are included in a single nucleic acid molecule.

243. The cell of claim 241, wherein the first nucleic acid sequence and the second nucleic acid sequence are included in two separate nucleic acid molecules.

244. A pharmaceutical composition comprising the cell of claim 241 and a pharmaceutically acceptable carrier.

245. A method of increasing the activity or persistence of a cell expressing a recombinant nucleic acid molecule comprising a sequence encoding a T cell receptor (TCR) fusion protein (TFP), the method comprising expressing an interleukin-15 (IL-15) polypeptide or a fragment thereof and/or an IL-15Rα polypeptide or a fragment thereof in the cell:
wherein the TFP comprises:
(a) a TCR subunit comprising:
(i) at least a portion of a TCR extracellular domain, and
(ii) a TCR transmembrane domain, and
(b) an antigen binding domain; and
wherein the TCR subunit and the antigen binding domain are operatively linked.

246. A method of treating a disease or a condition in a subject in need thereof, comprising administering to the subject a therapeutically effective amount of the pharmaceutical composition of claim 244.

247. The method of claim 246, wherein the disease or the condition is a cancer or a disease or a condition associated with expression of CD19, B-cell maturation antigen (BCMA), mesothelin (MSLN), CD20, CD70, MUC16, Trop-2, Nectin-4, or GPC3.


SEQUENCE LISTING (425 sequences)

SEQ ID NO: 1-15 - skipped (15 entries)

SEQ ID NO: 16 - protein, 178 aa, Homo sapiens
VEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 17 - protein, 56 aa, Mus musculus
ATYPSSDVPCDATLTEKSFETDMNLNFQNLSVMGLRILLLKVAGFNLLMTLRLWSS

SEQ ID NO: 18 - protein, 51 aa, Mus musculus
GRADCGITSASYQQGVLSATILYEILLGKATLYAVLVSTLVVMAMVKRKNS

SEQ ID NO: 19 - protein, 293 aa, Homo sapiens
PVDSGVTQTPKHLITATGQRVTLRCSPRSGDLSVSWYQQSLDQGLQFLIQYYNGEERAKGNILERFSAQQFPDLHSELNLSSLELGDSALYFCASSPRTGLNTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 20 - protein, 152 aa, Homo sapiens
SQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFF

SEQ ID NO: 21 - protein, 173 aa, Homo sapiens
DKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 22 - protein, 149 aa, Homo sapiens
SQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAK

SEQ ID NO: 23 - protein, 18 aa, Thosea asigna virus
EGRGSLLTCGDVEENPGP

SEQ ID NO: 24 - protein, 112 aa, Artificial Sequence
  Description of Artificial Sequence: Synthetic polypeptide
DVVMTQTPLSLPVSLGDQASISCRSSQSLVHSNGNTYLHWYLQKPGQSPKLLIYKVSNRFSGVPDRFSGSGSGTDFTLKITRVEAEDLGVFFCSQSTHVPFTFGSGTKLEIK

SEQ ID NO: 25 - DNA, 336 bp, Artificial Sequence
  Description of Artificial Sequence: Synthetic polynucleotide
gatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggactgatttcacactcaagatcaccagagtggaggctgaggatctgggagtttttttctgctctcaaagtacacatgttccattcacgttcggctcggggacaaagttggaaataaaa

SEQ ID NO: 26 - protein, 120 aa, Artificial Sequence
  Description of Artificial Sequence: Synthetic polypeptide
QVQLQQSGAELVRPGASVTLSCKASGYTFFDYEMHWVKQTPVHGLEWIGAIDPEIDGTAYNQKFKGKAILTADKSSSTAYMELRSLTSEDSAVYYCTDYYGSSYWYFDVWGTGTTVTVSS

SEQ ID NO: 27 - DNA, 359 bp, Artificial Sequence
  Description of Artificial Sequence: Synthetic polynucleotide
caggttcaactgcagcagtctggggctgagctggtgaggcctggggcttcagtgacgctgtcctgcaaggcttcgggctacacattttttgactatgaaatgcactgggtgaagcagacacctgtgcatggcctggaatggattggagctattgatcctgaaattgatggtactgcctacaatcagaagttcaagggcaaggccatactgactgcagacaaatcctccagcacagcctacatggagctccgcagcctgacatctgaggactctgccgtctattactgtacagattactacggtagtagctactggtacttcgatgtctggggcacagggaccacggtcaccgtctcctc

SEQ ID NO: 28 - protein, 112 aa, Artificial Sequence
  Description of Artificial Sequence: Synthetic polypeptide
DVMMTQTPLSLPVSLGDQASISCRSSQSLVHSNGNTYLHWFLQKPGQSPKLLIYKVSNRFSGVPDRFSGSGSGTDFTLKISRVEAEDLGVYFCSQTTHVPLTFGAGTKLELK

SEQ ID NO: 29 - DNA, 336 bp, Artificial Sequence
  Description of Artificial Sequence: Synthetic polynucleotide
gatgttatgatgacccaaactccactctccctgcctgtcagtcttggagatcaagcctccatctcttgcagatctagtcagagccttgtacacagtaatggaaacacctatttacattggttcctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaactacacatgttccgctcacgttcggtgctgggaccaagctggagctgaaa

SEQ ID NO: 30 - protein, 120 aa, Artificial Sequence
  Description of Artificial Sequence: Synthetic polypeptide
QVQLQQSGAELVRPGASVTLSCKASGYTFTDYEMHWVKQTPVHGLEWIGAIDPEIAGTAYNQKFKGKAILTADKSSSTAYMELRSLTSEDSAVYYCSRYGGNYLYYFDYWGQGTTLTVSS

SEQ ID NO: 31 - DNA, 360 bp, Artificial Sequence
  Description of Artificial Sequence: Synthetic polynucleotide
caggttcaactgcagcagtctggggctgagctggtgaggcctggggcttcagtgacgctgtcctgcaaggcttcgggctacacttttactgactatgaaatgcactgggtgaagcagacacctgtccatggcctggaatggattggagctattgatcctgaaattgctggtactgcctacaatcagaagttcaagggcaaggccatactgactgcagacaaatcctccagcacagcctacatggagctccgcagcctgacatctgaggactctgccgtctattactgttcaagatacggtggtaactacctttactactttgactactggggccaaggcaccactctcacagtctcctca

SEQ ID NO: 32 - protein, 112 aa, Artificial Sequence
  Description of Artificial Sequence: Synthetic polypeptide
DVLMTQIPLSLPVSLGDQASISCRSSQNIVYSNGNTYLEWYLQKPGQSPKLLIYKVSNRFSGVPDRFSGSGSGTDFTLKISRVEAEDLGVYYCFQGSHVPFTFGSGTKLEIK

SEQ ID NO: 33 - DNA, 336 bp, Artificial Sequence
  Description of Artificial Sequence: Synthetic polynucleotide
gatgttttgatgacccaaattccactctccctgcctgtcagtcttggagatcaagcctccatctcttgcagatctagtcagaacattgtgtatagtaatggaaacacctatttagagtggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccattcacgttcggctcggggacaaagttggaaataaaa

SEQ ID NO: 34 - protein, 118 aa, Artificial Sequence
  Description of Artificial Sequence: Synthetic polypeptide
QVQLQQSGAELVRPGASVTLSCKASGYTFTDYEMHWVKQTPVHGLEWIGAIDPEIGGSAYNQKFKGRAILTADKSSSTAYMELRSLTSEDSAVYYCTGYDGYFWFAYWGQGTLVTVSS

SEQ ID NO: 35 - DNA, 354 bp, Artificial Sequence
  Description of Artificial Sequence: Synthetic polynucleotide
caggttcaactgcagcagtccggggctgagctggtgaggcctggggcttcagtgacgctgtcctgcaaggcttcgggctacacatttactgactatgaaatgcactgggtgaagcagacacctgtgcatggcctggaatggattggagctattgatcctgaaattggtggttctgcctacaatcagaagttcaagggcagggccatattgactgcagacaaatcctccagcacagcctacatggagctccgcagcctgacatctgaggactctgccgtctattattgtacgggctatgatggttacttttggtttgcttactggggccaagggactctggtcactgtctcttca

SEQ ID NO: 36 - protein, 106 aa, Artificial Sequence
  Description of Artificial Sequence: Synthetic polypeptide
ENVLTQSPAIMSASPGEKVTMTCSASSSVSYMHWYQQKSSTSPKLWIYDTSKLASGVPGRFSGSGSGNSYSLTISSMEAEDVATYYCFQGSGYPLTFGSGTKLEIK

SEQ ID NO: 37 - DNA, 318 bp, Artificial Sequence
  Description of Artificial Sequence: Synthetic polynucleotide
gaaaatgttctcacccagtctccagcaatcatgtccgcatctccaggggaaaaggtcaccatgacctgcagtgctagctcaagtgtaagttacatgcactggtaccagcagaagtcaagcacctcccccaaactctggatttatgacacatccaaactggcttctggagtcccaggtcgcttcagtggcagtgggtctggaaactcttactctctcacgatcagcagcatggaggctgaagatgttgccacttattactgttttcaggggagtgggtacccactcacgttcggctcggggacaaagttggaaataaaa

SEQ ID NO: 38 - protein, 116 aa, Artificial Sequence
  Description of Artificial Sequence: Synthetic polypeptide
QVQLQQSGAELVRPGASVTLSCKASGYTFTDYEMHWVKQTPVHGLEWIGGIDPETGGTAYNQKFKGKAILTADKSSSTAYMELRSLTSEDSAVYYCTSYYGSRVFWGTGTTVTVSS

SEQ ID NO: 39 - DNA, 348 bp, Artificial Sequence
  Description of Artificial Sequence: Synthetic polynucleotide
caggttcaactgcagcagtctggggctgagctggtgaggcctggggcttcagtgacgctgtcctgcaaggcttcgggctacacatttactgactatgaaatgcactgggtgaaacagacacctgtgcatggcctggaatggattggaggtattgatcctgaaactggtggtactgcctacaatcagaagttcaagggtaaggccatactgactgcagacaaatcctccagcacagcctacatggagctccgcagcctgacatctgaggactctgccgtctattactgtacaagttactatggtagtagagtcttctggggcacagggaccacggtcaccgtctcctca

SEQ ID NO: 40 - protein, 108 aa, Artificial Sequence
  Description of Artificial Sequence: Synthetic polypeptide
QIVLSQSPAILSAFPGEKVTMTCRASSSVSYMHWYQQKPGSSPKPWIYATSNLASGVPARFSGSGSGTSYSLTISSVEAEDAATYYCQQWSSNPPTLTFGAGTKLELK

SEQ ID NO: 41 - DNA, 324 bp, Artificial Sequence
  Description of Artificial Sequence: Synthetic polynucleotide
caaattgttctctcccagtctccagcaatcctgtctgcatttccaggggagaaggtcactatgacttgcagggccagctcaagtgtaagttacatgcactggtaccagcagaagccaggatcctcccccaaaccctggatttatgccacatccaacctggcttctggagtccctgctcgcttcagtggcagtgggtctgggacctcttactctctcacaatcagcagtgtggaggctgaagatgctgccacttattactgccagcagtggagtagtaacccacccacgctcacgttcggtgctgggaccaagctggagctgaaa

SEQ ID NO: 42 - protein, 124 aa, Artificial Sequence
  Description of Artificial Sequence: Synthetic polypeptide
QVQLQQSGAELARPGASVKLSCKASGYTFTSYGISWVKQRTGQGLEWIGEIYPRSGNTYYNESFKGKVTLTADKSSGTAYMELRSLTSEDSAVYFCARWGSYGSPPFYYGMDYWGQGTSVTVSS

SEQ ID NO: 43 - DNA, 372 bp, Artificial Sequence
  Description of Artificial Sequence: Synthetic polynucleotide
caggttcagctgcagcagtctggagctgagctggcgaggcctggggcttcagtgaagctgtcctgcaaggcttctggctacaccttcacaagctatggtataagctgggtgaagcagaggactggacagggccttgagtggattggagagatttatcctagaagtggtaatacttactacaatgagagcttcaagggcaaggtcacactgaccgcagacaaatcttccggcacagcgtacatggagctccgcagcctgacatctgaggactctgcggtctatttctgtgcaagatggggctcctacggtagtccccccttttactatggtatggactactggggtcaaggaacctcagtcaccgtctcctca

SEQ ID NO: 44 - protein, 112 aa, Artificial Sequence
  Description of Artificial Sequence: Synthetic polypeptide
DVLMTQTPLSLPVSLGNQASISCRSSQSIVHSSGSTYLEWYLQKPGQSPKLLIYKVSNRFSGVPDRFSGSGSGTDFTLKISRVEAEDLGVYYCFQGSHVPYTFGGGTKLEIK

SEQ ID NO: 45 - DNA, 336 bp, Artificial Sequence
  Description of Artificial Sequence: Synthetic polynucleotide
gatgttttgatgacccaaactccactctccctgcctgtcagtcttggaaatcaagcctccatctcttgcagatctagtcagagcattgtacatagtagtggaagcacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggctcacatgttccatacacgttcggaggggggaccaagctggaaataaaa

SEQ ID NO: 46 - protein, 123 aa, Artificial Sequence
  Description of Artificial Sequence: Synthetic polypeptide
QVQLQQSGAELARPGTSVKVSCKASGYTFTSYGISWVKQRIGQGLEWIGEIHPRSGNSYYNEKIRGKATLTADKSSSTAYMELRSLISEDSAVYFCARLITTVVANYYAMDYWGQGTSVTVSS

SEQ ID NO: 47 - DNA, 369 bp, Artificial Sequence
  Description of Artificial Sequence: Synthetic polynucleotide
caggttcagctgcagcagtctggagctgagctggcgaggcctgggacttcagtgaaggtgtcctgcaaggcttctggctataccttcacaagttatggtataagctgggtgaagcagagaattggacagggccttgagtggattggagagattcatcctagaagtggtaatagttactataatgagaagatcaggggcaaggccacactgactgcagacaaatcctccagcacagcgtacatggagctccgcagcctgatatctgaggactctgcggtctatttctgtgcaaggctgattactacggtagttgctaattactatgctatggactactggggtcaaggaacctcagtcaccgtctcctca

SEQ ID NO: 48 - protein, 112 aa, Artificial Sequence
  Description of Artificial Sequence: Synthetic polypeptide
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLVTFGAGTKLELK

SEQ ID NO: 49 - DNA, 336 bp, Artificial Sequence
  Description of Artificial Sequence: Synthetic polynucleotide
gacattgtgatgtcacagtctccatcctccctggctgtgtcagcaggagagaaggtcactatgagctgcaaatccagtcagagtctgctcaacagtagaacccgaaagaactacttggcttggtaccagcagaaaccagggcagtctcctaaactgctgatctactgggcatccactagggaatctggggtccctgatcgcttcacaggcagtggatctgggacagatttcactctcaccatcagcagtgtgcaggctgaagacctggcagtttattactgcaaacaatcttataatctggtcacgttcggtgctgggaccaagctggagctgaaa

SEQ ID NO: 50 - protein, 120 aa, Artificial Sequence; identical to SEQ ID NO: 26
  Description of Artificial Sequence: Synthetic polypeptide

SEQ ID NO: 51 - DNA, 359 bp, Artificial Sequence; identical to SEQ ID NO: 27
  Description of Artificial Sequence: Synthetic polynucleotide

SEQ ID NO: 52 - protein, 106 aa, Artificial Sequence
  Description of Artificial Sequence: Synthetic polypeptide
QIVLTQSPAIMSASPGEKVTISCSASSSVSYMYWYQQKPGSSPKPWIYRTSNLASGVPARFSGSGSGTSYSLTISSMEAEDAATYYCQQYHSYPLTFGAGTKLELK

SEQ ID NO: 53 - DNA, 318 bp, Artificial Sequence
  Description of Artificial Sequence: Synthetic polynucleotide
caaattgttctcacccagtctccagcaatcatgtctgcatctccaggggagaaggtcaccatatcctgcagtgccagctcaagtgtaagttacatgtactggtaccagcagaagccaggatcctcccccaaaccctggatttatcgcacatccaacctggcttctggagtccctgctcgcttcagtggcagtgggtctgggacctcttactctctcacaatcagcagcatggaggctgaagatgctgccacttattactgccagcagtatcatagttacccactcacgttcggtgctgggaccaagctggagctgaaa

SEQ ID NO: 54 - protein, 109 aa, Artificial Sequence
  Description of Artificial Sequence: Synthetic polypeptide
QIVLTQSPAIMSASPGERVTMTCSASSSVSSSYLYWYQQKSGSSPKLWIYSISNLASGVPARFSGSGSGTSYSLTINSMEAEDAATYYCQQWSSNPQLTFGAGTKLELK

SEQ ID NO: 55 - DNA, 327 bp, Artificial Sequence
  Description of Artificial Sequence: Synthetic polynucleotide
caaattgttctcacccagtctccagcaatcatgtctgcatctcctggggaacgggtcaccatgacctgcagtgccagctcaagtgtaagttccagctacttgtactggtaccagcagaagtcaggatcctccccaaaactctggatttatagcatatccaacctggcttctggagtcccagctcgcttcagtggcagtgggtctgggacctcttactctctcacaatcaacagcatggaggctgaagatgctgccacttattactgccagcagtggagtagtaacccacagctcacgttcggtgctgggaccaagctggagctgaaa

SEQ ID NO: 56 - protein, 121 aa, Artificial Sequence
  Description of Artificial Sequence: Synthetic polypeptide
QVQLKQSGAELVKPGASVKISCKASGYTFTDYYINWVKQRPGQGLEWIGKIGPGSGSTYYNEKFKGKATLTADKSSSTAYMQLSSLTSEDSAVYFCARTGYYVGYYAMDYWGQGTSVTVSS

SEQ ID NO: 57 - DNA, 363 bp, Artificial Sequence
  Description of Artificial Sequence: Synthetic polynucleotide
caggtccagctgaagcagtctggagctgagctggtgaagcctggggcttcagtgaagatatcctgcaaggcttctggctacaccttcactgactactatataaactgggtgaagcagaggcctggacagggccttgagtggattggaaagattggtcctggaagtggtagtacttactacaatgagaagttcaagggcaaggccacactgactgcagacaaatcctccagcacagcctacatgcagctcagcagcctgacatctgaggactctgcagtctatttctgtgcaagaactggttactacgttggttactatgctatggactactggggtcaaggaacctcagtcaccgtctcctca

SEQ ID NO: 58 - protein, 118 aa, Artificial Sequence
  Description of Artificial Sequence: Synthetic polypeptide
QVQLQQSGAELARPGASVKLSCKASGYTFTIYGISWVKQRTGQGLEWIGEIYPRSDNTYYNEKFKGKATLTADKSSSTAYMELRSLTSEDSAVYFCARWYSFYAMDYWGQGTSVTVSS

SEQ ID NO: 59 - DNA, 354 bp, Artificial Sequence
  Description of Artificial Sequence: Synthetic polynucleotide
caggttcagctgcagcagtctggagctgagctggcgaggcctggggcttcagtgaagctgtcctgcaaggcttctggctacaccttcacaatctatggtataagctgggtgaaacagagaactggacagggccttgagtggattggagagatttatcctagaagtgataatacttactacaatgagaagttcaagggcaaggccacactgactgcagacaaatcctccagcacagcgtacatggagctccgcagcctgacatctgaggactctgcggtctatttctgtgcaagatggtactcgttctatgctatggactactggggtcaaggaacctcagtcaccgtctcctca

SEQ ID NO: 60 - protein, 10 aa, Artificial Sequence
  Description of Artificial Sequence: Synthetic peptide
GGDWSANFMY

SEQ ID NO: 61 - protein, 19 aa, Artificial Sequence
  Description of Artificial Sequence: Synthetic peptide
RISGRGVVDYVESVKGRFT

SEQ ID NO: 62 - protein, 3 aa, Artificial Sequence
  Description of Artificial Sequence: Synthetic peptide
ASY

SEQ ID NO: 63 - protein, 10 aa, Artificial Sequence
  Description of Artificial Sequence: Synthetic peptide
GSTSSINTMY

SEQ ID NO: 64 - protein, 19 aa, Artificial Sequence
  Description of Artificial Sequence: Synthetic peptide
FISSGGSTNVRDSVKGRFT

SEQ ID NO: 65 - protein, 11 aa, Artificial Sequence
  Description of Artificial Sequence: Synthetic peptide
YIPYGGTLHDF

SEQ ID NO: 66 - protein, 10 aa, Artificial Sequence
  Description of Artificial Sequence: Synthetic peptide
GSTFSIRAMR

SEQ ID NO: 67 - protein, 18 aa, Artificial Sequence
  Description of Artificial Sequence: Synthetic peptide
VIYGSSTYYADAVKGRFT

SEQ ID NO: 68 - protein, 9 aa, Artificial Sequence
  Description of Artificial Sequence: Synthetic peptide
DTIGTARDY

SEQ ID NO: 69 - protein, 111 aa, Artificial Sequence
  Description of Artificial Sequence: Synthetic polypeptide
EVQLVESGGGLVQPGGSLRLSCAASGGDWSANFMYWYRQAPGKQRELVARISGRGVVDYVESVKGRFTISRDNSKNTLYLQMNSLRAEDTAVYYCAVASYWGQGTLVTVSS

SEQ ID NO: 70 - protein, 119 aa, Artificial Sequence
  Description of Artificial Sequence: Synthetic polypeptide
EVQLVESGGGLVQPGGSLRLSCAASGSTSSINTMYWYRQAPGKERELVAFISSGGSTNVRDSVKGRFTISRDNSKNTLYLQMNSLRAEDTAVYYCNTYIPYGGTLHDFWGQGTLVTVSS

SEQ ID NO: 71 - protein, 116 aa, Artificial Sequence
  Description of Artificial Sequence: Synthetic polypeptide
QVQLVESGGGVVQAGGSLRLSCAASGSTFSIRAMRWYRQAPGTERDLVAVIYGSSTYYADAVKGRFTISRDNSKNTLYLQMNSLRAEDTAVYYCNADTIGTARDYWGQGTLVTVSS

SEQ ID NO: 72 - DNA, 24 bp, Artificial Sequence
  Description of Artificial Sequence: Synthetic oligonucleotide
agggcaagtcaggacattagtaaa

SEQ ID NO: 73 - protein, 8 aa, Artificial Sequence
  Description of Artificial Sequence: Synthetic peptide
RASQDISK

SEQ ID NO: 74 - DNA, 21 bp, Artificial Sequence
  Description of Artificial Sequence: Synthetic oligonucleotide
atctaccatacatcaagatta

SEQ ID NO: 75 - protein, 7 aa, Artificial Sequence
  Description of Artificial Sequence: Synthetic peptide
IYHTSRL

SEQ ID NO: 76 - DNA, 27 bp, Artificial Sequence
  Description of Artificial Sequence: Synthetic oligonucleotide
caacagggtaatacgcttccgtacacg

SEQ ID NO: 77 - protein, 9 aa, Artificial Sequence
  Description of Artificial Sequence: Synthetic peptide
QQGNTLPYT

SEQ ID NO: 78 - DNA, 30 bp, Artificial Sequence
  Description of Artificial Sequence: Synthetic oligonucleotide
ggggtctcattacccgactatggtgtaagc

SEQ ID NO: 79 - protein, 10 aa, Artificial Sequence
  Description of Artificial Sequence: Synthetic peptide
GVSLPDYGVS

SEQ ID NO: 80 - DNA, 42 bp, Artificial Sequence
  Description of Artificial Sequence: Synthetic oligonucleotide
gtaatatggggtagtgaaaccacatactataattcagctctc

SEQ ID NO: 81 - protein, 14 aa, Artificial Sequence
  Description of Artificial Sequence: Synthetic peptide
VIWGSETTYYNSAL

SEQ ID NO: 82 - DNA, 36 bp, Artificial Sequence
  Description of Artificial Sequence: Synthetic oligonucleotide
cattattactacggtggtagctatgctatggactac

SEQ ID NO: 83 - protein, 12 aa, Artificial Sequence
  Description of Artificial Sequence: Synthetic peptide
HYYYGGSYAMDY

SEQ ID NO: 84 - DNA, 321 bp, Artificial Sequence
  Description of Artificial Sequence: Synthetic polynucleotide
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataaca

SEQ ID NO: 85 - protein, 107 aa, Artificial Sequence
  Description of Artificial Sequence: Synthetic polypeptide
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 86 - DNA, 360 bp, Artificial Sequence
  Description of Artificial Sequence: Synthetic polynucleotide
gaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctca

SEQ ID NO: 87 - protein, 120 aa, Artificial Sequence
  Description of Artificial Sequence: Synthetic polypeptide
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 88 - protein, 9 aa, Artificial Sequence
  Description of Artificial Sequence: Synthetic peptide
SIFDIVRGS

SEQ ID NO: 89 - protein, 12 aa, Artificial Sequence
  Description of Artificial Sequence: Synthetic peptide
AIITSGGATNYA

SEQ ID NO: 90 - protein, 12 aa, Artificial Sequence
  Description of Artificial Sequence: Synthetic peptide
CNMESVRYRNYW

SEQ ID NO: 91 - protein, 116 aa, Artificial Sequence
  Description of Artificial Sequence: Synthetic polypeptide
QVQLQESGGGLVQTGGSLRLSCTASGSIFDIVRGSWYRQAPGNQRELVAIITSGGATNYADSVAGRFTISRDSAWKALYLQMNSLKPEDTAVYFCNMESVRYRNYWGQGTQVTVSS

SEQ ID NO: 92 - protein, 9 aa, Artificial Sequence
  Description of Artificial Sequence: Synthetic peptide
FTLEHYSIG

SEQ ID NO: 93 - protein, 13 aa, Artificial Sequence
  Description of Artificial Sequence: Synthetic peptide
SCITSSGGIPKYA

SEQ ID NO: 94 - protein, 21 aa, Artificial Sequence
  Description of Artificial Sequence: Synthetic peptide
CGAATPDDDCSVPGHYGLNYW

SEQ ID NO: 95 - protein, 126 aa, Artificial Sequence
  Description of Artificial Sequence: Synthetic polypeptide
QVQLQESGGGLVQPGGSLRLSCAASGFTLEHYSIGWFRQAPGKDLEGVSCITSSGGIPKYADSVKGRFIISRDNAKNTGYLQMNSLKPEDTAVYYCGAATPDDDCSVPGHYGLNYWGKGTQVTVSS

SEQ ID NO: 96 - protein, 9 aa, Artificial Sequence
  Description of Artificial Sequence: Synthetic peptide
FTFDAYAIG

SEQ ID NO: 97 - protein, 13 aa, Artificial Sequence
  Description of Artificial Sequence: Synthetic peptide
ICLSPSDGSTYYA

SEQ ID NO: 98 - protein, 16 aa, Artificial Sequence
  Description of Artificial Sequence: Synthetic peptide
CATPSWCSLKADFGSW

SEQ ID NO: 99 - protein, 121 aa, Artificial Sequence
  Description of Artificial Sequence: Synthetic polypeptide
QVQLQESGGGLVQAGGSLRLSCAAPGFTFDAYAIGWFRQAPGKEREGVICLSPSDGSTYYADSVKGRFTISSDNAKNTVYLQMNSLKPEDTAVYYCATPSWCSLKADFGSWGQGTQVTVSS

SEQ ID NO: 100 - protein, 9 aa, Artificial Sequence
  Description of Artificial Sequence: Synthetic peptide
SIFSATRME

SEQ ID NO: 101 - protein, 12 aa, Artificial Sequence
  Description of Artificial Sequence: Synthetic peptide
AIVTSGGRTNYA

SEQ ID NO: 102 - protein, 12 aa, Artificial Sequence
  Description of Artificial Sequence: Synthetic peptide
CKFERYDYVNYW

SEQ ID NO: 103 - protein, 116 aa, Artificial Sequence
  Description of Artificial Sequence: Synthetic polypeptide
QVQLQESGGGLVQPGGSLRLSCTASGSIFSATRMEWYRQAPGKQRELVAIVTSGGRTNYADSVNGRFTISRDNAKNTLYLQMNNLKPEDTAVYYCKFERYDYVNYWGRGTQVTVSS

SEQ ID NO: 104 - protein, 9 aa, Artificial Sequence
  Description of Artificial Sequence: Synthetic peptide
SIFSIARMN

SEQ ID NO: 105 - protein, 12 aa, Artificial Sequence
  Description of Artificial Sequence: Synthetic peptide
AILNRAGRTDYA

SEQ ID NO: 106 - protein, 12 aa, Artificial Sequence
  Description of Artificial Sequence: Synthetic peptide
CNLQTISYHDFW

SEQ ID NO: 107 - protein, 116 aa, Artificial Sequence
  Description of Artificial Sequence: Synthetic polypeptide
QVQLQESGGGLVQPGGSLRLSCVASGSIFSIARMNWYRQAPGKQRELVAILNRAGRTDYADSVKGRFTISSDNAKTTVYLQMNSLKPEDTALYYCNLQTISYHDFWGQGTQVTVSS

SEQ ID NO: 108 - protein, 9 aa, Artificial Sequence
  Description of Artificial Sequence: Synthetic peptide
SIFDIARGN

SEQ ID NO: 109 - protein, 12 aa, Artificial Sequence
  Description of Artificial Sequence: Synthetic peptide
AIITSGGATNYA

SEQ ID NO: 110 - protein, 12 aa, Artificial Sequence
  Description of Artificial Sequence: Synthetic peptide
CNMESLSYRHYW

SEQ ID NO: 111 - protein, 116 aa, Artificial Sequence
  Description of Artificial Sequence: Synthetic polypeptide
QVQLQESGGGLVQTGGSLRLSCTASGSIFDIARGNWYRQAPGKQRELVAIITSGGATNYADSVAGRFTISRDDAKNTVYLQMNGLKPEDTAVYFCNMESLSYRHYWGQGTQVTVSS

SEQ ID NO: 112 - protein, 9 aa, Artificial Sequence
  Description of Artificial Sequence: Synthetic peptide
SIIRDNVMA

SEQ ID NO: 113 - protein, 12 aa, Artificial Sequence
  Description of Artificial Sequence: Synthetic peptide
AIINTGGSANVD

SEQ ID NO: 114 - protein, 9 aa, Artificial Sequence
  Description of Artificial Sequence: Synthetic peptide
CNVYYRDLW

SEQ ID NO: 115 - protein, 113 aa, Artificial Sequence
  Description of Artificial Sequence: Synthetic polypeptide
QVQLQESGGGLVQAGGSLRLSCAASKSIIRDNVMAWHRQAPGKQRELVAIINTGGSANVDDSVKGRFTISRDNAKNMVYLQMNNLKPEDTAVYYCNVYYRDLWGQGTQVTVSS

SEQ ID NO: 116 - protein, 9 aa, Artificial Sequence
  Description of Artificial Sequence: Synthetic peptide
FTLDRYAVG

SEQ ID NO: 117 - protein, 13 aa, Artificial Sequence
  Description of Artificial Sequence: Synthetic peptide
SCISSSGDIIKYA

SEQ ID NO: 118 - protein, 21 aa, Artificial Sequence
  Description of Artificial Sequence: Synthetic peptide
CTAADPKDDCSVPGYYGLNYW

SEQ ID NO: 119 - protein, 126 aa, Artificial Sequence
  Description of Artificial Sequence: Synthetic polypeptide
QVQLQESGGGLVQPGGSLRLSCVASGFTLDRYAVGWFRQAPGKELEGVSCISSSGDIIKYADSAKGRFTIARDNAKNTAYLQMNSLKPEDTAVYYCTAADPKDDCSVPGYYGLNYWGKGTQVTVSS

SEQ ID NO: 120 - protein, 9 aa, Artificial Sequence
  Description of Artificial Sequence: Synthetic peptide
FTLDKYAIG

SEQ ID NO: 121 - protein, 13 aa, Artificial Sequence
  Description of Artificial Sequence: Synthetic peptide
SCITSSSGVVKYA

SEQ ID NO: 122 - protein, 21 aa, Artificial Sequence
  Description of Artificial Sequence: Synthetic peptide
CAAAGPPDDCSVPGYYGLNYW

SEQ ID NO: 123 - protein, 126 aa, Artificial Sequence
  Description of Artificial Sequence: Synthetic polypeptide
QVQLQESGGGLVQPGGSLRLSCVASGFTLDKYAIGWFRQAPGKELEGVSCITSSSGVVKYADSVKGRFIISRDNTNNRAFLQMSSLKPEDTAVYYCAAAGPPDDCSVPGYYGLNYWGKGTQVTVSS

SEQ ID NO: 124 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 125 - protein, 22 aa, Homo sapiens
MQSGTHWRVLGLCLLSVGVWGQ

SEQ ID NO: 126 - protein, 104 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 127 - protein, 26 aa, Homo sapiens
VMSVATIVIVDICITGGLLLLVYYWS

SEQ ID NO: 128 - protein, 55 aa, Homo sapiens
KNRKAKAKPVTRGAGAGGRQRGQNKERPPPVPNPDYEPIRKGQRDLYSGLNQRRI

SEQ ID NO: 129 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 130 - protein, 160 aa, Homo sapiens
QSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 131 - protein, 22 aa, Homo sapiens
MEQGKGLAVLILAIILLQGTLA

SEQ ID NO: 132 - protein, 94 aa, Homo sapiens
QSIKGNHLVKVYDYQEDGSVLLTCDAEAKNITWFKDGKMIGFLTEDKKKWNLGSNAKDPRGMYQCKGSQNKSKPLQVYYRMCQNCIELNAATIS

SEQ ID NO: 133 - protein, 21 aa, Homo sapiens
GFLFAEIVSIFVLAVGVYFIA

SEQ ID NO: 134 - protein, 45 aa, Homo sapiens
GQDGVRQSRASDKQTLLPNDQLYQPLKDREDDQYSHLQGNQLRRN

SEQ ID NO: 135 - protein, 172 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKS

SEQ ID NO: 136 - protein, 151 aa, Homo sapiens
FKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKS

SEQ ID NO: 137 - protein, 21 aa, Homo sapiens
MEHSTFLSGLVLATLLSQVSP

SEQ ID NO: 138 - protein, 84 aa, Homo sapiens [sequence omitted]

SEQ ID NO: 139 - protein, 21 aa, Homo sapiens
GIIVTDVIATLLLALGVFCFA

SEQ ID NO: 140 - protein, 45 aa, Homo sapiens
GHETGRLSGAADTQALLRNDQVYQPLRDRDDAQYSHLGGNWARNK

SEQ ID NO: 141 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 142 - protein, 140 aa, Homo sapiens
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 143 - protein, 115 aa, Homo sapiens
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLS

SEQ ID NO: 144 - protein, 23 aa, Homo sapiens
VIGFRILLLKVAGFNLLMTLRLW

SEQ ID NO: 145 - protein, 2 aa, Unknown
  Description of Unknown: TCR alpha chain intracellular domain sequence
SS

SEQ ID NO: 146 - protein, 137 aa, Mus sp.
  MOD_RES (1)..(1): Any amino acid
XIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 147 - protein, 22 aa, Mus sp.
MGLRILLLKVAGFNLLMTLRLW

SEQ ID NO: 148 - protein, 177 aa, Homo sapiens
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 149 - protein, 150 aa, Homo sapiens
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYE

SEQ ID NO: 150 - protein, 21 aa, Homo sapiens
ILLGKATLYAVLVSALVLMAM

SEQ ID NO: 151 - protein, 6 aa, Homo sapiens
VKRKDF

SEQ ID NO: 152 - protein, 173 aa, Mus sp.
EDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 153 - protein, 27 aa, Mus sp.
ILYEILLGKATLYAVLVSTLVVMAMVK

SEQ ID NO: 154 - protein, 5 aa, Mus sp.
KRKNS

SEQ ID NO: 155 - protein, 138 aa, Homo sapiens
DKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSA

SEQ ID NO: 156 - protein, 23 aa, Homo sapiens
YYMYLLLLLKSVVYFAIITCCLL

SEQ ID NO: 157 - protein, 12 aa, Homo sapiens
RRTAFCCNGEKS

SEQ ID NO: 158 - protein, 23 aa, Homo sapiens
LGLRMLFAKTVAVNFLLTAKLFF

SEQ ID NO: 159-194 - skipped (36 entries)

SEQ ID NO: 195 - protein, 338 aa, Artificial Sequence
  Description of Artificial Sequence: Synthetic polypeptide
MLLLVTSLLLCELPHPAFLLIPEVQLVESGGGLVQPGGSLRLSCAASGGDWSANFMYWYRQAPGKQRELVARISGRGVVDYVESVKGRFTISRDNSKNTLYLQMNSLRAEDTAVYYCAVASYWGQGTLVTVSSAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 196 - protein, 469 aa, Artificial Sequence
  Description of Artificial Sequence: Synthetic polypeptide
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 197 - DNA, 20 bp, Artificial Sequence
  Description of Artificial Sequence: Synthetic oligonucleotide
acactggtgtgcctggccac

SEQ ID NO: 198-206 - skipped (9 entries)

SEQ ID NO: 207 - protein, 136 aa, Mus musculus
IQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 208 - skipped

SEQ ID NO: 209 - protein, 172 aa, Mus musculus
DLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 210-242 - skipped (33 entries)

SEQ ID NO: 243 - protein, 153 aa, Homo sapiens
SQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 244-254 - skipped (11 entries)

SEQ ID NO: 255 - protein, 295 aa, Unknown
  Description of Unknown: TCR Gamma 9G115 sequence
AGHLEQPQISSTKTLSKTARLECVVSGITISATSVYWYRERPGEVIQFLVSISYDGTVRKESGIPSGKFEVDRIPETSTSTLTIHNVEKQDIATYYCALWEAQQELGKKIKVFGPGTKLIITDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 256 - protein, 290 aa, Unknown
  Description of Unknown: TCR Delta 2cl5 sequence
MQRISSLIHLSLFWAGVMSAIELVPEHQTVPVSIGVPATLRCSMKGEAIGNYYINWYRKTQGNTMTFIYREKDIYGPGFKDNFQGDIDIAKNLAVLKILAPSERDEGSYYCACDALKRTDTDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 257-264 - skipped (8 entries)

SEQ ID NO: 265 - protein, 129 aa, Homo sapiens
SQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTV

SEQ ID NO: 266 - skipped

SEQ ID NO: 267 - protein, 106 aa, Homo sapiens
DKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFP

SEQ ID NO: 268-360 - skipped (93 entries)

SEQ ID NO: 361 - protein, 814 aa, Artificial Sequence
  Description of Artificial Sequence: Synthetic polypeptide
MLLLVTSLLLCELPHPAFLLIPEVQLVESGGGLVQPGGSLRLSCAASGGDWSANFMYWYRQAPGKQRELVARISGRGVVDYVESVKGRFTISRDNSKNTLYLQMNSLRAEDTAVYYCAVASYWGQGTLVTVSSAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGEGRGSLLTCGDVEENPGPGMQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVIRSKRSRLLHSDYMNMTPRRPGPTRKHYQPYAPPRDFAAYRSKSRQTPPLASVEMEAMEALPVTWGTSSRDEDLENCSHHLGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINT

SEQ ID NO: 362 - protein, 22 aa, Artificial Sequence
  Description of Artificial Sequence: Synthetic peptide
MLLLVTSLLLCELPHPAFLLIP

SEQ ID NO: 363 - protein, 131 aa, Artificial Sequence
  Description of Artificial Sequence: Synthetic polypeptide
EVQLVESGGGLVQPGGSLRLSCAASGGDWSANFMYWYRQAPGKQRELVARISGRGVVDYVESVKGRFTISRDNSKNTLYLQMNSLRAEDTAVYYCAVASYWGQGTLVTVSSAAAGGGGSGGGGSGGGGSLE

SEQ ID NO: 364 - protein, 185 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 365 - protein, 21 aa, Artificial Sequence
  Description of Artificial Sequence: Synthetic peptide
GSGEGRGSLLTCGDVEENPGP

SEQ ID NO: 366 - protein, 20 aa, Artificial Sequence
  Description of Artificial Sequence: Synthetic peptide
MQIPQAPWPVVWAVLQLGWR

SEQ ID NO: 367 - protein, 14 aa, Artificial Sequence
  Description of Artificial Sequence: Synthetic peptide
PGWFLDSPDRPWNP

SEQ ID NO: 368 - protein, 111 aa, Artificial Sequence
  Description of Artificial Sequence: Synthetic polypeptide
PTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVT

SEQ ID NO: 369 - protein, 25 aa, Artificial Sequence
  Description of Artificial Sequence: Synthetic peptide
ERRAEVPTAHPSPSPRPAGQFQTLV

SEQ ID NO: 370 - protein, 21 aa, Artificial Sequence
  Description of Artificial Sequence: Synthetic peptide
VGVVGGLLGSLVLLVWVLAVI

SEQ ID NO: 371 - protein, 41 aa, Artificial Sequence
  Description of Artificial Sequence: Synthetic polypeptide
RSKRSRLLHSDYMNMTPRRPGPTRKHYQPYAPPRDFAAYRS

SEQ ID NO: 372 - protein, 39 aa, Artificial Sequence
  Description of Artificial Sequence: Synthetic polypeptide
KSRQTPPLASVEMEAMEALPVTWGTSSRDEDLENCSHHL

SEQ ID NO: 373 - protein, 22 aa, Artificial Sequence
  Description of Artificial Sequence: Synthetic peptide
GSGATNFSLLKQAGDVEENPGP

SEQ ID NO: 374 - protein, 29 aa, Artificial Sequence
  Description of Artificial Sequence: Synthetic peptide
MRISKPHLRSISIQCYLCLLLNSHFLTEA

SEQ ID NO: 375 - protein, 133 aa, Artificial Sequence
  Description of Artificial Sequence: Synthetic polypeptide
GIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 376 - protein, 631 aa, Artificial Sequence
  Description of Artificial Sequence: Synthetic polypeptide
MLLLVTSLLLCELPHPAFLLIPEVQLVESGGGLVQPGGSLRLSCAASGGDWSANFMYWYRQAPGKQRELVARISGRGVVDYVESVKGRFTISRDNSKNTLYLQMNSLRAEDTAVYYCAVASYWGQGTLVTVSSAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGEGRGSLLTCGDVEENPGPGMQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVIRSKRSRLLHSDYMNMTPRRPGPTRKHYQPYAPPRDFAAYRSKSRQTPPLASVEMEAMEALPVTWGTSSRDEDLENCSHHL

SEQ ID NO: 377 - protein, 722 aa, Artificial Sequence
  Description of Artificial Sequence: Synthetic polypeptide
MLLLVTSLLLCELPHPAFLLIPEVQLVESGGGLVQPGGSLRLSCAASGGDWSANFMYWYRQAPGKQRELVARISGRGVVDYVESVKGRFTISRDNSKNTLYLQMNSLRAEDTAVYYCAVASYWGQGTLVTVSSAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGEGRGSLLTCGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 378 - protein, 26 aa, Artificial Sequence
  Description of Artificial Sequence: Synthetic peptide
SGGGSGGGGSGGGGSGGGGSGGGSLQ

SEQ ID NO: 379 - protein, 175 aa, Artificial Sequence
  Description of Artificial Sequence: Synthetic polypeptide
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 380 - protein, 521 aa, Artificial Sequence
  Description of Artificial Sequence: Synthetic polypeptide
MLLLVTSLLLCELPHPAFLLIPEVQLVESGGGLVQPGGSLRLSCAASGGDWSANFMYWYRQAPGKQRELVARISGRGVVDYVESVKGRFTISRDNSKNTLYLQMNSLRAEDTAVYYCAVASYWGQGTLVTVSSAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGEGRGSLLTCGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 381 - protein, 808 aa, Artificial Sequence
  Description of Artificial Sequence: Synthetic polypeptide
MLLLVTSLLLCELPHPAFLLIPEVQLVESGGGLVQPGGSLRLSCAASGGDWSANFMYWYRQAPGKQRELVARISGRGVVDYVESVKGRFTISRDNSKNTLYLQMNSLRAEDTAVYYCAVASYWGQGTLVTVSSAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGEGRGSLLTCGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHLDYKDDDDKDYKDDDDKDYKDDDDK

SEQ ID NO: 382 - protein, 65 aa, Artificial Sequence
  Description of Artificial Sequence: Synthetic polypeptide
ITCPPPMSVEHADIWVKSYSLYSRERYICNSGFKRKAGTSSLTECVLNKATNVAHWTTPSLKCIR

SEQ ID NO: 383 - protein, 172 aa, Artificial Sequence
  Description of Artificial Sequence: Synthetic polypeptide
DPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 384 - protein, 24 aa, Artificial Sequence
  Description of Artificial Sequence: Synthetic peptide
DYKDDDDKDYKDDDDKDYKDDDDK

SEQ ID NO: 385 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 386 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 387 - protein, 20 aa, Artificial Sequence
  Description of Artificial Sequence: Synthetic peptide
AAAGGGGSGGGGSGGGGSLE

SEQ ID NO: 388 - protein, 30 aa, Artificial Sequence
  Description of Artificial Sequence: Synthetic polypeptide
AAAIEVMYPPPYLGGGGSGGGGSGGGGSLE

SEQ ID NO: 389 - protein, 396 aa, Artificial Sequence
  Description of Artificial Sequence: Synthetic polypeptide
GIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 390 - protein, 251 aa, Artificial Sequence
  Description of Artificial Sequence: Synthetic polypeptide
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVIRSKRSRLLHSDYMNMTPRRPGPTRKHYQPYAPPRDFAAYRSKSRQTPPLASVEMEAMEALPVTWGTSSRDEDLENCSHHL

SEQ ID NO: 391 - protein, 212 aa, Artificial Sequence
  Description of Artificial Sequence: Synthetic polypeptide
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVIRSKRSRLLHSDYMNMTPRRPGPTRKHYQPYAPPRDFAAYRS

SEQ ID NO: 392 - protein, 10 aa, Artificial Sequence
  Description of Artificial Sequence: Synthetic peptide
GYSFTSYWIG

SEQ ID NO: 393 - protein, 15 aa, Artificial Sequence
  Description of Artificial Sequence: Synthetic peptide
GIIYPGDSDTRYSPS

SEQ ID NO: 394 - protein, 12 aa, Artificial Sequence
  Description of Artificial Sequence: Synthetic peptide
AISRTESYVMDV

SEQ ID NO: 395 - protein, 119 aa, Artificial Sequence
  Description of Artificial Sequence: Synthetic polypeptide
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCAISRTESYVMDVWGQGTTVTVSS

SEQ ID NO: 396 - protein, 14 aa, Artificial Sequence
  Description of Artificial Sequence: Synthetic peptide
TGTSSDVGGYNYVS

SEQ ID NO: 397 - protein, 8 aa, Artificial Sequence
  Description of Artificial Sequence: Synthetic peptide
YDVTNRPS

SEQ ID NO: 398 - protein, 9 aa, Artificial Sequence
  Description of Artificial Sequence: Synthetic peptide
SSYAGSHEL

SEQ ID NO: 399 - protein, 109 aa, Artificial Sequence
  Description of Artificial Sequence: Synthetic polypeptide
QSALTQPRSVSGSPGQSVTISCTGTSSDVGGYNYVSWYQQHPGKAPKLMIYDVTNRPSGVPDRFSASKSDNTATLTVSGVQAEDEADYYCSSYAGSHELFGGGTKLTVL

SEQ ID NO: 400 - protein, 473 aa, Artificial Sequence
  Description of Artificial Sequence: Synthetic polypeptide
MLLLVTSLLLCELPHPAFLLIPQSALTQPRSVSGSPGQSVTISCTGTSSDVGGYNYVSWYQQHPGKAPKLMIYDVTNRPSGVPDRFSASKSDNTATLTVSGVQAEDEADYYCSSYAGSHELFGGGTKLTVLGSTSGSGKPGSGEGSTKGEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCAISRTESYVMDVWGQGTTVTVSSAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 401 - protein, 18 aa, Artificial Sequence
  Description of Artificial Sequence: Synthetic peptide
GSTSGSGKPGSGEGSTKG

SEQ ID NO: 402 - protein, 919 aa, Artificial Sequence
  Description of Artificial Sequence: Synthetic polypeptide
MLLLVTSLLLCELPHPAFLLIPQSALTQPRSVSGSPGQSVTISCTGTSSDVGGYNYVSWYQQHPGKAPKLMIYDVTNRPSGVPDRFSASKSDNTATLTVSGVQAEDEADYYCSSYAGSHELFGGGTKLTVLGSTSGSGKPGSGEGSTKGEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCAISRTESYVMDVWGQGTTVTVSSAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGEGRGSLLTCGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 403 - protein, 237 aa, Artificial Sequence
  Description of Artificial Sequence: Synthetic polypeptide
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 404 - protein, 919 aa, Artificial Sequence
  Description of Artificial Sequence: Synthetic polypeptide
MLLLVTSLLLCELPHPAFLLIPQSALTQPRSVSGSPGQSVTISCTGTSSDVGGYNYVSWYQQHPGKAPKLMIYDVTNRPSGVPDRFSASKSDNTATLTVSGVQAEDEADYYCSSYAGSHELFGGGTKLTVLGSTSGSGKPGSGEGSTKGEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCAISRTESYVMDVWGQGTTVTVSSAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGEGRGSLLTCGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLEITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 405 - protein, 26 aa, Artificial Sequence
  Description of Artificial Sequence: Synthetic peptide
SGGGSGGGGSGGGGSGGGGSGGGSLE

SEQ ID NO: 406 - DNA, 20 bp, Artificial Sequence
  Description of Artificial Sequence: Synthetic oligonucleotide
ctcgaccagcttgacatcac

SEQ ID NO: 407 - protein, 50 aa, Artificial Sequence
  Description of Artificial Sequence: Synthetic polypeptide
  SITE (1)..(50): This sequence may encompass 1-10 “Gly Gly Gly Gly Ser” repeating units
  See specification as filed for detailed description of substitutions and preferred embodiments
GGGGSGGGGSGGGGSGGGGSGGGGSGGGGSGGGGSGGGGSGGGGSGGGGS

SEQ ID NO: 408 - protein, 4 aa, Artificial Sequence
  Description of Artificial Sequence: Synthetic peptide
GGGS

SEQ ID NO: 409 - protein, 20 aa, Artificial Sequence
  Description of Artificial Sequence: Synthetic peptide
GGGGSGGGGSGGGGSGGGGS

SEQ ID NO: 410 - protein, 15 aa, Artificial Sequence
  Description of Artificial Sequence: Synthetic peptide
GGGGSGGGGSGGGGS

SEQ ID NO: 411 - protein, 25 aa, Artificial Sequence
  Description of Artificial Sequence: Synthetic peptide
  SITE (1)..(25): This sequence may encompass 2-5 “Gly Gly Gly Gly Ser” repeating units
GGGGSGGGGSGGGGSGGGGSGGGGS

SEQ ID NO: 412 - protein, 15 aa, Artificial Sequence
  Description of Artificial Sequence: Synthetic peptide
  SITE (1)..(15): This sequence may encompass 1-3 “Gly Gly Gly Gly Ser” repeating units
GGGGSGGGGSGGGGS

SEQ ID NO: 413 - RNA, 5000 nt, Artificial Sequence
  Description of Artificial Sequence: Synthetic polynucleotide
  SITE (1)..(5000): This sequence may encompass 50-5000 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 414 - protein, 30 aa, Artificial Sequence
  Description of Artificial Sequence: Synthetic polypeptide
  SITE (1)..(30): This sequence may encompass 1-6 “Gly Gly Gly Gly Ser” repeating units
  See specification as filed for detailed description of substitutions and preferred embodiments
GGGGSGGGGSGGGGSGGGGSGGGGSGGGGS

SEQ ID NO: 415 - protein, 20 aa, Artificial Sequence
  Description of Artificial Sequence: Synthetic peptide
  SITE (1)..(20): This sequence may encompass 2-4 “Gly Gly Gly Gly Ser” repeating units
GGGGSGGGGSGGGGSGGGGS

SEQ ID NO: 416 - protein, 5 aa, Artificial Sequence
  Description of Artificial Sequence: Synthetic peptide
GGGGS

SEQ ID NO: 417 - protein, 10 aa, Artificial Sequence
  Description of Artificial Sequence: Synthetic peptide
GGGGSGGGGS

SEQ ID NO: 418 - DNA, 30 bp, Artificial Sequence
  Description of Artificial Sequence: Synthetic oligonucleotide
ggtggcggaggttctggaggtggaggttcc

SEQ ID NO: 419 - protein, 6 aa, Artificial Sequence
  Description of Artificial Sequence: Synthetic 6xHis tag
HHHHHH

SEQ ID NO: 420 - protein, 20 aa, Artificial Sequence
  Description of Artificial Sequence: Synthetic peptide
  SITE (1)..(20): This sequence may encompass 1-4 “Gly Gly Gly Gly Ser” repeating units
GGGGSGGGGSGGGGSGGGGS

SEQ ID NO: 421 - RNA, 2000 nt, Artificial Sequence
  Description of Artificial Sequence: Synthetic polynucleotide
  SITE (1)..(2000): This sequence may encompass 50-2000 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 422 - DNA, 5000 bp, Artificial Sequence
  Description of Artificial Sequence: Synthetic polynucleotide
  SITE (1)..(5000): This sequence may encompass 50-5000 nucleotides
tttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttt

SEQ ID NO: 423 - DNA, 100 bp, Artificial Sequence
  Description of Artificial Sequence: Synthetic polynucleotide
tttttttttttttttttttttttttttttttttttttttttttttttttttttttttttttttttttttttttttttttttttttttttttttttttttt

SEQ ID NO: 424 - RNA, 5000 nt, Artificial Sequence; identical to SEQ ID NO: 413
  Description of Artificial Sequence: Synthetic polynucleotide
  SITE (1)..(5000): This sequence may encompass 100-5000 nucleotides

SEQ ID NO: 425 - RNA, 400 nt, Artificial Sequence
  Description of Artificial Sequence: Synthetic polynucleotide
  SITE (1)..(400): This sequence may encompass 100-400 nucleotides
  See specification as filed for detailed description of substitutions and preferred embodiments
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa